(19) United States
(12) Patent Application Publication
(10) Pub. No.: US 2006/0041123 A1
(43) Pub. Date: Feb. 23, 2006

(54) Antibacterial agents

(76) Inventors: Jeffrey MICHAEL Axten, Collegeville, PA (US); Gerald Brooks, Essex (GB); Pamela Brown, Essex (GB); David Thomas Davies, Essex (GB); Timothy Francis Gallagher, Collegeville, PA (US); Roger Edward Markwell, Essex (GB); William Henry Miller, Collegeville, PA (US); Neil David Pearson, Essex (GB); Mark Andrew Seefeld, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/538,931

(22) PCT Filed: Dec. 17, 2003
(86) PCT No.: PCT/US03/40032

§ 371 Date: Jun. 14, 2005

Related U.S. Application Data
(60) Provisional application No. 60/434,729, filed on Dec. 18, 2002.
(60) Provisional application No. 60/457,013, filed on Mar. 24, 2003.

(51) Int. Cl. C07D 513/02

(52) U.S. Cl. 544/48

(57) ABSTRACT

Quinoline and naphthyridine derivatives useful in the treatment of bacterial infections in mammals, particularly humans.

### FIELD OF THE INVENTION

This invention relates to novel compounds, compositions containing them and their use as antibacterials.

### BACKGROUND OF THE INVENTION

The emergence of pathogens resistant to known antibiotic therapy is becoming a serious global healthcare problem (Chu, et al., (1996) J. Med. Chem., 39: 3853-3874). Thus, there is a need to discover new broad spectrum antiobiotics useful in combating multidrug-resistant organisms. Importantly, it has now been discovered that certain compounds have antibacterial activity, and, therefore, may be useful for the treatment of bacterial infections in mammals, particularly in humans.

WO0208224, WO0256882, WO02/40474 and WO02/72572 disclose quinoline and naphthyridine derivatives having antibacterial activity.

### SUMMARY OF THE INVENTION

This invention comprises compounds of the formula (I), as described hereinafter, which are useful in the treatment of bacterial infections. It has surprisingly been found that quinoline and naphthyridine derivatives with a chloro or fluoro substituent in the 3-position have enhanced antibacterial activity over those derivatives that are unsubstituted in the 3-position. Quinoline and naphthyridine derivatives with a chloro group in the 3-position showed a 2 fold reduction in MIC levels against one or more of the following organisms, Staphylococcus. aureus, Staphylococcus pneumoniae, Staphylococcus. pyogenes, Enterococcus faecalis, Haemophilus influenzae, E. coli, and Moraxella catarrhalis Ravasio. Quinoline and naphthyridine derivatives with a fluoro group in the 3-position showed a 2 to 4 fold reduction in MIC levels against one or more of the following organisms, Staphylococcus. aureus, Staphylococcus pneumoniae, Staphylococcus. pyogenes, Enterococcus faecalis, Haemophilus influenzae, E. coli, and Moraxella catarrhalis Ravasio. This invention is also a pharmaceutical composition comprising a compound according to formula (I) and a pharmaceutically acceptable carrier. This invention is also a method of treating bacterial infections in mammals, particularly in humans.

### DETAILED DESCRIPTION OF THE INVENTION

This invention provides a compound of formula (I) or a pharmaceutically acceptable derivative thereof: 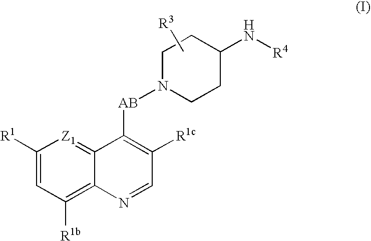
wherein:
Z₁ is N or CR^{1a};
R¹ and R^{1a} are independently selected from H, nitro, halogen, (C₁₋₃)alkylthio, (C₁₋₃)alkyl, and (C₁₋₃)alkoxy optionally substituted by (C₁₋₃)alkoxy; or R¹ and R^{1a} are joined together to form ethylenedioxy;
R^{1b} is H or halogen;
with the proviso that when Z₁ is N, then R^{1b} is H and when Z₁ is CR^{1a} then R¹ is not H;
R^{1c} is halogen;
AB is CHR⁶—CO or CHR⁶—CH₂;
R⁶ is H, NH₂, —CH₂OH, or hydroxy;
R³ is up to two substituents selected from H, halogen, (C₁₋₃)alkyl, hydroxy(C₁₋₃)alkyl, CONH₂, COOH, —CH₂CONH₂, —CH₂COOH, —CONHCH₃, and hydroxy in the 3-position optionally substituted by (C₁₋₃)alkyl;
R⁴ is a group —U—R⁵ where R⁵ is a substituted or unsubstituted bicyclic carbocyclic or heterocyclic ring system (A): 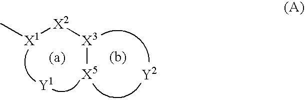
   containing up to four heteroatoms in each ring in which
   at least one of rings (a) and (b) is aromatic;
   X¹ is C or N when part of an aromatic ring or CR¹⁴ when part of a non aromatic ring;
   X² is N, NR¹³, O, S(O)ₓ, CO or CR¹⁴ when part of an aromatic or non-aromatic ring or may in addition be CR¹⁴R¹⁵ when part of a non aromatic ring;
   X³ and X⁵ are independently N or C;
   Y¹ is a 0 to 4 atom linker group each atom of which is independently selected from N, NR¹³, O, S(O)ₓ, CO and CR¹⁴ when part of an aromatic or non-aromatic ring or may additionally be CR¹⁴R¹⁵ when part of a non aromatic ring,
   Y² is a 2 to 6 atom linker group, each atom of Y² being independently selected from N, NR¹³, O, S(O)ₓ, CO and CR¹⁴ when part of an aromatic or non-aromatic ring or may additionally be CR¹⁴R¹⁵ when part of a non aromatic ring;
   each of R¹⁴ and R¹⁵ is independently selected from H; (C₁₋₄)alkylthio; halo; (C₁₋₄)alkyl; (C₂₋₄)alkenyl; hydroxy; hydroxy(C₁₋₄)alkyl; mercapto(C₁₋₄)alkyl; (C₁₋₄)alkoxy; trifluoromethoxy; nitro; cyano; carboxy; amino or aminocarbonyl optionally substituted by (C₁₋₄)alkyl;
   each R¹³ is independently H; trifluoromethyl; (C₁₋₄)alkyl optionally substituted by hydroxy, carboxy, (C₁₋₄)alkoxy, (C₁₋₆)alkylthio, halo or trifluoromethyl; (C₂₋₄)alkenyl; or aminocarbonyl wherein the amino group is optionally substituted (C₁₋₄)alkyl;
   each x is independently 0, 1 or 2; and
   U is CO, SO₂ or CH₂; or a pharmaceutically acceptable salt thereof.

Also included in this invention are pharmaceutically acceptable addition salts, complexes or prodrugs of the compounds of this invention. Prodrugs are considered to be any covalently bonded carriers which release the active parent drug according to formula (I) in vivo.

The invention also provides a pharmaceutical composition, in particular for use in the treatment of bacterial infections in mammals, particularly humans, comprising a compound of formula (I), or a pharmaceutically acceptable derivative thereof, and a pharmaceutically acceptable carrier.

The invention further provides a method of treatment of bacterial infections in mammals, particularly in humans, which method comprises the administration to a mammal in need of such treatment an effective amount of a compound of formula (I), or a pharmaceutically acceptable derivative thereof.

Preferably R¹ is F, Cl, OCH₃, methyl, or SCH₃ Most preferably R¹ is F, Cl, or OCH₃.

Preferably, R^{1a} is H, OCH₃, or OCH₂CH₂OCH₃. Most preferably R^{1a} is H or —OCH₃.

Preferably, R^{1b} is H or F. Most preferably R^{1b} is H.

Preferably, R^{1c} is Cl or F.

Preferably R³ is H, OH, OCH₃, or CH₂OH.

Preferably AB is CHR⁶—CH₂.

Preferably R⁶ is H or OH.

The group —U— is preferably —CH₂—.

Preferably R⁵ is an aromatic heterocyclic ring (A) having 8-11 ring atoms including 2-4 heteroatoms of which at least one is N or NR¹³, in which preferably Y² contains 2-3 heteroatoms, one of which is S and 1-2 are N, with one N bonded to X³.

Alternatively and preferably the heterocyclic ring (A) has ring (a) aromatic selected from optionally substituted benzo and pyrido and ring (b) non-aromatic and Y² has 3-5 atoms including a heteroatom bonded to X⁵ selected from NR¹³, O or S and NHCO bonded via N to X³, or O bonded to X³. Examples of rings (A) include optionally substituted:
(a) and (b) Aromatic
1H-pyrrolo[2,3-b]-pyridin-2-yl, 1H-pyrrolo[3,2-b]-pyridin-2-yl, 3H-imidazo[4,5-b]-pyrid-2-yl, 3H-quinazolin-4-one-2-yl, benzimidazol-2-yl, benzo[1,2,3]-thiadiazol-5-yl, benzo[1,2,5]-oxadiazol-5-yl, benzofur-2-yl, benzothiazol-2-yl, benzo[b]thiophen-2-yl, benzoxazol-2-yl, chromen-4-one-3-yl, imidazo[1,2-a]pyridin-2-yl, imidazo-[1,2-a]-pyrimidin-2-yl, indol-2-yl, indol-6-yl, isoquinolin-3-yl, [1,8]-naphthyridine-3-yl, oxazolo[4,5-b]-pyridin-2-yl, quinolin-2-yl, quinolin-3-yl, quinoxalin-2-yl, indan-2-yl, naphthalen-2-yl, 1,3-dioxo-isoindol-2yl, benzimidazol-2-yl, benzothiophen-2-yl, 1H-benzotriazol-5-yl, 1H-indol-5-yl, 3H-benzooxazol-2-one-6-yl, 3H-benzooxazol-2-thione-6-yl, 3H-benzothiazol-2-one-5-yl, 3H-quinazolin-4-one-2-yl, 3H-quinazolin-4-one-6-yl, 4-oxo-4H-pyrido[1,2-a]pyrimidin-3-yl, benzo[1,2,3]thiadiazol-6-yl, benzo[1,2,5]thiadiazol-5-yl, benzo[1,4]oxazin-2-one-3-yl, benzothiazol-5-yl, benzothiazol-6-yl, cinnolin-3-yl, imidazo[1,2-a]pyridazin-2-yl, imidazo[1,2-b]pyridazin-2-yl, pyrazolo[1,5-a]pyrazin-2-yl, pyrazolo[1,5-a]pyridin-2-yl, pyrazolo[1,5-a]pyrimidin-6-yl, pyrazolo[5,1-c][1,2,4]triazin-3-yl, pyrido[1,2-a]pyrimdin-4-one-2-yl, pyrido[1,2-a]pyrimidin-4-one-3-yl, quinazolin-2-yl, quinoxalin-6-yl, thiazolo[3,2-a]pyrimidin-5-one-7-yl, thiazolo[5,4-b]pyridin-2-yl, thieno[3,2-b]pyridin-6-yl, thiazolo[5,4-b]pyridin-6-yl, 4-oxo-4H-pyrido[1,2-a]pyrimidin-2-yl, 1-oxo-1,2-dihydro-isoquinolin-3-yl, thiazolo[4,5-b]pyridin-5-yl, [1,2,3]thiadiazolo[5,4-b]pyridin-6-yl, 2H-isoquinolin-1-one-3-yl
(a) is Non Aromatic
(2S)-2,3-dihydro-1H-indol-2-yl, (2S)-2,3-dihydro-benzo[1,4]dioxine-2-yl, 3-(R,S)-3,4-dihydro-2H-benzo[1,4]thiazin-3-yl, 3-(R)-2,3-dihydro-[1,4]dioxino[2,3-b]pyridin-3-yl, 3-(S)-2,3-dihydro-[1,4]dioxino[2,3-b]pyridin-3-yl, 2,3-dihydro-benzo[1,4]dioxan-2-yl, 3-substituted-3H-quinazolin-4-one-2-yl, 2,3-dihydro-benzo[1,4]dioxan-2-yl, 1-oxo-1,3,4,5-tetrahydrobenzo[c]azepin-2-yl.
(b) is Non Aromatic
1,1,3-trioxo-1,2,3,4-tetrahydro-1 I⁶-benzo[1,4] thiazin-6-yl, benzo[1,3]dioxol-5-yl, 2,3-dihydro-benzo[1,4]dioxin-6-yl, 2-oxo-2,3-dihydro-benzooxazol-6-yl, 4H-benzo[1,4]oxazin-3-one-6-yl (3-oxo-3,4-dihydro-2H-benzo[1,4]oxazin-6-yl), 4H-benzo[1,4]thiazin-3-one-6-yl (3-oxo-3,4-dihydro-2H-benzo[1,4]thiazin-6-yl), 4H-benzo[1,4]oxazin-3-one-7-yl, 4-oxo-2,3,4,5-tetrahydro-benzo[b][1,4]thiazepine-7-yl, 5-oxo-2,3-dihydro-5H-thiazolo[3,2-a]pyrimidin-6-yl, benzo[1,3]dioxol-5-yl, 2-oxo-2,3-dihydro-1H-pyrido[2,3-b][1,4]thiazin-7-yl, 2-oxo-2,3-dihydro-1H-pyrido[3,4-b][1,4]thiazin-7-yl, 3-oxo-3,4-dihydro-2H-pyrido[3,2-b][1,4]thiazin-6-yl, 2,3-dihydro-[1,4]dioxino[2,3-b]pyridin-6-yl, 2,3-dihydro-[1,4]dioxino[2,3-c]pyridin-7-yl, 2,3-dihydro-[1,4]dioxino[2,3-b]pyridin-7-yl, 6,7-dihydro-[1,4]dioxino[2,3-d]pyrimidin-2-yl, 3-oxo-3,4-dihydro-2H-pyrido[3,2-b][1,4]oxazin-6-yl, 2-oxo-2,3-dihydro-1H-pyrido[3,4-b][1,4]oxazin-7-yl, 2-oxo-2,3-dihydro-1H-pyrido[2,3-b][1,4]oxazin-7-yl, 6-oxo-6,7-dihydro-5H-8-thia-1,2,5-triaza-naphthalen-3-yl, 3,4-dihydro-2H-benzo[1,4]oxazin-6-yl, 3-substituted-3H-benzooxazol-2-one-6-yl, 3-substituted-3H-benzooxazole-2-thione-6-yl, 3-substituted-3H-benzothiazol-2-one-6-yl, 2,3-dihydro-1H-pyrido[2,3-b][1,4]thiazin-7-yl, 3,4-dihydro-2H-benzo[1,4]thiazin-6-yl, 3,4-dihydro-1H-quinolin-2-one-7-yl, 3,4-dihydro-1H-quinoxalin-2-one-7-yl, 6,7-dihydro-4H-pyrazolo[1,5-a]pyrimidin-5-one-2-yl, 5,6,7,8-tetrahydro-[1,8]naphthyridin-2-yl, 2-oxo-3,4-dihydro-1H-[1,8]naphthyridin-6-yl, 3,4-dihydro-2H-pyrido[3,2-b][1,4]thiazin-6-yl.

R¹³ is preferably H if in ring (a) or in addition (C₁₋₄)alkyl such as methyl or isopropyl when in ring (b). More preferably, in ring (b) R¹³ is H when NR¹³ is bonded to X³ and (C₁₋₄)alkyl when NR¹³ is bonded to X⁵.

R¹⁴ and R¹⁵ are preferably independently selected from hydrogen, halo, hydroxy, (C₁₋₄) alkyl, (C₁₋₄)alkoxy, trifluoromethoxy; nitro, cyano, aryl(C₁₋₄)alkoxy and (C₁₋₄)alkylsulphonyl.

More preferably R¹⁵ is hydrogen.

More preferably each R¹⁴ is selected from hydrogen, chloro, fluoro, hydroxy, methyl, methoxy, trifluoromethoxy, benzyloxy, nitro, cyano and methylsulphonyl. Most preferably R¹⁴ is selected from hydrogen, hydroxy, fluorine or nitro. Preferably 0-3 groups R¹⁴ are substituents other than hydrogen.

Preferred groups R⁵ include:
[1,2,3]thiadiazolo[5,4-b]pyridin-6-yl,
1H-Pyrrolo[2,3-b]pyridin-2-yl,
2,3-Dihydro-[1,4]dioxino[2,3-b]pyridin-6-yl,
2,3-Dihydro-[1,4]dioxino[2,3-b]pyridin-7-yl,
2,3-Dihydro-[1,4]dioxino[2,3-c]pyridin-7-yl,
2,3-dihydro-benzo[1,4]dioxin-6-yl,
2-oxo-2,3-dihydro-1H-pyrido[2,3-b][1,4]oxazin-7-yl,
2-oxo-2,3-dihydro-1H-pyrido[2,3-b][1,4]thiazin-7-yl,
3,4-dihydro-2H-benzo[1,4]oxazin-6-yl,
3-Methyl-2-oxo-2,3-dihydro-benzooxazol-6-yl,
3-oxo-3,4-dihydro-2H-benzo[1,4]oxazin-6-yl,
3-oxo-3,4-dihydro-2H-pyrido[3,2-b][1,4]oxazin-6-yl,
4H-benzo[1,4] thiazin-3-one-6-yl,
4-oxo-4H-pyrido[1,2-a]pyrimidin-2-yl,
6-nitro-benzo[1,3]dioxol-5-yl,
7-fluoro-3-oxo-3,4-dihydro-2H-benzo[1,4]oxazin-6-yl,
8-Hydroxy-1-oxo-1,2-dihydro-isoquinolin-3-yl,
8-hydroxyquinolin-2-yl,
benzo[1,2,3]thiadiazol-5-yl,
benzo[1,2,5]thiadiazol-5-yl,
benzothiazol-5-yl,
thiazolo-[5,4-b]pyridin-6-yl,
3-oxo-3,4-dihydro-2H-pyrido[3,2-b][1,4]thiazin-6-yl,
7-chloro-3-oxo-3,4-dihydro-2H-pyrido[3,2-b][1,4]thiazin-6-yl,
7-fluoro-3-oxo-3,4-dihydro-2H-pyrido[3,2-b][1,4]thiazin-6-yl, and
2-oxo-2,3-dihydro-1H-pyrido[3,4-b][1,4]thiazin-7-yl,
2,3-dihydro-1H-pyrido[3,4-b][1,4]thiazin-7-yl,
6-oxo-6,7-dihydro-5H-pyridazino[3,4-b][1,4]thiazin-3-yl,
2,3-dihydro[1,4]oxathiino[2,3-c]pyridin-7-yl,
[1,3]oxathiolo[5,4-c]pyridin-6-yl,
4-fluoro-1H-benzimidazol-2-yl,
cinnolin-3-yl,
1,5,6,7-tetrahydro-1,8-naphthyridin-2-yl,
2,1,3-benzothiadiazol-5-yl,
[1,3]thiazolo[5,4-b]pyridin-6-yl,
1,3-benzothiazol-5-yl,
[1,2,3]thiadiazolo[5,4-b]pyridin-6-yl,
3,4-dihydro-2H-pyrano[2,3-c]pyridin-6-yl, 2-oxo-3,4-dihydro-1,8-naphthyridin-7-yl,
4-oxo-2,3-dihydro-1,5-benzothiazepin-7-yl,
8-methoxy-2,3-dihydro-1,4-benzodiozin-6-yl,
7-methyl, 2,3-dihydro-1,4-benzodioxin-6-yl,
2,3-dihydro-1H-benzofuran-5yl,
benzo-1,3-dioxol-5-yl, and
1-oxo-8-methoxymethoxy-2H-isoquinolin-3-yl.

Most preferred groups R⁵ include:
benzo[1,2,5]thiadiazol-5-yl,
4H-benzo[1,4] thiazin-3-one-6-yl,
2,3-dihydro-benzo[1,4]dioxin-6-yl,
benzo[1,2,3]thiadiazol-5-yl,
3-oxo-3,4-dihydro-2H-benzo[1,4]oxazin-6-yl,
7-fluoro-3-oxo-3,4-dihydro-2H-benzo[1,4] oxazin-6-yl,
2-oxo-2,3-dihydro-1H-pyrido[2,3-b][1,4]thiazin-7-yl,
2,3-Dihydro-[1,4]dioxino[2,3-c]pyridin-7-yl,
3-oxo-3,4-dihydro-2H-pyrido[3,2-b][1,4]oxazin-6-yl,
[1,2,3]thiadiazolo[5,4-b]pyridin-6-yl,
3-oxo-3,4-dihydro-2H-pyrido[3,2-b][1,4]thiazin-6-yl,
7-chloro-3-oxo-3,4-dihydro-2H-pyrido[3,2-b][1,4]thiazin-6-yl,
7-fluoro-3-oxo-3,4-dihydro-2H-pyrido[3,2-b][1,4]thiazin-6-yl,
2-oxo-2,3-dihydro-1H-pyrido[3,4-b][1,4]thiazin-7-yl,

Most especially preferred groups R⁵ include:
3-oxo-3,4-dihydro-2H-pyrido[3,2-b][1,4]thiazin-6-yl,
3-oxo-3,4-dihydro-2H-pyrido[3,2-b][1,4]oxazin-6-yl, and
2,3-Dihydro-[1,4]dioxino[2,3-c]pyridin-7-yl,

Preferred compounds of this invention include:
6-({1-[(Racemic)-2-(3-Chloro-6-methoxy-[1,5]naphthyridin-4-yl)-2-hydroxy-ethyl]-piperidin-4-ylamino}-methyl)-4H-pyrido[3,2-b][1,4]oxazin-3-one Dihydrochloride;
(Racemic)-1-(3-Chloro-6-methoxy-[1,5]naphthyridin-4-yl)-2-{4-[(2,3-dihydro-[1,4]dioxino[2,3-c]pyridin-7-ylmethyl)-amino]-piperidin-1-yl}-ethanol Dihydrochloride;
{1-[2-(3-Chloro-6-methoxy-[1,5]naphthyridin-4-yl)-ethyl]-piperidin-4-yl}-(2,3-dihydro-[1,4]dioxino[2,3-c]pyridin-7-ylmethyl)-amine Dihydrochloride;
{1-[2-(3-Chloro-6-methoxy-quinolin-4-yl)-ethyl]-piperidin-4-yl}-(2,3-dihydro-[1,4]dioxino[2,3-c]pyridin-7-ylmethyl)-amine Dihydrochloride;
6-({(cis)-1-[2-(3-Chloro-6-methoxy-quinolin-4-yl)-ethyl]-3-hydroxy-piperidin-4-ylamino}-methyl)-4H-pyrido[3,2-b][1,4]oxazin-3-one Dihydrochloride Enantiomer 1;
6-({(cis)-1-[2-(3-Chloro-6-methoxy-quinolin-4-yl)-ethyl]-3-hydroxy-piperidin-4-ylamino}-methyl)-4H-pyrido[3,2-b][1,4]oxazin-3-one Dihydrochloride Enantiomer 2;
6-({(cis)-1-[2-(3-Chloro-6-methoxy-quinolin-4-yl)-ethyl]-3-hydroxy-piperidin-4-ylamino}-methyl)-4H-pyrido[3,2-b][1,4]thiazin-3-one Dihydrochloride Enantiomer 1;
6-({(cis)-1-[2-(3-Chloro-6-methoxy-quinolin-4-yl)-ethyl]-3-hydroxy-piperidin-4-ylamino}-methyl)-4H-pyrido[3,2-b][1,4]thiazin-3-one Dihydrochloride Enantiomer 2;
6-({(cis)-1-[2-(3-Chloro-6-methoxy-[1,5]naphthyridin-4-yl)-ethyl]-3-hydroxy-piperidin-4-ylamino}-methyl)-4H-pyrido[3,2-b][1,4]oxazin-3-one Dihydrochloride Enantiomer 1;
6-({(cis)-1-[2-(3-Chloro-6-methoxy-[1,5]naphthyridin-4-yl)-ethyl]-3-hydroxy-piperidin-4-ylamino}-methyl)-4H-pyrido[3,2-b][1,4]oxazin-3-one Dihydrochloride Enantiomer 2;
6-({(cis)-1-[2-(3-Chloro-6-methoxy-[1,5]naphthyridin-4-yl)-ethyl]-3-hydroxy-piperidin-4-ylamino}-methyl)-4H-pyrido[3,2-b][1,4]thiazin-3-one Dihydrochloride Enantiomer 1;
6-({(cis)-1-[2-(3-Chloro-6-methoxy-[1,5]naphthyridin-4-yl)-ethyl]-3-hydroxy-piperidin-4-ylamino}-methyl)-4H-pyrido[3,2-b][1,4]thiazin-3-one Dihydrochloride Enantiomer 2;
6-({1-[2-(3-Chloro-6-methoxyquinolin-4-yl)ethyl]piperidin-4-yl amino}methyl)-4H-pyrido[3,2-b][1,4]thiazin-3-one Trihydrochloride;
6-({1-[2-(3-chloro-6-methoxyquinolin-4-yl)ethyl]piperidin-4-yl amino}methyl)-4H-pyrido[3,2-b][1,4]oxazin-3-one Trihydrochloride;
6-({1-[2-(3-Chloro-6-methoxynaphthyridin-4-yl)ethyl]piperidin-4-yl amino}methyl)-4H-pyrido[3,2-b][1,4]thiazin-3-one Dihydrochloride;
6-({1-[2-(3-chloro-6-methoxynaphthyridin-4-yl)ethyl]piperidin-4-yl amino}methyl)-4H-pyrido[3,2-b][1,4]oxazin-3-one Dihydrochloride;
6-({(trans)-1-[2-(3-Chloro-6-methoxyquinolin-4-yl)ethyl]3-hydroxypiperidin-4-yl amino}methyl)-4H-pyrido[3,2-b][1,4]thiazin-3-one Trihydrochloride enantiomer 2;
6-({(trans)-1-[2-(3-Chloro-6-methoxyquinolin-4-yl)ethyl]3-hydroxypiperidin-4-yl amino}methyl)-4H-pyrido[3,2-b][1,4]oxazin-3-one Trihydrochloride enantiomer 2;
6-(trans)-1-[2-(3-Chloro-6-methoxyquinolin-4-yl)ethyl]3-hydroxypiperidin-4-yl amino}methyl)-4H-pyrido[3,2-b][1,4]thiazin-3-one Trihydrochloride enantiomer 1;
6-(trans)-1-[2-(3-Chloro-6-methoxyquinolin-4-yl)ethyl]3-hydroxypiperidin-4-yl amino}methyl)-4H-pyrido[3,2-b][1,4]oxazin-3-one Trihydrochloride enantiomer 1;
6-({1-[2-(3-Chloro-6-methoxyquinolin-4-yl)ethyl]4-hydroxymethylpiperidin-4-ylamino}methyl)-4H-pyrido[3,2-b][1,4]thiazin-3-one Dihydrochloride;
6-({1-[2-(3-Chloro-6-fluoro-5-methoxy-quinolin-4-yl)-ethyl]-piperidin-4-ylamino}-methyl)-4H-pyrido[3,2-b][1,4]thiazin-3-one Dihydrochloride;
6-({1-[2-(3-Chloro-6-methyl-[1,5]naphthyridin-4-yl)-ethyl]-piperidin-4-ylamino}-methyl)-4H-pyrido[3,2-b][1,4]thiazin-3-one Dihydrochloride;
{1-[2-(3-Chloro-6-methyl-[1,5]naphthyridin-4-yl)-ethyl]-piperidin-4-yl}-(2,3-dihydro[1,4]dioxino[2,3-c]pyridin-7-ylmethyl)-amine Dihydrochloride;
6-({1-[2-(3-Chloro-6-fluoro-quinolin-4-yl)-ethyl]-piperidin-4-ylamino}-methyl)-4H-pyrido[3,2-b][1,4]thiazin-3-one Dihydrochloride;
{1-[2-(3-Chloro-6-fluoro-quinolin-4-yl)-ethyl]-piperidin-4-yl}-(2,3-dihydro[1,4]dioxino[2,3-c]pyridin-7-ylmethyl)-amine Dihydrochloride;
6-({1-[2-(3,6-Dichloro-quinolin-4-yl)-ethyl]-piperidin-4-ylamino}-methyl)-4H-pyrido[3,2-b][1,4]thiazin-3-one Dihydrochloride;
{1-[2-(3,6-Dichloro-quinolin-4-yl)-ethyl]-piperidin-4-yl}-(2,3-dihydro[1,4]dioxino[2,3-c]pyridin-7-ylmethyl-amine Dihydrochloride;
(cis)-1-[2-(3-Chloro-6-methoxy-[1,5]naphthyridin-4-yl)-ethyl]-4-[(2,3-dihydro-[1,4]dioxino[2,3-c]pyridin-7-ylmethyl)-amino]-piperidin-3-ol Dihydrochloride Enantiomer 1;
(cis)-1-[2-(3-Chloro-6-methoxy-[1,5]naphthyridin-4-yl)-ethyl]-4-[(2,3-dihydro-[1,4]dioxino[2,3-c]pyridin-7-ylmethyl)-amino]-piperidin-3-ol Dihydrochloride Enantiomer 2;
6-({1-[2-(3-Fluoro-6-methoxyquinolin-4-yl)ethyl]piperidin-4-yl amino}methyl)-4H-pyrido[3,2-b][1,4]thiazin-3-one dihydrochloride;
{1-[2-(3-Fluoro-6-methoxy-quinolin-4-yl)-ethyl]-piperidin-4-yl}-(2,3-dihydro-[1,4]dioxino[2,3-c]pyridin-7-ylmethyl)-amine dihydrochloride;
cis-4-[(2,3-Dihydro-[1,4]dioxino[2,3-c]pyridin-7-ylmethyl)-amino]-1-[2-(3-fluoro-6-methoxy-quinolin-4-yl)-ethyl]-piperidin-3-ol Enantiomer 2 dihydrochloride;
cis-4-[(2,3-Dihydro-[1,4]dioxino[2,3-c]pyridin-7-ylmethyl)-amino]-1-[2-(3-fluoro-6-methoxy-quinolin-4-yl)-ethyl]-piperidin-3-ol dihydrochloride dihydrochloride Enantiomer 1;
{1-[2-(3-Chloro-6-methoxy-[1,5]naphthyridin-4-yl)-2-hydroxyethyl]-piperidin-4-yl}-(2,3-dihydro-[1,4]dioxino[2,3-c]pyridin-7-ylmethyl)-amine Dihydrochloride Enantiomer 1;
6-({1-[2-(3-Chloro-6-methoxy-[1,5]naphthyridin-4-yl)-2-hydroxy-ethyl]-piperidin-4-ylamino}-methyl)-4H-pyrido[3,2-b][1,4]thiazin-3-one Dihydrochloride Enantiomer 1;
6-({1-[2-(3-Chloro-6-methoxy-[1,5]naphthyridin-4-yl)-2-hydroxy-ethyl]-piperidin-4-ylamino}-methyl)-4H-pyrido[3,2-b][1,4]thiazin-3-one Dihydrochloride Enantiomer 2;
(6-(trans)-1-[2-(3-Chloro-6-methoxyquinolin-4-yl)ethyl]-3-hydroxypiperidin-4-yl)-(2,3-dihydro-[1,4]dioxino[2,3-c]pyridin-7-ylmethyl)amine Enantiomer 2;
(trans)-6-({(1-[2-(3-Chloro-6-methoxy-[1,5]naphthyridin-4-yl)-ethyl]-3-hydroxy-piperidin-4-ylamino}-methyl)-4H-pyrido[3,2-b][1,4]-thiazin-3-one Dihydrochloride Enantiomer 2;
trans-6-({1-[2-(3-Chloro-6-methoxy-[1,5]naphthyridin-4-yl)-ethyl]-3-hydroxy-piperidin-4-ylamino}-methyl)-4H-pyrido[3,2-b][1,4] oxazin-3-one Trihydrochloride Enantiomer 2;
trans-6-({1-[2-(3-Chloro-6-methoxy-[1,5]naphthyridin-4-yl)-ethyl]-3-hydroxy-piperidin-4-ylamino}-methyl)-4H-pyrido[3,2-b][1,4] thiazin-3-one dihydrochloride Enantiomer 1;
6-({(3R,4r,5S)-1-[2-(3-Chloro-6-methoxy-quinolin-4-yl)-ethyl]-3,5-dihydroxy-piperidin-4-ylamino)}-methyl)-4H-pyrido[3,2-b][1,4]oxazin-3-one dihydrochloride;
6-({1-[2-(3-Fluoro-6-methoxyquinolin-4-yl)ethyl]piperidin-4-yl amino}methyl)-4H-pyrido[3,2-b][1,4]oxazin-3-one dihydrochloride;
{1-[2-(3-Bromo-6-methoxy-quinolin-4-yl)-ethyl]-piperidin-4-yl}-(2,3-dihydro-[1,4]dioxino[2,3-c]pyridin-7-ylmethyl)-amine Dihydrochloride;
cis-1-[2-(3-Chloro-6-methoxy-quinolin-4-yl)-ethyl]-4-[(2,3-dihydro-[1,4]dioxino[2,3-c]pyridin-7-ylmethyl)-amino]-piperidin-3-ol Dihydrochloride Enantiomer 1;
cis-1-[2-(3-Chloro-6-methoxy-quinolin-4-yl)-ethyl]-4-[(2,3-dihydro-[1,4]dioxino[2,3-c]pyridin-7-ylmethyl)-amino]-piperidin-3-ol Dihydrochloride Enantiomer 2;
1-{2-[3,8-difluoro-6-(methoxy)-4-quinolinyl]ethyl}-N-(2,3-dihydro[1,4]dioxino[2,3-c]pyridin-7-ylmethyl)-4-piperidinamine dihydrochloride;
7-{[(1-{2-[3,8-Difluoro-6-(methoxy)-4-quinolinyl]ethyl}-4-piperidinyl)amino]methyl}-1H-pyrido[2,3-b][1,4]thiazin-2(3H)-one dihydrochloride;
6-{[(1-{2-[3,8-Difluoro-6-(methoxy)-4-quinolinyl]ethyl}-4-piperidinyl)amino]methyl}-2H-pyrido[3,2-b][1,4]oxazin-3(4H)-one dinydrochloride;
6-{[(1-{2-[3,8-difluoro-6-(methoxy)-4-quinolinyl]ethyl}-4-piperidinyl)amino]methyl}-2H-pyrido[3,2-b][1,4]thiazin-3(4H)-one dihydrochloride;
1-{2-[3,8-difluoro-6-(methoxy)-4-quinolinyl]ethyl}-N-([1,3]dioxolo[4,5-c]pyridin-6-ylmethyl)-4-piperidinamine dihydrochloride;
{1-[2-(9-Chloro-2,3-dihydro-[1,4]dioxino[2,3-f]quinolin-10-yl)-ethyl]-piperidin-4-yl}-(2,3-dihydro-[1,4]dioxino[2,3-c]pyridin-7-ylmethyl)-amine dihydrochloride;
N-(2,3-dihydro[1,4]dioxino[2,3-c]pyridin-7-ylmethyl)-1-{2-[3-fluoro-6-(methoxy)-1,5-naphthyridin-4-yl]ethyl}-4-piperidinamine dihydrochloride;
N-(2,3-Dihydro-1H-pyrido[3,4-b][1,4]thiazin-7-ylmethyl)-1-{2-[3-fluoro-6-(methoxy)-1,5-naphthyridin-4-yl]ethyl}-4-piperidinamine dihydrochloride;
6-{[(1-{2-[3-fluoro-6-(methoxy)-1,5-naphthyridin-4-yl]ethyl}-4-piperidinyl)amino]methyl}-2H-pyrido[3,2-b][1,4]oxazin-3(4H)-one dihydrochloride;
7-{[(1-{2-[3-fluoro-6-(methoxy)-1,5-naphthyridin-4-yl]ethyl}-4-piperidinyl)amino]methyl}-1H-pyrido[2,3-b][1,4]thiazin-2(3H)-one dihydrochloride;
3-{[(1-{2-[3-fluoro-6-(methoxy)-1,5-naphthyridin-4-yl]ethyl}-4-piperidinyl)amino]methyl}-8-hydroxy-1 (2H)-isoquinolinone dihydrochloride;
3-{[(1-{2-[3-fluoro-6-(methoxy)-1,5-naphthyridin-4-yl]ethyl}-4-piperidinyl)amino]methyl}-5H-pyridazino[3,4-b][1,4]thiazin-6(7H)-one dihydrochloride;
6-{[(1-{2-[3-Fluoro-6-(methoxy)-1,5-naphthyridin-4-yl]ethyl}-4-piperidinyl)amino]methyl}-2H-pyrido[3,2-b][1,4]thiazin-3(4H)-one dihydrochloride;
N-(2,3-Dihydro[1,4]oxathiino[2,3-c]pyridin-7-ylmethyl)-1-{2-[3-fluoro-6-(methoxy)-1,5-naphthyridin-4-yl]ethyl}-4-piperidinamine dihydrochloride;
1-{2-[3-Fluoro-6-(methoxy)-1,5-naphthyridin-4-yl]ethyl}-N-([1,3]oxathiolo[5,4-c]pyridin-6-ylmethyl)-4-piperidinamine dihydrochloride;
7-Fluoro-N-(1-{2-[3-fluoro-6-(methoxy)-1,5-naphthyridin-4-yl]ethyl}-4-piperidinyl)-3-oxo-3,4-dihydro-2H-pyrido[3,2-b][1,4]thiazine-6-carboxamide dihydrochloride;
N-(1-{2-[3-fluoro-6-(methoxy)-1,5-naphthyridin-4-yl]ethyl}-4-piperidinyl)-2-oxo-2,3-dihydro-1H-pyrido[2,3-b][1,4]thiazine-7-carboxamide dihydrochloride;
N-(1-{2-[3-fluoro-6-(methoxy)-1,5-naphthyridin-4-yl]ethyl}-4-piperidinyl)-3-oxo-3,4-dihydro-2H-pyrido[3,2-b][1,4]thiazine-6-carboxamide;
N-(1-{2-[3-fluoro-6-(methoxy)-1,5-naphthyridin-4-yl]ethyl}-4-piperidinyl)-3-oxo-3,4-dihydro-2H-pyrido[3,2-b][1,4]oxazine-6-carboxamide;
(3R,4S)-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinol dihydrochloride Enantiomer 1;
6-{[((3R,4S)-1-{2-[3-fluoro-6-(methoxy)-1,5-naphthyridin-4-yl]ethyl}-3-hydroxy-4-piperidinyl)amino]methyl}-2H-pyrido[3,2-b][1,4]thiazin-3(4H)-one dihydrochloride;
6-{[((3R,4S)-1-{2-[3-fluoro-6-(methoxy)-1,5-naphthyridin-4-yl]ethyl}-3-hydroxy-4-piperidinyl)amino]methyl}-2H-pyrido[3,2-b][1,4]oxazin-3(4H)-one dihydrochloride;
(3R,4S)-4-[(2,3-dihydro[1,4]dioxino[2,3-b]pyridin-7-ylmethyl)amino]-1-{2-[3-fluoro-6-(methoxy)-1,5-naphthyridin-4-yl]ethyl}-3-piperidinol dihydrochloride;
6-{[((3S,4R)-1-{2-[3-fluoro-6-(methoxy)-1,5-naphthyridin-4-yl]ethyl}-3-hydroxy-4-piperidinyl)amino]methyl}-2H-pyrido[3,2-b][1,4]thiazin-3(4H)-one dihydrochloride Enantiomer 2;
N-((3S,4R)-1-{2-[3-fluoro-6-(methoxy)-1,5-naphthyridin-4-yl]ethyl}-3-hydroxy-4-piperidinyl)-3-oxo-3,4-dihydro-2H-pyrido[3,2-b][1,4]thiazine-6-carboxamide hydrochloride Enantiomer 2;
7-{[((3R,4S)-1-{2-[3,8-difluoro-6-(methoxy)-4-quinolinyl]ethyl}-3-hydroxy-4-piperidinyl)amino]methyl}-1H-pyrido[2,3-b][1,4]thiazin-2(3H)-one dihydrochloride Enantiomer 1;
6-{[((3R,4S)-1-{2-[3,8-difluoro-6-(methoxy)-4-quinolinyl]ethyl}-3-hydroxy-4-piperidinyl)amino]methyl}-2H-pyrido[3,2-b][1,4]thiazin-3(4H)-one dihydrochloride Enantiomer 1;
(3R,4S)-1-{2-[3,8-difluoro-6-(methoxy)-4-quinolinyl]ethyl}-4-[(2,3-dihydro[1,4]dioxino[2,3-c]pyridin-7-ylmethyl)amino]-3-piperidinol dihydrochloride dihydrochloride Enantiomer 1;
6-{[((3R,4S)-1-{2-[3,8-difluoro-6-(methoxy)-4-quinolinyl]ethyl}-3-hydroxy-4-piperidinyl)amino]methyl}-2H-pyrido[3,2-b][1,4]oxazin-3(4H)-one dihydrochloride;
N-[(4-fluoro-1H-benzimidazol-2-yl)methyl]-1-{2-[3-fluoro-6-(methoxy)-4-quinolinyl]ethyl}-4-piperidinamine;
1-{2-[3-fluoro-6-(methoxy)-4-quinolinyl]ethyl}-N-(1,5,6,7-tetrahydro-1,8-naphthyridin-2-ylmethyl)-4-piperidinamine dihydrochloride;
N-(3-cinnolinylmethyl)-1-{2-[3-fluoro-6-(methoxy)-4-quinolinyl]ethyl}-4-piperidinamine dihydrochloride;
N-(2,1,3-benzothiadiazol-5-ylmethyl)-1-{2-[3-fluoro-6-(methoxy)-4-quinolinyl]ethyl}-4-piperidinamine dihydrochloride;
1-{2-[3-fluoro-6-(methoxy)-4-quinolinyl]ethyl}-N-([1,3]thiazolo[5,4-b]pyridin-6-ylmethyl)-4-piperidinamine dihydrochloride;
N-(3,4-dihydro-2H-pyrido[3,2-b][1,4]thiazin-6-ylmethyl)-1-{2-[3-fluoro-6-(methoxy)-4-quinolinyl]ethyl}-4-piperidinamine dihydrochloride;
N-(1,3-benzothiazol-5-ylmethyl)-1-{2-[3-fluoro-6-(methoxy)-4-quinolinyl]ethyl}-4-piperidinamine dihydrochloride;
1-{2-[3-fluoro-6-(methoxy)-4-quinolinyl]ethyl}-N-([1,2,3]thiadiazolo[5,4-b]pyridin-6-ylmethyl)-4-piperidinamine dihydrochloride;
7-{[(1-{2-[3-fluoro-6-(methoxy)-4-quinolinyl]ethyl}-4-piperidinyl)amino]methyl}-1H-pyrido[2,3-b][1,4]thiazin-2(3H)-one dihydrochloride;
N-(2,3-dihydro[1,4]dioxino[2,3-b]pyridin-7-ylmethyl)-1-{2-[3-fluoro-6-(methoxy)-4-quinolinyl]ethyl}-4-piperidinamine dihydrochloride;
N-(2,3-dihydro[1,4]oxathiino[2,3-c]pyridin-7-ylmethyl)-1-{2-[3-fluoro-6-(methoxy)-4-quinolinyl]ethyl}-4-piperidinamine dihydrochloride;
4-[(2,3-dihydro[1,4]dioxino[2,3-c]pyridin-7-ylmethyl)amino]-1-{2-[3-fluoro-6-(methoxy)-4-quinolinyl]ethyl}-N-methyl-4-piperidinecarboxamide dihydrochloride;
4-[(2,3-dihydro[1,4]dioxino[2,3-c]pyridin-7-ylmethyl)amino]-1-{2-[3-fluoro-6-(methoxy)-4-quinolinyl]ethyl}-4-piperidinecarboxamide dihydrochloride;
4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-N-methyl-4-piperidinecarboxamide dihydrochloride;
4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-4-piperidinecarboxamide dihydrochloride;
1-{2-[3-chloro-6-(methoxy)-1,5-naphthyridin-4-yl]ethyl}-4-[(2,3-dihydro[1,4]dioxino[2,3-c]pyridin-7-ylmethyl)amino]-4-piperidinecarboxamide dihydrochloride;
(4-[(2,3-dihydro[1,4]dioxino[2,3-c]pyridin-7-ylmethyl)amino]-1-{2-[3-fluoro-6-(methoxy)-4-quinolinyl]ethyl}-4-piperidinyl)methanol dihydrochloride;
N-[1-{2-[3-fluoro-6-(methoxy)-1,5-naphthyridin-4-yl]ethyl}-4-(hydroxymethyl)-4-piperidinyl]-3-oxo-3,4-dihydro-2H-pyrido[3,2-b][1,4]thiazine-6-carboxamide hydrochloride;
N-(1-{2-[3-fluoro-6-(methoxy)-4-quinolinyl]ethyl}-4-piperidinyl)-3-oxo-3,4-dihydro-2H-pyrido[3,2-b][1,4]oxazine-6-carboxamide hydrochloride;
N-(1-{2-[3-fluoro-6-(methoxy)-4-quinolinyl]ethyl}-4-piperidinyl)-3-oxo-3,4-dihydro-2H-pyrido[3,2-b][1,4]thiazine-6-carboxamide hydrochloride;
7-{[((3R,4S)-1-{2-[3-fluoro-6-(methoxy)-4-quinolinyl]ethyl}-3-hydroxy-4-piperidinyl)amino]methyl}-1H-pyrido[2,3-b][1,4]thiazin-2(3H)-one dihydrochloride Enantiomer 1;
6-{[((3R,4S)-1-{2-[3-chloro-8-fluoro-6-(methoxy)-4-quinolinyl]ethyl}-3-hydroxy-4-piperidinyl)amino]methyl}-2H-pyrido[3,2-b][1,4]thiazin-3(4H)-one dihydrochloride Enantiomer 1;
(3R,4S)-1-{2-[3-chloro-8-fluoro-6-(methoxy)-4-quinolinyl]ethyl}-4-[(2,3-dihydro[1,4]dioxino[2,3-c]pyridin-7-ylmethyl)amino]-3-piperidinol dihydrochloride;
2-{4-[(2,3-Dihydro[1,4]dioxino[2,3-c]pyridin-7-ylmethyl)amino]-1-piperidinyl)-1-[3-fluoro-6-(methoxy)-1,5-naphthyridin-4-yl]ethanol Dihydrochloride Hydrate Enantiomer 1;
2-{4-[(2,3-Dihydro[1,4]dioxino[2,3-c]pyridin-7-ylmethyl)amino]-1-piperidinyl)-1-[3-fluoro-6-(methoxy)-1,5-naphthyridin-4-yl]ethanol Dihydrochloride Hydrate Enantiomer 2;
racemic,cis 4-[(2,3-dihydro[1,4]dioxino[2,3-c]pyridin-7-ylmethyl)amino]-1-(2-[3-fluoro-6-(methoxy)-1,5-naphthyridin-4-yl]ethyl}-3-piperidinyl)methanol dihydrochloride;
racemic,cis-4-[(2,3-dihydro[1,4]dioxino[2,3-c]pyridin-7-ylmethyl)amino]-1-(2-[3-fluoro-6-(methoxy)-1,5-naphthyridin-4-yl]ethyl}-3-piperidinecarboxylic acid dihydrochloride;
racemic,cis-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinecarboxamide dihydrochloride;
1-{2-[3-chloro-6-(methoxy)-1,5-naphthyridin-4-yl]ethyl}-N-[(6-oxido-2,3-dihydro[1,4]dioxino[2,3-c]pyridin-7-yl)methyl]-4-piperidinamine dihydrochloride;
6-{[(1-{2-[3-chloro-6-(methoxy)-1,5-naphthyridin-4-yl]-3-hydroxypropyl}-4-piperidinyl)amino]methyl}-2H-pyrido[3,2-b][1,4]thiazin-3(4H)-one dihydrochloride;
6-[({1-[2-(3,6-difluoro-4-quinolinyl)ethyl]-4-piperidinyl}amino)methyl]-2H-pyrido[3,2-b][1,4]thiazin-3(4H)-one dihydrochloride;
1-[2-(3,6-difluoro-4-quinolinyl)ethyl]-N-(2,3-dihydro[1,4]dioxino[2,3-c]pyridin-7-ylmethyl)-4-piperidinamine hydrochloride dihydrochloride;
6-[({1-[2-(3,6-difluoro-4-quinolinyl)ethyl]-4-piperidinyl}amino)methyl]-2H-pyrido[3,2-b][1,4]oxazin-3(4H)-one dihydrochloride;
6-{[(1-{2-[3-chloro-6-fluoro-5-(methoxy)-4-quinolinyl]-1-methylethyl}-4-piperidinyl)amino]methyl}-2H-pyrido[3,2-b][1,4]oxazin-3(4H)-one dihydrochloride;
1-{2-[3-chloro-6-fluoro-5-(methoxy)-4-quinolinyl]ethyl}-N-(2,3-dihydro[1,4]dioxino[2,3-c]pyridin-7-ylmethyl)-4-piperidinamine dihydrochloride;
1-[2-(6-chloro-3-fluoro-4-quinolinyl)ethyl]-4-[(2,3-dihydro[1,4]dioxino[2,3-c]pyridin-7-ylmethyl)amino]-N-methyl-4-piperidinecarboxamide dihydrochloride;
2-{4-[(2,3-dihydro[1,4]dioxino[2,3-c]pyridin-7-ylmethyl)amino]-1-piperidinyl}-1-[3-fluoro-6-(methoxy)-4-quinolinyl]ethanol dihydrochloride Enantiomer 2;
6-{[trans-1-{2-[3-fluoro-6-(methoxy)-1,5-naphthyridin-4-yl]ethyl}-3-hydroxy-4-piperidinyl)amino]methyl}-2H-pyrido[3,2-b][1,4]thiazin-3(4H)-one dihydrochloride Enantiomer E2;
6-{[trans-1-{2-[3-fluoro-6-(methoxy)-1,5-naphthyridin-4-yl]ethyl}-3-hydroxy-4-piperidinyl)amino]methyl}-2H-pyrido[3,2-b][1,4]oxazin-3(4H)-one dihydrochloride Enantiomer E2;
trans-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinol dihydrochloride Enantiomer E2;
6-{[trans-1-{2-[3-fluoro-6-(methoxy)-4-quinolinyl]ethyl}-3-hydroxy-4-piperidinyl)amino]methyl}-2H-pyrido[3,2-b][1,4]thiazin-3(4H)-one-dihydrochloride Enantiomer E2;
trans-4-[(2,3-dihydro[1,4]dioxino[2,3-c]pyridin-7-ylmethyl)amino]-1-{2-[3-fluoro-6-(methoxy)-4-quinolinyl]ethyl}-3-piperidinol dihydrochloride;
N-trans-1-{2-[3-fluoro-6-(methoxy)-1,5-naphthyridin-4-yl]ethyl}-3-hydroxy-4-piperidinyl)-3-oxo-3,4-dihydro-2H-pyrido[3,2-b][1,4]thiazine-6-carboxamide hydrochloride Enantiomer E2;
N-trans-1-{2-[3-fluoro-6-(methoxy)-1,5-naphthyridin-4-yl]ethyl}-3-hydroxy-4-piperidinyl)-2,3-dihydro[1,4]dioxino[2,3-c]pyridine-7-carboxamide hydrochloride Enantiomer E2;
racemic, trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]thiazin-3(4H)-one dihydrochloride;
Trans-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-methyl-3-piperidinol dihydrochloride;
6-{[trans-1-{2-[3-fluoro-6-(methoxy)-1,5-naphthyridin-4-yl]ethyl}-3-hydroxy-4-methyl-4-piperidinyl)amino]methyl}-2H-pyrido[3,2-b][1,4]thiazin-3(4H)-one dihydrochloride Enantiomer E1;
Trans-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-4-methyl-3-piperidinol dihydrochloride;
6-{[trans-1-{2-[3-fluoro-6-(methoxy)-1,5-naphthyridin-4-yl]ethyl}-3-hydroxy-4-methyl-4-piperidinyl)amino]methyl}-2H-pyrido[3,2-b][1,4]thiazin-3(4H)-one dihydrochloride Enantiomer E2;
Trans-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-4-methyl-3-piperidinol dihydrochloride;
N-(3,4-dihydro-2H-pyrano[2,3-c]pyridin-6-ylmethyl)-1-{2-[3-fluoro-6-(methoxy)-1,5-naphthyridin-4-yl]ethyl}-4-piperidinamine dihydrochloride;
{[(1-{2-[3-Fluoro-6-(methoxy-5-naphthyridin-4-yl]ethyl}-4-piperidinyl)amino]methyl}-3,4-dihydro-1,8-naphthyridin-2-(1H)-one;
7-{[(1-{2-[3-fluoro-6-(methoxy)-1,5-naphthyridin-4-yl]ethyl}-4-piperidinyl)amino]methyl}-2,3-dihydro-1,5-benzothiazepin-4(5H)-one;
trans-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinol dihydrochloride Enantiomer E1;
6-{[(-1-{2-[3-fluoro-6-(methoxy)-1,5-naphthyridin-4-yl]ethyl}-3-hydroxy-4-piperidinyl)amino]methyl}-2H-pyrido[3,2-b][1,4]oxazin-3(4H)-one dihydrochloride;
trans-6-{[(1-{2-[3-fluoro-6-(methoxy)-4-quinolinyl]ethyl}-3-hydroxy-4-piperidinyl)amino]methyl}-2H-pyrido[3,2-b][1,4]thiazin-3(4H)-one Enantiomer E1;
trans-4-[(2,3-dihydro[1,4]dioxino[2,3-c]pyridin-7-ylmethyl)amino]-1-{2-[3-fluoro-6-(methoxy)-4-quinolinyl]ethyl}-3-piperidinol dihydrochloride;
trans-6-{[(1-{2-[3-fluoro-6-(methoxy)-4-quinolinyl]ethyl}-3-hydroxy-4-piperidinyl)amino]methyl}-2H-pyrido[3,2-b][1,4]oxazin-3(4H)-one dihydrochloride;
trans-N-(1-{2-[3-fluoro-6-(methoxy)-1,5-naphthyridin-4-yl]ethyl}-3-hydroxy-4-piperidinyl)-3-oxo-3,4-dihydro-2H-pyrido[3,2-b]([1,4]thiazine-6-carboxamide hydrochloride Enantiomer E1;
trans-N-((3R,4R)-1-{2-[3-fluoro-6-(methoxy)-1,5-naphthyridin-4-yl]ethyl}-3-hydroxy-4-piperidinyl)-3-oxo-3,4-dihydro-2H-pyrido[3,2-b][1,4]oxazine-6-carboxamide Isomer E1 hydrochloride;
trans-N-(1-{2-[3-fluoro-6-(methoxy)-1,5-naphthyridin-4-yl]ethyl}-3-hydroxy-4-piperidinyl)-2,3-dihydro[1,4]dioxino[2,3-c]pyridine-7-carboxamide Isomer E1 hydrochloride;
6-{[trans-1-{2-[3-fluoro-6-(methoxy)-1,5-naphthyridin-4-yl]ethyl}-3-hydroxy-4-piperidinyl)amino]methyl}-2H-pyrido[3,2-b][1,4]thiazin-3(4H)-one Enantiomer E1;
6-{[(1-{2-[3-fluoro-6-(methoxy)-1,5-naphthyridin-4-yl]ethyl}-4-methyl-4-piperidinyl)amino]methyl}-2H-pyrido[3,2-b][1,4]oxazin-3(4H)-one dihydrochloride;
6-{[(1-{2-[3-fluoro-6-(methoxy)-1,5-naphthyridin-4-yl]ethyl}-4-methyl-4-piperidinyl)amino]methyl}-2H-pyrido[3,2-b][1,4]thiazin-3(4H)-one dihydrochloride;
N-(2,3-dihydro[1,4]dioxino[2,3-c]pyridin-7-ylmethyl)-1-{2-[3-fluoro-6-(methoxy)-1,5-naphthyridin-4-yl]ethyl}-4-methyl-4-piperidinamine dihydrochloride;
N-(1-{2-[3-fluoro-6-(methoxy)-1,5-naphthyridin-4-yl]ethyl}-4-methyl-4-piperidinyl)-2,3-dihydro-1,4-benzodioxin-6-sulfonamide;
cis-6-{[(1-{2-[3-chloro-8-fluoro-6-(methoxy)-4-quinolinyl]ethyl}-3-fluoro-4-piperidinyl)amino]methyl}-2H-pyrido[3,2-b][1,4]thiazin-3(4H)-one dihydrochloride Enantiomer 1;
cis-1-{2-[3,8-difluoro-6-(methoxy)-4-quinolinyl]ethyl}-N-(2,3-dihydro[1,4]dioxino[2,3-c]pyridin-7-ylmethyl)-3-fluoro-4-piperidinamine dihydrochloride Enantiomer 1;
cis-1-{2-[3,8-difluoro-6-(methoxy)-4-quinolinyl]ethyl}-N-(2,3-dihydro[1,4]dioxino[2,3-c]pyridin-7-ylmethyl)-3-fluoro-4-piperidinamine dihydrochloride Enantiomer 2;
cis-6-{[(1-{2-[3,8-difluoro-6-(methoxy)-4-quinolinyl]ethyl-3-fluoro-4-piperidinyl)amino]methyl}-2H-pyrido[3,2-b][1,4]oxazin-3(4H)-one dihydrochloride, Enantiomer 1;
cis-6-{[(1-{2-[3,8-difluoro-6-(methoxy)-4-quinolinyl]ethyl}-3-fluoro-4-piperidinyl)amino]methyl}-2H-pyrido[3,2-b][1,4]oxazin-3(4H)-one dihydrochloride, Enantiomer 2;
cis-1-{2-[3,8-difluoro-6-(methoxy)-4-quinolinyl]ethyl}-N-(2,3-dihydro-1,4-benzodioxin-6-ylmethyl)-3-fluoro-4-piperidinamine dihydrochloride, Enantiomer 1;
cis-1-{2-[3,8-difluoro-6-(methoxy)-4-quinolinyl]ethyl}-N-(2,3-dihydro-1,4-benzodioxin-6-ylmethyl)-3-fluoro-4-piperidinamine dihydrochloride, Enantiomer 2;
cis-6-{[(−1-{2-[3,8-difluoro-6-(methoxy)-4-quinolinyl]ethyl}-3-fluoro-4-piperidinyl)amino]methyl}-2H-pyrido[3,2-b][1,4]thiazin-3(4H)-one dihydrochloride, Enantiomer 1;
cis-6-{[(−1-{2-[3,8-difluoro-6-(methoxy)-4-quinolinyl]ethyl}-3-fluoro-4-piperidinyl)amino]methyl}-2H-pyrido[3,2-b][1,4]thiazin-3(4H)-one dihydrochloride, Enantiomer 2;
cis-N-(1-{2-[3,8-difluoro-6-(methoxy)-4-quinolinyl]ethyl}-3-fluoro-4-piperidinyl)-3-oxo-3,4-dihydro-2H-pyrido[3,2-b][1,4]thiazine-6-carboxamide hydrochloride Enantiomer 1;
6-{[((3S,4R)-1-{2-[3-chloro-8-fluoro-6-(methoxy)-4-quinolinyl]ethyl}-3-hydroxy-4-piperidinyl)amino]methyl}-2H-pyrido[3,2-b][1,4]thiazin-3(4H)-one dihydrochloride Enantiomer E2;
trans-6-({1-[2-(3-Chloro-6-methoxy-[1,5]naphthyridin-4-yl)-ethyl]-3-hydroxy-piperidin-4-ylamino}-methyl)-4H-pyrido[3,2-b][1,4] oxazin-3-one trihydrochloride Enantiomer 1;
trans-1-[2-[3-chloro-6-(methoxy)-1,5-naphthyridin-4-yl]ethyl}-4-[(2,3-dihydro[1,4]dioxino[2,3-c]pyridin-7-ylmethyl)amino]-3-piperidinol Enantiomer 1;
trans-1-{2-[3-chloro-6-(methoxy)-1,5-naphthyridin-4-yl]ethyl}-4-[(2,3-dihydro[1,4]dioxino[2,3-c]pyridin-7-ylmethyl)amino]-3-piperidinol Enantiomer 2;
2-{4-[(2,3-dihydro[1,4]dioxino[2,3-c]pyridin-7-ylmethyl)amino]-1-piperidinyl}-1-[3-fluoro-6-(methoxy)-4-quinolinyl]ethanol dihydrochloride Enantiomer 1;
N-(2,3-dihydro-1,4-benzodioxin-6-ylmethyl)-1-{2-[3-fluoro-6-(methoxy)-1,5-naphthyridin-4-yl]ethyl}-4-piperidinamine;
(3S,4R)-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinol dihydrochloride Enantiomer 2;
(3R,4S)-1-{2-[3-fluoro-6-(methoxy)-1,5-naphthyridin-4-yl]ethyl}-4-[([1,3]oxathiolo[5,4-c]pyridin-6-ylmethyl)amino]-3-piperidinol dihydrochloride Enantiomer E1;
6-{[(1-{2-[3-chloro-8-fluoro-6-(methoxy)-4-quinolinyl]ethyl}-4-piperidinyl)amino]methyl}-2H-pyrido[3,2-b][1,4]thiazin-3(4H)-one;
1-{2-[3-chloro-8-fluoro-6-(methoxy)-4-quinolinyl]ethyl}-N-(2,3-dihydro[1,4]dioxino[2,3-c]pyridin-7-ylmethyl)-4-piperidinamine;
(3S,4R)-1-[2-(3,6-dichloro-4-quinolinyl)ethyl]-4-[(2,3-dihydro[1,4]dioxino[2,3-c]pyridin-7-ylmethyl)amino]-3-piperidinol dihydrochloride Enantiomer E2;
6-[({(3S,4R)-1-[2-(3,6-dichloro-4-quinolinyl)ethyl]-3-hydroxy-4-piperidinyl}amino)methyl]-2H-pyrido[3,2-b][1,4]thiazin-3(4H)-one dihydrochloride Enantiomer E2;
(3S,4R)-1-[2-(3-chloro-6-fluoro-4-quinolinyl)ethyl]-4-[(2,3-dihydro[1,4]dioxino[2,3-c]pyridin-7-ylmethyl)amino]-3-piperidinol dihydrochloride Enantiomer E2;
6-[({(3S,4R)-1-[2-(3-chloro-6-fluoro-4-quinolinyl)ethyl]-3-hydroxy-4-piperidinyl}amino)methyl]-2H-pyrido[3,2-b][1,4]thiazin-3(4H)-one dihydrochloride Enantiomer E2;
N-(1-{2-[3-fluoro-6-(methoxy)-1,5-naphthyridin-4-yl]ethyl}-4-methyl-4-piperidinyl)-2,3-dihydro[1,4]dioxino[2,3-c]pyridine-7-carboxamide dihydrochloride;
N-(1-{2-[3-fluoro-6-(methoxy)-1,5-naphthyridin-4-yl]ethyl}-4-methyl-4-piperidinyl)-3-oxo-3,4-dihydro-2H-pyrido[3,2-b][1,4]oxazine-6-carboxamide;
N-(1-{2-[3-fluoro-6-(methoxy)-1,5-naphthyridin-4-yl]ethyl}-4-methyl-4-piperidinyl)-3-oxo-3,4-dihydro-2H-pyrido[3,2-b][1,4]thiazine-6-carboxamide;
trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]oxazin-3(4H)-one dihydrochloride Enantiomer E1;
trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]thiazin-3(4H)-one dihydrochloride Enantiomer E1;
trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]oxazin-3(4H)-one dihydrochloride Enantiomer E2;
trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]thiazin-3(4H)-one dihydrochloride Enantiomer E2;
trans-4-[(2,3-dihydro-1,4-benzodioxin-6-ylmethyl)amino]-1-{2-[3-fluoro-6-(methoxy)-4-quinolinyl]ethyl}-3-piperidinol hydrochloride Enantiomer E1;
trans 4-[(2,3-dihydro-1,4-benzodioxin-6-ylmethyl)amino]-1-{2-[3-fluoro-6-(methoxy)-1,5-naphthyridin-4-yl]ethyl}-3-piperidinol dihydrochloride Enantiomer E2;
trans 4-[(2,3-dihydro-1,4-benzodioxin-6-ylmethyl)amino]-1-{2-[3-fluoro-6-(methoxy)-1,5-naphthyridin-4-yl]ethyl}-3-piperidinol dihydrochloride Enantiomer E1;
(3S,4R)-1-{2-[3,8-difluoro-6-(methoxy)-4-quinolinyl]ethyl}-4-[(2,3-dihydro[1,4]dioxino[2,3-c]pyridin-7-ylmethyl)amino]-3-piperidinol dihydrochloride Enantiomer E2;
(3S,4R)-1-{2-[3,8-difluoro-6-(methoxy)-4-quinolinyl]ethyl}-4-[(2,3-dihydro-1,4-benzodioxin-6-ylmethyl)amino]-3-piperidinol dihydrochloride Enantiomer E2;
N-(2,3-dihydro-1-benzofuran-5-ylmethyl)-1-{2-[3-fluoro-6-(methoxy)-1,5-naphthyridin-4-yl]ethyl}-4-piperidinamine dihydrochloride;
6-{[(1-{2-[3-fluoro-6-(methoxy)-4-quinolinyl]-2-hydroxyethyl}-4-piperidinyl)amino]methyl}-2H-pyrido[3,2-b][1,4]oxazin-3(4H)-one dihydrochloride Enantiomer E1;
6-{[(1-{2-[3-fluoro-6-(methoxy)-4-quinolinyl]-2-hydroxyethyl}-4-piperidinyl)amino]methyl}-2H-pyrido[3,2-b][1,4]oxazin-3(4H)-one dihydrochloride Enantiomer E2;
6-{[(1-{2-[3-fluoro-6-(methoxy)-1,5-naphthyridin-4-yl]-2-hydroxyethyl}-4-piperidinyl)amino]methyl}-2H-pyrido[3,2-b][1,4]oxazin-3(4H)-one dihydrochloride Enantiomer E2; 6-{[(1-{2-[3-fluoro-6-(methoxy)-1,5-naphthyridin-4-yl]-2-hydroxyethyl}-4-piperidinyl)amino]methyl}-2H-pyrido[3,2-b][1,4]oxazin-3(4H)-one dihydrochloride Enantiomer E1;
6-{[(1-{2-[3-chloro-8-fluoro-6-(methoxy)-4-quinolinyl]-2-hydroxyethyl}-4-piperidinyl)amino]methyl}-2H-pyrido[3,2-b][1,4]oxazin-3(4H)-one dihydrochloride Enantiomer E1;
6-{[(1-{2-[3-chloro-8-fluoro-6-(methoxy)-4-quinolinyl]-2-hydroxyethyl}-4-piperidinyl)amino]methyl}-2H-pyrido[3,2-b][1,4]oxazin-3(4H)-one dihydrochloride Enantiomer E2;
1-[3-chloro-8-fluoro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E1;
1-[3-chloro-8-fluoro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E2;
1-[3,8-difluoro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E2;
1-[3,8-difluoro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E1;
1-[3-chloro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E2;
1-[3-chloro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E1;
1-[3-chloro-6-(methoxy)-1,5-naphthyridin-4-yl]-2-{4-[(2,3-dihydro[1,4]dioxino[2,3-c]pyridin-7-ylmethyl)amino]-1-piperidinyl}ethanol dihydrochloride Enantiomer E2;
2-{4-[(2,3-dihydro[1,4]dioxino[2,3-c]pyridin-7-ylmethyl)amino]-3-fluoro-1-piperidinyl}-1-[3-fluoro-6-(methoxy)-1,5-naphthyridin-4-yl]ethanol dihydrochloride Enantiomer E2;
2-{4-[(2,3-dihydro[1,4]dioxino[2,3-c]pyridin-7-ylmethyl)amino]-3-fluoro-1-piperidinyl}-1-[3-fluoro-6-(methoxy)-1,5-naphthyridin-4-yl]ethanol dihydrochloride Enantiomer E1;
7-{[(1-{2-[3,8-difluoro-6-(methoxy)-4-quinolinyl]ethyl}-3-fluoro-4-piperidinyl)amino]methyl}-1H-pyrido[2,3-b][1,4]thiazin-2(3H)-one dihydrochloride Enantiomer E2;
1-{2-[3-fluoro-6-(methoxy)-1,5-naphthyridin-4-yl]ethyl}-N-([8-(methoxy)-2,3-dihydro-1,4-benzodioxin-6-yl]methyl}-4-piperidinamine; and
1-{2-[3-fluoro-6-(methoxy)-1,5-naphthyridin-4-yl]ethyl}-N-[(7-methyl-2,3-dihydro-1,4-benzodioxin-6-yl)methyl]-4-piperidinamine;
   or a pharmaceutically acceptable salt thereof.

Most preferred compounds of this invention are:
cis-4-[(2,3-Dihydro-[1,4]dioxino[2,3-c]pyridin-7-ylmethyl)-amino]-1-[2-(3-fluoro-6-methoxy-quinolin-4-yl)-ethyl]-piperidin-3-ol dihydrochloride dihydrochloride Enantiomer 1;
(trans)-6-({1-[2-(3-Chloro-6-methoxy-[1,5]naphthyridin-4-yl)-ethyl]-3-hydroxy-piperidin-4-ylamino}-methyl)-4H-pyrido[3,2-b][1,4]-thiazin-3-one Dihydrochloride Enantiomer 2;
1-{2-[3,8-difluoro-6-(methoxy)-4-quinolinyl]ethyl}-N-(2,3-dihydro[1,4]dioxino[2,3-c]pyridin-7-ylmethyl)-4-piperidinamine dihydrochloride;
6-{[(1-{2-[3,8-Difluoro-6-(methoxy)-4-quinolinyl]ethyl}-4-piperidinyl)amino]methyl}-2H-pyrido[3,2-b][1,4]oxazin-3(4H)-one dinydrochloride;
N-(2,3-dihydro[1,4]dioxino[2,3-c]pyridin-7-ylmethyl)-1-{2-[3-fluoro-6-(methoxy)-1,5-naphthyridin-4-yl]ethyl}-4-piperidinamine dihydrochloride; (3R,4S)-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinol dihydrochloride Enantiomer 1;
6-{[((3S,4R)-1-{2-[3-fluoro-6-(methoxy)-1,5-naphthyridin-4-yl]ethyl}-3-hydroxy-4-piperidinyl)amino]methyl}-2H-pyrido[3,2-b][1,4]thiazin-3(4H)-one dihydrochloride Enantiomer 2;
(3R,4S)-1-{2-[3,8-difluoro-6-(methoxy)-4-quinolinyl]ethyl}-4-[(2,3-dihydro[1,4]dioxino[2,3-c]pyridin-7-ylmethyl)amino]-3-piperidinol dihydrochloride dihydrochloride Enantiomer 1; and
2-{4-[(2,3-Dihydro[1,4]dioxino[2,3-c]pyridin-7-ylmethyl)amino]-1-piperidinyl)-1-[3-fluoro-6-(methoxy)-1,5-naphthyridin-4-yl]ethanol Dihydrochloride Hydrate Enantiomer 1;
   or a pharmaceutically acceptable salt thereof.

Unless otherwise defined, the term (C₁₋₃)alkyl when used alone or when forming part of other groups (such as the ‘alkoxy’ group) includes substituted or unsubstituted, straight or branched chain alkyl groups containing 1 to 3 carbon atoms. Examples of (C₁₋₃)alkyl include methyl, ethyl, n-propyl, and isopropyl groups.

The term (C₂₋₄)alkenyl means a substituted or unsubstituted alkyl group of 2 to 4 carbon atoms, wherein one carbon-carbon single bond is replaced by a carbon-carbon double bond. Examples of (C₂₋₄)alkenyl include ethylene, 1-propene, 2-propene, 1-butene, 2-butene, and isobutene. Both cis and trans isomers are included.

The term (C₃₋₇)cycloalkyl refers to subsituted or unsubstituted carbocyclic system of three to seven carbon atoms, which may contain up to two unsaturated carbon-carbon bonds. Examples of (C₃₋₇)cycloalkyl include cyclopropyl, cyclobutyl, cyclopentyl, cyclopentenyl, cyclohexyl, cyclohexenyl, and cycloheptyl.

Unless otherwise defined, suitable substituents for any (C₁₋₃)alkyl, (C₁₋₃)alkoxy, (C₂₋₄)alkenyl, and (C₃₋₇)cycloalkyl groups includes up to three substituents selected from the group consisting of hydroxy, halogen, nitro, cyano, carboxy, amino, amidino, sulphonamido, unsubstituted (C₁₋₃)alkoxy, trifluromethyl, and acyloxy.

Halo or halogen includes fluoro, chloro, bromo and iodo.

Haloalkyl moieties include 1-3 halogen atoms.

Unless otherwise defined, the term “heterocyclic” as used herein includes optionally substituted aromatic and non-aromatic, single and fused, rings suitably containing up to four hetero-atoms in each ring selected from oxygen, nitrogen and sulphur, which rings may be unsubstituted or C-substituted by, for example, up to three groups selected from (C₁₋₄)alkylthio; halo; halo(C₁₋₄)alkoxy; halo(C₁₋₄)alkyl; (C₁₋₄)alkyl; (C₂₋₄)alkenyl; hydroxy; hydroxy(C₁₋₄)alkyl; mercapto(C₁₋₄)alkyl; (C₁₋₄)alkoxy; nitro; cyano, carboxy; amino or aminocarbonyl; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl.

Each heterocyclic ring suitably has from 4 to 7, preferably 5 or 6, ring atoms.

A fused heterocyclic ring system may include carbocyclic rings and need include only one heterocyclic ring.

Compounds within the invention containing a heterocyclyl group may occur in two or more tautometric forms depending on the nature of the heterocyclyl group; all such tautomeric forms are included within the scope of the invention.

Where an amino group forms part of a single or fused non-aromatic heterocyclic ring as defined above suitable optional substituents in such substituted amino groups include H; trifluoromethyl; (C₁₋₄)alkyl optionally substituted by hydroxy, (C₁₋₄)alkoxy, (C₁₋₄)alkylthio, (C₂₋₄)alkenyl; halo or trifluoromethyl;
When used herein the term “aryl”, includes optionally substituted phenyl and naphthyl.

Aryl groups may be optionally substituted with up to five, preferably up to three, groups selected from (C₁₋₄)alkylthio; halo; halo(C₁₋₄)alkoxy; halo(C₁₋₄)alkyl; (C₁₋₄)alkyl; (C₂₋₄)alkenyl; hydroxy; hydroxy(C₁₋₄)alkyl; mercapto(C₁₋₄)alkyl; (C₁₋₄)alkoxy; nitro; cyano; carboxy; amino or aminocarbonyl optionally substituted by (C₁₋₄)alkyl; (C₁₋₄)alkylsulphonyl; or (C₂₋₄)alkenylsulphonyl.

The term “acyl” includes formyl and (C₁₋₄)alkylcarbonyl group.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Since the compounds of formula (I) are intended for use in pharmaceutical compositions it will readily be understood that they are each provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the formula (I) or pharmaceutically acceptable derivative thereof.

Pharmaceutically acceptable derivatives of the above-mentioned compounds of formula (I) include the free base form or their acid addition or quaternary ammonium salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids. Compounds of formula (I) may also be prepared as the N-oxide. Compounds of formula (I) having a free carboxy group may also be prepared as an in vivo hydrolysable ester. The invention extends to all such derivatives.

Examples of suitable pharmaceutically acceptable in vivo hydrolysable ester-forming groups include those forming esters which break down readily in the human body to leave the parent acid or its salt. Suitable groups of this type include those of part formulae (i), (ii), (iii), (iv) and (v): 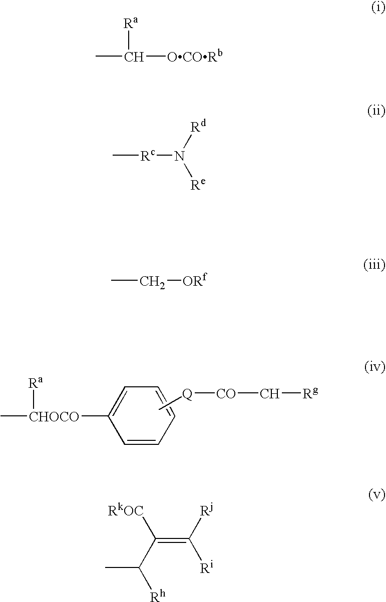
wherein R^{a} is hydrogen, (C₁₋₆)alkyl, (C₃₋₇)cycloalkyl, methyl, or phenyl, R^{b} is (C₁₋₆)alkyl, (C₁₋₆)alkoxy, phenyl, benzyl, (C₃₋₇)cycloalkyl, (C₃₋₇)cycloalkyloxy, (C₁₋₆)alkyl(C₃₋₇) cycloalkyl, 1-amino(C₁₋₆) alkyl, or 1-(C₁₋₆alkyl)amino (C₁₋₆)alkyl; or R^{a} and R^{b} together form a 1,2-phenylene group optionally substituted by one or two methoxy groups; R^{c} represents (C₁₋₆)alkylene optionally substituted with a methyl or ethyl group and R^{d} and R^{e} independently represent (C₁₋₆)alkyl; R^{f} represents (C₁₋₆)alkyl; R^{g} represents hydrogen or phenyl optionally substituted by up to three groups selected from halogen, (C₁₋₆)alkyl, or (C₁₋₆)alkoxy; Q is oxygen or NH; R^{h} is hydrogen or (C₁₋₆)alkyl; R¹ is hydrogen, (C₁₋₆)alkyl optionally substituted by halogen, (C₂₋₆)alkenyl, (C₁₋₆)alkoxycarbonyl, aryl or heteroaryl; or R^{h} and R¹ together form (C₁₋₆)alkylene; R¹ represents hydrogen, (C₁₋₆)alkyl or (C₁₋₆)alkoxycarbonyl; and R^{k} represents (C₁₋₈)alkyl, (C₁₋₈)alkoxy, (C₁₋₆)alkoxy(C₁₋₆)alkoxy or aryl.

Examples of suitable in vivo hydrolysable ester groups include, for example, acyloxy(C₁₋₆)alkyl groups such as acetoxymethyl, pivaloyloxymethyl, α-acetoxyethyl, α-pivaloyloxyethyl, 1-(cyclohexylcarbonyloxy)prop-1-yl, and (1-aminoethyl)carbonyloxymethyl; (C₁₋₆)alkoxycarbonyloxy(C₁₋₆)alkyl groups, such as ethoxycarbonyloxymethyl, α-ethoxycarbonyloxyethyl and propoxycarbonyloxyethyl; di(C₁₋₆)alkylamino(C₁₋₆)alkyl especially di(C₁₋₄)alkylamino(C₁₋₄)alkyl groups such as dimethylaminomethyl, dimethylaminoethyl, diethylaminomethyl or diethylaminoethyl; 2-((C₁₋₆)alkoxycarbonyl)-2-(C₂₋₆)alkenyl groups such as 2-(isobutoxycarbonyl)pent-2-enyl and 2-(ethoxycarbonyl)but-2-enyl; lactone groups such as phthalidyl and dimethoxyphthalidyl.

A further suitable pharmaceutically acceptable in vivo hydrolysable ester-forming group is that of the formula: 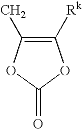
wherein R^{k} is hydrogen, C₁₋₆alkyl or phenyl.
R is preferably hydrogen.

Compounds of formula (I) may also be prepared as the corresponding N-oxides.

Certain of the compounds of formula (I) may exist in the form of optical isomers, e.g. diastereoisomers and mixtures of isomers in all ratios, e.g. racemic mixtures. The invention includes all such forms, in particular the pure isomeric forms. For example the invention includes compound in which an A-B group CH(OH)—CH₂ is in either isomeric configuration, the R-isomer is preferred. The different isomeric forms may be separated or resolved one from the other by conventional methods, or any given isomer may be obtained by conventional synthetic methods or by stereospecific or asymmetric syntheses.

In a further aspect of the invention there is provided a process for preparing compounds of formula (I), and pharmaceutically acceptable derivatives thereof, which process comprises reacting a compound of formula (IV) with a compound of formula (V): 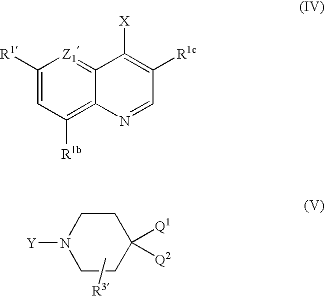
wherein Z^{1′}, R^{1′}, R^{1b′}, R^{1c′} and R^{3′} are Z¹, R¹, R^{1b}, R^{1c} and R³ as defined in formula (I) or groups convertible thereto.
Q¹ is NHR^{4′} or a group convertible thereto wherein R^{4′} is R⁴ as defined in formula (I) or groups convertible thereto and Q² is H or R^{3′} or Q¹ and Q² together form an optionally protected oxo group;
(i) X is A′-COW, Y is H;
(ii) X is CH═CH₂, Y is H;
(iii) X is oxirane, Y is H;
(iv) one of X and Y is CO₂R^{y} and the other is CH₂CO₂R^{x};
   in which W is a leaving group, e.g. halo or imidazolyl; R^{x} and R^{y} are (C₁₋₄)alkyl; A′ is A as defined in formula (I), or groups convertible thereto; and oxirane is: 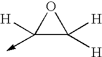
   and thereafter optionally or as necessary converting Q¹ and Q² to NHR^{4′}; converting A′, Z^{1′}, R^{1′}, R^{1b′}, R^{1c′}, R^{3′}, and R^{4′} to A, Z¹, R¹, R^{1b}, R^{1c}, R³, and R⁴; converting A-B to other A-B, interconverting R¹, R^{1b}, R^{1c}, R³, and/or R⁴, and/or forming a pharmaceutically acceptable derivative thereof.

Process variant (i) initially produces compounds of formula (I) wherein A-B is A′-CO.

Process variant (ii) initially produces compounds of formula (I) wherein A-B is CH₂CH₂.

Process variant (iii) initially produces compounds of formula (I) wherein A-B is CH(OH)—CH₂.

Process variant (iv) initially produces compounds of formula (I) wherein A-B is CO—CH₂ or CH₂—CO.

In process variant (i) the reaction is a standard amide formation reaction involving e.g.:
1. Activation of a carboxylic acid (e.g. to an acid chloride, mixed anhydride, active ester, O-acyl-isourea or other species), and treatment with an amine (Ogliaruso, M. A.; Wolfe, J. F. in The Chemistry of Functional Groups (Ed. Patai, S.) Suppl. B: The Chemistry of Acid Derivatives, Pt. 1 (John Wiley and Sons, 1979), pp 442-8; Beckwith, A. L. J. in The Chemistry of Functional Groups (Ed. Patai, S.) Suppl. B: The Chemistry of Amides (Ed. Zabricky, J.) (John Wiley and Sons, 1970), p 73 ff. The acid and amine are preferably reacted in the presence of an activating agent such as 1-(dimethylaminopropyl)-3-ethylcarbodiimide hydrochloride (EDC) or 1-hydroxybenzotriazole (HOBT) or O-(7-azabenzotriazol-1-yl)-N,N,N′,N′-tetramethyluronium hexafluorophosphate (HATU); or
in situ conversion of the acid component into the acid chloride under neutral conditions (Villeneuve, G. B.; Chan, T. H., Tetrahedron. Lett. 1997, 38, 6489).

A′ may be, for example, protected hydroxymethylene.

The process variant (ii) is a standard addition reaction using methods well known to those skilled in the art. The process is preferably carried out in a polar organic solvent e.g. acetonitrile, DMF or chloroform optionally in the presence of an organic base e.g. triethylamine. In some cases an elevated temperature such as 40-150° C. may be beneficial.

In process variant (iii) the coupling may be effected in the absence of solvent, or in a suitable solvent such as acetonitrile, chloroform or dimethylformamide at room temperature optionally in the presence of one equivalent of lithium perchlorate as catalyst (general method of J. E. Chateauneuf et al, J. Org. Chem., 56, 5939-5942, 1991) or with ytterbium triflate in dichloromethane. In some cases an elevated temperature such as 40-70° C. may be beneficial. Alternatively, the piperidine may be treated with a base, such as one equivalent of butyl lithium, and the resulting salt reacted with the oxirane in an inert solvent such as tetrahydrofuran, preferably at an elevated temperature such as 80° C. Use of a chiral epoxide will afford single diastereomers. Alternatively, mixtures of diastereomers may be separated by preparative HPLC or by conventional resolution through crystallisation of salts formed from chiral acids.

In process variant (iv) the process is two step: firstly a condensation using a base, preferably sodium hydride or alkoxide, sodamide, alkyl lithium or lithium dialkylamide, preferably in an aprotic solvent e.g. ether, THF or benzene; secondly, hydrolysis using an inorganic acid, preferably HCl in aqueous organic solvent at 0-100° C. Analogous routes are described in DE330945, EP31753, EP53964 and H. Sargent, J. Am. Chem. Soc. 68, 2688-2692 (1946). Similar Claisen methodology is described in Soszko et. al., Pr. Kom. Mat. Przyr. Poznan. Tow. Przyj. Nauk., (1962), 10, 15.

Reduction of a carbonyl group of A or B to CHOH can be readily accomplished using reducing agents well known to those skilled in the art, e.g. sodium borohydride in aqueous ethanol or methanol, or lithium aluminium hydride in ethereal solution. This is analogous to methods described in EP53964, U.S. Pat. No. 384,556 and J. Gutzwiller et al, J. Amer. Chem. Soc., 1978, 100, 576.

The carbonyl group of A or B may be reduced to CH₂ by treatment with a reducing agent such as hydrazine in ethylene glycol, at e.g. 130-160° C., in the presence of potassium hydroxide.

A hydroxy group on A or B may be oxidised to a carbonyl group by oxidants well known to those skilled in the art, for example, manganese dioxide, pyridinium chlorochromate or pyridinium dichromate.

A hydroxyalkyl A-B group CHR⁶CHOH or CR⁶(OH)CH₂ may be dehydrated to give the group CR⁶═CH by treatment with an acid anhydride such as acetic anhydride.

Methods for conversion of CH═CH by reduction to CH₂CH₂ are well known to those skilled in the art, for example using hydrogenation over palladium on carbon as catalyst. Methods for conversion of CR⁶═CH to give the A-B group CR⁶(OH)CH₂ are well known to those skilled in the art for example by epoxidation and subsequent reduction by metal hydrides.

An amide carbonyl group may be reduced to the corresponding amine using a reducing agent such as lithium aluminium hydride.

A hydroxy group in A or B may be converted to azido by activation and displacement e.g. under Mitsunobu conditions using hydrazoic acid or by treatment with diphenylphosphorylazide and base, and the azido group in turn may be reduced to amino by hydrogenation.

When Q¹ Q² together form a protected oxo group this may be an acetal such as ethylenedioxy which can subsequently be removed by acid treatment to give a compound of formula (VI): 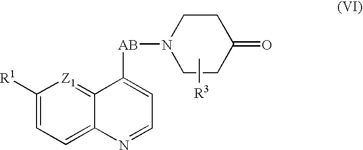
wherein the variables are as described for formula (I).

The ketone of formula (VI) is reacted with an amine HNH′R^{4′} by conventional reductive alkylation using, e.g., sodium borohydride or sodium triacetoxyborohydride (Gribble, G. W. in Encyclopedia of Reagents for Organic Synthesis (Ed. Paquette, L. A.) (John Wiley and Sons, 1995), p 4649).

Examples of groups Z^{1′} convertible to Z¹, include CR^{1a} where R^{1a′} is a group convertible to R^{1a}, R^{1a′}, R^{1′}, R^{1b′} and R^{1c′} are preferably R^{1a}, R¹, R^{1b}, and R^{1c}. R^{3′} is R³ or a group convertible thereto. R^{4′} is R⁴ or more preferably H or an N-protecting group such as t-butoxycarbonyl, benzyloxycarbonyl or 9-fluorenylmethoxycarbonyl. R^{1b} is preferably H or F. R^{1c} is preferably Cl or F.

Conversions of R^{1′}, R^{1b′}, R^{1c′}, R^{3′} and R^{4′} and interconversions of R¹, R^{1b}, R^{1c}, R³ and R⁴ are conventional. In compounds which contain an optionally protected hydroxy group, suitable conventional hydroxy protecting groups which may be removed without disrupting the remainder of the molecule include acyl and alkylsilyl groups. N-protecting groups are removed by conventional methods.

For example R^{1′} or R^{1a′} methoxy is convertible to R¹ or R^{1a} hydroxy by treatment with HBr or lithium and diphenylphosphine (general method described in Ireland et al, J. Amer. Chem. Soc., 1973, 7829) or HBr. Alkylation of the hydroxy group with a suitable (C₁₋₄)alkyl or (C₁₋₄)alkoxy derivative bearing a leaving group such as halide will produce R^{1′} is (C₁₋₄)alkoxy or R^{1a} is (C₁₋₄)alkoxy substituted by (C₁₋₄)alkoxy. R^{3′} alkenyl is convertible to hydroxyalkyl by hydroboration using a suitable reagent such as 9-borabicyclo[3.3.1]nonane, epoxidation and reduction or oxymercuration.

Carboxy groups within R³ may be prepared by Jones' oxidation of the corresponding alcohols CH₂OH using chromium acid and sulphuric acid in water/methanol (E. R. H. Jones et al, J. Chem. Soc., 1946, 39). Other oxidising agents may be used for this transformation such as sodium periodate catalysed by ruthenium trichloride (G. F. Tutwiler et al, J. Med. Chem., 1987, 30(6), 1094), chromium trioxide-pyridine (G. Just et al, Synth. Commun., 1979, 9(7), 613), potassium permanganate (D. E. Reedich et al, J. Org. Chem., 1985, 50(19), 3535), and pyridinium chlorochromate (D. Askin et al, Tetrahedron Lett., 1988, 29(3), 277).

The carboxy group may alternatively be formed in a two stage process, with an initial oxidation of the alcohol to the corresponding aldehyde using for instance dimethyl sulphoxide activated with oxalyl chloride (N. Cohen et al, J. Am. Chem. Soc., 1983, 105, 3661) ordicyclohexylcarbodiimide (R. M. Wengler, Angew. Chim. Int. Ed. Eng., 1985, 24(2), 77), or oxidation with tetrapropylammonium perruthenate (Ley et al, J. Chem. Soc. Chem Commun., 1987, 1625). The aldehyde may then be separately oxidised to the corresponding acid using oxidising agents such as silver (II) oxide (R. Grigg et al, J. Chem. Soc. Perkin1, 1983, 1929), potassium permanganate (A. Zurcher, Helv. Chim. Acta., 1987, 70 (7), 1937), sodium periodate catalysed by ruthenium trichloride (T. Sakata et al., Bull. Chem. Soc. Jpn., 1988, 61(6), 2025), pyridinium chlorochromate (R. S. Reddy et al, Synth. Commun., 1988, 18(51), 545) or chromium trioxide (R. M. Coates et al, J. Am. Chem. Soc., 1982, 104, 2198).

Other routes to the synthesis of carboxy groups within R³ are well known to those skilled in the art.

R³ groups containing a carboxy group may also be prepared by conversion of an alcohol to a suitable leaving group such as the corresponding tosylate by reaction with para-toluenesulphonyl chloride (M. R. Bell, J. Med. Chem., 1970, 13, 389), or the iodide using triphenylphosphine, iodine, and imidazole (G. Lange, Synth. Commun., 1990, 20, 1473). The second stage is the displacement of the leaving group with cyanide anion (L. A. Paquette et al, J. Org. Chem., 1979, 44(25), 4603; P. A. Grieco et al, J. Org. Chem., 1988, 53(16), 3658. Finally acidic hydrolysis of the nitrile group gives the desired acids (H. Rosemeyer et al, Heterocycles, 1985, 23 (10), 2669). The hydrolysis may also be carried out with base e.g. potassium hydroxide (H. Rapoport, J. Org. Chem., 1958, 23, 248) or enzymatically (T. Beard et al, Tetrahedron Asymmetry, 1993, 4 (6), 1085).

R³ cis or trans hydroxy may be introduced by the methods of van Deale et al., Drug Development Research 8:225-232 (1986) or Heterocycles 39(1), 163-170 (1994). For trans hydroxy, a suitable method converts N-protected tetrahydropyridine to the epoxide by treatment with metachloroperbenzoic acid, followed by opening of the epoxide with a suitable amine NR^{2′}R^{4′}.

Other functional groups in R³ may be obtained by conventional conversions of hydroxy, carboxy or cyano groups.

Other substituents on R³ alkyl or alkenyl may be interconverted by conventional methods, for example hydroxy may be derivatised by etherification. Primary and secondary hydroxy groups can be oxidised to an aldehyde or ketone respectively and alkylated with a suitable agent such as an organometallic reagent to give a secondary or tertiary alcohol as appropriate. A carboxylate group may be converted to an hydroxymethyl group by reduction of an ester of this acid with a suitable reducing agent such as lithium aluminium hydride.

An NH₂ substituent on piperidine is converted to NHR⁴ by conventional means such as amide or sulphonamide formation with an acyl derivative R⁵COW or R⁵SO₂W, for compounds where U is CO or SO₂ or, where U is CH₂, by alkylation with an alkyl halide R⁵CH₂-halide in the presence of base, acylation/reduction with an acyl derivative R⁵COW or reductive alkylation with an aldehyde R⁵CHO.

Where one of R³ or R⁶ contains a carboxy group and the other contains a hydroxy or amino group they may together form a cyclic ester or amide linkage. This linkage may form spontaneously during coupling of the compound of formula (IV) and the piperidine moiety or in the presence of standard peptide coupling agents.

It will be appreciated that under certain circumstances interconvertions may interfere, for example, A or B hydroxy groups in A or B and the piperidine substituent NH₂ will require protection e.g. as a carboxy- or silyl-ester group for hydroxy and as an acyl derivative for piperidine NH₂, during conversion of R^{1′}, R^{3′} or R⁴, or during the coupling of the compounds of formulae (IV) and (V).

Compounds of formulae (IV) and (V) are known compounds, (see for example Smith et al, J. Amer. Chem. Soc., 1946, 68, 1301) or prepared analogously.

4-Alkenyl compounds of formula (IV) may be prepared by conventional procedures from a corresponding 4-halogeno-derivative by e.g. a Heck synthesis as described in e.g. Organic Reactions, 1982, 27, 345 or via 2,4,6-trivinylcyclotroboroxane (J. Org. Chem. 2002, 67, 4968-4971).

4-Halogeno derivatives of compounds of formula (IV) are commercially available, or may be prepared by methods known to those skilled in the art. For example, a 4-chloroquinoline is prepared from the corresponding quinolin-4-one by reaction with phosphorus oxychloride (POCl₃) or phosphorus pentachloride, PCl₅ and 4-bromoquinoline is prepared similarly with phosphorous oxybromide or more preferably phosphorous tribromide in N,N-dimethylformamide (see M. Schmittel et al, Synlett, 1997, (9), 1096 and K. Gould et al, J. Med., Chem., 1988, 31 (7), 1445). 4-Carboxy derivatives of compounds of formula (IV) are commercially available or may be prepared by conventional procedures for preparation of carboxy heteroaromatics well known to those skilled in the art.

A 4-oxirane derivative of compounds of formula (IV) is conveniently prepared from the 4-carboxylic acid by first conversion to the acid chloride with oxalyl chloride and then reaction with trimethylsilyldiazomethane to give the diazoketone derivative. Subsequent reaction with 5M hydrochloric acid gives the chloromethylketone. Reduction with sodium borohydride in aqueous methanol gives the chlorohydrin which undergoes ring closure to afford the epoxide on treatment with base, e.g. potassium hydroxide in ethanol-tetrahydrofuran.

Alternatively and preferably, 4-oxirane derivatives can be prepared from bromomethyl ketones which can be obtained from 4-hydroxy compounds by other routes well known to those skilled in the art. For example, hydroxy compounds can be converted to the corresponding 4-trifluoromethanesulphonates by reaction with trifluoromethanesulphonic anhydride under standard conditions (see K. Ritter, Synthesis, 1993, 735). Conversion into the corresponding butyloxyvinyl ethers can be achieved by a Heck reaction with butyl vinyl ether under palladium catalysis according to the procedure of W. Cabri et al, J. Org. Chem, 1992, 57 (5), 1481. (Alternatively, the same intermediates can be attained by Stille coupling of the trifluoromethanesulphonates or the analaogous chloro derivatives with (1-ethoxyvinyl)tributyl tin, T. R. Kelly, J. Org. Chem., 1996, 61, 4623.) The alkyloxyvinyl ethers are then converted into the corresponding bromomethylketones by treatment with N-bromosuccinimide in aqueous tetrahydrofuran in a similar manner to the procedures of J. F. W. Keana, J. Org. Chem., 1983, 48, 3621 and T. R. Kelly, J. Org. Chem., 1996, 61, 4623.

The 4-hydroxyderivatives can be prepared from an aminoaromatic by reaction with methylpropiolate and subsequent cyclisation, analogous to the method described in N. E. Heindel et al, J. Het. Chem., 1969, 6, 77. For example, 5-amino-2-methoxy pyridine can be converted to 4-hydroxy-6-methoxy-[1,5]naphthyridine using this method.

If a chiral reducing agent such as (+) or (−)-B-chlorodiisopinocamphenylborane [‘DIP-chloride’] is substituted for sodium borohydride, the prochiral chloromethylketone may be converted into the chiral chlorohydrin [see C. Bolm et al, Chem. Ber. 125, 1169-1190, (1992)].

Recrystallisation of the chiral epoxide or chiral HPLC gives material with enhanced optical purity (typically ee >95%).

The chiral )-epoxide, when reacted with a piperidine derivative gives ethanolamine compounds as single diastereomers with -corresponding chiral stereochemistry at the benzylic position.

Alternatively, the chiral epoxide can be prepared from the 4-vinyl derivative by an osmium-catalysed asymmetric dihydroxylation using either AD-mix-β or AD-mix-α (see K. B. Sharpless et al. J. Org. Chem. 1992, 57, 2768-2771) giving chiral diols, (typically ee values of 40-65% for 3-fluoro-naphthyridines/quinolines) which can be converted to the mono-tosyl-derivative by reaction with tosyl chloride (DCM-THF-Et₃N) (conveniently catalysed by dibutyltinoxide—see M. J. Martinelli et al. J.A.C.S. 2002, 124, 3578-3585), followed by reaction with a base such as anhydrous potassium carbonate in methanol.

Alternatively, the epoxide may be prepared from the 4-carboxaldehyde by a Wittig approach using trimethylsulfonium iodide [see G. A. Epling and K-Y Lin, J. Het. Chem., 1987, 24, 853-857], or by epoxidation of a 4-vinyl derivative. 4-Hydroxy-1,5-naphthyridines can be prepared from 3-aminopyridine derivatives by reaction with diethyl ethoxymethylene malonate to produce the 4-hydroxy-3-carboxylic acid ester derivative with subsequent hydrolysis to the acid, followed by thermal decarboxylation in quinoline (as for example described for 4-Hydroxy-[1,5]naphthyridine-3-carboxylic acid, J. T. Adams et al., J. Amer. Chem. Soc., 1946, 68, 1317).

Compounds of formula (IV) are available by the sequence described below, starting from an aromatic or heterocyclic amine (1), with at least one free CH position adjacent to the amine. Reaction with Meldrum's acid and trimethyl orthformate in ethanol at reflux affords the corresponding 2,2-dimethyl-5-phenylaminomethylene-[1,3]dioxane-4,6-dione derivatives (2). These can be cyclised at elavated temperatures (180-220° C.) in inert solvents such as Dowtherm to give the corresponding 1H-quinolin-4-one or heterocyclic derivatives (3). These processes are well-established and are described by Walz and Sundberg (J. Org. Chem., 2000, 65 (23), 8001) and by Todter and Lackner (Synthesis, 1997 (5) 576).

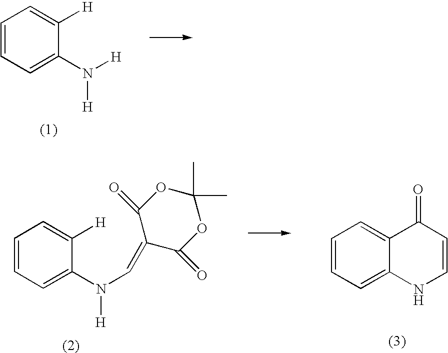

A 4-hydroxy-[1,5]naphthyridine can be converted to the 4-chloro derivative by heating in phosphorus oxychloride, or to the 4-methanesulphonyloxy or 4-trifluoromethanesulphonyloxy derivative by reaction with methanesulphonyl chloride or trifluoromethanesulphonic anhydride, respectively, in the presence of an organic base. Activation of the quinolone species related to (3) into the corresponding 4-quinolyl bromides (4) can be accomplished with phosphorous oxybromide or more preferably phosphorous tribromide in N,N-dimethylformamide (see M. Schmittel et al, Synlett, 1997, (9), 1096 and K. Gould et al, J. Med., Chem., 1988, 31 (7), 1445). The corresponding chlorides (5) are available by using phosphoryl oxychloride (for instance C. W. Wright et al, J. Med., Chem., 2001, 44 (19), 3187). 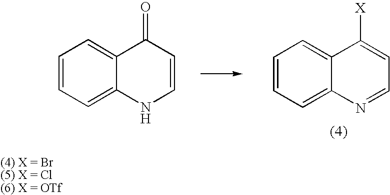
Alternatively, the quinolone species may be activated to the corresponding 1,1,1-trifluoro-methanesulfonic acid quinolin-4-yl esters (6) by the action of agents such as triflic anhydride or more preferably N-trifluoromethanesulphonimide (see for example M. Alvarez et al, Tet 2000, 56 (23) 3703; M. Alvarez et al, Eur. J. Org., Chem., 2000, (5), 849; J. Joule et al, Tet, 1998, 54 (17), 4405; J. K. Stille et al, J.A.C.S., 1988, 110 (12), 4051).

1,5-Naphthyridines may be prepared by other methods well known to those skilled in the art (for examples see P. A. Lowe in “Comprehensive Heterocyclic Chemistry” Volume 2, p 581-627, Ed A. R. Katritzky and C. W. Rees, Pergamon Press, Oxford, 1984).

3-Chloro-4-hydroxyquinolines or naphthyridines may be prepared by chlorination of the 4-hydroxyquinoline or naphthyridine with a suitable reagent eg. N-chlorosuccinimide in acetic acid. The 4-hydroxy group may then be converted into the trifluoromethylsulfonate ester by treatment with a sulfonation reagent eg. N-phenyltrifluoromethanesulfonimide, or into the 4-bromo compound by treatment with phosphorus tribromide in dimethylformamide.

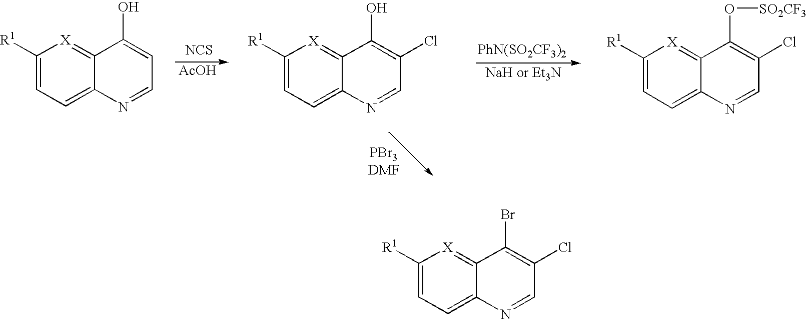

3-bromo-4-hydroxyquinolines or naphthyridines may be prepared, in a similar mannar as given above, by bromination of the 4-hydroxyquinoline or naphthyridine with a suitable reagent eg. N-bromosuccinimide in acetic acid. The 4-hydroxy group may then be converted into the trifluoromethylsulfonate ester by treatment with a sulfonation reagent eg. N-phenyltrifluoromethanesulfonimide, or into the 4-bromo compound by treatment with phosphorus tribromide in dimethylformamide.

3-Fluoro-4-chloroquinolines may be prepared from the 3-amino-4-chloro compounds by conversion into the diazonium tetrafluoroborate salt, using sodium nitrite and tetrafluoroboric acid or nitrosonium tetrafluoroborate in a suitable solvent (EP 430,434), followed by thermal decomposition (WO 98/13350 and WO 02/072578). The 3-amino compounds may be prepared either from the 3-carboxylic acid by heating with diphenylphosphoryl azide in the presence of triethylamine and tert-butanol, followed by deprotection of the resulting tert-butyl carbamate with acid (WO 02/072578), or from the 3-nitro compound by reduction, for example with hydrogen in the presence of Raney nickel (WO 98/13350).

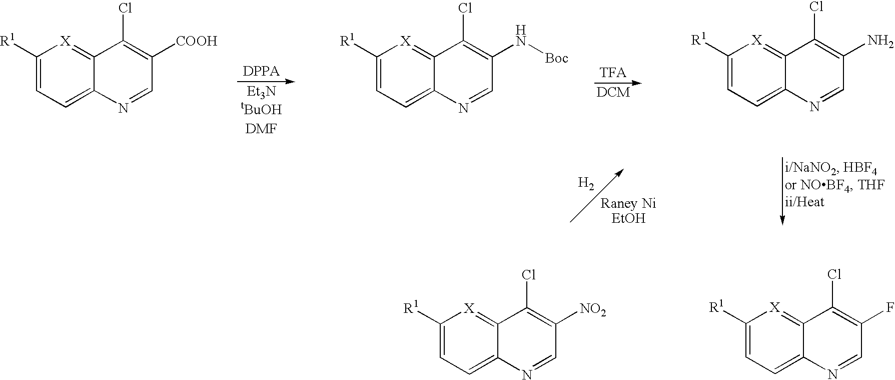

For compounds of formula (V), suitable amines may be prepared from the corresponding 4-substituted piperidine acid or alcohol. In a first instance, an N-protected piperidine containing an acid bearing substituent, can undergo a Curtius rearrangement and the intermediate isocyanate can be converted to a carbamate by reaction with an alcohol. Conversion to the amine may be achieved by standard methods well known to those skilled in the art used for amine protecting group removal. For example, an acid substituted N-protected piperidine can undergo a Curtius rearrangement e.g. on treatment with diphenylphosphoryl azide and heating, and the intermediate isocyanate reacts in the presence of 2-trimethylsilylethanol to give the trimethylsilylethylcarbamate (T. L. Capson & C. D. Poulter, Tetrahedron Lett., 1984, 25, 3515). This undergoes cleavage on treatment with tetrabutylammonium fluoride to give the 4-amine substituted N-protected piperidine.

In a second instance, an N-protected piperidine containing an alcohol bearing substituent undergoes a Mitsunobu reaction (for example as reviewed in Mitsunobu, Synthesis, (1981), 1), for example with succinimide in the presence of diethyl azodicarboxylate and triphenylphosphine to give the phthalimidoethylpiperidine. Removal of the phthaloyl group, for example by treatment with methylhydrazine, gives the amine of formula (V).

R⁵CH₂-halides, acyl derivative R⁵COW and R⁵SO₂W or aldehydes R⁵CHO are commercially available or are prepared conventionally. The aldehydes may be prepared by partial reduction of the R⁵-ester with lithium aluminium hydride or di-isobutylaluminium hydride or more preferably by reduction to the alcohol, with lithium aluminium hydride or sodium borohydride or lithium triethylborohydride (see Reductions by the Alumino- and Borohydrides in Organic Synthesis, 2nd ed., Wiley, N.Y., 1997; JOC, 3197, 1984; Org. Synth. Coll., 102, 1990; 136, 1998; JOC, 4260, 1990; TL, 995, 1988; JOC, 1721, 1999; Liebigs Ann./Recl., 2385, 1997; JOC, 5486, 1987), followed by oxidation to the aldehyde with manganese (II) dioxide. The aldehydes may also be prepared from carboxylic acids in two stages by conversion to a mixed carbonate for example by reaction with isobutyl chloroformate followed by reduction with sodium borohydride (R. J. Alabaster et al., Synthesis, 598, 1989) to give the hydroxymethyl substituted heteroaromatic or aromatic and then oxidation with a standard oxidising agent such as pyridinium dichromate or manganese (II) dioxide. Acyl derivative R⁵COW may be prepared by activation of the R⁵-ester. R⁵CH₂-halides such as bromides may be prepared from the alcohol R⁵CH₂OH by reaction with phosphorus tribromide in DCM/triethylamine.

Alternatively the aldehyde R⁵CHO and sulphonic acid derivative R⁵SO₂W may be generated by treatment of the R⁵H heterocycle with suitable reagents. For example benzoxazinones, or more preferably their N-methylated derivatives can be formylated with hexamine in either trifluoroacetic acid or methanesulfonic acid, in a modified Duff procedure [O. I. Petrov et al. Collect. Czech. Chem. Commun. 62, 494-497 (1997)]. 4-Methyl-4H-benzo[1,4]oxazin-3-one may also be formylated using dichloromethyl methyl ether and aluminium chloride giving exclusively the 6-formyl derivative. Reaction of a R⁵H heterocycle with chlorosulphonic acid gives the sulphonic acid derivative (by methods analogous to Techer et. al., C. R. Hebd. Seances Acad. Sci. Ser. C; 270, 1601, 1970).

The aldehyde R⁵CHO may be generated by conversion of an R⁵ halogen or R⁵ trifluoromethane sulphonyloxy derivative into an olefin with subsequent oxidative cleavage by standard methods. For example, reaction of a bromo derivative under palladium catalysis with trans-2-phenylboronic acid under palladium catalysis affords a styrene derivative which upon ozonolysis affords the required R⁵CHO (Stephenson, G. R., Adv. Asymmetric Synth. (1996), 275-298. Publisher: Chapman & Hall, London).

R⁵ heterocycles are commercially available or may be prepared by conventional methods. For example where a benzoxazinone is required, a nitrophenol may be alkylated with for example ethyl bromoacetate and the resulting nitro ester reduced with Fe in acetic acid (alternatively Zn/AcOH/HCl or H₂ Pd/C or H₂ Raney Ni). The resulting amine will undergo spontaneous cyclisation to the required benzoxazinone. Alternatively a nitrophenol may be reduced to the aminophenol, which is reacted with chloroacetyl chloride [method of X. Huang and C. Chan, Synthesis 851 (1994)] or ethyl bromoacetate in DMSO [method of Z. Moussavi et al. Eur. J. Med. Chim. Ther. 24, 55-60 (1989)]. The same general routes can be applied to prepare benzothiazinones [See for example F. Eiden and F. Meinel, Arch. Pharm. 312, 302-312 (1979), H. Fenner and R Grauert Liebigs. Ann. Chem. 193-313 (1978)]]. A variety of routes are available to prepare aza analogues of benzothiazinones via the key corresponding aldehydes. For instance, 2-oxo-2,3-dihydro-1H-pyrido[3,4-b][1,4]thiazine-7-carbaldehyde may be accessed from 5-fluoro-2-picoline (E. J. Blanz, F. A. French, J. R. DoAmaral and D. A. French, J. Med. Chem. 1970, 13, 1124-1130) by constructing the thiazinone ring onto the pyridyl ring then functionalising the methyl substituent. The dioxin analogue of this aza substitution patern, 2,3-dihydro-[1,4]dioxino[2,3-c]pyridine-7-carbaldehyde is accessible from Kojic acid by aminolysis from pyrone to pyridone then annelating the dioxin ring. Other aza substitution patterns with pyridothiazin-3-one, pyridooxazin-3-one, and pyridodioxin ring systems are also accessible. Ortho-aminothiophenols may be conveniently prepared and reacted as their zinc complexes [see for example V. Taneja et al Chem. Ind. 187 (1984)]. Benzoxazolones may be prepared from the corresponding aminophenol by reaction with carbonyl diimidazole, phosgene ot triphosgene. Reaction of benzoxazolones with diphosporus pentasulfide affords the corresponding 2-thione. Thiazines and oxazines can be prepared by reduction of the corresponding thiazinone or oxazinone with a reducing agent such as lithium aluminium hydride.

The amines R^{4′}NH₂ are available commercially or prepared conventionally. For example amines R⁵CH₂NH₂ may be prepared from a bromomethyl derivative by reaction with sodium azide in dimethylformamide (DMF), followed by hydrogenation of the azidomethyl derivative over palladium-carbon. An alternative method is to use potassium phthalimide/DMF to give the phthalimidomethyl derivative, followed by reaction with hydrazine in DCM to liberate the primary amine.

Conversions of R^{1a′}, R^{1b′}, R^{1c′}, R^{3′} and R^{4′} may be carried out on the intermediates of formulae (IV), and (V) prior to their reaction to produce compounds of formula (I) in the same way as described above for conversions after their reaction.

Another method of synthesizing compound of formula (I) is outlined in Scheme I.

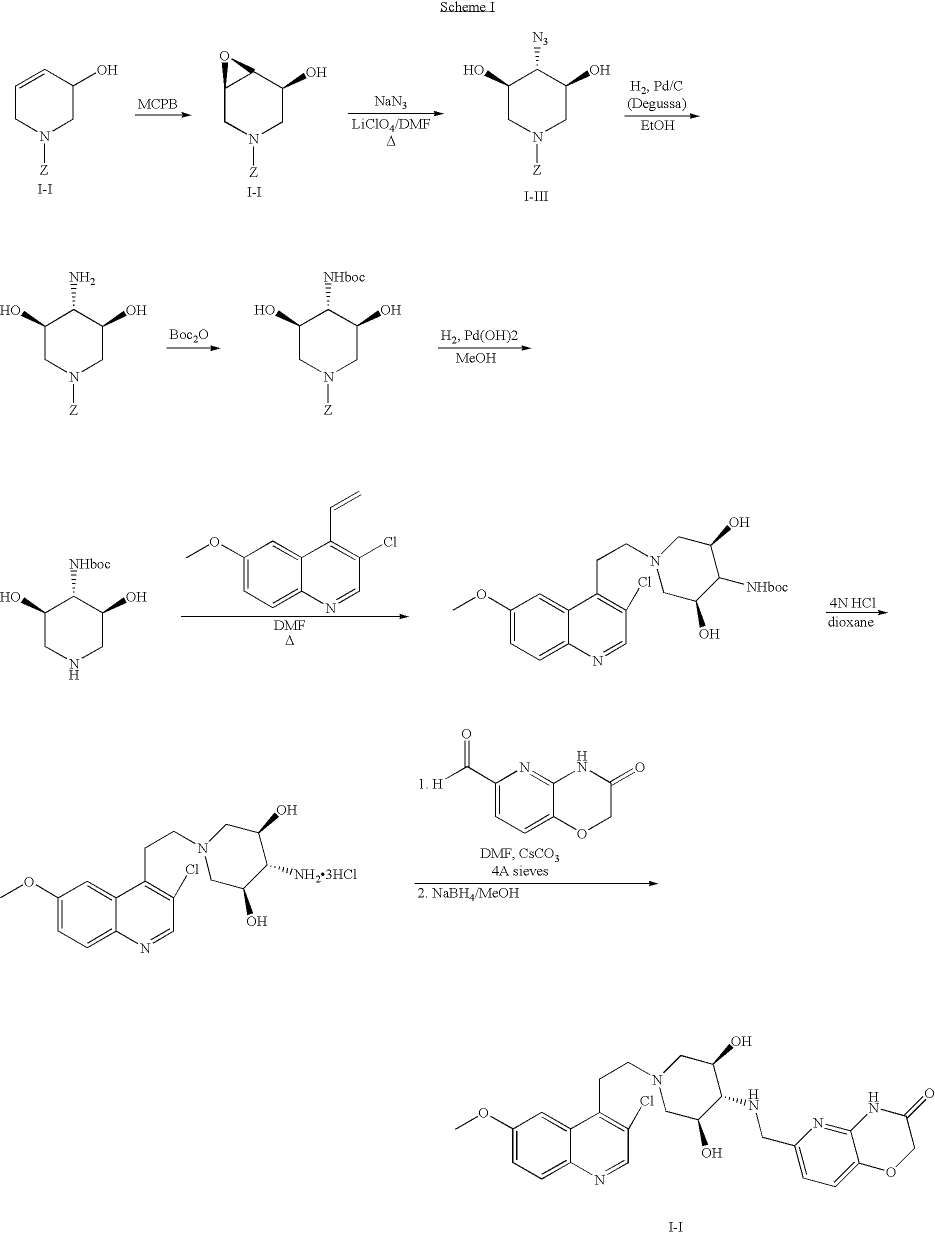

Allylic alcohol (I-I) can be prepared by procedures outlined in either Heterocycles 1992, 33, 349 or Synthesis 2000, 521, 33, 349. Oxidation of (I-I) with MCPBA cleanly affords cis epoxide (I-II). Treatment of (I-II) with NaN₃ in DMF containing LiClO₄ at elevated temperatures affords a mixture of dihydroxy azides with isomer (I-III) predominating. The isomers can be easily separated by column chromatography and the structure of (I-III) confirmed by COSY NMR. Conversion of (I-III) to target compounds such as (I-IV) can be accomplished using the same procedures used to prepare the mono-hydroxy derivatives described herein.

Further details for the preparation of compounds of formula (I) are found in the examples.

The compounds of formula (I) may be prepared singly or as compound libraries comprising at least 2, for example 5 to 1,000 compounds, and more preferably 10 to 100 compounds of formula (I). Libraries of compounds of formula (I) may be prepared by a combinatorial “split and mix” approach or by multiple parallel synthesis using either solution phase or solid phase chemistry, by procedures known to those skilled in the art.

Thus according to a further aspect of the invention there is provided a compound library comprising at least 2 compounds of formula (I) or pharmaceutically acceptable derivatives thereof.

Novel intermediates of formulae (IV) and (V) are also part of this invention.

The antibacterial compounds according to the invention may be formulated for administration in any convenient way for use in human or veterinary medicine, by analogy with other antibacterials.

The pharmaceutical compositions of the invention include those in a form adapted for oral, topical or parenteral use and may be used for the treatment of bacterial infection in mammals including humans.

The composition may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

No toxicological effects are indicated when a compound of formula (I) or a pharmaceutically acceptable derivative thereof is administered in the above-mentioned dosage range.

The compound of formula (I) may be the sole therapeutic agent in the compositions of the invention or a combination with other antibacterials. If the other antibacterial is α-lactam then a β-lactamase inhibitor may also be employed.

Compounds of formula (I) are active against a wide range of organisms including both Gram-negative and Gram-positive organisms.

All publications, including but not limited to patents and patent applications, cited in this specification are herein incorporated by reference as if each individual publication were specifically and individually indicated to be incorporated by reference herein as though fully set forth.

The following examples illustrate the preparation of certain compounds of formula (I) and the activity of certain compounds of formula (I) against various bacterial organisms.

Abbreviations in the examples:

RT=room temperature
ES=Electrospray mass spec.
LCMS=Liquid chromatography mass spec.
APCl+=Atmospheric pressure chemical ionisation mass spec.

Certain reagents are also abbreviated herein. DCC refers to dicyclohexylcarbodiimide, DMAP refers to dimethylaminopyridine, DIEA refers to diisopropylethyl amine, EDC refers to 1-(3-dimethylaminopropyl)-3-ethylcarbodiimide, hydrochloride. HOBt refers to 1-hydroxybenzotriazole, THF refers to tetrahydrofuran, DIEA refers to diisopropylethylamine, DEAD refers to diethyl azodicarboxylate, PPh₃ refers to triphenylphosphine, DIAD refers to diisopropyl azodicarboxylate, DME refers to dimethoxyethane, DMF refers to dimethylformamide, NBS refers to N-bromosuccinimide, Pd/C refers to a palladium on carbon catalyst, PPA refers to polyphosphoric acid, DPPA refers to diphenylphosphoryl azide, BOP refers to benzotriazol-1-yloxy-tris(dimethyl-amino)phosphonium hexafluorophosphate, HF refers to hydrofluoric acid, TEA refers to triethylamine, TFA refers to trifluoroacetic acid, PCC refers to pyridinium chlorochromate.

### EXAMPLES AND EXPERIMENTAL

### General

Proton nuclear magnetic resonance (¹H NMR) spectra were recorded at 300 MHz, and chemical shifts are reported in parts per million (δ) downfield from the internal standard tetramethylsilane (TMS). Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. CDCl₃ is deuteriochloroform, DMSO-d₆ is hexadeuteriodimethylsulfoxide, and CD₃OD is tetradeuteriomethanol. Mass spectra were obtained using electrospray (ES) ionization techniques. Elemental analyses were performed by Quantitative Technologies Inc., Whitehouse, N.J. Melting points were obtained on a Thomas-Hoover melting point apparatus and are uncorrected. All temperatures are reported in degrees Celsius. E. Merck Silica Gel 60 F-254 thin layer plates were used for thin layer chromatography. Flash chromatography was carried out on E. Merck Kieselgel 60 (230-400 mesh) silica gel. Analytical HPLC was performed on Beckman chromatography systems. Preparative HPLC was performed using Gilson chromatography systems. ODS refers to an octadecylsilyl derivatized silica gel chromatographic support. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev. Celite® is a filter aid composed of acid-washed diatomaceous silica, and is a registered trademark of Manville Corp., Denver, Colo.

### Example 1

### 6-({1-[(Racemic)-2-(3-Chloro-6-methoxy-[1,5]naphthyridin-4-yl)-2hydroxy-ethyl]-piperidin-4-ylamino}-methyl)-4H-pyrido[3,2-b][1,4]oxazin-3-one Dihydrochloride

### (a) 3-Chloro-6-methoxy-[1,5]naphthyridin-4-ol

6-Methoxy-[1,5]naphthyridin-4-ol (12 g) in acetic acid (200 mL) was sonicated and warmed until all had dissolved, and then it was treated with N-chlorosuccinimide (10.01 g) and the mixture was heated at 35° C. for 18 hr, cooled, and the solid collected and washed with acetic acid and dried in vacuo at 40° C. overnight, to give a white solid (9.5 g).

MS (ES) m/z 211/213 (M+H)⁺.

### (b) 1,1,1-Trifluoro-methanesulfonic acid 3-chloro-6-methoxy-[1,5]naphthyridin-4-yl ester

A suspension of 60% sodium hydride in oil (3.08 g) was washed with hexane, the hexane solution decanted, and dry DMF (200 mL) added followed by the phenol (1a) (11.62 g). The mixture was stirred at room temperature for 1 hr, cooled in ice, N-phenyltrifluoromethanesulphonimide (21.62 g) added and the mixture was allowed to stir at room temperature overnight. It was evaporated, azeotroped with toluene, taken up in ether-DCM and washed with sodium carbonate solution, dried (sodium sulfate) and evaporated to give a solid (15 g).

MS (+ve ion electrospray) m/z 343/345 (MH+).

### (c) 8-(1-Butoxy-vinyl)-7-chloro-2-methoxy-[1,5]naphthyridine

The triflate (1b) (8.8 g) in DMF (80 mL) with triethylamine (7.2 mL) butyl vinyl ether (19.3 mL), palladium (II) acetate (0.584 g) and 1,3-bis(diphenylphosphino)propane (1.06 g) was heated at 65-70° C. for 30 hours then evaporated, azeotroped with toluene, and chromatographed on silica gel (dichloromethane-hexane) to give a solid (3.7 g).

MS (ES) m/z 293/295 (M+H)⁺.

### (d) 2-Bromo-1-(3-chloro-6-methoxy-[1,5]naphthyridin-4-yl)-ethanone

The vinyl ether (1c) (6.51 g) was dissolved in THF (100 mL), and water (9 mL) and treated with N-bromosuccinimide (6.51 g) for 5 hour, then evaporated and chromatographed on silica gel (dichloromethane-hexane) to give the ketone as a solid (8.9 g).

MS (ES) m/z 315/317 (M+H)⁺.

### (e) 7-Chloro-2-methoxy-8-(R/S)-oxiranyl-[1,5]naphthyridine

The ketone (1d) (10.5 g) in methanol (160 mL) and water (40 mL) was cooled in ice and sodium borohydride (2.59 g) was added and the solution stirred at room temperature for 1.5 hr. Water was added and it was extracted with chloroform and dried over sodium sulfate and evaporated to give the bromo-alcohol as yellow solid, which was dissolved in methanol (50 mL) and treated with anhydrous potassium carbonate (5.07 g). The mixture was stirred for 3 hr at room temperature then diluted with water and extracted with chloroform, dried and evaporated and chromatographed on silica gel (hexane-DCM then chloroform) to afford a solid, which was recrystallised from ether-hexane to give a solid (3.6 g).

MS (ES) m/z 237/239 (M+H)⁺.

### (f) {1-[(Racemic)-2-(3-Chloro-6-methoxy-[1,5]naphthyridin-4-yl)-2-hydroxy-ethyl]-piperidin-4-yl}-carbamic acid tert-butyl ester

A mixture of epoxide (1e) (0.99 g) and piperidin-4-yl-carbamic acid tert-butyl ester (0.84 g) was heated at 100-105° C. for 3 hr, and one drop of DMF was added and heating was continued for a further 1 hr. The product was dissolved in chloroform and chromatographed on silica gel (methanol-DCM) to afford the solid product (0.78 g) containing ca. 20% of the epoxide ‘wrong-opening’ isomer.

### (g) 1-[(R/S)-2-(3-Chloro-6-methoxy-[1,5]naphthyridin-4-yl)-2-hydroxy-ethyl]-piperidin-4-ylamine

The ester (1f) (0.69 g) in DCM (20 mL) was treated with TFA (20 mL) at room temperature for 3 hr and evaporated. Water and sodium carbonate were added and the solution was extracted with 10% methanol-chloroform, dried (sodium sulfate) and evaporated to afford the product as a foam containing ca. 20% of the ‘epoxide wrong-opening’ isomer.

### (h) 2-Bromo-5-hydroxy-6-nitropyridine

3-Hydroxy-2-nitropyridine (20 g, 0.143 mole) was dissolved in methanol (400 mL) and a solution of 25% sodium methoxide in methanol (33 mL, 0.13 mole) was added at room temperature. The mixture was stirred for 30 min, then was cooled to 0° C., and bromine (7.2 mL, 0.14 mole) was added slowly. The reaction was stirred at 0° C. for 30 min, then was quenched with glacial AcOH (2.5 mL). The solvent was removed in vacuo to afford material (30 g, 96%), which was used without further purification.

MS (ES) m/z 219.0 (M+H)⁺.

### (i) Ethyl (6-bromo-2-nitro-pyridin-3-yloxy)acetate

The hydroxypyridine (1h) (30 g, 0.14 mole) was suspended in acetone (200 ml), and potassium carbonate (39 g, 0.28 mole) was added, followed by ethyl bromoacetate (15.7 ml, 0.14 mmole). The reaction was heated at reflux for 10 hr, then was cooled to room temperature and diluted with Et₂O. The precipitate was removed by suction filtration, and the filtrate was concentrated in vacuo to afford material (38 g, 89%), which was used without further purification.

MS (ES) m/z 305.0 (M+H)⁺.

### (j) 6-Bromo-4H-pyrido[3,2-b][1,4]oxazin-3-one

The nitropyridine (1i) (38 g, 0.125 mole) was dissolved in glacial AcOH (150 mL), and iron powder (20 g, 0.36 mole) was added. The mixture was mechanically stirred and heated at 90° C. for 5 hr, then was cooled to room temperature and diluted with EtOAc (300 mL). The mixture was filtered through a pad of silica gel and the filtrate was concentrated in vacuo and the residue recrystallized from MeOH (15 g, 52%).

MS (ES) m/z 229.0 (M+H)⁺.

### (k) 6-((E)-Styryl)-4H-pyrido[3,2-b][1,4]oxazin-3-one

The bromopyridine (1j) (6.0 g, 26.3 mmole) and trans-2-phenylvinylboronic acid (3.9 g, 26.3 mmole) were dissolved in 1,4-dioxane (150 mL) and the solution was degassed with argon. (Ph₃P)₄Pd (230 mg, 0.2 mmole) was added, followed by a solution of potassium carbonate (6.9 g, 50 mmole) in H₂O (20 mL). The reaction was heated at reflux under argon overnight, then was cooled to room temperature and diluted with EtOAc (200 mL). The solution was washed sequentially with H₂O and brine, dried (Na₂SO₄), and concentrated in vacuo. The solid residue was purified by flash chromatography on silica gel (5-10% EtOAc/CHCl₃) to afford a solid (2.5 g, 38%).

MS (ES) m/z 253.0 (M+H)⁺.

### (l) 3-Oxo-3,4-dihydro-2H-pyrido[3,2-b][1,4]oxazine-6-carboxaldehyde

The pyridine (1k) (1.2 g, 4.8 mmole) was dissolved in CH₂Cl₂ (200 mL) and the solution was cooled to −78° C. Ozone was bubbled through the solution with stirring until a pale blue color appeared, then the excess ozone was removed by bubbling oxygen through the solution for 15 min. Dimethylsulfide (1.76 mL, 24 mmole) was added to the solution, and the reaction was stirred at −78° C. for 3 hr, then at room temperature overnight. The solvent was removed in vacuo, and the residue was triturated with Et₂O (50 mL). The collected solid was washed with additional Et₂O and dried to afford a solid (700 mg, 82%).

MS (ES) m/z 179.0 (M+H)⁺.

### (m) Title Compound

The amine (1g) (0.4 g) and aldehyde (II) (0.212 g) were dissolved in DMF (7 mL), methanol (7 mL) and acetic acid (0.7 mL) and heated with 3 A molecular sieves for 2 hr at 75-80° C. for 2 hr, cooled, and treated with sodium cyanoborohydride (0.30 g) and the mixture was stirred overnight at room temperature. Chloroform was added and the mixture was filtered, treated with sodium carbonate solution and extracted with methanol-chloroform, dried (sodium sulfate), evaporated and chromatographed on silica gel (methanol-DCM) to afford a solid (0.45 g) which was recrystallised from methanol-ether to afford the pure racemic title compound (0.30 g) as the free base.

MS (ES) m/z 499/501 (M+H)⁺.

¹H NMR δH (CDCl₃, 400 MHz), 1.40-1.70 (2H, m), 1.88 (2H, br. d), 2.25 (2H, q), 2.52 (1H, m), 2.65 (1H, dd), 3.00 (2H, br t), 3.07 (1H, dd), 3.80 (2H, s), 4.03 (3H, s), 4.65 (2H, s), 5.67 (1H, m), 6.42 (1H, brd), 6.95 (1H, d), 7.15 (2H, 2×d), 8.21 (1H, d), 8.70 (1H, s).

This material, as a solution in chloroform/methanol, was treated with an excess of 4M HCl in dioxan and evaporated to dryness. The solid was triturated under ether, filtered and dried under vacuum to provide the title compound.

### Example 2

### (Racemic)-1-(3-Chloro-6-methoxy-[1,5]naphthyridin-4-yl)-2-{4-[(2,3-dihydro-[1,4]dioxino[2,3-c]pyridin-7-ylmethyl)-amino]-piperidin-1-yl}-ethanol Dihydrochloride

### (a) 5-Benzyloxy-2-hydroxymethyl-1H-pyridin-4-one

A mixture of 5-benzyloxy-2-hydroxymethyl-4-pyrone (prepared from Kojic acid by the method of D. Erol, J. Med. Chem., 1994, 29, 893) (9.7 g, 40 mmol), concentrated aqueous (880) ammonia (100 mL), and ethanol (20 mL) was heated to reflux overnight. The mixture was allowed to cool to room temperature then filtered. The resultant solid was washed with ether and dried in vacuo (5.9 g).

MS (APCl+) m/z 232 (MH+).

### (b) (2,3-Dihydro-[1,4]dioxino[2,3-c]pyridin-7-yl)-methanol

A solution of (2a) (2 g, 8.7 mmol) in water (220 mL) containing sodium hydroxide (17 mmol) was hydrogenated over 10% palladium on charcoal (1 g) for 4 hours. The mixture was filtered and evaporated to give a white solid. This solid was dissolved in N,N-dimethylformamide (8 mL) then treated with potassium carbonate (2.9 g) and 1,2-dibromoethane (0.6 mL, 7 mmol). The mixture was heated at 85° C. overnight. The cooled mixture was evaporated onto silica and chromatographed eluting with 10-30% methanol in ethyl acetate affording a white solid (250 mg, 21%).

MS (APCl+) m/z 168 (MH+).

### (c) 2,3-Dihydro-[1,4]dioxino[2,3-c]pyridine-7-carboxaldehyde

A solution of (2b) (250 mg, 1.5 mmol) in dichloromethane (5 mL) was treated with manganese dioxide (650 mg, 7.5 mmol). After 3 days the mixture was filtered and evaporated affording a white solid (150 mg, 61%).

MS (APCl+) m/z 166 (MH+).

### (d) Title Compound

The amine (1g) (0.57 g) and aldehyde (2c) (0.285 g) were dissolved in DMF (10 mL) and sodium triacetoxyborohydride (1.078 g) added and the solution was stirred overnight at room temperature. Chloroform was added and the mixture was filtered, treated with sodium carbonate solution and extracted with methanol-chloroform, dried (sodium sulfate), evaporated and chromatographed on silica gel (methanol-DCM) to afford the free base of the title compound as a solid (0.52 g), containing ca. 20% of the unwanted ‘epoxide wrong-opening’ isomer.

LC/MS (ES) two peaks Rt 1.31 and 1.21 minutes m/z 486/488 (M+H)⁺.

¹H NMR δH (CDCl₃, 400 MHz), 1.40-1.70 (2H, m), 1.88 (2H, br. d), 2.25 (2H, q), 2.52 (1H, m), 2.65 (1H, dd), 3.00 (2H, m), 3.10 (1H, dd), 3.80 (2H, s), 4.05 (3H, s), 4.25-4.35 (4H, m), 5.67 (1H, m), 6.38 (1H, br s), 6.83 (1H, s), 7.15 (1H, d), 8.05 (1H, s), 8.23 (1H, d), 8.70 (1H, s) (plus impurity peaks).

This material, as a solution in chloroform/methanol, was treated with an excess of 4M HCl in dioxan and evaporated to dryness. The solid was recrystallised from methanol to give the pure racemic title compound (0.395 g).

LC/MS (ES) single peak with Rt 1.31 minutes with m/z 486/88 (M+H)⁺.

### Example 3

### {1-[2-(3-Chloro-6-methoxy-[1,5]naphthyridin-4-yl)-ethyl]-piperidin-4-yl}-(2,3-dihydro-[1,4]dioxino[2,3-c]pyridin-7-ylmethyl)-amine Dihydrochloride

### (a) 7-Chloro-2-methoxy-8-vinyl-[1,5]naphthyridine

The triflate (1b) (1 g) in DME (20 mL) under argon, was treated with tetrakis(triphenylphosphine)palladium(0) (0.21 g) and the mixture stirred at room temperature for 20 minutes. Anhydrous potassium carbonate (0.403 g), water (6 mL), and vinylborane:pyridine complex (see F. Kerins and D O'Shea J. Org. Chem. 2002, 67, 4968-4971) (1.056 g) were added and the mixture was heated at 100° C. for 1.5 hr. It was cooled, diluted with water and extracted with ether, dried (sodium sulfate), evaporated and chromatographed on silica gel, eluting with DCM then chloroform to afford a white solid (0.53 g).

MS (ES) m/z 221/223 (M+H)⁺.

### (b) {1-[2-(3-Chloro-6-methoxy-[1,5]naphthyridin-4-yl)-ethyl]-piperidin-4-yl}-carbamic acid tert butyl ester

A mixture of the vinyl-naphthyridine (3a) (0.53 g) and piperidin-4-yl-carbamic acid tert-butyl ester (0.482 g) was heated at 95-100° C. for 10 hr, then the product was dissolved in chloroform and chromatographed on silica gel (DCM then methanol-DCM) to afford the solid product (0.31 g)

MS (ES) m/z 421/423 (M+H)⁺.

### (c) 1-[2-(3-Chloro-6-methoxy-[1,5]naphthyridin-4-yl)-ethyl]-piperidin-4-ylamine

The ester (3b) was dissolved in DCM (20 mL) and trifluoroacetic acid (20 mL) was added and the solution was left at room temperature for 1 hr then evaporated to dryness. It was treated with water and sodium carbonate and extracted with 1% methanol-chloroform, dried (sodium sulfate) and evaporated to give a foam (0.24 g).

MS (ES) m/z 321/323 (M+H)⁺.

### (d) Title Compound

The amine (3c) (0.24 g) and aldehyde (2c) (0.124 g) were dissolved in DMF (10 mL) and sodium triacetoxyborohydride (0.48 g) added and the solution was stirred overnight at room temperature. Chloroform was added and the mixture was filtered, treated with sodium carbonate solution and extracted with methanol-chloroform, dried (sodium sulfate), evaporated and chromatographed on silica gel (methanol-DCM) to afford the free base of the title compound as a solid (0.22 g).

MS (ES) m/z 470/472 (M+H)⁺.

¹H NMR δH (CDCl₃, 400 MHz), 1.40-1.60 (2H, m), 1.95 (2H, br. d), 2.25 (2H, t), 2.54 (1H, m), 2.70 (2H, m), 3.07 (2H, m), 3.55 (2H, m), 3.78 (2H, s), 4.05 (3H, s), 4.25-4.35 (4H, m), 6.82 (1H, s), 7.09 (1H, d), 8.10 (1H, s), 8.15 (1H, d), 8.65 (1H, s)

This material, as a solution in chloroform/methanol, was treated with an excess of 4M HCl in dioxan and evaporated to dryness. The solid was trituated with ether to give the title compound (0.224 g).

### Example 4

### {1-[2-(3-Chloro-6-methoxy-quinolin-4-yl)-ethyl]-piperidin-4-yl}-(2,3-dihydro-[1,4]dioxino[2,3-c]pyridin-7-ylmethyl)-amine Dihydrochloride

### (a) 3-Chloro-6-methoxy-quinolin-4-ol

6-Methoxy-quinolin-4-ol (18.5 g) in acetic acid (750 mL) was treated with N-chlorosuccinimide (15.52 g) and the mixture was heated at 60° C. for 4.5 hr, cooled, and evaporated. Excess sodium bicarbonate solution was added and the solid collected and washed with water and dried in vacuo at 40° C. overnight, to give a yellow solid (21.3 g).

MS (ES) m/z 210/212 (M+H)⁺.

### (b) 4-Bromo-3-chloro-6-methoxy-quinoline

The quinolin-4-ol (4a) in dry DMF (80 mL) was cooled in ice and phosphorus tribromide (15.6 mL) added drop-wise, and the mixture was stirred, with ice-cooling for 30 minutes then allowed to warm to room temperature and stirred for a further 3.5 hours. It was cooled in ice and sodium carbonate solution was added and the solid was collected, washed well with water, and dried in vacuo, to afford a pale yellow solid (13.2 g).

MS (ES) m/z 272/274/276 (M+H)⁺.

### (c) 7-Chloro-2-methoxy-8-vinyl-quinoline

The bromide (4b) (0.5 g) in DME (14 mL) under argon, was treated with tetrakis(triphenylphosphine)palladium(0) (0.104 g) and the mixture stirred at room temperature for 20 minutes. Anhydrous potassium carbonate (0.25 g), water (4 mL), and vinylborane:pyridine complex was added and the mixture was heated at 100° C. for 1 hr. It was cooled, diluted with water and extracted with ether, dried (sodium sulfate) and evaporated to dryness. As starting material (4b) was still present the crude reaction product was reacted again, as above, and heated for a further 6 hours. After work-up the product was chromatographed on silica gel, eluting with DCM to afford a white solid (0.35 g).

MS (ES) m/z 220/222 (M+H)⁺.

### (d) {1-[2-(3-Chloro-6-methoxy-quinolin-4-yl)-ethyl]-piperidin-4-yl-carbamic acid tert butyl ester

A mixture of the vinyl-quinoline (4c) (1.1 g) and piperidin-4-yl-carbamic acid tert-butyl ester (1.17 g) in chloroform (2 mL) was heated at 150° C. for 3 days, then the product was dissolved in DCM and chromatographed on silica gel (ethyl acetate-DCM) to afford the solid product (0.59 g)

MS (ES) m/z 420/422 (M+H)⁺.

### (e) 1-[2-(3-Chloro-6-methoxy-quinolin-4-yl)-ethyl]-piperidin-4-ylamine dihydrochloride

The ester (4d) (0.59 g) was dissolved in chloroform (15 mL) and a solution of 4M HCl in dioxan (3.5 mL) was added and the solution was stirred at room temperature for 2.5 hr then evaporated to dryness and azeotroped with toluene to give the product.

MS (ES) m/z 320/322 (M+H)⁺.

### (f) Title Compound

The amine (4e) (0.45 g) and aldehyde (2c) (0.24 g) were dissolved in DMF (15 mL) and triethylamine (0.78 mL) added followed by sodium triacetoxyborohydride (1.2 g) and the solution was stirred for 2 days at room temperature. The mixture was quenched with 2N HCl, basified with sodium bicarbonate solution, and extracted with methanol-DCM, dried (sodium sulfate), evaporated and chromatographed on silica gel (methanol-DCM) to afford the free base of the title compound as a solid (0.273 g).

MS (ES) m/z 469/471 (M+H)⁺.

¹H NMR δH (CD₃OD, 250 MHz), 1.55-1.80 (2H, m), 2.10 (2H, br. d), 2.25 (2H, t), 2.68 (2H, m), 2.91 (1H, m), 3.30 (2H, m), 3.45 (2H, m), 3.98 (3H, s), 4.04 (2H, s), 4.25-4.40 (4H, m), 6.95 (1H, s), 7.40 (1H, s) overlapping with 7.42 (1H, dd), 8.10 (1H, s), 8.60 (1H, s).

This material, as a solution in chloroform/methanol, was treated with an excess of 4M HCl in dioxan and evaporated to dryness. The solid was triturated with ether to give the title compound (0.33 g).

### Example 5

### 6-({(cis)-1-[2-(3-Chloro-6-methoxy-quinolin-4-yl)-ethyl]-3-hydroxy-piperidin-4-ylamino}-methyl)-4H-pyrido[3,2-b][1,4]oxazin-3-one Dihydrochloride Enantiomer 1

(a) cis-4-tert-Butoxycarbonylamino-3-hydroxy-piperidine-1-carboxylic acid benzyl ester. Racemic cis-4-tert-butoxycarbonylamino-3-hydroxy-piperidine-1-carboxylic acid benzyl ester was prepared according to the procedure outlined by Kim et al. [Syn. Comm. 2001, 31, 1081-1089] starting from 3,6-Dihydro-2H-pyridine-1-carboxylic acid benzyl ester.

MS (ES) m/z 351 (M+H)⁺.

### (b) cis-4-tert-Butoxycarbonylamino-3-hydroxy-piperidine-1-carboxylic acid benzyl ester enantiomer 1 and cis-4-tert-Butoxycarbonylamino-3-hydroxy-piperidine-1-carboxylic acid benzyl ester enantiomer 2

71.0 g of the racemate (5a) was dissolved in methanol (710 mL) and resolved through multiple injections (1×8 g substrate injection; 5×10 g substrate injection; 1×7 g substrate injection; and 1×6 g substrate injection) on a Chiralpak AD column (77×250 mm) eluting with 100% methanol at a flow rate of 280 mL/minute with UV detection at 254 nm. 31.15 g of cis-4-tert-butoxycarbonylamino-3-hydroxy-piperidine-1-carboxylic acid benzyl ester fast running enantiomer (>99% ee, retention time 3.8 minutes (sharp), designated enantiomer 1) and 26.75 g of cis-4-tert-butoxycarbonylamino-3-hydroxy-piperidine-1-carboxylic acid benzyl ester slow running enantiomer (>99% ee, retention time 8.0 minutes (very broad), designated enantiomer 2) were obtained.

### (c) cis-(3-Hydroxy-piperidin-4-yl)-carbamic acid tert-butyl ester enantiomer 1 and cis-(3-Hydroxy-piperidin-4-yl)-carbamic acid tert-butyl ester enantiomer 2

10.0 g of cis-4-tert-Butoxycarbonylamino-3-hydroxy-piperidine-1-carboxylic acid benzyl ester fast running (5b, enantiomer 1), was dissolved in methanol (350 mL) and was degassed. Pearlman's catalyst (palladium hydroxide on carbon, 20 wt % Pd (dry basis), ≦50% water, 500 mg) was added and the mixture was purged with hydrogen and stirring continued under a balloon of hydrogen for 12 hours. The mixture was degassed with argon, filtered through a pad of Celite, and evaporated to dryness to afford 6.2 g (100%) a white solid.

MS (ES) m/z 217 (M+H)⁺.

Similarly, the corresponding slower running enantiomer (5b, enantiomer 2) was converted to cis-(3-hydroxy-piperidin-4-yl)-carbamic acid tert-butyl ester (5c, enantiomer 2)

### (d) cis-{1-[2-(3-Chloro-6-methoxy-quinolin-4-yl)-ethyl]-3-hydroxy-piperidin-4-yl}-carbamic acid tert-butyl ester enantiomer 1

cis-(3-Hydroxy-piperidin-4-yl)-carbamic acid tert-butyl ester enantiomer 1 (5c, enantiomer 1) (473 mg, 2.2 mmole) and vinyl quinoline (4c) (480 mg, 2.2 mmole) were dissolved in a minimal amount of chloroform (2 mL) and then heated at 90° C. overnight. Purification by silica gel chromatography with 5% methanol/chloroform gave an oil (550 mg, 58%).

MS (ES) m/z 436 (M+H)⁺.

### (e) cis-4-Amino-1-[2-(3-chloro-6-methoxy-quinolin-4-yl)-ethyl]-piperidin-3-ol dihydrochloride dioxane solvate enantiomer 1

To a stirred solution of cis{1-[2-(3-chloro-6-methoxy-quinolin-4-yl)-ethyl]-3-hydroxy-piperidin-4-yl}-carbamic acid tert-butyl ester enantiomer 1 (5d, enantiomer 1), (550 mg, 1.3 mmole) in chloroform (2 mL) was added 4M HCl in dioxane (5 mL). Stirring was continued for 2 hours then toluene was added and the mixture concentrated under reduced pressure then dried under high vacuum to afford an off white solid (645 mg, 100%).

MS (ES) m/z 336 (M+H)⁺.

### (f) Title Compound

A solution of cis-4-Amino-1-[2-(3-chloro-6-methoxy-quinolin-4-yl)-ethyl]-piperidin-3-ol dihydrochloride dioxane solvate enantiomer 1 (335 mg, 0.68 mmole) in dichloromethane (6 mL) and methanol (2 mL) was treated with triethylamine (0.47 mL, 3.4 mmole) followed by 3-oxo-3,4-dihydro-2H-pyrido[3,2-b][1,4]oxazine-6-carboxaldehyde (1l) (120 mg, 0.68 mmole). After stirring overnight the mixure was cooled to 0° C. and sodium borohydride (26 mg, 0.68 mmole) was added. The reaction was stirred 2 hours at 0° C., then diluted with chloroform and washed with saturated aqueous sodium bicarbonate. The aqueous layer was extracted with chloroform, and the combined organics were dried over sodium sulfate, filtered and evaporated. The title compound was purified by silica gel chromatography eluting with 90:10:1 CHCl₃:MeOH:NH₄OH_{(aq)} to afford the free base of the title compound (259 mg, 83%).

¹H NMR δH (400 MHz, CDCl₃) [symbol] 8.64 (s, 1H), 7.99 (d, J=9.2 Hz, 1H), 7.35 (dd, J=2.7, 9.2 Hz, 1H), 7.23 (d, J=2.7 Hz, 1H), 7.20 (d, J=8.1Hz, 1H), 4.61 (s, 2H), 3.95 (s, 3H), 3.87 (m, 2H), 3.45 (bs, 1H), 3.35 (m, 2H), 3.19 (d, J=10.3, 1H), 3.02 (d, J=10.5, 1H), 2.60-2.80 (m, 4H), 2.35 (d, J=11.4, 1H), 2.21 (m, 1H), 1.70-1.93 (m, 3H). MS (ES) m/z 571.9 (M+H)⁺.

This material was converted to the dihydrochloride by dissolving in chloroform and adding 2 equivalents of 1M HCl/ether then evaporating to dryness.

### Example 6

### 6-({(cis)-1-[2-(3-Chloro-6-methoxy-quinolin-4-yl)-ethyl]-3-hydroxy-piperidin-4-ylamino}-methyl)-4H-pyrido[3,2-b][1,4]oxazin-3-one Dihydrochloride Enantiomer 2

The free base was prepared as in Example (5) from (5b) cis-(3-hydroxy-piperidin-4-yl)-carbamic acid tert-butyl ester (5c, enantiomer 2)

¹H NMR δH (400 MHz, CDCl₃) [symbol] 8.64 (s, 1H), 7.99 (d, J=9.2 Hz, 1H), 7.35 (dd, J=2.7, 9.2 Hz, 1H), 7.23 (d, J=2.7 Hz, 1H), 7.20 (d, J=8.1Hz, 1H), 4.61 (s, 2H), 3.95 (s, 3H), 3.87 (m, 2H), 3.45 (bs, 1H), 3.35 (m, 2H), 3.19 (d, J=10.3, 1H), 3.02 (d, J=10.5, 1H), 2.60-2.80 (m, 4H), 2.35 (d, J=11.4, 1H), 2.21 (m, 1H), 1.70-1.93 (m, 3H). MS (ES) m/z 571.9 (M+H)⁺.

This material was converted to the dihydrochloride by dissolving in chloroform and adding 2 equivalents of 1M HCl/ether then evaporating to dryness.

### Example 7

### 6-({(cis)-1-[2-(3-Chloro-6-methoxy-quinolin-4-yl)-ethyl]-3-hydroxy-piperidin-4-ylamino}-methyl)-4H-pyrido[3,2-b][1,4]thiazin-3-one Dihydrochloride Enantiomer 1

### (a) Methyl 3-oxo-3,4-dihydro-2H-pyrido[3,2-b][1,4]thiazine-6-carboxylate

A solution of ethyl 2-mercaptoacetate (1.473 mL) in DMF (48 mL) was ice-cooled and treated with sodium hydride (540 mg of a 60% dispersion in oil). After 1 hour methyl 6-amino-5-bromopyridine-2-carboxylate (3g) (T. R. Kelly and F. Lang, J. Org. Chem. 61, 1996, 4623-4633) was added and the mixture stirred for 16 hours at room temperature. The solution was diluted with EtOAc (1 litre), washed with water (3×300 mL), dried and evaporated to about 10 mL. The white solid was filtered off and washed with a little EtOAc to give the ester (0.95 g).

MS (APCl⁻) m/z 223 ([M−H]⁻, 100%)

### (b) 3-Oxo-3,4-dihydro-2H-pyrido[3,2-b][1,4]thiazine-6-carboxylic acid

A solution of ester (7a) (788 mg) in dioxan (120 ml)/water (30 mL) was treated dropwise over 2 hours with 0.5M NaOH solution (8 mL) and stirred overnight. After evaporation to approx. 3 ml, water (5 mL) was added and 2M HCl to pH4. The precipitated solid was filtered off, washed with a small volume of water and dried under vacuum to give a solid (636 mg).

MS (APCl⁻) m/z 209 ([M−H]⁻, 5%), 165([M-COOH]⁻, 100%)

### (c) 6-Hydroxymethyl-3-oxo-3,4-dihydro-2H-pyrido[3,2-b][1,4]thiazine

A solution of the carboxylic acid (7b) (500 mg) in THF (24 mL) with triethylamine (0.396 mL) was cooled to −10° C. and isobutyl chloroformate (0.339 ml) added. After 20 minutes the suspension was filtered through kieselguhr into an ice-cooled solution of sodium borohydride (272 mg) in water (8 mL), the mixture stirred 30 minutes and the pH reduced to 7 with dilute HCl. The solvent was evaporated and the residue triturated under water. The product was filtered and dried under vacuum to give a white solid (346 mg). MS (APCl⁻) m/z 195 ([M−H]⁻, 50%), 165(100%)

### (d) 3-Oxo-3,4-dihydro-2H-pyrido[3,2-b][1,4]thiazine-6-carboxaldehyde

A solution of the alcohol (7c) (330 mg) in dichloromethane (30 mL)/THF (30 mL) was treated with manganese dioxide (730 mg) and stirred at room temperature.

Further manganese dioxide was added after 1 hour (730 mg) and 16 hours (300 mg). After a total of 20 hours the mixture was filtered through kieselguhr and the filtrate evaporated. The product was triturated with EtOAc/hexane (1:1) and collected to give a solid (180 mg). MS (APCl⁻) m/z 195 ([M−H]⁻, 95%), 165 (100%)

### (e) Title Compound

The free base of the title compound was prepared from (5e) as in Example (5f), using the carboxaldehyde (7d).

¹H NMR δH (400 MHz, CDCl₃) δ 8.64 (s, 1H), 7.99 (d, J=9.2 Hz, 1H), 7.57 (d, J=7.8 Hz, 1H), 7.35 (dd, J=2.7, 9.2 Hz, 1H), 7.22 (d, J=2.7 Hz, 1H), 6.99(d, J=7.8 Hz, 1H), 3.95 (s, 3H), 3.88 (m, 2H), 3.46 (s, 2H), 3.35 (m, 3H), 3.19 (d, J=10.3, 1H), 3.02 (d, J=10.5, 1H), 2.60-2.80 (m, 3H), 2.34 (d, J=11.2, 1H), 2.20 (m, 1H), 1.70-1.93 (m, 4H). MS (ES) m/z 587.9 (M+H)⁺.

This material was converted to the dihydrochloride by dissolving in chloroform and adding 2 equivalents of 1M HCl/ether then evaporating to dryness.

### Example 8

### 6-({(cis)-1-[2-(3-Chloro-6-methoxy-quinolin-4-yl)-ethyl]-3-hydroxy-piperidin-4-ylamino}-methyl)-4H-pyrido[3,2-b][1,4]thiazin-3-one Dihydrochloride Enantiomer 2

The free base of the title compound was prepared by the method of Example (7), using instead cis-(3-hydroxy-piperidin-4-yl)-carbamic acid tert-butyl ester (5c, enantiomer 2)

¹H NMR δH (400 MHz, CDCl₃) [symbol] 8.64 (s, 1H), 7.99 (d, J=9.2 Hz, 1H), 7.57 (d, J=7.8 Hz, 1H), 7.35 (dd, J=2.7, 9.2 Hz, 1H), 7.22 (d, J=2.7 Hz, 1H), 6.99(d, J=7.8 Hz, 1H), 3.95 (s, 3H), 3.88 (m, 2H), 3.46 (s, 2H), 3.35 (m, 3H), 3.19 (d, J=10.3, 1H), 3.02 (d, J=10.5, 1H), 2.60-2.80 (m, 3H), 2.34 (d, J=11.2, 1H), 2.20 (m, 1H), 1.70-1.93 (m, 4H). MS (ES) m/z 587.9(M+H)⁺.

This material was converted to the dihydrochloride by dissolving in chloroform and adding 2 equivalents of 1M HCl/ether then evaporating to dryness.

### Example 9

### 6-({(cis)-1-[2-(3-Chloro-6-methoxy-[1,5]naphthyridin-4-yl)-ethyl]-3-hydroxy-piperidin-4-ylamino}-methyl)-4H-pyrido[3,2-b][1,4]oxazin-3-one Dihydrochloride Enantiomer 1

The free base of the title compound was prepared from 7-chloro-2-methoxy-8-vinyl-[1,5]naphthyridine (3a) in place of 7-chloro-2-methoxy-8-vinyl-quinoline by the method described in Example (5).

¹H NMR δH (CDCl₃, 400 MHz), 8.66 (s, 1H), 8.16 (d, J=9 Hz, 1H), 7.20 (d, J=8 Hz, 1H), 7.10 (d, J=9 Hz, 1H), 6.95 (d, J=8 Hz, 3H), 4.63 (s, 2H), 4.08 (s, 3H), 3.91-3.78 (m, 3H), 3.52 (t, J=8 Hz, 2H), 3.15 (m, 2H), 2.99 (m, 1H), 2.76 (dd, 13 Hz, 7 Hz), 2.53 (m, 2H), 2.38 (d, J=12 Hz, 1H), 2.25 (m, 1H), 1.70 (m, 1H), 1.03 (t, J=7 Hz, 1H). MS (ES) m/z 499 (M+H)⁺.

This material was converted to the dihydrochloride by dissolving in chloroform and adding 2 equivalents of 1M HCl/ether then evaporating to dryness.

### Example 10

### 6-({(cis)-1-[2-(3-Chloro-6-methoxy-[1,5]naphthyridin-4-yl)-ethyl]-3-hydroxy-piperidin-4-ylamino}-methyl)-4H-pyrido[3,2-b][1,4]oxazin-3-one Dihydrochloride Enantiomer 2

The free base of the title compound was prepared as described for Example (9) using cis-(3-hydroxy-piperidin-4-yl)-carbamic acid tert-butyl ester (5c, enantiomer 2)

¹H NMR δH (CDCl₃, 400 MHz), 8.66 (s, 1H), 8.16 (d, J=9 Hz, 1H), 7.20 (d, J=8 Hz, 1H), 7.10 (d, J=9 Hz, 1H), 6.95 (d, J=8 Hz, 3H), 4.63 (s, 2H), 4.08 (s, 3H), 3.91-3.78 (m, 3H), 3.52 (t, J=8 Hz, 2H), 3.15 (m, 2H), 2.99 (m, 1H), 2.76 (dd, 13 Hz, 7 Hz), 2.53 (m, 2H), 2.38 (d, J=12 Hz, 1H), 2.25 (m, 1H), 1.70 (m, 1H), 1.03 (t, J=7 Hz, 1H). MS (ES) m/z 499 (M+H)⁺.

This material was converted to the dihydrochloride by dissolving in chloroform and adding 2 equivalents of 1M HCl/ether then evaporating to dryness.

### Example 11

### 6-({(cis)-1-[2-(3-Chloro-6-methoxy-[1,5]naphthyridin-4-yl)-ethyl]-3-hydroxy-piperidin-4-ylamino}-methyl)-4H-pyrido[3,2-b][1,4]thiazin-3-one Dihydrochloride Enantiomer 1

The free base of the title compound was prepared as described in Example (7) starting with 7-chloro-2-methoxy-8-vinyl-[1,5]naphthyridine (3a) in place of 7-chloro-2-methoxy-8-vinyl-quinoline.

¹H NMR δH (CDCl₃, 400 MHz), 8.66 (s, 1H), 8.50 (bs, 1H), 8.16 (d, J=9 Hz, 1H), 7.57 (d, J=8 Hz, 1H), 7.10 (d, J=9 Hz, 1H), 7.00 (d, J=8 Hz, 1H), 4.08 (s, 3H), 3.91-3.81 (m, 3H), 3.52 (t, J=8 Hz, 2H), 3.46 (s, 2H), 3.15 (m, 1H), 2.99 (m, 1H), 2.75 (m, 2H), 2.55 (d, J=9 Hz, 1H), 2.37 (d, J=12 Hz, 1H), 2.25 (m, 1H), 1.70 (m, 3H).

MS (ES) m/z 515 (M+H)⁺.

This material was converted to the dihydrochloride by dissolving in chloroform and adding 2 equivalents of 1M HCl/ether then evaporating to dryness.

### Example 12

### 6-({(cis)-1-[2-(3-Chloro-6-methoxy-[1,5]naphthyridin-4-yl)-ethyl]-3-hydroxy-piperidin-4-ylamino}-methyl)-4H-pyrido[3,2-b][1,4]thiazin-3-one Dihydrochloride Enantiomer 2

The free base of the title compound was prepared as described for Example (11) using cis-(3-hydroxy-piperidin-4-yl)-carbamic acid tert-butyl ester (5c, enantiomer 2)

¹H NMR δH (CDCl₃, 400 MHz), 8.66 (s, 1H), 8.50 (bs, 1H), 8.16 (d, J=9 Hz, 1H), 7.57 (d, J=8 Hz, 1H), 7.10 (d, J=9 Hz, 1H), 7.00 (d, J=8 Hz, 1H), 4.08 (s, 3H), 3.91-3.81 (m, 3H), 3.52 (t, J=8 Hz, 2H), 3.46 (s, 2H), 3.15 (m, 1H), 2.99 (m, 1H), 2.75 (m, 2H), 2.55 (d, J=9 Hz, 1H), 2.37 (d, J=12 Hz, 1H), 2.25 (m, 1H), 1.70 (m, 3H). MS (ES) m/z 515 (M+H)⁺.

This material was converted to the dihydrochloride by dissolving in chloroform and adding 2 equivalents of 1M HCl/ether then evaporating to dryness.

### Example 13

### 6-({1-[2-(3-Chloro-6-methoxyquinolin-4-yl)ethyl]piperidin-4-yl amino}methyl)-4H-pyrido[3,2-b][1,4]thiazin-3-one Trihydrochloride

### (a) {1-[2-(3-Chloro-6-methoxyquinolin-4-yl)ethyl]piperidin-4-yl}carbamic acid tert-butyl ester

To a solution of 4-N-Boc-aminopiperidine (0.60 g, 3.01 mmole) in DMF (5 mL) at RT was added 3-chloro-6-methoxy-4-vinyl quinoline (0.60 g, 2.74 mmole). After 18 h at 100° C., the reaction solution was concentrated under vacuum and purified by flash chromatography on silica gel (CHCl₃MeOH containing 5% NH₄OH, 9:1) to afford a tan solid (0.97 g, 85%).

LC-MS (ES) m/z 420 (M+H)⁺

### (b) {1-[2-(3-Chloro-6-methoxyquinolin-4-yl)ethyl]piperidin-4-ylamine

To a solution of {1-[2-(3-chloro-6-methoxyquinolin-4-yl)ethyl]piperidin-4-yl)carbamic acid tert-butyl ester (13a) (0.97 g, 2.33 mmole) in CH₂Cl₂ at RT was added TFA (1:1, v/v). After 2 hrs, the solution was concentrated to dryness under vacuum and the residue redissolved in CH₂Cl₂/MeOH (9:1, v/v). The solution was washed with saturated aqueous NaHCO₃ solution, dried over Na₂SO₄, and concentrated under vacuum to give a waxy yellow solid (0.68 g, 92%).

LC-MS (ES) m/z 320 (M+H)⁺.

### (c) Title Compound

To a solution of {1-[2-(3-chloro-6-methoxyquinolin-4-yl)ethyl]piperidin-4-ylamine (13b) (0.16 g, 0.50 mmole) in CH₂Cl₂ (25 mL) and EtOH (25 mL) was added Na₂SO₄ (50 mg) and 3-oxo-3,4-dihydro-2H-pyrido[1,4]thiazine-6-carboxaldehyde (7d) (0.11 g, 0.55 mmole). After 12 hr at RT, NaBH₄ (21 mg, 0.55 mmole) was added and the reaction solution was allowed to stir overnight. Silica gel (−5 g) was added to the reaction solution and the contents were concentrated under vacuum. The silica-adsorbed reaction contents were added directly to a silica gel column and eluted (CHCl₃/MeOH containing 5% NH₄OH, 9:1) to give the free base of the title compound (0.21 g, 86%) as an off-white solid:

¹H NMR (400 MHz, d₄-MeOH) 8.76 (s, 1H), 8.00 (d, J=9.2 Hz, 1H), 7.84 (d, J=7.5 Hz, 1H), 7.60 (s, 1H), 7.52 (d, J=9.3 Hz, 1H), 7.18 (d, J=7.5 Hz, 1H), 4.47 (s, 2H), 4.12 (s, 3H), 4.01 (m, 2H), 3.91 (m, 2H), 3.57 (s, 2H), 3.37 (m, 5H), 2.59 (m, 2H), 2.33 (m, 2H). LC-MS (ES) m/z 498 (M+H)⁺.

This material was converted to the hydrochloride salt by dissolving in chloroform and adding 3 equivalents of 1M HCl/ether then evaporating to dryness.

### Example 14

### 6-({1-[2-(3-chloro-6-methoxyquinolin-4-yl)ethyl]piperidin-4-yl amino}methyl)-4H-pyrido[3,2-b][1,4]oxazin-3-one Trihydrochloride

This was prepared by the procedure of Example (13c), except substituting 3-oxo-3,4-dihydro-2H-pyrido[3,2-b][1,4]oxazine-6-carboxaldehyde (1l) (0.10 g, 0.55 mmole) for 3-oxo-3,4-dihydro-2H-pyrido[3,2-b][1,4]thiazine-6-carboxaldehyde, giving the free base of the title compound (0.19 g, 81%) as an off-white solid following flash chromatography on silica gel (CHCl₃/MeOH, 9:1, containing 5% NH₄OH).

¹H NMR (400 MHz, d₄-MeOH) 8.85 (s, 1H), 8.03 (d, J=9.2 Hz, 1H), 7.93 (s, 1H), 7.60 (d, J=7.5 Hz, 1H), 7.57 (m, 1H), 7.17 (m, 1H), 4.40 (s, 2H), 4.13 (s, 3H), 4.09 (m, 2H), 3.95 (m, 2H), 3.71 (m, 2H), 3.53 (s, 2H), 3.37 (m, 3H), 2.59 (m, 2H), 2.32 (m, 2H). LC-MS (ES) m/z 482 (M+H)⁺.

This material was converted to the hydrochloride salt by dissolving in chloroform and adding 3 equivalents of 1M HCl/ether then evaporating to dryness.

### Example 15

### 6-({1-[2-(3-Chloro-6-methoxynaphthyridin-4-yl)ethyl]piperidin-4-yl amino}methyl)-4H-pyrido[3,2-b][1,4]thiazin-3-one Dihydrochloride

This was prepared by the procedure of Example (13c), except substituting 1-[2-(3-chloro-6-methoxynaphthyridin-4-yl)ethyl]piperidin-4-ylamine (0.18 g, 0.56 mmole) [prepared from 4-N-Boc-aminopiperidine and 7-chloro-2-methoxy-8-vinyl-[1,5]naphthyridine (3a)] by the method of Examples (13a/b) to give the free base of the title compound (0.15 g, 53%), as an off-white solid following flash chromatography on silica gel (CHCl₃/MeOH, 9:1, containing 5% NH₄OH).

¹H NMR (400 MHz, d₆-DMSO) 8.84 (s, 1H), 8.33 (d, J=9.0 Hz, 1H), 7.91 (d, J=7.8 Hz, 1H), 7.34 (d, J=9.0 Hz, 1H), 7.29 (d, J=7.8 Hz, 1H), 4.25 (m, 2H), 4.12 (s, 3H), 3.81 (m, 4H), 3.61 (s, 2H), 3.49 (m, 1H), 3.27 (m, 2H), 3.11 (m, 2H), 2.51 (m, 2H), 2.20 (m, 2H). LC-MS (ES) m/z 499 (M+H)⁺.

This material was converted to the hydrochloride salt by dissolving in chloroform and adding 2 equivalents of 1M HCl/ether then evaporating to dryness.

### Example 16

### 6-({1-[2-(3-chloro-6-methoxynaphthyridin-4-yl)ethyl]piperidin-4-yl amino}methyl)-4H-pyrido[3,2-b][1,4]oxazin-3-one Dihydrochloride

This was prepared according to the procedure of Example (13c), except substituting 1-[2-(3-chloro-6-methoxynaphthyridin-4-yl)ethyl]piperidin-4-ylamine (0.18 g, 0.56 mmole) for 1-[2-(3-chloro-6-methoxyquinolinyl-4-yl)ethyl]piperidin-4-ylamine, and substituting 3-oxo-3,4-dihydro-2H-pyrido[3,2-b][1,4]oxazine-6-carboxaldehyde (1l) (0.10 g, 0.56 mmole) for 3-oxo-3,4-dihydro-2H-pyrido[3,2-b][1,4]thiazine-6-carboxaldehyde, to give the free base of the title compound (0.23 g, 84%), as an off-white solid following flash chromatography on silica gel (CHCl₃/MeOH, 9:1, containing 5% NH₄OH).

¹H NMR (400 MHz, d₆-DMSO) 8.84 (s, 1H), 8.33 (d, J=9.0 Hz, 1H), 7.47 (d, J=8.0 Hz, 1H), 7.34 (d, J=9.1Hz, 1H), 7.28 (d, J=8.0 Hz, 1H), 4.70 (m, 2H), 4.18 (m, 2H), 4.12 (s, 3H), 3.81 (m, 4H), 3.42 (m, 1H), 3.38 (m, 2H), 3.25 (m, 2H), 2.40 (m, 2H), 2.18 (m, 2H). LC-MS (ES) m/z 483 (M+H)⁺.

This material was converted to the hydrochloride salt by dissolving in chloroform and adding 2 equivalents of 1M HCl/ether then evaporating to dryness.

### Example 17

### 6-({(trans)-1-[2-(3-Chloro-6-methoxyquinolin-4-yl)ethyl]3-hydroxypiperidin-4-yl amino}methyl)-4H-pyrido[3,2-b][1,4]thiazin-3-one Trihydrochloride enantiomer 2

### (a) N-Carbobenzoxy-1,2,3,6-tetrahydropyridine

20 g (0.24 mole) of 1,2,3,6 tetrahydropyridine was added to 25 mL of 10% aqueous Na₂CO₃ and cooled to 0 C. 34.3 mL (0.24 mole) of benzyl chloroformate was added dropwise over 1 hr. The contents were allowed to stir overnight, coming to room temperature during the interim. The reaction mixture was diluted with 500 mL of brine, and extracted several times with Et₂O. The organic layers were combined, dried over MgSO₄, filtered, and evaporated to dryness. The crude material was purified by flash chromatography on silica gel using 10% EtOAc/Hexanes as the eluent to give (24.5 g, 47%).

¹H NMR (MeOD, 400 MHz) [symbol] 7.38-7.29 (m, 5H), 6.04-5.93 (m, 1H), 5.83-5.72 (m, 1H). 5.15 (s, 2H), 4.09-3.98 (m, 2H), 3.72-3.62 (m, 2H), 2.24-2.18 (m, 2H). LC-MS (ES) m/z 218 (M+H)⁺.

### (b) N-Carbobenzoxy-3,4-epoxypiperidine

To a cooled (0° C.) solution of N-carbobenzoxy-1,2,3,6-tetrahydropyridine (17a) (24.5 g, 0.11 mole) in 200 mL of DCM, was added a solution of m-chloroperbenzoic acid (27 g, 0.16 mole) in 200 mL of DCM dropwise over 30 min. The contents were allowed to warm to room temperature and continue to stir for 4 hrs. The reaction mixture was then washed (3×300 mL) with 5% aq. K₂CO₃ and (3×300 mL) with brine. The organic fraction was dried with MgSO₄, filtered and evaporated to a colorless oil. The crude material was purified by flash chromatography on silica gel using 20% EtOAc/hexanes as the eluent to give 23.1 g (91%).

¹H NMR (MeOD, 400 MHz) δ 7.38-7.29 (m, 5H), 5.12 (s, 2H), 4.01-3.87 (m, 2H) 3.43-3.25 (m, 4H), 2.15-2.90 (m, 2H). LC-MS (ES) m/z 234 (M+H)⁺

### (c) N-Carbobenzoxy-trans-3-hydroxy-4-azidopiperidine

10.6 g (0.2 mole) of NH₄Cl was dissolved in 30 mL of water. This solution was then diluted to 8:1 with MeOH (240 mL). To the solution was added 23.7 g (0.1 mole) of N-carbobenzoxy-3,4-epoxypiperidine (17b), followed by 6.5 g (0.12 mole) of sodium azide. The contents were heated to 65° C. overnight. The contents were concentrated down by rotary evaporation (approx. 50 mL), and partioned between EtOAc (300 mL) and water (300 mL). The organic layer was further washed with water (1×200 mL) and brine (2×250 mL). Organic layer was then dried over MgSO₄, filtered and evaporated to dryness. Crude material was purified by flash chromatography on silica gel using 30% EtOAc/hexanes to give 20.5 g (74%).

¹H NMR (DMSO, 400 MHz) δ7.24-7.15 (m, 5H), 5.48-5.47 (m, 1H), 4.90 (s, 2H) 3.84-3.70 (m, 2H), 3.32-3.12 (m, 2H), 2.95-2.55 (m, 2H), 1.73-1.69 (m, 1H), 1.16-1.06 (m, 1H). LC-MS (ES) m/z 277 (M+H)⁺

### (d) N-Carbobenzoxy-trans-3-hydroxy-4-aminopiperidine

20 g of N-Carbobenzoxy-trans-3-hydroxy-4-azidopiperidine (17c) was dissolved in 300 mL of EtOAc and degassed several times from alternating vacuum/N₂. 1.0 g of 5% Pd/C (Degussa type) was added and the contents were degassed again before being placed under atmospheric H₂ overnight. The following day, a tlc sample indicated the reaction was not complete. 500 mg of 10% Pd/C was added, the contents degassed and placed under atmospheric H₂ for 4 hrs. Reaction was nearly complete by tlc. The contents were filtered through a pad of Celite, and the Celite washed with MeOH. The solution was evaporated to dryness and purified by flash chromatography on silica gel using 10% MeOH/DCM and going to 90:10:1 DCM/MeOH/NH₄OH as the elution system to give 11.4 g (63%).

¹H NMR (CDCl₃, 400 MHz) [symbol] 7.16-7.07 (m, 5H), 4.89 (s, 2H), 4.19-3.91 (m, 2H), 3.12-3.02 (m, 1H), 2.78-2.68 (m, 1H), 2.60-2.47 (m, 2H), 1.83-1.76 (m, 1H), 1.33-1.25 (m, 1H).

LC-MS (ES) m/z 251 (M+H)⁺

### (e) racemic trans-4-tert-Butoxycarbonylamino-3-hydroxy-piperidine-1-carboxylic acid benzyl ester

11.4 g (45.6 mmol) of N-carbobenzoxy-trans-3-hydroxy-4-aminopiperidine (17d) was dissolved in 200 mL of DCM. A solution of di-tert-butyl dicarbonate (9.94 g, 45.6 mmol) in 50 mL of DCM was added slowly via addition funnel. The contents were allowed to stir overnight at room temperature. The contents were evaporated to dryness, to give (16g) (quant).

¹H NMR (DMSO, 400 MHz) [symbol] 7.38-7.32 (m, 5H), 6.83 (d, 1H), 5.06 (s, 2H), 5.01 (m, 1H), 3.98-3.79 (m, 2H), 3.34-3.26 (m, 2H), 3.95-3.62 (m, 2H), 1.95-1.90 (m, 1H), 1.38 (s, 9H), 1.32-1.25 (m, 1H). LC-MS (ES) m/z 351 (M+H)⁺

### (f) trans-4-tert-Butoxycarbonylamino-3-hydroxy-piperidine-1-carboxylic acid benzyl ester enantiomer 1 and trans-4-tert-Butoxycarbonylamino-3-hydroxy-piperidine-1-carboxylic acid benzyl ester enantiomer 2

14.0 g of the racemic trans-4-tert-butoxycarbonylamino-3-hydroxy-piperidine-1-carboxylic acid benzyl ester (17e) was dissolved in methanol (288 mL) and resolved through multiple injections (2×1 g substrate injection; 6×2 g substrate injection) on a Chiralpak AD column (77×250 mm) eluting with 100% methanol at a flow rate of 280 mL/minute with UV detection at 254 nm. 6.23 g of trans-4-tert-butoxycarbonylamino-3-hydroxy-piperidine-1-carboxylic acid benzyl ester fast running enatiomer (99% ee, retention time 3.8 minutes, designated enantiomer 1) and 6.10 g of trans-4-tert-butoxycarbonylamino-3-hydroxy-piperidine-1-carboxylic acid benzyl ester slow running enantiomer (99% ee, retention time 6.4 minutes, designated enantiomer 2) were obtained.

### (g) Title Compound

This was prepared by hydrogenation of piperidine (17f, enantiomer 2) (0.31 g) over Pearlman's catalyst by the method of Example (5c), followed by reaction with the vinyl quinoline (4c), removal of the Boc protecting group, and reaction with the carboxaldehyde (7d) by the methods of Example (5d-f) to give the free base of the title compound (0.39 g, 86%) as an off-white solid following flash chromatography on silica gel (CHCl₃/MeOH, 9:1, containing 5% NH₄OH)

¹H NMR (400 MHz, d₄-MeOH) 8.66 (s, 1H), 7.94 (m, 1H), 7.82 (d, J=7.8 Hz, 1H), 7.53 (s, 1H), 7.45 (d, J=9.2 Hz, 1H), 7.19 (d, J=7.8 Hz), 4.53 (s, 2H), 4.45 (m, 1H), 4.09 (s, 3H), 3.85 (m, 4H), 3.59 (m, 1H), 3.53 (s, 2H), 3.42 (m, 3H), 3.21 (m, 1H), 2.67 (m, 1H), 2.32 (m, 1H). LC-MS (ES) m/z 514 (M)⁺.

This material was converted to the hydrochloride salt by dissolving in chloroform and adding 3 equivalents of 1M HCl/ether then evaporating to dryness.

### Example 18

### 6-({(trans)-1-[2-(3-Chloro-6-methoxyquinolin-4-yl)ethyl]3-hydroxypiperidin-4-yl amino}methyl)-4H-pyrido[3,2-b][1,4]oxazin-3-one Trihydrochloride enantiomer 2

This was prepared from trans-4-amino-1-[2-(3-chloro-6-methoxyquinolin-4-yl)ethyl]piperidin-3-ol enantiomer 2 [prepared from Example (17f, enantiomer 2) by hydrogenation] (0.31 g) by the method of Example (17g) using 3-oxo-3,4-dihydro-2H-pyrido[3,2-b][1,4]oxazine-6-carboxaldehyde (1l) to give the free base of the title compound (0.37 g, 81%), as an off-white solid following flash chromatography on silica gel (CHCl₃/MeOH, 9:1, containing 5% NH₄OH).

¹H NMR (400 MHz, d₄-MeOH) 8.60 (s, 1H), 7.93 (m, 1H), 7.45 (m, 3H), 7.14 (d, J=8.1Hz), 4.70 (s, 2H), 4.43 (s, 1H), 4.20 (m, 1H), 4.05 (s, 3H), 3.68 (m, 4H), 3.32 (m, 2H), 3.14 (m, 2H), 2.80 (m, 2H), 2.49 (m, 1H), 2.05 (m, 1H). LC-MS (ES) m/z 498 (M+H)⁺.

This material was converted to the hydrochloride salt by dissolving in chloroform and adding 3 equivalents of 1M HCl/ether then evaporating to dryness.

### Example 19

### 6-(trans)-1-[2-(3-Chloro-6-methoxyquinolin-4-yl)ethyl]3-hydroxypiperidin-4-yl amino}methyl)-4H-pyrido[3,2-b][1,4]thiazin-3-one Trihydrochloride enantiomer 1

This was prepared from trans-4-amino-1-[2-(3-chloro-6-methoxyquinolin-4-yl)ethyl]piperidin-3-ol enantiomer 1 (0.31 g) [prepared from Example (17f, enantiomer 1) by hydrogenation] by the method of Example (17g) to give the free base of the title compound (0.39 g, 86%), as an off-white solid following flash chromatography on silica gel (CHCl₃/MeOH, 9:1, containing 5% NH₄OH).

¹H NMR (400 MHz, d₄-MeOH) 8.66 (s, 1H), 7.94 (m, 1H), 7.82 (d, J=7.8 Hz, 1H), 7.53 (s, 1H), 7.45 (d, J=9.2 Hz, 1H), 7.19 (d, J=7.8 Hz), 4.53 (s, 2H), 4.45 (m, 1H), 4.09 (s, 3H), 3.85 (m, 4H), 3.59 (m, 1H), 3.53 (s, 2H), 3.42 (m, 3H), 3.21 (m, 1H), 2.67 (m, 1H), 2.32 (m, 1H). LC-MS (ES) m/z 514 (M)+

This material was converted to the hydrochloride salt by dissolving in chloroform and adding 3 equivalents of 1M HCl/ether then evaporating to dryness.

### Example 20

### 6-(trans)-1-[2-(3-Chloro-6-methoxyquinolin-4-yl)ethyl]3-hydroxypiperidin-4-yl amino}methyl)-4H-pyrido[3,2-b][1,4]oxazin-3-one Trihydrochloride enantiomer 1

This was prepared from trans-4-amino-1-[2-(3-chloro-6-methoxyquinolin-4-yl)ethyl]piperidin-3-ol enantiomer 1 [prepared from Example (17f, enantiomer 1) by hydrogenation] (0.31 g) by the method of Example (17g) using 3-oxo-3,4-dihydro-2H-pyrido[3,2-b][1,4]oxazine-6-carboxaldehyde (1l) to give the free base of the title compound (0.37 g, 81%), as an off-white solid following flash chromatography on silica gel (CHCl₃/MeOH, 9:1, containing 5% NH₄OH).

¹H NMR (400 MHz, d₄-MeOH) 8.60 (s, 1H), 7.93 (m, 1H), 7.45 (m, 3H), 7.14 (d, J=8.1Hz), 4.70 (s, 2H), 4.43 (s, 1H), 4.20 (m, 1H), 4.05 (s, 3H), 3.68 (m, 4H), 3.32 (m, 2H), 3.14 (m, 2H), 2.80 (m, 2H), 2.49 (m, 1H), 2.05 (m, 1H). LC-MS (ES) m/z 498 (M+H)⁺.

This material was converted to the hydrochloride salt by dissolving in chloroform and adding 3 equivalents of 1M HCl/ether then evaporating to dryness.

### Example 21

### 6-({1-[2-(3-Chloro-6-methoxyquinolin-4-yl)ethyl]4-hydroxymethylpiperidin-4-ylamino}methyl)-4H-pyrido[3,2-b][1,4]thiazin-3-one Dihydrochloride

### (a) 4-Benzyloxycarbonylaminopiperidine-1,4-dicarboxylic acid mono-tert-butyl ester

To a solution of 4-aminopiperidine-1,4-dicarboxylic acid mono-tert-butyl ester (10.0 g, 40.9 mmole) in 300 mL H₂O, 50 mL 1 N NaOH and 50 mL DME was added Cbz-succinimide (15.3 g, 61.4 mmole). After 12 h, the reaction solution was readjusted to pH=9 with 1N NaOH. After a total of 36 hrs, the reaction solution was concentrated under vacuum, washed with Et₂O (3×200 mL) and acidified to pH=4 with 1M HCl. The reaction contents were extracted with EtOAc (4×200 mL) and the organics washed with H₂O, brine and then dried over Na₂SO₄ and concentrated. Et₂O was added to the residue for trituration and the remaining solid was filtered to give a white solid (12.0 g, 78%). LC-MS (ES) m/z 379 (M+H)⁺.

### (b) 4-Benzyloxycarbonylaminopiperidine-1,4-dicarboxylic acid-1-tert-butyl ester-4-methyl ester

To a solution of 4-benzyloxycarbonylaminopiperidine-1,4-dicarboxylic acid mono-tert-butyl ester (21a) (12.0 g, 31.7 mmole) in acetone at RT was added K₂CO₃ (8.75 g, 63.4 mmole) and methyl iodide (4.95 g, 34.9 mmole). After 36 h, the reaction solution was filtered through a sinter-glass funnel and the filtrate partitioned between CH₂Cl₂/H₂O (400 mL, 1:1, v/v). The phases were separated and the organic phase was washed with 1N HCl, brine and then concentrated under vacuum. The residual oil was purified on silica (hexanes/EtOAc, 1:1) to give a colorless oil (11.2 g, 90%).

LC-MS (ES) m/z 393 (M+H)⁺.

### (c) 4-Benzyloxycarbonylaminopiperidine-4-carboxylic acid methyl ester

To a solution of 4-benzyloxycarbonylaminopiperidine-1,4-dicarboxylic acid-1-tert-butyl ester-4-methyl ester (21b) (11.2 g, 28.5 mmole) in CH₂Cl₂ (250 mL) at RT was added TFA (50 mL). After 3 h, the reaction solution was concentrated under vacuum and the residue dissolved in CH₂Cl₂ (200 mL) and MeOH (20 mL). The solution was washed with saturated NaHCO₃ solution, dried over Na₂SO₄ and concentrated to a waxy off-white solid which was used directly without further purification.

LC-MS (ES) m/z 293 (M+H)⁺.

### (d) 4-Benzyloxycarbonylamino-1-[2-(3-chloro-6-methoxyquinolin-4-yl)ethyl]piperidine-4-carboxylic acid methyl ester

To a solution of 4-benzyloxycarbonylaminopiperidine-4-carboxylic acid methyl ester (21c) (1.33 g, 4.56 mmole) in DMF (5 mL) at RT was added 7-chloro-2-methoxy-8-vinyl-quinoline (4c) (1.0 g, 4.56 mmole). After 18 h at 100° C., the reaction solution was concentrated under vacuum and purified by flash chromatography on silica gel (CHCl₃/MeOH containing 5% NH₄OH, 9:1) to afford an off-white solid (1.84 g, 79%).

LC-MS (ES) m/z 512 (M+H)⁺

### (e) (4-amino-1-[2-(3-chloro-6-methoxyquinolin-4-yl)ethyl]piperidin-4-yl)methanol

To a solution of 4-benzyloxycarbonylamino-1-[2-(3-chloro-6-methoxyquinolin-4-yl)ethyl]piperidine-4-carboxylic acid methyl ester (21d) (0.11 g, 0.21 mmole) in EtOH (40 mL) at RT was added Pd(OH)₂. After 12 hrs under a balloon of H₂, the reaction solution was filtered through Celite (MeOH) and the filtrate concentrated to dryness under vacuum. The colorless residue was dissolved in THF (10 mL), cooled to 0° C. and LiAlH₄ (0.21 mmole, 1M in THF) was added. After 1.5 h, 1M NaOH solution (10 mL) was added and the solution extracted with CH₂Cl₂. The organic solution was dried over Na₂SO₄, and concentrated under vacuum to give a colorless oil, which was used directly in the following step.

LC-MS (ES) m/z 350 (M+H)⁺.

### (f) Title Compound

To a solution of {4-amino-1-[2-(3-chloro-6-methoxyquinolin-4-yl)ethyl]piperidin-4-yl}methanol (21e) (0.05 g, 0.14 mmole) in CH₂Cl₂ (25 mL) and EtOH (25 mL) was added Na₂SO₄ (50 mg) and 3-oxo-3,4-dihydro-2H-pyrido[1,4]thiazine-6-carboxaldehyde (7d) (0.04 g, 0.2 mmole). After 12 hr at RT, NaBH₄ (5 mg, 0.14 mmole) was added and the reaction solution was allowed to stir overnight. Silica gel (−5 g) was added to the reaction solution and the contents were concentrated under vacuum. The silica-adsorbed reaction contents were added directly to a silica gel column and eluted (CHCl₃/MeOH containing 5% NH₄OH, 9:1) to give the free base of the title compound (0.06 g, 82%) as an off-white solid.

¹H NMR (400 MHz, d₄-MeOH) 8.64 (s, 1H), 7.95 (m, 2H), 7.83 (d, J=7.8 Hz, 1H), 7.52 (s, 1H), 7.43 (d, J=9.2 Hz, 1H), 7.20 (d, J=7.8 Hz, 1H), 4.41 (s, 2H), 4.09 (s, 3H), 3.90 (m, 2H), 3.83 (m, 2H), 3.58 (s, 2H), 3.33 (m, 4H), 2.50 (m, 4H), 2.33 (m, 2H). LC-MS (ES) m/z 528 (M+H)⁺.

This material was converted to the hydrochloride salt by dissolving in chloroform and adding 2 equivalents of 1M HCl/ether then evaporating to dryness

### Example 22

### 6-({1-[2-(3-Chloro-6-fluoro-5-methoxy-quinolin-4-yl)-ethyl]-piperidin-4-ylamino}-methyl)-4H-pyrido[3,2-b][1,4]thiazin-3-one Dihydrochloride

### (a) Carbonic acid 4-bromo-2-fluoro-phenyl ester ethyl ester

A solution of 4-bromo-2-fluorophenol (25 g, 130 mmol) and triethylamine (21.6 mL, 155 mmol) in dichloromethane (120 mL) was treated at 0° C. with ethyl chloroformate (14.8 mL, 155 mmol). The reaction mixture was stirred at ambient temperature for 1.5 hours then washed with water, dried and evaporated affording an oil (32 g, 93%). MS (+ve ion electrospray) m/z 264 (MH⁺).

### (b) 4-Bromo-2-fluoro-5-nitro-phenol

A solution of (22a) (32 g, 122 mmol) in concentrated sulphuric acid (55 mL) was added dropwise to fuming nitric acid (8.4 mL, 195 mmol) while maintaing the temperature between 10-20° C. by the use of an ice-water cooling bath (CAUTION—careful temperature monitoring required). After 2 hours the reaction mixture was poured onto ice-water and extracted several times with ethyl acetate. The combined organic extracts were dried and evaporated affording an oil (35g). This was dissolved in methanol (200 mL) and treated with sodium hydrogen carbonate (19 g, 227 mmol). The mixture was stirred at 60° C. for 4 hours then concentrated to near-dryness. Water (60 mL) was added and 5M hydrochloric acid added until pH 5 was attained. The reaction mixture was extracted several times with ethyl acetate. The combined organic extracts were dried and evaporated affording an oil (29 g, 83%).

MS (+ve ion electrospray) m/z 237 (MH⁺).

### (c) 1-Bromo-5-fluoro-4-methoxy-2-nitro-benzene

A solution of (22b) (25 g, 106 mmol) in DMF (200 mL) was treated with potassium carbonate (27 g, 198 mmol) and methyl iodide (12 mL, 198 mmol) then heated at 60° C. for 5 hours. The mixture was evaporated and the residue was partitioned between ethyl acetate and water. The aqueous extract was further extracted with ethyl acetate and the combined organic extracts dried and evaporated affording an oil (25.6 g, 97%). MS (+ve ion electrospray) m/z 251 (MH⁺).

### (d) 2-Bromo-4-fluoro-5-methoxy-phenylamine

A mixture of (22c) (25.5 g, 96 mmol), acetic acid (250 mL), ethanol (250 mL) and iron powder (21.5 g, 385 mmol) was heated at 100° C. for 4 hours. After allowing to cool to room temperature, the mixture was diluted with water (500 mL) and neutralised with solid potassium carbonate. The mixture was filtered through Kieselguhr and extracted (3 times) with dichloromethane. This was concentrated to approximately 300 mL and passed through a plug of silica gel. Evaporation afforded an orange solid (15.0, 67%). MS (+ve ion electrospray) m/z 221 (MH⁺).

### (e) 5-[(2-Bromo-4-fluoro-5-methoxy-phenylamino)-methylene]-2,2-dimethyl-[1,3]dioxane-4,6-dione

A mixture of amine (22d) (15 g, 68 mmol), triethyl orthoformate (13.6 mL, 82 mmol) and 2,2-dimethyl-[1,3]dioxane-4,6-dione (Meldrums acid) (11.8 g, 82 mmol) in ethanol (70 mL) was heated to reflux under argon for 2 hours. The resulting precipitate was isolated by filtration then washed with cold ethanol then ether and dried in vacuo to afford a yellow solid (23.3 g, 92%). MS (+ve ion electrospray) m/z 374 (MH⁺).

### (f) 8-Bromo-6-fluoro-5-methoxy-1H-quinolin-4-one

Dowtherm® A (30 mL) was heated to reflux under a gentle stream of argon and (22e) (10 g, 26.3 mmol) was added portionwise over 2 minutes (CAUTION—rapid evolution of carbon dioxide and acetone). The mixture was heated for a further 2 minutes then allowed to cool to room temperature. A solid was filtered off, which was dissolved with dichloromethane/methanol and dry-loaded onto silica. The filtrate was also added to the column, then elution with dichloromethane afforded a yellow solid (2.5 g, 34%). MS (+ve ion electrospray) m/z 272 (MH⁺).

### (g) 6-Fluoro-5-methoxy-1H-quinolin-4-one

A solution of (22f) (3.5 g, 12.8 mmol) in aqueous sodium hydroxide solution (2M, 13 mL, 26 mmol)/dioxan (300 mL)/water (100 mL) was hydrogenated over 10% palladium on charcoal (1.5 g) for 60 hours. The mixture was filtered through Kieselguhr and acidified to pH7 with concentrated hydrobromic acid. The mixture was evaporated and the residue partitioned between ethyl acetate and water. The ethyl acetate extract was dried and concentrated whereupon crystallisation commenced. Filtration and drying under vacuum afforded a white crystalline solid (1.5 g, 60%). MS (+ve ion electrospray) m/z 194 (MH⁺).

### (h) 3-Chloro-6-fluoro-5-methoxy-1H-quinolin-4-one

6-Fluoro-5-methoxy-1H-quinolin-4-one (22f) (0.4 g) in acetic acid (8 mL) was sonicated and warmed until all had dissolved, and then it was treated with N-chlorosuccinimide (281 mg) and the mixture was heated at 50° C. for 3 hr, cooled, and the solid collected and washed with acetic acid and dried in vacuo to give a white solid (0.33 g). MS (ES) m/z 228/230 (M+H)⁺.

### (i) 4-Bromo-3-chloro-6-methoxy-quinoline

3-Chloro-6-fluoro-5-methoxy-1H-quinolin-4-one (22h) (0.33 g) in dry DMF (5 mL) was cooled in ice and phosphorus tribromide (0.2 mL) added drop-wise, and the mixture was stirred, with ice-cooling for 30 minutes then allowed to warm to room temperature and stirred for a further 2 hours. It was cooled in ice and sodium bicarbonate solution was added and the mixture was extracted with ethyl acetate, dried (magnesium sulfate), evaporated and dried in vacuo, to afford a yellow solid (0.16 g). MS (ES) m/z 290/292/294 (M+H)⁺.

### (j) 3-Chloro-6-fluoro-5-methoxy-4-vinyl-quinoline

The bromide (22i) (0.16 g) in DME (5 mL) under argon, was treated with tetrakis(triphenylphosphine)palladium(0) (0.072 g) and the mixture stirred at room temperature for 20 minutes. Anhydrous potassium carbonate (0.083 g), water (1.5 mL), and vinylborane:pyridine complex (150 mg) was added and the mixture was heated at 100° C. for 1 hr. It was cooled, diluted with water and extracted with ether, dried (magnesium sulfate) and evaporated to dryness. After work-up the product was chromatographed on silica gel (hexane-ethyl acetate) to afford a white solid (0.14 g). MS (ES) m/z 238/240 (M+H)⁺.

### (k) {1-[2-(3-Chloro-6-fluoro-5-methoxy-quinolin-4-yl)-ethyl]-piperidin-4-yl}-carbamic acid tert-butyl ester

A mixture of the vinyl-quinoline (22j) (0.14 g) and piperidin-4-yl-carbamic acid tert-butyl ester (0.12 g) in chloroform (1 mL) was heated at 150° C. for 3 days, then the product was dissolved in DCM and chromatographed on silica gel (ethyl acetate-hexane) to afford the solid product (0.02 g). MS (ES) m/z 438/440 (M+H)⁺.

### (l) 1-[2-(3-Chloro-6-fluoro-5-methoxy-quinolin-4-yl}-ethyl]-piperidin-4-ylamine dihydrochloride

The ester (22k) (0.02 g) was dissolved in chloroform (0.5 mL) and a solution of 4M HCl in dioxan (1.0 mL) was added and the solution was stirred at room temperature for 1 hr then evaporated to dryness and azeotroped with toluene to give the product. MS (ES) m/z 338/340 (M+H)⁺.

### (m) Title Compound

The amine (22l) (0.015 g) and aldehyde (7d) (0.012 g) were dissolved in dichloromethane (4 ml), methanol (1 ml) and triethylamine (0.042 ml) and stirred for 18 hours. Methanol (1 ml) and sodium borohydride (0.002 g) were added and the solution was stirred for 15 min at room temperature. The mixture was quenched with 2N HCl, basified with sodium bicarbonate solution, and extracted with methanol-DCM, dried (magnesium sulfate), evaporated and chromatographed on silica gel (methanol-DCM) to afford the free base of the title compound as a solid (0.011 g).

¹H NMR (400 MHz, d₄-MeOH) 8.71 (s, 1H), 7.78 (m, 1H), 7.71 (d, J=7.8 Hz, 1H), 7.65 (m, 1H), 7.04 (d, J=7.8 Hz, 1H), 4.12 (s, 3H), 3.92 (s, 2H), 3.71 (m, 2H), 3.52 (m, 2H), 3.31 (m, 2H), 3.15 (m, 2H), 2.71 (m, 2H), 2.31 (m, 2H), 2.04 (m, 2H). LC-MS (ES) m/z 516/518 (M+H)⁺.

This material, as a solution in chloroform/methanol, was treated with an excess of 4M HCl in dioxan and evaporated to dryness. The solid was triturated with ether to give the title compound (0.012 g).

### Example 23

### 6-({1-[2-(3-Chloro-6-methyl-[1,5]naphthyridin-4-yl)-ethyl]-piperidin-4-ylamino}-methyl)-4H-pyrido[3,2-b][1,4]thiazin-3-one Dihydrochloride

### (a) 6-Methyl-pyridin-3-ylamine

Bromine (19.0 ml) was added to a solution of NaOH (50 g) in water (990 ml) at 0° C. with stirring. 6-Methyl-nicotinamide was then added in small portions keeping the temperature below 5° C. The mixture was heated at 80° C. for 18 h and then cooled and extracted with dichloromethane (6×200 ml). The combined organics were then dried (MgSO4) and then evaporated to give the desired product (58%). MS (+ve ion electrospray) m/z 108 (MH⁺).

### (b) 2,2,-Dimethyl-5-[(6-methyl-pyridin-3-ylamino)-methylene-[1,3]dioxane-4,6-dione

A mixture of amine (23a) (46.5 g), triethyl orthoformate (72 ml) and 2,2-dimethyl-[1,3]dioxane-4,6-dione (Meldrums acid) (62 g) in ethanol (300 mL) was heated to reflux under argon for 2 hours. The resulting precipitate was isolated by filtration then washed with cold ethanol then ether and dried in vacuo to afford a yellow solid (89 g, 80%). MS (+ve ion electrospray) m/z 261 (MH⁺).

### (c) 6-Methyl-1H-[1,5]naphthyridin-4-one

Dowtherm® A (100 mL) was heated to reflux under a gentle stream of argon and (23b) (18 g) was added portionwise over 2 minutes (CAUTION—rapid evolution of carbon dioxide and acetone). The mixture was heated for a further 2 minutes then allowed to cool to room temperature. A solid was filtered off, which was dissolved with dichloromethane/methanol and dry-loaded onto silica. The filtrate was also added to the column, then elution with dichloromethane afforded a yellow solid (6.4 g, 30%). MS (+ve ion electrospray) m/z 160 (MH⁺).

### (d) 3-Chloro-6-methyl-1H-[1,5]naphthyridin-4-one

6-Methyl-1H[1,5]naphthyridin-4-one (23c) (14 g) in acetic acid (250 mL) was sonicated and warmed until all had dissolved, and then it was treated with N-chlorosuccinimide (12 g) and the mixture was heated at 50° C. for 3 hr, cooled, and the solid collected and washed with acetic acid and dried in vacuo to give a white solid (7.2 g, 41%). MS (ES) m/z 194/196 (M+H)⁺.

### (e) 8-Bromo-7-chloro-2-methyl-[1,5]naphthyridine

The naphthyridin-4-one (23e) (7.2 g) in dry DMF (90 mL) was cooled in ice and phosphorus tribromide (4.2 mL) added drop-wise, and the mixture was stirred, with ice-cooling for 30 minutes then allowed to warm to room temperature and stirred for a further 2 hours. It was cooled in ice and sodium bicarbonate solution was added and the mixture was extracted with ethyl acetate, dried (magnesium sulfate), evaporated and dried in vacuo, to afford a yellow solid (1.91 g). MS (ES) m/z 258/260/262 (M+H)⁺.

### (f) 7-Chloro-2-methyl-8-vinyl[1,5]naphthyridine

The bromide (23e) (1.0 g) in DME (30 mL) under argon, was treated with tetrakis(triphenylphosphine)palladium(0) (0.090 g) and the mixture stirred at room temperature for 20 minutes. Anhydrous potassium carbonate (0.534 g), water (9 mL), and vinylborane:pyridine complex (375 mg) was added and the mixture was heated at 100° C. for 4 h. It was cooled, diluted with water and extracted with ether, dried (magnesium sulfate) and evaporated to dryness. After work-up the product was chromatographed on silica gel (hexane-ethyl acetate) to afford a white solid (0.70 g, 88%). MS (ES) m/z 205/207 (M+H)⁺.

### (g) {1-[2-(3-Chloro-6-methyl-[1,5]-naphthyridin-4-yl)-ethyl]-piperidin-4-yl}-carbamic acid tert-butyl ester

A mixture of the vinyl compound (23f) (0.36 g) and piperidin-4-yl-carbamic acid tert-butyl ester (0.35 g) in chloroform (1 ml) was heated at 100° C. for 48 h, then the product was dissolved in DCM and chromatographed on silica gel (ethyl acetate-hexane) to afford the solid product (0.41 g, 58%). MS (ES) m/z 405/407 (M+H)⁺.

### (h) 1 [2-(3-Chloro-6-methyl-[1,5]-naphthyridin-4-yl)ethyl]-piperidin-4-ylamine

The compound (23g) (0.41 g) was dissolved in chloroform (4 ml) and a solution of 4M HCl in dioxan (12 ml) was added and the solution was stirred at room temperature for 1 hr then evaporated to dryness. The resultant solid was dissolved in 10% MeOH/DCM (100 ml), basified with saturated NaHCO₃ (5 ml) and further extracted with 10% MeOH/DCM (2×100 ml). The combined organics were dried (MgSO₄) and evaporated to give the desired compound (0.31 g, 100%). MS (ES) m/z 305/307 (M+H)⁺.

### (i) Title Compound

The amine (23h) (0.102 g) and aldehyde (7d) (0.065 g) were dissolved in chloroform (2 ml) and methanol (2 ml) with 3 A molecular sieves and refluxed for 4 hours. Sodium triacetoxyborohydride (0.140 g) was added and the solution was stirred for 18 h at room temperature. The mixture was evaporated and chromatographed on silica gel (methanol-DCM) to afford the free base of the title compound as a solid (0.14 g, 80%).

¹H NMR (400 MHz, d₄-MeOH) 8.77 (s, 1H), 8.22 (d, J=7.8 Hz, 1H), 7.74 (d, J=7.6 Hz, 1H), 7.62 (d, J=7.6 Hz, 1H), 7.05 (d, J=7.8 Hz, 1H), 4.12 (s, 2H), 3.67 (m, 2H), 3.53 (s, 2H), 3.31-3.36 (m, 2H), 3.02-3.015 (m, 1H), 2.87-2.91 (m, 2H), 2.91 (s, 3H), 2.75-2.84 (m, 2H), 2.19-2.44 (m, 2H), 1.64-1.74 (m, 2H). LC-MS (ES) m/z 483/485 (M+H)⁺.

This material, as a solution in chloroform/methanol, was treated with an excess of 4M HCl in dioxan and evaporated to dryness. The solid was triturated with ether to give the title compound (0.146 g).

### Example 24

### {1-[2-(3-Chloro-6-methyl-[1,5]naphthyridin-4-yl)-ethyl]-piperidin-4-yl}-(2,3-dihydro[1,4]dioxino[2,3-c]pyridin-7-ylmethyl)-amine Dihydrochloride

This was prepared from amine (23h) and aldehyde (2c) by the method of Example (23i) to give the free base of the title compound (45%).

¹H NMR (400 MHz, d₄-MeOH) 8.78 (s, 1H), 8.23 (d, J=7.5 Hz, 1H), 8.06 (s, 1H), 7.62 (d, J=7.5 Hz, 1H), 6.98 (s, 1H), 4.30-4.41 (m, 4H), 4.04 (s, 2H), 3.62-3.72 (m, 2H), 3.28-3.33 (m, 2H), 2.92-3.04 (m, 1H), 2.84-2.87 (m, 2H), 2.76 (s, 3H), 2.32-2.37 (m, 2H), 2.08-2.12 (m, 2H), 1.58-1.68 (m, 2H). LC-MS (ES) m/z 454/456 (M+H)⁺.

This material, as a solution in chloroform/methanol, was treated with an excess of 4M HCl in dioxan and evaporated to dryness. The solid was triturated with ether to give the title compound.

### Example 25

### 6-({1-[2-(3-Chloro-6-fluoro-quinolin-4-yl)-ethyl]-piperidin-4-ylamino}-methyl)-4H-pyrido[3,2-b][1,4]thiazin-3-one Dihydrochloride

### (a) 5-[4-Fluoro-phenyl-amino)-methylene]-2,2-dimethyl-[1,3]dioxane-4,6-dione

This was prepared (89%) from 4-fluoro-aniline using the method of Example (23b) MS (+ve ion electrospray) m/z 264 (MH⁺).

### (b) 6-Fluoro-1-H-quinolin-4-one

This was prepared (54%) from (24a) by the method of Example (23c)

MS (+ve ion electrospray) m/z 163 (MH⁺).

### (c) 3-Chloro-6-fluoro-1-H-quinolin-4-one

This was prepared (95%) from (24b) by the method of Example (23d)

MS (+ve ion electrospray) m/z 197/199 (MH⁺).

### (d) 4-Bromo-3-chloro-6-fluoro-quinoline

This was prepared (69%) from (24c) by the method of Example (23e)

MS (+ve ion electrospray) m/z 260/262/264 (MH⁺).

### (e) 3-Chloro-6-fluoro-4-vinyl-quinoline

This was prepared (86%) from (24d) by the method of Example (23f)

MS (+ve ion electrospray) m/z 207/209 (MH⁺).

### (f) {1-[2-(3-Chloro-6-fluoro-quinolin-4-yl)-ethyl]-piperidin-4-yl}-carbamic acid tert butyl ester

This was prepared (29%) from (24e) by the method of Example (23g)

MS (+ve ion electrospray) m/z 407/409 (MH⁺).

### (g) 1-[2-(3-Chloro-6-fluoro-quinolin-4-yl)-ethyl]-piperidin-4-ylamine

This was prepared (80%) from (24f) by the method of Example (23h)

LC-MS (ES) m/z 307/309 (M+H)⁺.

### (h) Title Compound

This was prepared as the free base (88%) from (24g) and aldehyde (7d) by the method of Example (23i)

¹H NMR (400 MHz, d₄-MeOH) 8.75 (s, 1H), 8.106-8.12 (m, 1H), 7.83-7.86 (m, 1H), 7.77 (d, J=7.6 Hz, 1H), 7.61-7.76 (m, 1H), 7.07 (d, J=7.6 Hz, 1H), 4.11 (s, 2H), 3.54 (s, 2H), 3.44-3.53 (m, 2H), 3.23-3.30 (m, 2H), 2.27-3.05 (m, 1H), 2.71-2.74 (m, 2H) 2.25-2.31(m, 2H), 2.11-2.15 (m, 2H), 1.64-1.71 (d, 2H).

LC-MS (ES) m/z 485/487 (M+H)⁺.

This material, as a solution in chloroform/methanol, was treated with an excess of 4M HCl in dioxan and evaporated to dryness. The solid was triturated with ether to give the title compound.

### Example 26

### {1-[2-(3-Chloro-6-fluoro-quinolin-4-yl)-ethyl]-piperidin-4-yl}(2,3-dihydro[1,4]dioxino[2,3-c]pyridin-7-ylmethyl)-amine Dihydrochloride

This was prepared as the free base (78%) from the amine (25g) and aldehyde (2c) by the method of Example (23i).

¹H NMR (400 MHz, d₄-MeOH) 8.71 (s, 1H), 8.02-8.09 (m, 2H), 7.78-7.82 (m, 1H), 7.58-7.59 (m, 1H), 7.00 (s, 1H), 4.30-4.40 (m, 4H), 4.04 (s, 2H), 3.40-3.44 (m, 2H), 3.17-3.19 (m, 2H), 2.86-3.01 (m, 1H), 2.64-2.70 (m, 2H), 2.25-2.33 (m, 2H), 2.01-2.14 (m, 2H), 1.67-1.74 (m, 2H). LC-MS (ES) m/z 456/458 (M+H)⁺.

This material, as a solution in chloroform/methanol, was treated with an excess of 4M HCl in dioxan and evaporated to dryness. The solid was triturated with ether to give the title compound.

### Example 27

### 6-({1-[2-(3,6-Dichloro-quinolin-4-yl)-ethyl]-piperidin-4-ylamino}-methyl)-4H-pyrido[3,2-b][1,4]thiazin-3-one Dihydrochloride

### (a) 5-[4-Chloro-phenyl-amino)-methylene]-2,2-dimethyl-[1,3]dioxane-4,6-dione

This was prepared (95%) from 4-chloro-aniline using the method of Example (23b).

MS (+ve ion electrospray) m/z 283/285 (MH⁺).

### (b) 6-chloro-1-H-quinolin-4-one

This was prepared from (27a) (56%) by the method of Example (23c)

MS (+ve ion electrospray) m/z 179/181 (MH⁺).

### (c) 3,6-Dichloro-1-H-quinolin-4-one

This was prepared from (27b) (60%) by the method of Example (23d).

MS (+ve ion electrospray) m/z 214/216/218 (MH⁺).

### (d) 4-Bromo-3,6-dichloro-quinoline

This was prepared from (27c) (69%) by the method of Example (23e).

MS (+ve ion electrospray) m/z 294/296/298/300 (MH⁺).

### (e) 3,6-Dichloro-4-vinyl-quinoline

This was prepared from (27d) (75%) by the method of Example (23f).

MS (+ve ion electrospray) m/z 223/225/227 (MH⁺).

### (f) {1-[2-(3,6-Dichloro-quinolin-4-yl)-ethyl]-piperidin-4-yl}-carbamic acid tert butyl ester

This was prepared from (27e) (20%) by the method of Example (23g).

MS (+ve ion electrospray) m/z 423/425/427 (MH⁺).

### (g) 1-[2-(3,6-Dichloro-quinolin-4-yl)-ethyl]-piperidin-4-ylamine

This was prepared from (27f) (100%) by the method of Example (23h).

LC-MS (ES) m/z 323/325/327 (M+H)⁺.

### (i) Title Compound

The free base of the title compound was prepared (45%) from (27g) and aldehyde (7d) by the method of Example (23h).

¹H NMR (400 MHz, d₄-MeOH) 8.80 (s, 1H), 8.18-8.20 (m, 1H), 8.02 (d, J=7.6 Hz, 1H), 7.61-7.76 (m, 2H), 7.02 (d, J=7.6 Hz, 1H), 3.88 (s, 2H), 3.61 (s, 2H), 3.42-3.50 (m, 2H), 3.20-3.35 (m, 2H), 3.09-3.14 (m, 1H), 2.63-2.66 (m, 2H) 2.22-2.27(m, 2H), 2.04-2.17 (m, 2H), 1.56-1.62 (d, 2H). LC-MS (ES) m/z 501/503/505 (M+H)⁺.

This material, as a solution in chloroform/methanol, was treated with an excess of 4M HCl in dioxan and evaporated to dryness. The solid was triturated with ether to give the title compound.

### Example 28

### {1-[2-(3,6-Dichloro-quinolin-4-yl)-ethyl]-piperidin-4-yl}-(2,3-dihydro[1,4]dioxino[2,3-c]pyridin-7-ylmethyl-amine Dihydrochloride

This was prepared as the free base (28%) from the amine (27g) and aldehyde (2c) by the method of Example (23i).

¹H NMR (400 MHz, d₄-MeOH) 8.75 (s, 1H), 8.18-8.19 (m, 2H), 7.99-8.02 (m, 1H), 7.71-7.75 (m, 1H), 6.96 (s, 1H), 4.28-4.76 (m, 4H), 3.79 (s, 2H), 3.39-3.45 (m, 2H), 3.29-3.35 (m, 2H), 3.07-3.12 (m, 2H), 2.56-2.63 (m, 3H), 2.15-2.19 (m, 2H), 2.00-2.10 (m, 2H), 1.44-1.53 (m, 2H). LC-MS (ES) m/z 472/474/476 (M+H)⁺.

This material, as a solution in chloroform/methanol, was treated with an excess of 4M HCl in dioxan and evaporated to dryness. The solid was triturated with ether to give the title compound.

### Example 29

### (cis)-1-[2-(3-Chloro-6-methoxy-[1,5]naphthyridin-4-yl)-ethyl]-4-[(2,3-dihydro-[1,4]dioxino[2,3-c]pyridin-7-ylmethyl)-amino]-piperidin-3-ol Dihydrochloride Enantiomer 1

### (a) cis-4-Amino-1-[2-(3-chloro-6-methoxy-[1,5]naphthyridin-4-yl)-ethyl]-piperidin-3-ol Enantiomer 1

This was prepared from 7-chloro-2-methoxy-8-vinyl-[1,5]naphthyridine (3a) and cis-(3-hydroxy-piperidin-4-yl)-carbamic acid tert-butyl ester enantiomer 1 (5c) by the method of Example (5d) followed by removal of the protecting group by treatment with trifluoroacetic acid in DCM, by the method of Example (1g).

### (b) Title Compound

This was prepared as the free base (0.346 g) from the amine (29a) (0.377 g) and aldehyde (2c) (0.18 g) by the method of Example (3d), except that the compound was chromatographed on silica gel eluting with methanol/DCM then 0.5% ammonia in 10% methanol/DCM.

¹H NMR δH (CDCl₃, 400 MHz), 8.66 (s, 1H), 8.16 (d, 1H), 8.09 (1H, s), 7.10 (d, 1H), 6.84 (d, 1H), 4.30 (m, 4H), 4.08 (s, 3H), 3.88 (1H, s), 3.84 (s, 2H), 3.52 (t, 2H), 3.15 (m, 1H), 3.00 (m, 1H), 2.78 (dd, 2H), 2.60 (m, 1H), 2.20-2.45 (m, 3H), 1.75 (m, 2H).

MS (ES) m/z 486/488 (M+H)⁺.

This material, as a solution in chloroform/methanol, was treated with an excess of 4M HCl in dioxan and evaporated to dryness. The solid was triturated with ether to give the title compound (0.429 g).

### Example 30

### (cis)-1-[2-(3-Chloro-6-methoxy-[1,5]naphthyridin-4-yl)-ethyl]-4 [(2,3-dihydro-[1,4]dioxino[2,3-c]pyridin-7-ylmethyl)-amino]-piperidin-3-ol Dihydrochloride Enantiomer 2

### (a) cis-4-Amino-1-[2-(3-chloro-6-methoxy-[1,5]naphthyridin-4-yl)-ethyl]-piperidin-3-ol Enantiomer 2

This was prepared from 7-chloro-2-methoxy-8-vinyl-[1,5]naphthyridine (3a) and cis-(3-hydroxy-piperidin-4-yl)-carbamic acid tert-butyl ester enantiomer 2 [prepared from 5b Enantiomer 2 by the method of Example (5c)] by the method of Example (5d) followed by removal of the protecting group by treatment with trifluoroacetic acid in DCM, by the method of Example (1g).

### (b) Title Compound

This was prepared as the free base (0.34 g) from the amine (30a) (0.26 g) and aldehyde (2c) (0.125 g) by the method of Example (3d).

¹H NMR δH (CDCl₃, 400 MHz), 8.68 (s, 1H), 8.17 (d, 1H), 8.09 (1H, s), 7.10 (d, 1H), 6.84 (d, 1H), 4.30 (m, 4H), 4.09 (s, 3H), 3.88 (1H, s), 3.84 (s, 2H), 3.52 (t, 2H), 3.13 (m, 1H), 2.98 (m, 1H), 2.76 (dd, 2H), 2.58 (m, 1H), 2.40 (d, 1H), 2.25 (1H, m), 2.25 (m, 1H), 1.74 (m, 2H). MS (ES) m/z 486/488 (M+H)⁺.

This material, as a solution in chloroform/methanol, was treated with an excess of 4M HCl in dioxan and evaporated to dryness. The solid was triturated with ether to give the title compound (0.39 g).

### Example 31

### 6-({1-[2-(3-Fluoro-6-methoxyquinolin-4-yl)ethyl]piperidin-4-yl amino}methyl)-4H-pyrido[3,2-b][1,4]thiazin-3-one dihydrochloride

### (a) 4-Chloro-6-methoxyquinoline-3-carboxylic acid

Ethyl 4-chloro-6-methoxyquinoline-3-carboxylate [R. Fryer et al J. Med. Chem. 36, 1669-1673 (1993)] (64.9 g) was partially dissolved in THF (1 L) and treated dropwise with aqueous 2M sodium hydroxide (195 mL). After overnight stirring, the mixture was neutralised with dilute HCl and THF was removed in vacuo. The residue was dissolved in water and acidified with dil. HCl. The solid product was collected under suction, washed well with water and dried in vacuo to give a white solid (56.2 g, 99%). MS (ES) m/z 238/240 (M+H)⁺.

### (b) (4-Chloro-6-methoxy-quinolin-3-yl)-carbamic acid tert-butyl ester

To a solution of the acid (31a) (10 g, 41.9 mmol), triethylamine (49 mL) and tert-butanol (63 mL) in dry dimethylformamide (140 mL) was added diphenylphosphoryl azide (10 ml, 45.7 mmol). The mixture was heated at 100° C. for 1 h, then cooled and evaporated. The residue was dissolved in dichloromethane and washed with water (some insoluble material was removed by filtration). The aqueous phase was extracted with dichloromethane and the combined organics were dried and evaporated. Chromatography on silica (1:1 ether/light petroleum ether) gave the carbamate (10.11 g, 78%). MS (ES) m/z 309/311 (M+H)⁺, 253/255 (M+H-C₄H₈)⁺

### (c) 3-Amino-4-chloro-6-methoxyquinoline

The carbamate (31b) (10.11 g, 32.8 mmol) was dissolved in dichloromethane (100 mL) and treated with trifluoroacetic acid (100 mL). After 1.75 h standing at room temperature, the mixture was evaporated and the residue was dissolved in water and basified with aq. sodium carbonate. The precipitate was filtered off, dried and recrystalised from dichloromethane (in two crops, with a third crop obtained by addition of light petrol) to give a white solid (5.91 g, 86%).

MS (ES) m/z 209/211 (M+H)⁺

### (d) 4-Chloro-3-fluoro-6-methoxyquinoline

The amine (31c) (10.52 g, 50.5 mmol) was dissolved in dry THF and cooled to −8° C. Nitrosonium tetrafluoroborate (6.48 g, 55.5 mol) was added in portions over 30 min. at <−2° C. The mixture was then stirred at −5 to −2° C. for 30 min., then the yellow precipitate was filtered off, washed with cold THF and dried, to give a diazonium tetrafluoroborate salt (13.94 g, 90%).

A suspension of this salt (13.51 g) in decahydronaphthalene (mixed isomers, 270 mL) was heated to 175-180° C., held at this temperature for 10 min., then cooled. The decahydronaphthalene was decanted off, and the residue was washed twice with light petrol. Addition of the washings to the decanted liquor gave a precipitate which was collected and dissolved in dichloromethane. The gummy residue was extracted twice with dichloromethane, the extracts being diluted with ether and filtered, then combined with the material precipitated from the liquor and evaporated. Chromatography on silica (0-2% methanol/dichloromethane) gave a white solid (2.45 g, 28%). MS (ES) m/z 212 (M+H)+

### (e) 3-Fluoro-6-methoxy-4-vinylquinoline

The 4-chloro-3-fluoroquinoline (31d) (2.25 g, 10.7 mmol) was dissolved in 1,2-dimethoxyethane (80 mL), tetrakis(triphenylphosphine)palladium(0) (0.61 g, 0.53 mmol) was added and the mixture was stirred under argon for 20 min. Water (30 mL), potassium carbonate (1.48 g, 10.7 mmol) and 2,4,6-trivinylcyclotriboroxane-pyridine complex (F. Kerins & D. F. O'Shea, J. Org. Chem., 2002, 67, 4968)(1.93 g, 8.0 mmol) were added and the mixture was heated under reflux for 24 h. After cooling, ether was added and the phases were separated. The aqueous phase was extracted well with ether, and the combined extracts were dried and evaporated. Chromatography on silica (10-20% ether/light petroleum ether) gave a waxy solid (1.73 g, 80%). MS (ES) m/z 204 (M+H)+

### (f) {1-[2-(3-Fluoro-6-methoxy-quinolin-4-yl)-ethyl]-piperidin-4-yl}-carbamic acid tert-butyl ester

The 4-vinylquinoline (31e) (0.80 g, 3.9 mmol) was heated with piperidin-4-yl-carbamic acid tert-butyl ester (1.58 g, 7.8 mmol)) and dimethylformamide (1 mL) at 100° C. for 24 h. After cooling, water was added and the mixture was extracted with ether and ethyl acetate. The extracts were dried and evaporated.

Chromatography on silica (2% methanol/dichloromethane) gave the product (0.80 g, 51%). MS (ES) m/z 404 (M+H)+

### (g) 1-[2-(3-Fluoro-6-methoxy-quinolin-4-yl}-ethyl]-piperidin-4-ylamine

The carbamate (31f) (0.051 g, 0.13 mmol) was dissolved in dichloromethane (1 mL) and treated with trifluoroacetic acid (1 mL). The solution was allowed to stand at room temperature for 1.75 h, then evaporated. The residue was triturated twice with ether, then dissolved in 10% methanol/dichloromethane and stirred with polymer-bound carbonate (MP-carbonate resin, Argonaut Technologies Inc.: 2.8 mmol/g, 0.24 g) for 3 h. The resin was filtered off and washed several times alternately with 10% methanol/dichloromethane and methanol. Evaporation of solvent gave the amine (0.044 g, >100%), probably still containing some trifluoroacetate salt. MS (ES) m/z 304 (M+H)+

### (h) Title Compound

The crude amine (31g) (assumed 0.13 mmol) and aldehyde (7d) (0.027 g, 0.14 mmol) were mixed in dry chloroform (5 mL) and methanol (0.5 mL) and heated under reflux for 5 h. The mixture was cooled, treated with sodium triacetoxyborohydride (0.13 g) and stirred at room temperature overnight. The mixture was washed with aq. sodium bicarbonate, the aqueous phase was re-extracted with 10% methanol/dichloromethane and the combined organics were washed with brine, dried and evaporated. Chromatography on silica (2-10% methanol/dichloromethane) gave the free-base of the title compound (0.032 g, 51%).

1H NMR (250 MHz, CDCl3) δ8.58 (1H, d), 8.45 (1H, broad), 8.00 (1H, d), 7.57 (1H,d), 7.31 (1H, d), 7.22 (1H, d), 6.99 (1H, d), 3.95 (3H,s), 3.85 (2H, s), 3.47 (2H, s), 3.24 (2H, m), 3.04 (2H, m), 2.67 (2H, m), 2.55 (1H, m), 2.19 (2H, m), 1.95 (2H, m), 1.51 (2H, m). MS (ES) m/z 482 (M+H)+

The free base in dichloromethane/methanol was treated with 2 equivalents of HCl (0.4M in 1,4-dioxan), followed by evaporation of solvent and trituration with ether to give the dihydrochloride salt.

### Example 32

### {1-[2-(3-Fluoro-6-methoxy-quinolin-4-yl)-ethyl]-piperidin-4-yl}-(2,3-dihydro-[1,4]dioxino[2,3-c]pyridin-7-ylmethyl)-amine dihydrochloride

The crude amine (31g), [prepared from 1.98 mmol carbamate (Example 31f)], and aldehyde (2c) (0.32 g, 1.98 mmol) were dissolved in dimethylformamide (20 mL). Sodium triacetoxyborohydride (1.22 g, 5.76 mmol) was added and the mixture was stirred at room temperature overnight. After addition of a small volume (<1 ml) of 5M HCl, approximately half the solvent was removed by evaporation and the residue was treated with sat.aq. sodium carbonate and water (20 mL. each). The final pH was adjusted to 10-11 and the mixture was refrigerated before filtering off the solid, which was washed with water and dried to give the free-base of the title compound (0.55 g, 61%).

¹H NMR (250 MHz, CDCl₃) δ8.58 (1H, d), 8.11 (1H, s), 8.00 (1H, d), 7.31 (1H, d), 7.22 (1H, d), 6.83 (1H, s), 4.33 (2H, m), 4.27 (2H, m), 3.95 (3H,s), 3.81 (2H, s), 3.24 (2H, m), 3.02 (2H, m), 2.64(2H, m), 2.54 (1H, m), 2.17 (2H, m), 1.95 (2H, m), 1.50 (2H, m). MS (ES) m/z 453 (M+H)⁺

The free base in dichloromethane was treated with 2 equivalents of HCl (4M in 1,4-dioxan), followed by evaporation of solvent and trituration with ether to give the dihydrochloride salt.

### Example 33

### cis-4-[(2,3-Dihydro-[1,4]dioxino[2,3-c]pyridin-7-ylmethyl)-amino]-1-[2-(3-fluoro-6-methoxy-quinolin-4-yl)-ethyl]-piperidin-3-ol Enantiomer 2 dihydrochloride

### (a) cis-{1-[2-(3-Fluoro-6-methoxy-quinolin-4-yl)-ethyl]-3-hydroxy-piperidin-4-yl]-carbamic acid tert-butyl ester enantiomer 2

The vinyl quinoline (31e) (0.38 g, 1.85 mmol) and cis-4-tert-butoxycarbonylamino-3-hydroxy-piperidine enantiomer 2, prepared from the benzyl carbamate (5b, enantiomer2) (0.40 g, 1.85 mmol) by the method of Example 5(c), were heated with dimethylformamide (0.5 mL) at 100° C. for 48 h, with addition of 1,1,5,5-tetramethylguanidine (5 drops) after 24 h. Work up as for Example (31f) followed by chromatography on silica (0-4% methanol/dichloromethane) gave a yellow gum (0.33 g, 43%), plus some recovered vinyl compound (60 mg). MS (ES) m/z 420 (M+H)⁺

### (b) cis-4-Amino-1-[2-(3-chloro-6-methoxy-quinolin-4-yl)-ethyl]-piperidin-3-ol enantiomer 2

The tert-butyl carbamate (33a) was deprotected by the method of Example (31g) to give the crude amine. MS (ES) m/z 320 (M+H)⁺

### (c) Title Compound

The crude amine (33b) (prepared from 1.79 mmol carbamate) and aldehyde (2c) (0.28 g, 1.70 mmol) were mixed in dry chloroform (5 mL) and methanol (0.5 mL) and heated under reflux for 5.5 h, with 4 A molecular sieves added after 4 h. The mixture was cooled, treated with sodium triacetoxyborohydride (0.38 g) and stirred at room temperature over 2 days. A further portion of the borohydride (0.2 g) was added and stirring continued for 8 h. A few drops of 5M HCl were added, then the mixture was washed with aq. sodium bicarbonate, the aqueous phase was re-extracted with 10% methanol/dichloromethane and the combined organics were washed with brine, dried and evaporated. Chromatography on silica (5-10% methanol/dichloromethane) gave the free-base of the title compound (0.44 g, 52%).

¹H NMR (250 MHz, CDCl₃) δ8.58 (1H, d), 8.10 (1H, s), 7.99 (1H, d), 7.31 (1H, dd), 7.19 (1H, d), 6.83 (1H, s), 4.33 (2H, m), 4.28 (2H, m), 3.95 (3H,s), 3.88 (1H, m), 3.84 (2H, s), 3.24 (2H, m), 3.12 (1H, m), 2.94(1H, m), 2.64 (3H, m), 2.33 (1H, m), 2.21 (1H, m), 1.75 (2H, m). MS (ES) m/z 469 (M+H)⁺

### Example 34

### cis-4-[(2,3-Dihydro-[1,4]dioxino[2,3-c]pyridin-7-ylmethyl)-amino]-1-[2-(3-fluoro-6-methoxy-quinolin-4-yl)-ethyl]-piperidin-3-ol dihydrochloride dihydrochloride Enantiomer 1

### (a) cis-{1-[2-(3-Fluoro-6-methoxy-quinolin-4-yl)-ethyl]-3-hydroxy-piperidin-4-yl)-carbamic acid tert-butyl ester enantiomer 1

This was prepared from the vinyl quinoline (31e) (0.50 g) and cis-4-tert-butoxycarbonylamino-3-hydroxy-piperidine enantiomer 1, prepared from the benzyl carbamate (5b, enantiomer1) (0.53 g,) by the method of Example 5(c), were heated with dimethylformamide (0.6 mL) and 1,1,5,5-tetramethylguanidine (2 drops) at 100-105° C. for 72 h. Work up as for Example (31f) followed by chromatography on silica (0-4% methanol/dichloromethane) gave an oil (0.44 g), plus some recovered vinyl compound (90 mg). MS (ES) m/z 420 (M+H)⁺

### (b) cis-4-Amino-1-[2-(3-chloro-6-methoxy-quinolin-4-yl)-ethyl]-piperidin-3-ol enantiomer 1

The tert-butyl carbamate (34a) was deprotected by the method of Example (31g) to give the crude amine (0.37 g). MS (ES) m/z 320 (M+H)⁺

### (c) Title Compound

The crude amine (34b) (0.34 g) was reacted with aldehyde (2c) (0.167 g) in dry chloroform (3 mL) and methanol (3 mL) under reflux for 4 h, with 3 A molecular sieves. The mixture was cooled, treated with sodium triacetoxyborohydride (0.642 g) and stirred at room temperature over 2 days, then the mixture was washed with aqueous sodium carbonate and the aqueous phase was re-extracted with 10% methanol/chloroform and the combined organics were dried and evaporated. Chromatography on silica (DCM then 2-10% methanol/dichloromethane) gave the free-base of the title compound (0.42 g).

¹H NMR (400 MHz, CDCl₃) δ8.59 (1H, s), 8.10 (1H, s), 7.98 (1H, d), 7.32 (1H, dd), 7.20 (1H, d), 6.83 (1H, s), 4.32 (2H, m), 4.28 (2H, m), 3.97 (3H,s), 3.90 (1H, br s), 3.84 (2H, s), 3.25 (2H, t), 3.12 (1H, m), 2.95(1H, m), 2.55-2.70 (3H, m), 2.33 (1H, d), 2.23 (1H, m), 1.77 (2H, m). MS (ES) m/z 469 (M+H)⁺

The free base in dichloromethane was treated with an excess of HCl (4M in 1,4-dioxan), followed by evaporation of solvent and trituration with ether to give the title compound (0.49 g).

### Example 35

### {1-[2-(3-Chloro-6-methoxy-[1,5]naphthyridin-4-yl)-2-hydroxyethyl]-piperidin-4-yl}-(2,3-dihydro-[1,4]dioxino[2,3-c]pyridin-7-ylmethyl)-amine Dihydrochloride Enantiomer 1

### (a) 7-Chloro-2-methoxy-8-oxiranyl-[1,5]naphthyridine Enantiomer 1 and Enantiomer 2

The racemic oxirane (1e) (3.55 g) was subjected to preparative HPLC on a Chiralpak AD 20 um column (77 mm×250 mm) eluting with 90:10 hexane:ethanol (isocratic) (flow rate 280 mL/min) to afford the fast-running isomer (Enantiomer 1) (1.67 g; 99% ee; retention time 9.4 min.) and the slow running enantiomer (Enantiomer 2) (1.62 g; 97% ee; retention time 12.9 min.).

### (b) {1-[(2-(3-Chloro-6-methoxy-[1,5]naphthyridin-4-yl)-2-hydroxy-ethyl]-piperidin-4-yl}-carbamic acid tert-butyl ester Enantiomer 1

A mixture of epoxide (35a; enantiomer 1) (0.813 g) and piperidin-4-yl-carbamic acid tert-butyl ester (0.69 g) was heated in DMF (5 drops) at 100° C. for 6 hr. The product was dissolved in chloroform and chromatographed on silica gel (methanol-DCM) to afford the solid product (1.0 g) containing ca. 20% of the epoxide ‘wrong-opening’ isomer.

### (c) 1-[2-(3-Chloro-6-methoxy-[1,5]naphthyridin-4-yl)-2-hydroxy-ethyl]-piperidin-4-ylamine Enantiomer 1

The ester (35b) (0.69 g) was deprotected by the method of Example (31g) to give a foam (0.68 g) containing ca. 20% of the ‘epoxide wrong-opening’ isomer.

### (d) Title Compound

The amine (35c) (0.68 g) and aldehyde (2c) (0.334 g) were heated in chloroform (4 mL) and methanol (4 mL) with 3 A molecular sieves for 1.5 hr at 80° C., cooled, and treated with sodium triacetoxyborohydride (1.28 g) and the mixture was stirred overnight at room temperature. Chloroform was added and the mixture was filtered, treated with sodium carbonate solution and extracted with methanol-chloroform, dried (sodium sulfate), evaporated and chromatographed on silica gel (methanol-DCM) to afford the free base of the title compound (0.65 g), containing ca 20% of the epoxide ‘wrong-opening’ isomer

LC-MS (ES) m/z 486/488 (M+H)⁺ (2 peaks with retention time 1.13 and 1.23 min.)

¹H NMR δH (CDCl₃, 400 MHz), 1.40-1.70 (2H, m), 1.88 (2H, br. d), 2.25 (2H, q), 2.52 (1H, m), 2.65 (1H, dd), 3.00 (2H, m), 3.10 (1H, dd), 3.80 (2H, s), 4.05 (3H, s), 4.25-4.35 (4H, m), 5.67 (1H, m), 6.38 (1H, brs), 6.83 (1H, s), 7.15 (1H, d), 8.05 (1H, s), 8.23 (1H, d), 8.70 (1H, s) (plus impurity peaks).

This material, as a solution in chloroform/methanol, was treated with an excess of 4M HCl in dioxan and evaporated to dryness. The solid was recrystallised several times (cold methanol), triturated with ether, filtered and dried under vacuum to provide the pure title compound (60 mg), [LC-MS (ES) single peak with retention time 1.23 min.]

### Example 36

### 6-({1-[2-(3-Chloro-6-methoxy-[1,5]naphthyridin-4-yl)-2-hydroxy-ethyl]-piperidin-4-ylamino}-methyl)-4H-pyrido[3,2-b][1,4]thiazin-3-one Dihydrochloride Enantiomer 1

The amine (35c) (0.78 g) and aldehyde (7d) (0.45 g) were dissolved in DMF (2 mL), methanol (2 mL) and acetic acid (0.2 mL) and heated with 3 A molecular sieves for 2 hr at 80° C., cooled, and treated with sodium cyanoborohydride (0.44 g) and the mixture was stirred overnight at room temperature. Chloroform was added and the mixture was filtered, treated with sodium carbonate solution and extracted with methanol-chloroform, dried (sodium sulfate), evaporated and chromatographed on silica gel (methanol-DCM) to afford the free base of the title compound as a solid (0.64 g), containing 15-20% of the ‘epoxide wrong-opening’ isomer.

LC-MS (ES) m/z 499/501 (M+H)⁺ (2 peaks retention time 1.22 and 1.30 min.)

¹H NMR δH (CDCl₃, 400 MHz), 1.40-1.70 (2H, m), 1.88 (2H, br. d), 2.25 (2H, q), 2.52 (1H, m), 2.65 (1H, dd), 3.00 (2H, br t), 3.07 (1H, dd), 3.80 (2H, s), 4.03 (3H, s), 4.65 (2H, s), 5.67 (1H, m), 6.42 (1H, br d), 6.95 (1H, d), 7.15 (2H, 2×d), 8.21 (1H, d), 8.70 (1H, s).

This material, as a solution in chloroform/methanol, was treated with an excess of 4M HCl in dioxan and evaporated to dryness. The solid was recrystallised from methanol-water, washed with a small amount of water then ether, and dried under vacuum to provide the pure title compound [LC-MS (ES) single peak with retention time 1.30 min.].

### Example 37

### 6-({1-[2-(3-Chloro-6-methoxy-[1,5]naphthyridin-4-yl)-2-hydroxy-ethyl]-piperidin-4-ylamino}-methyl)-4H-pyrido[3,2-b][1,4]thiazin-3-one Dihydrochloride Enantiomer 2

### (a) {1-[(2-(3-Chloro-6-methoxy-[1,5]naphthyridin-4-yl)-2-hydroxy-ethyl]-piperidin-4-yl)-carbamic acid tert-butyl ester Enantiomer 2

This was prepared from a mixture of epoxide (35a Enantiomer 2) (0.74 g) and piperidin-4-yl-carbamic acid tert-butyl ester (0.63 g) by the method of Example (35b) to afford the product (0.71 g) containing ca. 20% of the epoxide ‘wrong-opening’ isomer.

### (b) 1-[(2-(3-Chloro-6-methoxy-[1,5]naphthyridin-4-yl)-2-hydroxy-ethyl]-piperidin-4-ylamine Enantiomer 2

This was prepared from the ester (37a) (0.71 g) by the method of Example (35c) to give the product as an oil (0.52 g) containing ca. 20% of the ‘epoxide wrong-opening’ isomer.

### (c) Title Compound

This was prepared from the amine (37b) (0.52 g) and aldehyde (7d) (0.30 g) by the method of Example (36c) to afford the free base of the title compound as a solid (0.42 g), containing 15-20% of the ‘epoxide wrong-opening’ isomer.

LC-MS (ES) m/z 499/501 (M+H)⁺ (2 peaks with retention time 1.22 and 1.30 min.)

¹H NMR δH (CDCl₃, 400 MHz), 1.40-1.70 (2H, m), 1.88 (2H, br. d), 2.25 (2H, q), 2.52 (1H, m), 2.65 (1H, dd), 3.00 (2H, br t), 3.07 (1H, dd), 3.80 (2H, s), 4.03 (3H, s), 4.65 (2H, s), 5.67 (1H, m), 6.42 (1H, br d), 6.95 (1H, d), 7.15 (2H, 2×d), 8.21 (1H, d), 8.70 (1H, s).

This material, as a solution in chloroform/methanol, was treated with an excess of 4M HCl in dioxan and evaporated to dryness. The solid was recrystallised from methanol-water, washed with a small amount of water then ether, and dried under vacuum to provide the pure title compound [LC-MS (ES) single peak with retention time 1.30 min.].

### Example 38

### {6-(trans)-1-[2-(3-Chloro-6-methoxyquinolin-4-yl)ethyl]-3-hydroxypiperidin-4-yl}-(2,3-dihydro-[1,4]dioxino[2,3-c]pyridin-7-ylmethyl)amine Enantiomer 2

### (a) trans-4-amino-1-[2-(3-chloro-6-methoxyquinolin-4-yl)ethyl]piperidin-3-ol enantiomer 2

This was prepared by hydrogenation of piperidine (17f, enantiomer 2) by the method of Example (5c) followed by reaction with 7-chloro-2-methoxy-8-vinyl-quinoline and removal of the Boc protecting group (see Example 5d-e) (0.055 g).

### (b) Title Compound

This was prepared by the general procedure of Example (5f) from amine (38a) and 2,3-dihydro-[1,4]dioxino[2,3-c]pyridine-7-carboxaldehyde (Example 2c) and sodium borohydride, to give a yellow solid (0.0266 g, 37%) following flash chromatography on silica gel (9:1 CHCl₃/MeOH containing 1% NH₄OH).

¹H NMR (400 MHz, d₄-MeOH) δ 8.59 (s, 1H), 8.02 (s, 1H), 7.92 (d, J=9 Hz, 1H) 7.42 (m, 2H), 6.98 (s, 1H), 4.38 (m, 2H), 4.32 (m, 2H), 4.00 (s, 3H), 3.88 (d, J=13.8 Hz, 1H), 3.75 (m, d, J=13.8 Hz, 1H), 3.55 (m, 1H), 3.43 (t, J=8.2 Hz, 2H), 3.33 (m, 2H), 3.21 (m, 1H), 3.08 (m, 1H), 2.66 (m, 2H), 2.38 (m, 1H), 2.12 (m, 3H), 1.48 (m, 1H). LC/MS (ES) m/z 485 (M+H)⁺.

### Example 39

### (trans)-6-({1-[2-(3-Chloro-6-methoxy-[1,5]naphthyridin-4-yl)-ethyl]-3-hydroxy-piperidin-4-ylamino}-methyl)-4H-pyrido[3,2-b][1,4]-thiazin-3-one Dihydrochloride Enantiomer 2

### (a) trans-{1-[2-(3-Chloro-6-methoxy-[1,5]naphthyridin-4-yl)-ethyl]-3-hydroxy-piperidin-4-yl)-carbamic acid tert-butyl ester Enantiomer 2

To a solution of 7-chloro-2-methoxy-8-vinyl-[1,5] naphthyridine (as prepared in Example (3a)) (1.14 g, 5.16 mmol) in anhydrous DMF (5 mL) was added trans-4-tert-butoxycarbonylamino-3-hydroxy-piperidine enantiomer 2 (1.2 g, 5.16 mmol) [prepared from Example (17f, enantiomer 2) by hydrogenation]. After heating the mixture at 85° C. for 18 h, the reaction mixture was cooled to room temperature and concentrated in vacuo. The crude product was purified by column chromatography on silica gel (gradient elution: 50% EtOAc/hexanes to 100% EtOAc) to afford an off-white solid (1.1 g, 49%).

¹H NMR (400 MHz, CDCl₃) δ8.67 (s, 1H), 8.17 (d, 1H, J=9 Hz), 7.12 (d, 1H, J=9 Hz), 4.63 (m, 1H), 4.09 (s, 3H), 3.74 (m, 1H), 3.52 (m, 4H), 3.44 (m, 1H), 3.28 (m, 1H), 2.97 (m, 1H), 2.81 (m, 2H), 2.3 (m, 1H), 2.24 (m, 1H), 1.96 (m, 1H), 1.47 (s, 9H); LC/MS (ES) m/z 437.4 (M+H)⁺.

### (b) trans-4-Amino-1-[2-(3-chloro-6-methoxy-[1,5]naphthyridin-4-yl)-ethyl]-piperidin-3-ol enantiomer 2 trihydrochloride

To a solution of carbamate (39a) (1.1 g, 2.52 mmol) in dichloromethane (15 mL) was added 4 N HCl in dioxane (6.3 mL, 25.2 mmol). After stirring for 1 h, the reaction mixture was concentrated in vacuo to obtain a pale yellow solid (1 g, 89%) LC/MS (ES) m/z 337.4 (M+H)⁺.

### (c) Title Compound

To a solution of amine (39b) (0.5 g, 1.12 mmol) in anhydrous dichloromethane (20 mL) and absolute ethanol (40 mL) was added 3-oxo-3,4-dihydro-2H-pyrido[3,2-b][1,4]thiazine-6-carboxaldehyde (7d) (0.218 g, 1.12 mmol) and triethylamine (5.6 mmol, 0.780 mL). To this reaction mixture was added anhydrous sodium sulfate and the reaction was stirred at RT for 18 h under N₂, then sodium borohydride (43 mg, 1.12 mmol) was added and stirring was continued for an additional 2 h. The crude product was filtered through a cake of Celite®, washing with 10% methanol/dichloromethane, and the filtrate was concentrated in vacuo. Purification by column chromatography on silica gel (10% methanol/dichloromethane containing 5% NH₄OH in methanol) gave the title compound (260 mg, 45%) as the free base. This was dissolved in dichloromethane and 4 N HCl in dioxane (1.01 mmol, 0.252 mL) was added. The solid was triturated with diethyl ether and evaporated to dryness to afford the title compound (0.3 g, 45%) as a yellow solid:

¹H NMR (400 MHz, CD₃OD) δ8.67 (s, 1H), 8.15 (d, 1H, J=9 Hz), 7.66 (d, 1H, J=7.8 Hz), 7.17 (d, 1H, J=9 Hz), 7.02 (d, 1H, J=7.8 Hz), 4.37 (m, 3H), 4.09 (s, 3H), 3.86 (m, 2H), 3.73 (m, 2H), 3.52 (m, 1H), 3.43 (m, 2H), 3.37 (m, 2H), 3.28 (m, 1H), 3.13 (m, 1H), 2.45 (m, 1H), 2.25 (m, 1H). LC/MS (ES) m/z 515.4 (M+H)⁺.

### Example 40

### trans-6-({1-[2-(3-Chloro-6-methoxy-[1,5]naphthyridin-4-yl)ethyl]-3-hydroxy-piperidin-4-ylamino}-methyl)-4H-pyrido[3,2-b][1,4] oxazin-3-one Trihydrochloride Enantiomer 2

To a solution of amine (39b) (0.499 g, 1.12 mmol) in anhydrous dichloromethane (20 mL) and absolute ethanol (40 mL) was added 3-oxo-3,4-dihydro-2H-pyrido[3,2-b][1,4] oxazine-6-carboxaldehyde (1l) (0.200 g, 1.12 mmol) and triethylamine (5.6 mmol, 0.780 mL). To this reaction mixture was added anhydrous sodium sulfate and the reaction was stirred at RT for 18 h under N₂, then sodium borohydride (44 mg, 1.12 mmol) was added and stirring was continued for an additional 2 h. The crude product was filtered through a cake of Celite, washing with 10% methanol/dichloromethane, and the filtrate was concentrated in vacuo. Purification by column chromatography on silica gel (10% methanol/dichloromethane containing 5% NH₄OH in methanol) gave the title compound (130 mg, 23%) as the free base. This was dissolved in dichloromethane and 4 N HCl in dioxane (0.782 mmol, 0.195 mL) was added. The solid was triturated with diethyl ether and evaporated to dryness to afford the title compound (25%) as an off-white solid:

¹H NMR (400 MHz, DMSO-d₆) δ 9.81 (s, 1H), 9.31 (s, 1H), 8.84 (s, 1H), 8.33 (d, 1H, J=9 Hz), 7.46 (d, 1H, J=8.1Hz), 7.34 (d, 1H, J=9 Hz), 7.23 (d, 1H, J=8.1 Hz), 4.70 (s, 2H), 4.38 (m, 7H), 4.12 (s, 3H), 3.81 (m, 3H), 3.56 (m, 1H), 3.43 (m, 3H), 3.18 (m, 1H), 2.99 (m, 1H), 2.56 (m, 1H), 2.18 (m, 1H). LC-MS (ES) m/z 499.4 (M+H)⁺.

### Example 41

### trans-6-({1-[2-(3-Chloro-6-methoxy-[1,5]naphthyridin-4-yl]ethyl}-3-hydroxy-piperidin-4-ylamino}-methyl)-4H-pyrido[3,2-b][1,4] thiazin-3-one dihydrochloride Enantiomer 1

### (a) trans-4-Amino-1-[2-(3-chloro-6-methoxy-[1,5]naphthyridin-4-yl)-ethyl]-piperidin-3-ol Enantiomer 1

To a solution of 7-chloro-2-methoxy-8-vinyl-[1,5] naphthyridine (3a) (1.2 g, 5.5 mmol) in anhydrous DMF (2.5 mL) was added trans-4-tert-butoxycarbonylamino-3-hydroxy-piperidine enantiomer 1 (1.2 g, 5.5 mmol) [prepared from (17f, enantiomer 1) by hydrogenation]. The mixture was heated at 85° C. for 18 h, then was cooled to room temperature and concentrated in vacuo. To the crude product was added dioxan (5 mL) followed by 4N HCl in dioxan (10 mL). After stirring for 1 h, the reaction mixture was concentrated in vacuo. The crude product was purified by flash chromatography on silica gel (gradient elution: 4% MeOH in CH₂Cl₂, then 90:10:1 CH₂Cl₂/MeOH/conc. NH₄OH, then 80:20:2 CH₂Cl₂/MeOH/conc. NH₄OH) to afford an off-white solid (1.3 g, 71% for two steps). LC/MS (ES) m/z 337 (M+H)⁺.

### (b) Title Compound

To a solution of amine (41a) (1.1 g, 3.9 mmol) in DMF (15 mL) containing 4 A molecular sieves was added 3-oxo-3,4-dihydro-2H-pyrido[3,2-b][1,4]thiazine-6-carboxaldehyde (7d) (0.64 g, 3.9 mmol). The mixture was stirred at RT under N₂ for 18 h, then was filtered. The filtrate was concentrated to dryness and the residue was dissolved in MeOH (15 mL). Sodium borohydride (0.15 g, 3.9 mmol) was added and the reaction mixture was stirred for an additional 2 h. The solvent was evaporated and the residue was purified by column chromatography on silica gel (gradient elution: 4% MeOH/CH₂C[2, then 90:10:1 CH₂Cl₂/MeOH/conc. NH₄OH). Recrystallization of the purified product from MeOH/H₂O gave the free base of the title compound (1.1 g, 54%). The title compound was obtained by adding 2 equivalents of 1N HCl to a solution of the free base (0.90 g) in MeOH. Evaporation of the solvent and drying in high vacuum @ 40° C. for 2-days, followed by trituration with Et₂O, afforded the title compound as a yellow solid (0.90 g). LC/MS (ES) m/z 515 (M+H)⁺.

### Example 42

### 6-({(3R,4r,5S)-1-[2-(3-Chloro-6-methoxy-quinolin-4-yl)-ethyl]-3,5-dihydroxy-piperidin-4-ylamino)}-methyl)-4H-pyrido[3,2-b][1,4]oxazin-3-one dihydrochloride

### (a) (+/−) (1R,5S,6S)-5-Hydroxy-7-oxa-3-aza-bicyclo[4.1.0]heptane-3-carboxylic acid benzyl ester

To a solution of (+/−) 3-hydroxy-3,6-dihydro-2H-pyridine-1-carboxylic acid benzyl ester (Heterocycles 1992, 33, 349, or Synthesis 2000, 521; 1.4 g, 6.0 mmol) in CH₂Cl₂ (25 mL) at 0° C. was added MCPBA (60% by weight, 1.7 g, 6.0 mmol).

After stirring at this temperature for 18 h, the reaction mixture was poured into a solution of saturated Na₂CO₃ and extracted with EtOAc (2×). The combined extracts were washed with brine, dried (MgSO₄), and concentrated to afford a clear oil (quantitative yield). LC/MS (ES) m/z 250 (M+H)⁺.

### (b) (3S,4r,5R)-4-Azido-3,5-dihydroxy-piperidine-1-carboxylic acid benzyl ester

To a solution of (+/−) (1R,5S,6S)-5-hydroxy-7-oxa-3-aza-bicyclo[4.1.0]heptane-3-carboxylic acid benzyl ester (1.6 g, 6.4 mmol) in DMF (25 mL) containing LiClO₄ (0.76 g, 7.1 mmol) was added NaN₃ (0.46 g, 7.1 mmol). The reaction mixture was heated at 80° C. for 1 h then the solvent was evaporated. The residue was purified by flash chromatography on silica gel (gradient elution: 33% EtOAc/hexanes then 50% EtOAc/hexanes) to afford a white solid (0.70 g, 37%).

¹H NMR (MeOH-d₄): δ 7.26-7.18 (m, 5H), 5.01 (s, 3H), 4.09-4.06 (m, 2H), 3.26-3.20 (m, 2H), 3.04 (dd, 1H, J=9.4, 3.4); COSY45 showed that only the methines on carbon bearing oxygen correlated to the methylenes indicating epoxide opening as indicated.

### (c) (3S,4r,5R)-4-Amino-3,5-dihydroxy-piperidine-1-carboxylic acid benzyl ester

To a degassed solution of (3S,4r,5R)-4-azido-3,5-dihydroxy-piperidine-1-carboxylic acid ethyl ester (0.50 g, 1.7 mmol) in EtOAc (50 mL) was added 5% Pd/C (Degussa-type, 0.10 g). After stirring under hydrogen (1 atm) for 18 h, the reaction mixture was degassed and filtered through Celite®, and the filtrate was concentrated to afford a clear oil, which was used in the next step without purification. LC/MS (ES) m/z 267 (M+H)⁺.

### (d) (3S,4r,5R)-4-tert-Butyloxycarbonylamino-3,5-dihydroxy-piperidine-1-carboxylic acid benzyl ester

To a solution of amine (42c) (1.7 mmol) in EtOAc (25 mL) at RT was added di-tert-butyl dicarbonate After stirring at RT for 18 h, the reaction was concentrated and the residue was triturated with Et₂O to afford a white solid (0.42 g, 68% for two steps).

### (e) ((3S,4r,5R)-3,5-Dihydroxy-piperidin-4-yl}carbamic acid tert-butyl ester

To a degassed solution of benzyl ester (42d) (0.32 g, 0.87 mmol) in MeOH (15 mL) was added 20% Pd(OH)₂/C (0.030 g). After stirring under hydrogen (1 atm) for 18 h, the reaction mixture was degassed and filtered through Celite®, and the filtrate was concentrated to afford a clear oil (0.17 g, 84%). LC/MS (ES) m/z 267 (M+H)⁺.

### (f) (3R,4r,5S)-1-[2-(3-Chloro-6-methoxy-quinolin-4-yl)-ethyl]-3,5-dihydroxy-piperidin-4-yl}-carbamic acid tert-butyl ester

To a solution of 7-chloro-2-methoxy-8-vinyl-quinoline (4c) (0.10 g, 0.45 mmol) in DMF (2.5 mL) was added piperidine (42e) (0.094 g, 0.45 mmol). After heating to 100° C. for 4 days, the reaction was concentrated and the residue was purified by flash chromatography on silica gel (4% MeOH/CH₂Cl₂) to afford an oil (0.055 g, 27%).

### (g) (3R,4r,5S)-4-Amino-1-[2-(3-chloro-6-methoxy-quinol-4-yl)-ethyl]-piperidine-3,5-diol trihydrochloride

To a solution of ester (42f) (0.055 g, 0.12 mmol) in dioxan (5.0 mL) was added 4 N HCl in dioxan (5 mL). After stirring at RT for 2.5 h, the reaction was concentrated. The residue was subjected to high vacuum at 40° C. for 18 h to afford a yellow solid, which was used in the next step without purification.

### (h) Title Compound

To a solution of amine (42g) (0.12 mmol) in DMF (2.5 mL) containing Cs₂CO₃ (0.098 g, 0.30 mmol) and 4 A molecular sieves was added 3-oxo-3,4-dihydro-2H-pyrido[3,2-b][1,4]oxazine-6-carboxaldehyde (1l) (0.026 g, 0.13 mmol). The reaction was stirred at RT for 18 h, then the solvent was evaporated. MeOH (10 mL) was added to the residue, followed by NaBH₄ (0.049 g, 0.13 mmol). The reaction mixture was stirred at RT for 1 h and then concentrated. The residue was purified by flash chromatography on silica gel (gradient elution: 4% MeOH/CH₂Cl₂, then 10% MeOH/CH₂Cl₂, then 90:10:1 CH₂Cl₂/MeOH/conc. NH₄OH). Fractions containing only the desired product were combined and concentrated, and the residue was dissolved in MeOH containing 1N HCl. The solvent was evaporated and the residue was triturated with Et₂O to afford the title compound (0.010 g, 14% over three steps) as a light-yellow solid.

¹H NMR (MeOH-d₄): δ 8.49 (s, 1H); 7.82 (d, 1H, J=9.1Hz), 7.34-7.28 (m, 2H), 7.15 (d, 1H, J=8.0 Hz), 6.86 (d, 1H, J=8.0 Hz); 4.53 (s, 2H), 3.95 (s, 2H), 3.50 (m, 2H); 3.35 (m, 2H,), 3.04 (dd, 2H, J=10.7, 4.0 Hz), 2.62 (m, 2H), 2.24 (t, 1H, J=9.4 Hz); 2.03 (t, 2H, J=10.5 Hz). LC/MS (ES) m/z 514 (M+H)⁺.

### Example 43

### 6-({1-[2-(3-Fluoro-6-methoxyquinolin-4-yl)ethyl]piperidin-4-yl amino}methyl)-4H-pyrido[3,2-b][1,4]oxazin-3-one dihydrochloride)

This compound was prepared from amine crude amine (31g), prepared from 1.84 mmol carbamate (31h), and aldehyde (1l) (0.32 g, 1.80 mmol) by the method of Example (31f). Chromatography on silica (5-15% methanol/dichloromethane) gave the free-base (0.77 g, 90%).

¹H NMR (250 MHz, CDCl₃) δ8.58 (1H, d), 7.99 (1H, d), 7.30 (1H,d), 7.25 (1H, d), 7.20 (1H, d), 6.92 (1H, d), 4.62 (2H, s), 3.96 (3H, s), 3.84 (2H, s), 3.31 (2H, m), 3.12 (2H, m), 2.73 (2H, m), 2.66 (1H, m), 2.34 (2H, m), 2.00 (2H, m), 1.65 (2H, m). MS (ES) m/z 466 (M+H)⁺

The free base in dichloromethane/methanol was treated with 2 equivalents of HCl (4M in 1,4-dioxan), followed by evaporation of solvent and trituration with ether to give the title compound.

### Example 44

### {1-[2-(3-Bromo-6-methoxy-quinolin-4-yl)-ethyl]-piperidin-4-yl}-(2,3-dihydro-[1,4]dioxino[2,3-c]pyridin-7-ylmethyl)-amine Dihydrochloride

### (a) 3-Bromo-6-methoxy-quinolin-4-ol

6-Methoxy-quinolin-4-ol (4.0 g) in acetic acid (65 mL) was treated with N-bromosuccinimide (4.5 g) and the mixture was heated at 35° C. for 4 hr, cooled, and the solid collected and dried in vacuo to give a solid (4.0 g).

MS (ES) m/z 255/257 (M+H)⁺.

### (b) 1,1,1-Trifluoro-methanesulfonic acid 3-bromo-6-methoxy-quinolin-4-yl ester

Dry DMF (25 mL) was added to a suspension of 60% sodium hydride in oil (0.47 g). It was cooled to 0° C., the phenol (44a) (2.0 g) was added and the mixture was stirred for 15 min. N-phenyltrifluoromethanesulphonimide (3.0 g) was added and the mixture was allowed to stir at room temperature overnight. It was evaporated, and chromatographed on silica gel (petroleum ether/DCM) and washed with sodium bicarbonate solution, dried (magnesium sulfate) and evaporated to give a solid (1.95 g). MS (+ve ion electrospray) m/z 387/389 (MH+).

### (c) 3-Bromo-6-methoxy-4-vinyl-quinoline

This was prepared from the triflate (44b) (0.40 g) to give a solid (0.20 g) by the method of Example (4c), heating for 2 hr at 100° C. MS (ES) m/z 264/266 (M+H)⁺.

### (d) {1-[2-(3-Bromo-6-methoxy-quinolin-4-yl)-ethyl]-piperidin-4-yl}-carbamic acid tert butyl ester

A mixture of the vinyl-quinoline (44c) (0.20 g) and piperidin-4-yl-carbamic acid tert-butyl ester (0.152 g) in chloroform (0.35 mL) was heated at 100° C. for 4 days, then the product was dissolved in DCM and chromatographed on silica gel (methanol-EtOAc) to afford the solid product (0.23 g). MS (ES) m/z 464/466 (M+H)⁺.

### (e) 1-[2-(3-Bromo-6-methoxy-quinolin-4-yl}-ethyl]-piperidin-4-ylamine

The ester (44d) (0.23 g) was dissolved in chloroform (6 mL) and trifluoroacetic acid (6 mL) and the solution was stirred at room temperature for 0.5 hr then evaporated to dryness, basified (sodium bicarbonate) and the solid product collected, washed with water and dried in vacuo. MS (ES) m/z 364/366 (M+H)⁺.

### (f) Title Compound

The amine (44e) (0.158 g) and aldehyde (2c) (0.72 g) were dissolved in chloroform (3 mL) and methanol (3 mL) with 3 A molecular sieves and heated at 70° C. for 2 hr., cooled and sodium triacetoxyborohydride (0.27 g) was added and the solution was stirred overnight at room temperature. The mixture was filtered and evaporated, re-dissolved in DCM and chromatographed on silica gel (methanol-ammonia-EtOAc) to afford the free base of the title compound as a solid (0.13 g). MS (ES) m/z 513/515 (M+H)⁺.

This material, as a solution in chloroform/methanol, was treated with an excess of 4M HCl in dioxan and evaporated to dryness. The solid was triturated with ether to give the title compound (0.07 g).

### Example 45

### cis-1-[2-(3-Chloro-6-methoxy-quinolin-4-yl)-ethyl]-4-[(2,3-dihydro-[1,4]dioxino[2,3-c]pyridin-7-ylmethyl)-amino]-piperidin-3-ol Dihydrochloride Enantiomer 1

A solution of cis-4-amino-1-[2-(3-chloro-6-methoxy-quinolin-4-yl)-ethyl]-piperidin-3-ol enantiomer 1 [(5e; free base) prepared from (5d) by reaction with trifluoroacetic acid/DCM followed by a basic work-up] (229 mg) and carboxaldehyde (2c) (0.113 g) in DMF (7 mL) was treated with sodium triacetoxyborohydride (0.45 g) portionwise and the mixture was stirred at room temperature overnight. It was quenched with 2N HCl, basified with sodium bicarbonate and extracted with 5% methanol-DCM, dried (magnesium sulfate), evaporated and purified by silica gel chromatography eluting with EtOAc:MeOH:NH₄OH_{(aq)} and then by preparative HPLC (to remove a small quantity of bis-alkylated material) to afford the free base of the title compound.

This material was converted to the title compound (100 mg) by dissolving in chloroform and adding 2 equivalents of 1M HCl/ether then evaporating to dryness.

¹H NMR δH (250 MHz, CD₃OD) 8.96 (1H, s), 8.36 (1H, s), 8.10 (1H, d), 7.55 (2H, m), 7.35 (1H, d), 4.60 (1H, m), 4.50 (2H, m), 4.45 (2H, s), 4.40 (2H, m), 4.10(3H, s), 4.00-3.85 (4H, m), 3.75 (1H, m), 3.50-3.30 (4H, m), 2.55-2.30 (2H, m).

MS (ES) m/z 485/487 (M+H)⁺.

### Example 46

### cis-1-[2-(3-Chloro-6-methoxy-quinolin-4-yl)-ethyl]-4-[(2,3-dihydro-[1,4]dioxino[2,3-c]pyridin-7-ylmethyl)-amino]-piperidin-3-ol Dihydrochloride Enantiomer 2

This was prepared from cis-4-amino-1-[2-(3-chloro-6-methoxy-quinolin-4-yl)-ethyl]-piperidin-3-ol enantiomer 2 (243 mg) [(prepared from cis-4-tert-butoxycarbonylamino-3-hydroxy-piperidine-1-carboxylic acid benzyl ester enantiomer 2 (5b) by the methods described for Example 45] to give, after silica gel chromatography the free base of the title compound.

This material was converted to the title compound (120 mg) by dissolving in chloroform and adding 2 equivalents of 1M HCl/ether then evaporating to dryness.

¹H NMR δH (250 MHz, (CD₃)₂SO) [symbol] 8.74 (1H, s), 8.22 (1H, s), 8.00 (1H, d), 7.60 (1H, d), 7.50 (1H, dd), 7.00 (1H, s), 6.56 (1H, brs), 4.45 (1H, m), 4.40 (2H, m), 4.32 (2H, m), 4.25 (2H, m), 4.05 (3H, s), 3.90-3.50 (5H, m), 3.40-3.05 (4H, m), 2.30-2.10 (2H, m). MS (ES) m/z 485/487 (M+H)⁺.

### Example 47

### 1-{2-[3,8-difluoro-6-(methoxy)-4-quinolinyl]ethyl}-N-(2,3-dihydro[1,4]dioxino[2,3-c]pyridin-7-ylmethyl)-4-piperidinamine dihydrochloride

### (a) 3-fluoro-4-nitrophenyl methyl ether

A solution of 3-fluoro-4-nitrophenol (25 g, 0.159 mmol) in acetonitrile (500 mL) and methanol (500 mL) was treated with diisopropyl ethylamine (28 mL). The reaction mixture was cooled in an ice-bath and after 30 minutes, trimethylsilyidiazomethane was added dropwise. The mixture was stirred at room temperature for 18 hours then evaporated under vacuum to afford the product as an oil (29.4 g, 100%). MS (+ve ion electrospray) m/z 172 (MH+).

### (b) 2-fluoro-4-(methoxy)aniline

A solution of (a) (28.1 g, 164 mmol) in ethanol (200 mL) was hydrogenated with palladium on charcoal. The reaction mixture was filtered through Kieselguhr and evaporated under vacuum to afford the product as an oil (22.8 g, 98%).

MS (+ve ion electrospray) m/z 141 (MH+).

### (c) ethyl 8-fluoro-6-(methoxy)-4-oxo-1,4-dihydro-3-quinolinecarboxylate

A mixture of aniline (b) (22.8 g, 162 mmol) and diethyl [(ethyloxy)methylidene]propanedioate (32.6 mL) were heated to reflux in Dowtherm A under a flow of argon. After 15 minutes (when all ethanol was removed), the mixture was allowed to cool down and was diluted with pentane. A precipitate was formed which was triturated with pentane, filtered and dried under vacuum to afford the product as an oil (33.06 g, 77%).

MS (+ve ion electrospray) m/z 265 (MH+).

### (d) ethyl 4-bromo-8-fluoro-6-(methoxy)-3-quinolinecarboxylate

To a solution of quinolone (c) (12 g, 45 mmol) in DMF (56 ml) was added dropwise phosphorus tribromide (4.5 ml, 47 mmol) over fifteen minutes (slightly exothermic). The reaction was held at 0° C., with an ice bath, for one hour and allowed to warm to room temperature then stirred for a further 2 hours. The mixture was then diluted with water (400 mL). A solution of sodium bicarbonate was added to reach pH 7. The reaction mixture was stirred for one hour at 0° C. then filtered. The precipitate was washed with water and dried in vacuo to afford the product as a yellow solid (12.2 g, 82%).

MS (+ve ion electrospray) m/z 329 (MH+).

### (e) 4-bromo-8-fluoro-6-(methoxy)-3-quinolinecarboxylic acid

A solution of bromide (d) (12.2 g, 37.3 mmol) in tetrahydrofuran (450 mL) was diluted by addition of a solution of sodium hydroxide 2N (27 mL) in water (75 mL). The reaction mixture was stirred overnight at room temperature then acidified to pH 3 with a solution of hydrogen chloride 5N. The solvents were evaporated to half the volume in vacuo. The reaction mixture was acidified to pH 1 by further addition of hydrogen chloride 5N, cooled to 4° C. for 30 minutes then filtered. The precipitate was dried in vacuo to afford the product as a white solid (10.1 g, 90%).

MS (+ve ion electrospray) m/z 301 (MH+).

### (f) 1,1-dimethylethyl [4-bromo-8-fluoro-6-(methoxy)-3-quinolinyl]carbamate

A solution of carboxylic acid (e) (7.5 g, 25 mmol) in butanol (40 mL) and DMF (88 mL) was treated with triethylamine (30 mL) then diphenylphosphoryl azide (5.8 mL, 27.5 mmol). The reaction mixture was heated at 100° C. for two hours under argon atmosphere. The mixture was then cooled down to room temperature and evaporated to half the volume in vacuo. Water (100 mL) was added to the mixture under vigorous stirring. A precipitate was formed, filtered and dried in vacuo. This crude product was chromatographed on silica gel eluting with 10% methanol in dichloromethane to afford the product as a white solid (6.4 g, 69%).

MS (+ve ion electrospray) m/z 372 (MH+).

### (g) 4-bromo-8-fluoro-6-(methoxy)-3-quinolinamine

Carbamate (f) (6.4 g, 17.3 mmol)) was treated with trifluoroacetic acid (50 ml) in dichloromethane (50 ml) at room temperature for two hours then evaporated to dryness. The residue was basified with sodium bicarbonate. A precipitate was formed which was filtered and dried in vacuo to afford the product as a white solid (4.7 g, 100%). MS (+ve ion electrospray) m/z 272 (MH+).

### (h) 4-bromo-6-methoxy-8-fluoroquinolin-3-yl-diazonium tetrafluoroborate

A solution of quinolinamine (g) (3 g, 11.1 mmol) in anhydrous THF (40 mL) cooled down to −9° C., with an ethanol/ice bath, was treated with nitrosonium tetrafluoroborate (1.4 g, 12.2 mmol) added portionwise over 20 minutes. The reaction mixture was stirred for 30 minutes at −2° C. under argon atmosphere. A precipitate was formed which was filtered, washed with cold THF and dried in vacuo overnight to afford the product as a yellow solid (3.2 g, 79%). MS (+ve ion electrospray) m/z 370 (MH+).

### (i) 4-bromo-3,8-difluoro-6-(methoxy)quinoline

Diazonium salt (h) (2.4 g, 6.5 mmol) was added to hot Decalin® (45 mL). The reaction mixture was maintained at 170° C. for 5 minutes. Cold Decalin® (20 mL) was added and the reaction mixture was cooled down with an ice bath. The Decalin® layer was decanted off the dark residue and washed with a solution of sodium bicarbonate, brine and water. The organic layer was dried over magnesium sulfate. Solvents from the work-up were evaporated under vacuum and and the Decalin® layer was cooled down to 4° C. A precipitate was formed (product) which was filtered off. The decalin filtrate and the dark residue obtained before work-up were combined and chromatographed eluting with dichloromethane to afford the further product as a white solid (combined yield, 0.75 g, 42%). MS (+ve ion electrospray) m/z 275 (MH+).

### (j) 4-ethenyl-3,8-difluoro-6-(methoxy)quinoline

Bromide (i) (0.63 g, 2.3 mmol) in DME (26 mL) under argon, was treated with tetrakis(triphenylphosphine)palladium(0) (0.13 g, 0.115 mmol) and the mixture stirred at room temperature for 20 minutes. Anhydrous potassium carbonate (0.32 g, 2.3 mmol), water (7 mL), and vinylborane:pyridine complex (see F. Kerins and D O'Shea J. Org. Chem. 2002, 67, 4968-4971) (0.22 g, 0.92 mmol) were added and the mixture was heated at 100° C. for 2 hr. It was cooled, diluted with water and extracted with ether, dried over magnesium sulfate and evaporated to dryness. After work-up the product was chromatographed on silica gel, eluting with 10% methanol in DCM to afford a white solid (0.46 g, 90%). MS (+ve ion electrospray) m/z 221 (MH+).

### (k) 1,1-dimethylethyl (1-{2-[3,8-difluoro-6-(methoxy)-4-quinolinyl]ethyl}-4-piperidinyl)carbamate

A mixture of the vinyl-quinoline 0) (0.46 g, 2.08 mmol), piperidin-4-yl-carbamic acid tert-butyl ester (0.62 g, 3.12 mmol) in DMF (0.7 mL) and tetramethylguanidine (5 drops) was heated at 100° C. for 18 hours. It was cooled, diluted with water and extracted with ethyl acetate, dried over magnesium sulfate and evaporated to dryness. After work-up the product was chromatographed on silica gel, eluting with methanol-DCM to afford the desired product as a white solid (0.5 g, 62%). MS (+ve ion electrospray) m/z 421 (MH+).

### (l) 1-{2-[3,8-difluoro-6-(methoxy)-4-quinolinyl]ethyl}-4-piperidinamine

The carbamate (k) (0.5 g, 1.3 mmol)) was treated with trifluoroacetic acid (14 ml) in dichloromethane (14 ml) at room temperature for two hours then evaporated to dryness. The residue was basified to pH 8 with sodium bicarbonate and extracted several times with a solution of 10% methanol in dichloromethane. The combined organic layers were dried over magnesium sulfate and evaporated to dryness to afford the product as a white solid (0.4 g, 100%). MS (+ve ion electrospray) m/z 321 (MH+).

### (m) Title Compound

The amine (I) (0.43 g, 1.35 mmol) and aldehyde (2c) (0.22 g, 1.35 mmol) were dissolved in DMF (14 mL) and sodium triacetoxyborohydride (0.87 g, 4.05 mmol) added. The solution was stirred overnight at room temperature. The reaction mixture was quenched with 2N HCl, basified with sodium bicarbonate solution, and extracted with 5% methanol in dichloromethane. The residue was chromatographed eluting with 0-10% methanol in dichloromethane to afford the free base of the product as a white solid (0.23 g, 37%).

¹H NMR δH (d4-MeOD) 8.57 (1H, s), 8.00 (1H, s), 7.23 (1H, dd), 7.15 (1H, dd), 6.96 (1H, s), 4.31 (4H, m), 3.98 (3H, s), 3.79 (2H, s), 3.10 (2H, m), 2.65 (2H, m), 2.62 (1H, m), 2.19 (2H, m), 1.96 (2H, m), 1.51 (2H, m). MS (+ve ion electrospray) m/z 471 (MH+).

This material, as a solution in chloroform/methanol, was treated with an excess of 1M HCl in ether and evaporated to dryness. The solid was triturated under ether, filtered and dried under vacuum to provide the title compound.

The following examples were prepared by analogous method to Example 47, using the aldehyde shown:

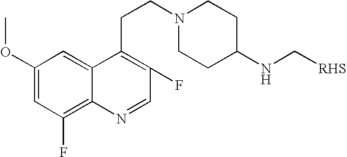

| Example | |
|---|---|
| 48 | 7-{[(1-{2-[3,8-Difluoro-6-(methoxy)-4-quinolinyl]ethyl}-4- |
| | piperidinyl)amino]methyl}-1H-pyrido[2,3-b][1,4]thiazin-2(3H)-one |
| | dihydrochloride |
| | 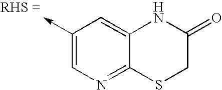 |
| | Preparation of 2-Oxo-2,3-dihydro-1H-pyrido[2,3-b][1,4]thiazine-7- |
| | carbaldehyde |
| | 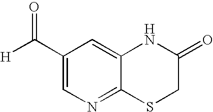 |
| | (a) 6-Methoxycarbonylmethylsulfanyl-5-nitro-nicotinic acid methyl |
| | ester |
| | A solution of 6-chloro-5-nitro-nicotinic acid methyl ester (1.0 g) |
| | [prepared as described by A. H. Berrie et al. J. Chem. Soc. |
| | 2590-2594 (1951)] in dichloromethane (10 mL) containing triethylamine |
| | (0.76 mL) was treated with mercapto-acetic acid methyl ester (0.44 |
| | mL) and the solution was stirred at room temperature for 1 hour and |
| | evaporated to dryness. Sodium bicarbonate solution was added and |
| | the mixture was extracted with dichloromethane, dried (anhydrous |
| | sodium sulfate) and evaporated to afford a solid (1.0 g). |
| | MS (+ve ion electrospray) m/z 287 (MH+). |
| | (b) 2-Oxo-2,3-dihydro-1H-pyrido[2,3-b][1,4]thiazine-7-carboxylic acid |
| | methyl ester |
| | The ester (a) (1.0 g) in acetic acid (50 mL) was treated with |
| | iron powder (10 g) and the mixture was stirred and heated at 60° C. for |
| | 1 hour, cooled and filtered. The filtrate was evaporated, treated with |
| | sodium bicarbonate solution and extracted with warm chloroform, It |
| | was dried (anhydrous sodium sulfate) and evaporated to give a white |
| | solid (0.85 g). |
| | MS (+ve ion electrospray) m/z 225 (MH+). |
| | (c) 2-Oxo-2,3-dihydro-1H-pyrido[2,3-b][1,4]thiazine-7-carboxylic acid |
| | A solution of ester (b) (2.8 g) in dioxan was treated dropwise |
| | with aqueous sodium hydroxide then acidified with 2 M HCl. After |
| | partial evaporation, a precipitate was formed, filtered and dried under |
| | vacuum to afford the product as a solid (2.5 g) |
| | MS (−ve ion electrospray) m/z 209 (M − H⁻). |
| | (d) 7-Hydroxymethyl-1H-pyrido[2,3-b][1,4]thiazin-2-one |
| | The carboxylic acid (c) (2.48 g) in THF with triethylamine was |
| | cooled to −10° C. and isobutylchloroformate was added. After 20 |
| | minutes the suspension was filtered through Kieselguhr into an ice- |
| | cooled solution of sodium borohydride in water. The mixture was |
| | stirred 30 minutes and the pH reduced to 7 with dilute HCl. The |
| | solvents were evaporated and the residue triturated under water. The |
| | product was filtered and dried under vacuum to afford a solid (1.3 g), |
| | after recrystallisation from chloroform-methanol (9:1). |
| | MS (+ve ion electrospray) m/z 197 (MH+). |
| | (e) 2-Oxo-2,3-dihydro-1H-pyrido[2,3-b][1,4]thiazine-7-carboxaldehyde |
| | A solution of alcohol (d) (1.22 g) was oxidised with |
| | manganese dioxide by the method of Example (2c) to afford a solid |
| | (0.7 g). |
| | MS (−ve ion electrospray) m/z 193 (M − H⁻). |
| 49 | 6-{[(1-(2-[3,8-Difluoro-6-(methoxy)-4-quinolinyl]ethyl}-4- |
| | piperidinyl)amino]methyl}-2H-pyrido[3,2-b][1,4]oxazin-3(4H)-one |
| | dinydrochloride |
| | 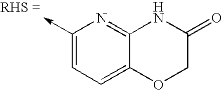 |
| | Aldehyde is 3-Oxo-3,4-dihydro-2H-pyrido[3,2-b][1,4]oxazine-6- |
| | carboxaldehyde as in example (1l) |
| 50 | 6-{[(1-{2-[3,8-difluoro-6-(methoxy)-4-quinolinyl]ethyl}-4- |
| | piperidinyl)amino]methyl}-2H-pyrido[a,2-b][1,4]thiazin-3(4H)-one |
| | dihydrochloride |
| | 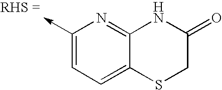 |
| | Aldehyde is 3-Oxo-3,4-dihydro-2H-pyrido[3,2-b][1,4]thiazine-6- |
| | carboxaldehyde as in example (7d) |
| 51 | 1-{2-[3,8-difluoro-6-(methoxy)-4-quinolinyl]ethyl}-N- |
| | ([1,3]dioxolo[4,5-c]pyridin-6-ylmethyl)-4- |
| | piperidinamine dihydrochloride |
| | 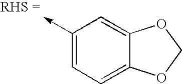 |
| | 1,3-benzodioxole-5-carbaldehyde is commercially available |

### Example 52

### {1-[2-(9-Chloro-2,3-dihydro-[1,4]dioxino[2,3-f]quinolin-10-yl)-ethyl]-piperidin-4-yl}-(2,3-dihydro-[1,4]dioxino[2,3-c]pyridin-7-ylmethyl)-amine dihydrochloride

### (a) 7-Bromo-2,3-dihydro-benzo[1,4]dioxin-6-ylamine

A solution of 2,3-dihydro-benzo[1,4]dioxin-6-ylamine (80g) in tetrahydrofuran (1 litre) at −78° C. was treated with concentrated sulfuric acid (80 drops) then N-bromosuccinimide was added over 0.5 hour. After the addition the mixture was stirred at −78° C. for 1 hour then treated with solid sodium carbonate (12g). The mixture was evaporated and the residue partitioned between ether and water. The organic extract was dried, filtered and evaporated to give to an oil that was chromatographed on silica gel eluting with dichloromethane to afford an oil (141 g, 92%). MS (+ve ion electrospray) m/z 231 (MH+).

### (b) 5-[(7-Bromo-2,3-dihydro-benzo[1,4]dioxin-6-ylamino)-methylene]-2,2-dimethyl-[1,3]dioxane-4,6-dione

A mixture of aniline (a) (14.8 g, 64.3 mmol), triethyl orthoformate (12.7 mL, 77.2 mmol) and 2,2-dimethyl-[1,3]dioxane-4,6-dione (Meldrum's acid) (11.1 g, 77.2 mmol) in ethanol (70 mL) was heated to reflux. After 1 hour the mixture was allowed to cool to room temperature then filtered, washing with ethanol then ether, to afford a white solid (22.9 g, 93%). MS (+ve ion electrospray) m/z 385 (MH+).

### (c) 6-Bromo-2,3-dihydro-7H-[1,4]dioxino[2,3-f]quinolin-10-one

Enamine (b) (22.9 g) was added portionwise to refluxing Dowtherm A® (45 mL) over 3 minutes. After a further 3 minutes at reflux the mixture was cooled to room temperature. Ethyl acetate/hexane (10 mL/20 mL) was added and a black solid isolated by filtration. This residue was dissolved in hot methanol (400 mL) and filtered through Keiselguhr. Water (800 mL) was added and the mixture stored at 5° C. overnight. Filtration and drying afforded a pale yellow solid (10.3 g, 61%). MS (APCl⁻) m/z 281 [M−H]

### (d) 2,3-Dihydro-7H-[1,4]dioxino[2,3-f]quinolin-10-one

A suspension of (c) (3.4 g, 12 mmol) in water/dioxan (150 mL/80 mL) was treated with 1M aqueous sodium hydroxide solution then hydrogenated over 10% palladium on charcoal (1.5 g) for 20 hours. The mixture was filtered then acidified with 5M aqueous hydrochloric acid. On concentrating to ca 100 mL, a solid began to crystallise out. The mixture was stored at 5° C. overnight. Filtration and drying afforded a pale yellow solid (2.8 g, 100%). MS (APCl⁻) m/z 202 [M−H]-(e) 9-Chloro-2,3-dihydro-[1,4]dioxino[2,3-f]quinolin-10-ol

The quinolone (d) (5.05 g) in acetic acid (70 mL) was sonicated and warmed until all had dissolved, and then it was treated with N-chlorosuccinimide (3.64 g) and the mixture was heated at 35° C. for 18 hr, cooled and the solid collected and washed with acetic acid and dried in vacuo at 40° C. overnight, to give a white solid (1.65 g). MS (ES) m/z 238/240 (M+H)⁺

### (f) 10-Bromo-9-chloro-2,3-dihydro-[1,4]dioxino[2,3-f]quinoline

The quinolin-4-ol (e) in dry DMF (8 mL) was cooled in ice and phosphorus tribromide (0.7 mL) added drop-wise, and the mixture was stirred, with ice-cooling for 30 minutes then allowed to warm to room temperature and stirred for a further 2 hours. It was cooled in ice and sodium carbonate solution was added and the solid was collected, washed well with water, and dried in vacuo, to afford a pale yellow solid (1.65 g). MS (ES) m/z 301/303/304 (M+H)⁺.

### (g) 9-Chloro-10-vinyl-2,3-dihydro-[1,4]dioxino[2,3-f]quinoline

The bromide (f) (1.65 g) in DME (60 mL) under argon, was treated with tetrakis(triphenylphosphine)palladium(0) (0.32 g) and the mixture stirred at room temperature for 20 minutes. Anhydrous potassium carbonate (0.76 g), water (18 mL), and vinylborane:pyridine complex (see F. Kerins and D O'Shea J. Org. Chem. 2002, 67, 4968-4971) was added and the mixture was heated at 100° C. for 2 hr. It was cooled, diluted with water and extracted with ether, dried (magnesium sulfate) and evaporated to dryness. After work-up the product was chromatographed on silica gel, eluting with methanol-DCM, to afford a white solid (1.35 g).

MS (ES) m/z 248/250 (M+H)⁺.

### (h) {1-[2-(9-Chloro-2,3-dihydro-[1,4]dioxino[2,3-f]quinolin-10-yl)-ethyl]-piperidin-4-yl}-carbamic acid tert-butyl ester

A mixture of the vinyl-quinoline (g) (680 mg) and piperidin-4-yl-carbamic acid tert-butyl ester (815.mg) in DMF (0.9 mL) and tetramethylguanidine (5 drops) was heated at 100° C. for 18 hours. It was cooled, diluted with water and extracted with ethyl acetate, dried (magnesium sulfate) and evaporated to dryness. After work-up the product was chromatographed on silica gel, eluting with methanol-DCM to afford the desired product (0.82 g). MS (ES) m/z 448 (M+H)⁺.

### (i) 1-[2-(9-Chloro-2,3-dihydro-[1,4]dioxino[2,3-f]quinolin-10-yl)-ethyl]-piperidin-4-ylamine

The carbamate (h) (0.82 g) in DCM (21 mL) was treated with TFA (21 mL) at room temperature for 1 hr and evaporated. Water and sodium carbonate were added and the solution was extracted with 10% methanol in ethyl acetate, dried (magnesium sulfate) and evaporated to afford the product (0.53 g). MS (ES) m/z 348 (M+H)⁺.

### (j) Title Compound

The amine (i) (0.53 g) and aldehyde (2c) (0.25 g) were dissolved in DMF (16 mL) and sodium triacetoxyborohydride (0.96 g) added and the solution was stirred overnight at room temperature. The reaction mixture was quenched with 2N HCl, basified with sodium bicarbonate solution, and extracted with methanol-DCM to afford the free base of the title compound (0.25 g).

This material, as a solution in chloroform/methanol, was treated with an excess of 1M HCl in ether and evaporated to dryness. The solid was triturated under ether, filtered and dried under vacuum to provide the title compound (0.33 g).

¹H NMR of the hydrochloride salt δH (d6-DMSO) 9.60 (2H, bs), 8.73 (1H, s), 8.20 (1H, s), 7.60 (1H, d), 7.45 (1H, d), 7.20 (1H, s), 4.50 (2H, m), 4.40 (4H, m), 4.32 (2H, m), 4.25 (2H, m), 3.90-3.70 (3H, m), 3.40-3.10 (6H, m), 2.35-2.05 (4H, m)

MS (+ve ion electrospray) m/z 497 (MH+).

### Example 53

### (2,3-dihydro[1,4]dioxino[2,3-c]pyridin-7-ylmethyl)-1-{2-[3-fluoro-6-(methoxy)-1,5-naphthyridin-4-yl]ethyl}-4-piperidinamine dihydrochloride

### (a) 2-[(6-Methoxypyridin-3-ylamino)-methylene]-malonic acid diethyl ester

5-Amino-2-methoxypyridine (100 g, 0.806 mole) in ethanol (1 litre) was treated with diethyl ethoxymethylenemalonate (Aldrich) (163 ml, 1 equivalent), refluxed 4 hours and cooled. The solvent was evaporated to dryness to afford the product (238 g, quantitative). MS (ES) m/z 295 (M+H)⁺.

### (b) 6-Methoxy-4-oxo-1,4-dihydro-[1,5]naphthyridine-3-carboxylic acid ethyl ester

Dowtherm A® (500 ml) in a 2 litre 3-neck flask fitted with still-head and condenser was brought just to boiling using an isomantle. Ester (a) (100 g) was added portionwise over 5 minutes and the solution boiled a further 10-15 minutes, allowing some solvent to distil over. The solution was cooled to room temperature, stirred and treated with n-pentane (750 ml) and cooled in ice for 1 hour. The brown solid was filtered off, washed with n-pentane and dried under vacuum to give the product (61.72 g, 73%). MS (ES) m/z 249 (M+H)⁺.

### (c) 4-Bromo-6-methoxy-[1,5]naphthyridine-3-carboxylic acid ethyl ester

A suspension of 6-methoxy-4-oxo-1,4-dihydro-[1,5]naphthyridine-3-carboxylic acid ethyl ester (b) (74.57 g, 300 mmole) in dry DMF (260 ml) under argon was stirred efficiently in a water bath. Phosphorus tribromide (30.0 ml, 316 mmole, 1.05 equiv.) was added dropwise over 15 minutes, stirring continued for 30 minutes and water (1 litre) added, followed by saturated sodium carbonate solution to pH7. The solid was filtered off, washed with water and dried under vacuum over phosphorus pentoxide to give product (83.56 g, 90%). MS (ES) m/z 312 (M+H)⁺.

### (d) 4-Bromo-6-methoxy-[1,5]naphthyridine-3-carboxylic acid

A solution of 4-bromo-6-methoxy-[1,5]naphthyridine-3-carboxylic acid ethyl ester (2c) (83.56 g, 268 mmole) in tetrahydrofuran (835 ml) was stirred and treated dropwise with 2N sodium hydroxide solution (300 ml, 600 mmole) over 30 minutes. Stirring was continued overnight. 2N HCl was added to pH6 and the THF was evaporated under vacuum. 2N HCl was then added to pH2, followed by 250 ml of water and the mixture was ice-cooled. The solid was filtered off, washed with water and dried under vacuum over phosphorus pentoxide to give product (76.7 g, slightly over quantitative, presumed to contain a small amount of inorganics, but used in this state). MS (ES) m/z 284 (M+H)⁺.

### (e) 4-Bromo-6-methoxy-[1,5]naphthyridin-3-ylamine

A suspension of 4-bromo-6-methoxy-[1,5]naphthyridine-3-carboxylic acid (d) (50 g, 177 mmole) in dry DMF (600 ml) was treated with triethylamine (222.5 ml), t-butanol (265 ml) and diphenylphosphoryl azide (41.75 ml, 194 mmole, 1.1 equiv.) and stirred under argon at 100° C. for 1 hour. The mixture was cooled and evaporated to low volume. Ethyl acetate and excess aqueous sodium bicarbonate solution were added, shaken and some insoluble solid filtered off. The layers were separated, the organic washed twice with water and dried over magnesium sulfate. Evaporation to dryness gave a crude mixture of 4-bromo-6-methoxy-[1,5]naphthyridin-3-ylamine (minor product) and (4-bromo-6-methoxy-[1,5]naphthyridin-3-ylamine)carbamic acid t-butyl ester (major product) along with impurities.

This mixture was dissolved in dichloromethane (150 ml) and treated with trifluoroacetic acid (100 ml), stirred 3 hours and evaporated. The residue was partitioned between chloroform and saturated sodium bicarbonate solution, the layers separated and the aqueous re-extracted with chloroform. The combined organic was dried over magnesium sulfate and evaporated to low volume. The solid was filtered, washed with a small volume of chloroform and dried under vacuum (31.14 g, clean by NMR). The filtrate was applied to a silica column and eluted with 30% ethyl acetate/chloroform to obtain futher material (2.93 g). (Total yield of product 34.07 g, 76%). MS (ES) m/z 255 (M+H)⁺.

### (f) 8-bromo-2-methoxy-1,5-naphthyridin-7-yl-diazonium tetrafluoroborate

A solution of aminonaphthyridine (e) (50.4 g, 198 mmol) in dry THF (800 mL) was stirred under argon atmosphere and maintained at −10° C. Nitrosonium tetrafluoroborate (26 g, 222 mmol) was added portionwise over one hour and the resulting supsension stirred a further 30 minutes. After completion of the reaction, the suspension was filtered cold, the solid washed with cold THF (250 mL) and dried under vacuum to afford the product (45.2 g, 65%). MS (ES) m/z 255 (M+H)⁺.

### (g) 8-bromo-7-fluoro-2-(methoxy)-1,5-naphthyridine

A suspension of diazonium fluoroborate (f) (40.7 g, 115 mmol in decalin (750 mL) was stirred well and heated in an oil bath until decomposition was complete. On completion (about 2 minutes), the reaction mixture was removed from heat and cooled in an ice/water bath. Chloroform (750 mL) was added to keep the product in solution. A black solid was formed which was triturated and sonicated for 30 minutes then chromatographed on a silica gel column eluting with 5% ethyl acetate in dichloromethane to obtain the product as a yellow solid (16.8 g, 57%).

MS (ES) m/z 258 (M+H)⁺.

### (h) 8-ethenyl-7-fluoro-2-(methoxy)-1,5-naphthyridine

Bromide (g) (10 g) in DME (310 mL) under argon, was treated with tetrakis(triphenylphosphine)palladium(0) (2.26 g, 0.05 eq) and the mixture stirred at room temperature for 20 minutes. Anhydrous potassium carbonate (5.37 g, 1 eq), water, and vinylborane:pyridine complex (see F. Kerins and D O'Shea J. Org. Chem. 2002, 67, 4968-4971) (5.85 g, 0.5 eq) was added and the mixture was heated at 80° C. for 4 hours. Further tetrakis(triphenylphosphine)palladium(0) (0.045 g), anhydrous potassium carbonate (0.54 g) and vinylborane:pyridine complex (0.6 g) were added and the reaction mixture was stirred at 80° C. for a further 4 hours. It was cooled, diluted with ethyl acetate, washed with a solution of sodium bicarbonate, dried over magnesium sulfate and evaporated to dryness. The residue was chromatographed on silica gel, eluting with 6% ethyl acetate in hexane to afford a white solid (6.4 g, 80%). MS (ES) m/z 205 (M+H)⁺.

### (i) 1-{2-[3-fluoro-6-(methoxy)-1,5-naphthyridin-4-yl]ethyl}-4-piperidinamine

A mixture of the vinyl-naphthyridine (h) (1 g, 5 mmol) and piperidin-4-yl-carbamic acid tert-butyl ester (1.3 g, 6.5 mmol) in DMF (6 mL) was heated at 105° C. for 22 hours, then at 110° C. for a further 7 hours. It was cooled, evaporated to dryness and chromatographed on silica gel, eluting with methanol-chloroform to afford the desired product as an oil.

The oil was redissolved in dichloromethane (30 mL) and the solution was treated with TFA (24 mL) and stirred at room temperature for 30 minutes. Solvents were evaporated under vacuum. Water and sodium carbonate were added and the solution was extracted with 15% methanol in chloroform, dried (magnesium sulfate) and evaporated to afford the product (390 mg, 59% over two steps). MS (ES) m/z 304 (M+H)⁺.

### (j) Title Compound

The amine (i) (0.45 g, 1.48 mmol) and aldehyde (2c) (0.24 g, 1.48 mmol) were dissolved in a mixture of chloroform (8 mL) and methanol (8 mL) in the presence of 3 A molecular sieves. The mixture was stirred at 70° C. for 4 hours cooled down and sodium triacetoxyborohydride (0.63 g, 2.96 mmol) was added. The reaction mixture was stirred overnight at room temperature. It was then filtered through Kieselguhr and partitioned between sodium bicarbonate and 10% methanol in chloroform. The organic layer was dried over magnesium sulfate, evaporated under vacuum and the residue was chromatographed eluting with chloroform/methanol/NH₄OH to afford the free base of the product as a white solid (0.61 g, 91%).

1H NMR δH(CDCl₃) 8.56 (1H, s), 8.16 (1H, d), 8.10 (1H, s), 7.06 (1H, d), 6.84 (1H, s), 4.20-4.35 (4H, m), 4.08 (3H, s), 3.80 (2H, s), 3.35-3.42 (2H, m), 3.00-3.06 (2H, m), 2.70-2.75 (2H, m), 2.45-2.55 (1H, m), 2.18 (2H, bt), 1.92 (2H, bd), 1.47 (2H, bq). MS (ES) m/z 454 (M+H)⁺.

This material, as a solution in chloroform/methanol, was treated with an excess of 1M HCl in ether and evaporated to dryness. The solid was triturated under ether, filtered and dried under vacuum to provide the title compound.

The following examples were prepared by analogous methods to Example 53, using the aldehydes shown:

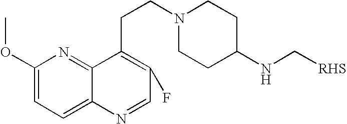

| Example | |
|---|---|
| 54 | N-(2,3-Dihydro-1H-pyrido[3,4-b][1,4]thiazin-7-ylmethyl)-1-{2-[3- |
| | fluoro-6-(methoxy)-1,5-naphthyridin-4-yl]ethyl}-4-piperidinamine |
| | dihydrochloride |
| | 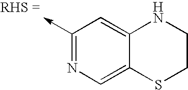 |
| | Preparation of 2,3-dihydro-1H-pyrido[3,4-b][1,4]thiazine-7- |
| | carbaldehyde |
| | 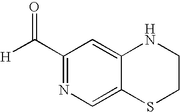 |
| | (a) 5-Fluoro-2-picoline N-oxide |
| | Preparation of 5-fluoro-2-picoline was based on E. J. Blanz, F. |
| | A. French, J. R. DoAmaral and D. A. French, J. Med. Chem. |
| | 1970, 13, 1124-1130. |
| | 5-Amino-2-picoline (12.5 g) in ethanol (105 ml) and 50% |
| | fluoroboric acid (44.5 ml) was stirred at −5° C. and treated |
| | dropwise over 45 minutes with n-butyl nitrite (31.25 ml). The |
| | solution was maintained at this temperature for 3 hours, treated |
| | with ether (100 ml, precooled to −20° C.) and the solid filtered off, |
| | quickly transferred to a flask and covered with hexane |
| | (precooled to −20° C.). After allowing to warm to approx. 20° C. |
| | and standing for 3 days the hexane was decanted and 2 M |
| | NaOH solution added until basic (pH10). The mixture was |
| | filtered and the filtrate extracted with dichloromethane |
| | (10 × 200 ml). The organic solution was dried, evaporated to 200 |
| | ml and treated with m-chloroperbenzoic acid (26.5 g). After |
| | stirring 16 hours the solution was washed with excess aqueous |
| | sodium bicarbonate and the aqueous re-extracted with |
| | dichloromethane (10 × 200 ml). The organic was dried and |
| | evaporated and the residue chromatographed (15% |
| | EtOH/EtOAc) to give title compound (5.5 g). |
| | MS (APCl⁺) m/z 128 (MH⁺, 100%) |
| | (b) 5-Fluoro-4-nitro-2-picoline N-oxide |
| | N-oxide (a) (2.12 g) was treated with an ice-cooled |
| | mixture of fuming nitric acid (7.1 ml) and conc. sulfuric acid |
| | (7.1 ml), heated at 35-40° C. for 1 hour and 65-70° C. for 5.5 |
| | hours, cooled and ice (45 g) added. 10 M NaOH was added to |
| | pH10 and the mixture extracted with EtOAc (3 × 30 ml). The |
| | oganic was dried and evaporated to give title compound as a |
| | yellow solid (2.16 g). |
| | MS (APCl⁺) m/z 173 (MH⁺, 30%), 127 (100%) |
| | (c) 5-Ethoxycarbonylmethylthio-4-nitro-2-picoline N-oxide |
| | Ethyl mercaptoacetate (1.51 g) in dioxan (15.6 ml) under |
| | argon was treated with sodium hydride (550 mg of a 60% |
| | dispersion in oil) and stirred for 1 hour. 5-Fluoro-4-nitro-2- |
| | picoline N-oxide (2.16 g) was added and stirring continued 3 |
| | days. Water (50 ml) was added and the mixture extracted with |
| | chloroform (3 × 50 ml). The organic was dried and evaporated to |
| | give a yellow solid (3.31 g). |
| | MS (APCl⁺) m/z 273 (MH⁺, 80%), 125 (100%) |
| | d) 2-Acetoxymethyl-5-ethoxycarbonylmethylthio-4-nitropyridine |
| | N-oxide (C) (3.31 g) in acetic anhydride (43 ml) was |
| | heated to 80° C. for 6 hours, evaporated, xylene (100 ml) added |
| | and evaporated. Chromatography of the residue (eluent |
| | EtOAc/hexane 1:1) gave title compound (1.03 g). |
| | (e) 7-Acetoxymethyl-2-oxo-2,3-dihydro-1H-pyrido[3,4- |
| | b][1,4]thiazine |
| | Nitropyridine (d) (1.03 g) in glacial acetic acid (27.5 ml) |
| | was treated with iron powder (1.75 g), stirred at 60° C. for 3 |
| | hours, filtered through kieselguhr and evaporated to dryness. |
| | Saturated aqueous sodium bicarbonate (300 ml) was added |
| | and extracted with EtOAc (3 × 200 ml), the organic was dried and |
| | evaporated. The residue was redissolved in acetic acid (30 ml), |
| | heated to 100° C. for 24 hours, evaporated and |
| | chromatographed (eluent EtOAc/hexane 1:1) to give title |
| | compound (340 mg). |
| | MS (APCl⁻) m/z 237 ([M − H]⁻, 90%), 195 (100%) |
| | (f) 7-Hydroxymethyl-2-oxo-2,3-dihydro-1H-pyrido[3,4- |
| | b][1,4]thiazine |
| | A solution of 7-acetoxymethyl-2-oxo-2,3-dihydro-1H- |
| | pyrido[3,4-b][1,4]thiazine (e) (340 mg) in dioxan (9 ml) was |
| | treated dropwise over 2 hours with 0.5 M NaOH (3.7 ml), stirred |
| | 18 hours and evaporated. Water (10 ml) was added and the |
| | white solid filtered off, washed with water and dried under |
| | vacuum to give title compound (231 mg). |
| | MS (APCl⁻) m/z 195 ([M − H]⁻, 100%) |
| | (g) 2-Oxo-2,3-dihydro-1H-pyrido[3,4-b][1,4]thiazine-7- |
| | carbaldehyde |
| | A mixture of alcohol (f) (226 mg), manganese dioxide |
| | (600 mg), THF (22.5 ml) and 1,2-dichloroethane (22.5 ml) was |
| | heated at 65° C. for 18 hours under argon. Filtration through |
| | kieselguhr and evaporation of solvent gave title compound as |
| | an off-white solid (173 mg). |
| | MS (APCl⁻) m/z 193 ([M − H]⁻, 100%) |
| | (h) 3,4-dihydro-2H-1,4-benzothiazin-6-ylmethanol |
| | A suspension of carboxaldehyde (g) (600 mg, 3.08 |
| | mmol) in dry THF (35 mL) was treated with 1 M solution of |
| | lithium aluminium hydride in THF (9 mL, 9 mmol). The mixture |
| | was refluxed for 5 hours under argon, cooled and treated with |
| | water (0.34 mL), a 2 N solution of sodium hydroxide (0.64 mL) |
| | and water again (0.72 mL). The reaction mixture was stirred |
| | for 15 minutes at room temperature and filtered. The filtrate |
| | was evaported to afford the product (432 mg, 77%) |
| | MS (+ve ion electrospray) m/z 182 (MH+). |
| | (i) 3,4-dihydro-2H-1,4-benzothiazine-6-carbaldehyde |
| | A solution of alcohol (h) (382 mg, 2.1 mmol) in |
| | acetonitrile (25 mL) was treated with 2-iodoxybenzoic acid (2 g) |
| | and heated at 80° C. for 2 hours. The mixture was filtered hot. |
| | The precipitate was boiled in acetonitrile (25 mL) and filtered. |
| | The combined filtrates were evaporated. The residue was |
| | sonicated in chloroform for 10 minutes chromatographed on a |
| | silica gel column eluting with 50% chloroform in ethyl acetate to |
| | afford the product (153 mg, 40%). |
| | MS (+ve ion electrospray) m/z 180 (MH+). |
| 55 | 6-{[(1-{2-[3-fluoro-6-(methoxy)-1,5-naphthyridin-4-yl]ethyl}-4- |
| | piperidinyl)amino]methyl}-2H-pyrido[3,2-b][1,4]oxazin-3(4H)- |
| | one dihydrochloride |
| | 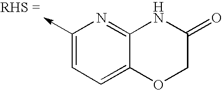 |
| | Aldehyde is 3-Oxo-3,4-dihydro-2H-pyrido[3,2-b][1,4]oxazine-6- |
| | carboxaldehyde as in example (1l) |
| 56 | 7-{[(1-(2-[3-fluoro-6-(methoxy)-1,5-naphthyridin-4-yl]ethyl}-4- |
| | piperidinyl)amino]methyl}-1H-pyrido[2,3-b][1,4]thiazin-2(3H)- |
| | one dihydrochloride |
| | 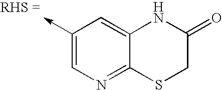 |
| | Aldehyde is 2-Oxo-2,3-dihydro-1H-pyrido[2,3-b][1,4]thiazine-7- |
| | carbaldehyde as in example 48 |
| 57 | 3-{[(1-{2-[3-fluoro-6-(methoxy)-1,5-naphthyridin-4-yl]ethyl)-4- |
| | piperidinyl)amino]methyl}-8-hydroxy-1(2H)-isoquinolinone |
| | dihydrochloride |
| | 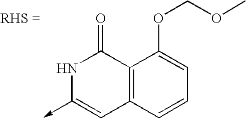 |
| | Preparation of 8-{[(methoxy)methyl]oxy}-1-oxo-1,2-dihydro-3- |
| | isoquinolinecarbaldehyde |
| | 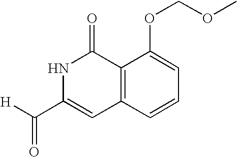 |
| | (a) Ethyl 2-methoxymethoxy-6-methylbenzoic acid |
| | A solution of ethyl 2-hydroxy-6-methylbenzoic acid |
| | (4.56 g, 25.3 mmol) and diisopropylethylamine (13.2 mL, 76 |
| | mmol) in dry dichloromethane (30 mL) was cooled in an ice- |
| | bath. Chloromethyl methyl ether (3.83 mL, 50.6 mmol) was |
| | added slowly and the mixture was allowed to stand at 0° C., |
| | warming slowly to room temperature. After 36 hours a further |
| | portion of chloromethyl methyl ether (1.9 mL) was added and |
| | the mixture was left at room temperature overnight. The |
| | mixture was then washed with 10% citric acid, water and brine, |
| | dried and evaporated to give the title compound (6.34 g, 100%). |
| | MS (+ve ion electrospray) m/z 225 (MH+). |
| | (b) 8-Methoxymethoxy-1-oxo-1H-isochromene-3-carboxylic |
| | acid ethyl ester |
| | n-Butyllithium (1.6 M in hexanes, 16.0 mL, 25.5 mmol) |
| | was added to a solution of diisopropylamine (3.64 mL, 25.5 |
| | mmol) and N,N,N′,N′-tetramethylethylenediamine (4.01 mL, 25.5 |
| | mmol) in dry tetrahydrofuran (36 mL) at −78° C. After 10 min a |
| | solution of the ester (a), (5.10 g, 22.8 mmol) in dry |
| | tetrahydrofuran (18 mL) was added dropwise, keeping the |
| | internal temperature <−60° C. The deep red solution was stirred |
| | at −78° C. for 40 min, then diethyl oxalate (3.10 mL, 22.8 mmol) |
| | in tetrahydrofuran (18 mL) was added over 5 min. The mixture |
| | was stirred at −78° C. for 6.5 hours, then treated with 10% citric |
| | acid. After warming to room temperature the phases were |
| | separated and the aqueous phase was extracted with ethyl |
| | acetate. The combined organic phases were washed with |
| | brine, dried and evaporated. Chromatography on silica gel |
| | (20-40% ethyl acetate/hexane) gave the product (2.05 g, 32%). |
| | MS (+ve ion electrospray) m/z 235 (loss of methoxymethyl from |
| | MH+). |
| | (c) 8-Methoxymethoxy-1,2-dihydro-1-oxo-isoquinoline-3- |
| | carboxylic acid ethyl ester |
| | The isochromene (b), (2.04 g, 7.34 mmol) was heated |
| | under reflux with ammonium acetate (4.99 g) in ethanol |
| | (200 mL) for 24 hours. Solvent was evaporated and the residue |
| | was dissolved in ethyl acetate and water. The aqueous phase |
| | was extracted with ethyl acetate and combined organics were |
| | washed with water, dried and evaporated. Chromatography on |
| | silica gel (50-100% ethyl acetate/hexane) gave impure product |
| | and recovered isochromene. The latter was treated again with |
| | ammonium acetate (1.3 g) in ref luxing ethanol (50 mL) for 48 |
| | hours, then worked up as before. The crude material was |
| | combined with the initial impure product for chromatography on |
| | silica gel (0-2% methanol/dichloromethane). Eluted material |
| | was re-chromatographed (50-100% ethyl acetate/hexane) to |
| | give the title compound (0.87 g, 42%). |
| | MS (+ve ion electrospray) m/z 278 (MH+). |
| | (d) 8-Methoxymethoxy-3-hydroxymethyl-2H-isoquinolin-1-one |
| | The ester (c), (0.66 g, 2.38 mmol) and sodium |
| | borohydride (0.14 g, 3.6 mmol) were heated in refluxing tert- |
| | butanol (3 mL) while methanol (0.6 mL) was added over 1 hour. |
| | Heating was continued for 2 hours ,then the cooled mixture |
| | was partitioned between ethyl acetate and water. The aqueous |
| | phase was re-extracted with ethyl acetate and the combined |
| | organics were washed with brine, dried and evaporated to give |
| | the title compound (0.51 g, 91%). |
| | MS (+ve ion electrospray) m/z 236 (MH+). |
| | (e) 8-{[(methoxy)methyl]oxy}-1-oxo-1,2-dihydro-3- |
| | isoquinolinecarbaldehyde |
| | The alcohol (d), (0.51 g, 2.17 mol) was stirred with manganese |
| | (IV) oxide (3.12 g) in 1:1 dichloromethane/tetrahydrofuran |
| | (40 mL) at room temperature for 5 hours. The mixture was |
| | filtered and evaporated to give the aldehyde (0.32 g, 63%). |
| | MS (−ve ion electrospray) m/z 232 (M − H⁻). |
| | After the reductive alkylation, the methoxymethyl protecting |
| | group was removed (to liberate the free phenol) with aqueous |
| | hydrochloric acid/dioxan, in quantitative yield giving the free |
| | base of the title compound |
| 58 | 3-{[(1-{2-[3-fluoro-6-(methoxy)-1,5-naphthyridin-4- |
| | yl]ethyl}-4-piperidinyl)amino]methyl}-5H- |
| | pyridazino[3,4-b][1,4]thiazin-6(7H)-one |
| | dihydrochloride |
| | 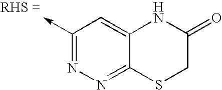 |
| | Preparation of 6-oxo-6,7-dihydro-5H-pyridazino[3,4- |
| | b][1,4]thiazine-3-carbaldehyde |
| | 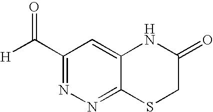 |
| | (a) 4-Amino-3,6-dichloropyridazine |
| | A suspension of 3,4,6-trichloropyridazine (prepared by |
| | the method of B. Kasnar et al, Nucleosides and Nucleotides, |
| | 1994, 13, 459) (10.0 g) in conc. aqueous ammonia (1L) was |
| | heated at 75° C. for 16 h. The mixture was concentrated to a |
| | small volume and extracted several times with ethyl acetate. |
| | The extracts were washed with brine, dried and evaporated. |
| | The crude product was recrystallised from ethyl acetate to give |
| | the title compound (5.03 g) |
| | (b) 3-Chloro-6-oxo-6,7-dihydro-5H-pyridazino[3,4- |
| | b][1,4]thiazine |
| | To a well-stirred suspension of sodium hydride (60% in |
| | mineral oil, 0.35 g, 8.5 mmol) in anhydrous dimethylformamide |
| | (10 mL) at 0° C. was added methyl mercaptoacetate (0.70 mL, |
| | 7.9 mmol). After stirring at this temperature for 20 min, a |
| | solution of 4-amino-3,6-dichloropyridazine (a), (1.29 g, 7.87 |
| | mmol) in dimethylformamide (10 mL) was added. The mixture |
| | was stirred at room temperature for 16 h, then most of the |
| | solvent was removed in vacuo. The residue was diluted with |
| | water, the precipitate was filtered off, washed with water and |
| | dried. Chromatography on silica (0-2% |
| | methanol/dichloromethane) gave the product (0.21 g, 13%). |
| | MS (+ve ion electrospray) m/z 202/204 (MH⁺) |
| | (c) 6-Oxo-3-vinyl-6,7-dihydro-5H-pyridazino[3,4-b][1,4]thiazine |
| | To a mixture of pyridazinothiazine (b) (0.15 g, 0.75 |
| | mmol), bis(triphenylphosphine)palladium(II) chloride (84 mg, |
| | 0.12 mmol) and lithium chloride (63 mg, 1.2 mmol) in |
| | dimethylformamide (3 mL) was added tributyl(vinyl)tin (0.36 mL, |
| | 1.2 mmol). The mixture was heated at 110-120° C. for 16 h, then |
| | evaporated. The residue was partitioned between water and |
| | ethyl acetate, the aqueous phase was extracted further with |
| | ethyl acetate and the combined organics were dried and |
| | evaporated. Chromatography on silica (0-3% |
| | methanol/dichloromethane) gave the product (45 mg, 31%). |
| | MS (+ve ion electrospray) m/z 194 (MH⁺) |
| | (d) 6-Oxo-6,7-dihydro-5H-pyridazino[3,4-b][1,4]thiazine-3- |
| | carboxaldehyde |
| | To a suspension of vinyl compound (c) (0.65 g, 3.35 |
| | mmol) in 1,4-dioxan (60 mL) was added osmium tetroxide (4% |
| | in water, 2 mL, 0.335 mmol), sodium periodate (1.43 g, 6.7 |
| | mmol) and water (20 mL). The mixture was stirred at room |
| | temperature for 7 h, then diluted with water and |
| | dichloromethane and phases separated. The aqueous phase |
| | was extracted twice with 10% methanol/dichloromethane and |
| | the combined organics were dried and evaporated. |
| | Chromatography on silica (0-2% methanol/dichloromethane) |
| | gave the aldehyde (0.206 g), containing some of the |
| | corresponding methyl hemiacetal. |
| | MS (+ve ion electrospray) m/z 196 (MH⁺). |
| 59 | 6-{[(1-{2-[3-Fluoro-6-(methoxy)-1,5-naphthyridin-4- |
| | yl]ethyl}-4-piperidinyl)amino]methyl}-2H-pyrido[3,2- |
| | b][1,4]thiazin-3(4H)-one dihydrochloride |
| | 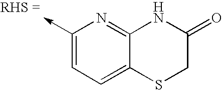 |
| | Aldehyde is 3-Oxo-3,4-dihydro-2H-pyrido[3,2-b][1,4]thiazine-6- |
| | carboxaldehyde as in example (7d) |
| 60 | N-(2,3-Dihydro[1,4]oxathiino[2,3-c]pyridin-7- |
| | ylmethyl)-1-{2-[3-fluoro-6-(methoxy)-1,5- |
| | naphthyridin-4-yl]ethyl}-4-piperidinamine |
| | dihydrochloride |
| | 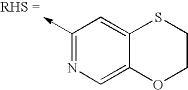 |
| | Preparation of 2,3-dihydro[1,4]oxathiino[2,3-c]pyridine-7- |
| | carbaldehyde |
| | 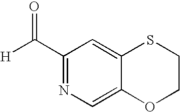 |
| | (a) 2-(hydroxymethyl)-5-({[4-(methoxy)phenyl]methyl}oxy)- |
| | 4(1H)-pyrone |
| | To a solution of Kojic acid (50 g, 0.352 mol) in DMF (650 mL) |
| | under an argon atmosphere, cooled to 0° C., was added a |
| | solution of potassium t-butoxide (39.5 g, 0.352 mol) in DMF |
| | (100 mL) and the resultant suspension was vigourously stirred |
| | (overhead stirring) for 1 hour at 5-10° C. 4-methoxybenzyl |
| | chloride was added dropwise and the mixture was heated to |
| | 50° C. for 30 hours, followed by 90° C. for 5 hours, after which |
| | the mixture was evaporated to a minimum volume of DMF. 750 |
| | mL of distilled water was added and the mixture refridgerated |
| | overnight. The resultant solid was collected by filtration and |
| | dried in vacuo at 50° C. to afford the product as a light brown |
| | solid (85 g, 64%). |
| | MS (+ve ion electrospray) m/z 263 (MH+) |
| | (b) 2-(hydroxymethyl)-5-({[4-(methoxy)phenyl]methyl}oxy) |
| | 4(1H)-pyridinone |
| | To a suspension of pyrone (a) (40 g, 153 mmol) in |
| | ethanol (105 mL) was added concentrated aqueous ammonia |
| | (295 mL) and refluxed for 18 hours. The mixture was cooled, |
| | then refridgerated for 3 hours, and cooled in an ice-bath for 45 |
| | minutes. The solid was filtered off, washed with cold ethanol, |
| | follwed by cold petroleum ether and dried in vacuo to afford the |
| | product as brown solid (26.21 g, 66%). |
| | (c) [5-({[4-(methoxy)phenyl]methyl}oxy)-4-oxo-1,4-dihydro- |
| | 2-pyridinyl]methyl acetate |
| | A solution of pyridone (b) (26 g, 0.1 mol) in pyridine |
| | (150 mL) was cooled to 5° C. and treated with acetyl chloride |
| | (10.48 ml, 0.149 mol). The reaction mixture was stirred and |
| | allowed to warm to room temperature then heated at 60° C. for |
| | 18 hours. Pyridine was evaporated under vacuum and the |
| | residue was triturated with water (250 mL), cooled in an ice- |
| | bath for 30 minutes. The solid formed was filtered off, washed |
| | with cold water and dried in vacuo to afford the product as a |
| | solid (15.7 g, 50%). |
| | MS (+ve ion electrospray) m/z 304 (MH+). |
| | (d) (5-({[4-(methoxy)phenyl]methyl}oxy)-4- |
| | {[(trifluoromethyl)sulfonyl]oxy}-2-pyridinyl)methyl acetate |
| | Pyridone (c) (25 g, 82 mmol) was dissolved in dry |
| | dichloromethane (600 mL). Triethylamine (23 mL, 164 mmol) |
| | was added and the reaction cooled to 0° C. Trifluoromethane |
| | sulfonic anhydride (21 mL, 123 mmol) was added dropwise and |
| | the reaction left to stir at room temperature overnight. The |
| | reaction was poured into water, the organic layer collected and |
| | dried (Mg SO₄). The crude product was chromatographed on |
| | silica eluting with 10-20% Ethyl acetate in hexane. Product |
| | containing fractions were combined and dried to afford the |
| | product as a solid (24.95 g, 70%). |
| | MS (+ve ion electrospray) m/z 436 (MH+). |
| | (e) [4-[(1,1-dimethylethyl)thio]-5-({[4- |
| | (methoxy)phenyl]methyl}oxy)-2-pyridinyl]methyl acetate |
| | To a solution of triflate (d) (10 g, 23 mmol) in anhydrous |
| | toluene, (R)-(+)-2,2 bis(diphenylphosphino)-1,1-binaphthyl (312 |
| | mg, 0.4 mmol) was added. The reaction mixture was degassed |
| | before adding palladium acetate (103 mg, 0.4 mmol). Sodium |
| | 2-methyl-2-propanethiolate was added, the system degassed |
| | again and the reaction mixture was strirred at 60° C. for 3 hours, |
| | under argon atmosphere then at 70° C. for a further 18 hours. |
| | The reaction mixture was filtered and the filtrate was |
| | evaporated under vacuum. The residue was partitioned |
| | between ethyl acetate and water. The aqueous layer was |
| | extracted several times with ethyl acetate. The combined |
| | organic extracts were dried over magnesium sulfate and |
| | evaporated under vacuum. The residue was chromatographed |
| | on silica gel eluting with 20-35% ethyl acetate in hexane to |
| | afford the product as an oil (9.1 g, 100%). |
| | MS (+ve ion electrospray) m/z 376 (MH+). |
| | (f) (4-[(1,1-dimethylethyl)thio]-5-hydroxy-2-pyridinyl}methyl |
| | acetate |
| | A solution of (e) (9 g, 24 mmol) in dichloromethane (100 |
| | mL) was treated with triethylsilane (3.86 mL, 24 mmol). The |
| | reaction mixture was stirred for 10 minutes before adding |
| | trifluoroacetic acid (10 mL). The reaction mixture was stirred at |
| | room temperature for 3 hours under argon atmosphere. The |
| | solvents were evaporated under vacuum. The residue was |
| | taken up in dichloromethane and chromatographed on silica gel |
| | eluting with 10%-30% ethyl acetate in hexane to afford the |
| | product as an oil (5.1 g, 83%). |
| | MS (+ve ion electrospray) m/z 256 (MH+). |
| | (g) 6-(hydroxymethyl)-4-mercapto-3-pyridinol |
| | Acetate (f) (2.5 g, 9.8 mmol) was dissolved in concentrated |
| | HCl and the mixture was heated at 80° C. for 18 hours. The |
| | solvent was evaporated under vacuum and the residue was |
| | triturated with diethyl ether to afford the product as a solid (1.35 |
| | g, 88%). |
| | MS (+ve ion electrospray) m/z 158 (MH+). |
| | (h) 2,3-dihydro[1,4]oxathiino[2,3-c]pyridin-7-ylmethanol |
| | To a solution of mercaptopyridinol (g) (500 mg, 3.2 mmol) |
| | in anhydrous DMF, potassium carbonate was added. The |
| | reaction mixture was stirred for 10 minutes and dibromoethane |
| | (0.55 mL, 6.4 mmol) was added. The reaction mixture was |
| | stirred at 70° C. for 18 hours under an argon atmosphere. DMF |
| | was removed in vacuo and the residue was partitioned |
| | between 5% MeOH in dichloromethane and water. The |
| | aqueous layer was extracted several times with 5% methanol in |
| | dichloromethane. The combined organic extracts were dried |
| | over magnesium sulfate and evaporated under vacuum. The |
| | residue was chromatographed on silica gel eluting with 3-5% |
| | methanol in dichloromethane to afford the product as a solid |
| | (381 mg, 70%). |
| | MS (+ve ion electrospray) m/z 184 (MH+). |
| | (i) 2,3-dihydro[1,4]oxathiino(2,3-c]pyridine-7-carbaldehyde |
| | Alcohol (h) was treated with manganese (IV) oxide as in |
| | example (2c) to afford the aldehyde as a solid. |
| | MS (+ve ion electrospray) m/z 182 (MH+). |
| 61 | 1-{2-[3-Fluoro-6-(methoxy)-1,5-naphthyridin-4-yl]ethyl}-N- |
| | ([1,3]oxathiolo[5,4-c]pyridin-6-ylmethyl)-4-piperidinamine |
| | dihydrochloride |
| | 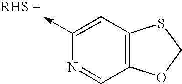 |
| | [1,3]Oxathiolo[5,4-c]pyridine-6-carbaldehyde was prepared |
| | from Example (60 g) (6-(hydroxymethyl)-4-mercapto-3-pyridinol) |
| | by reaction with dibromomethane and oxidation to the aldehyde |
| | using the same methodology as in Example (60). |

### Example 62

### 7-Fluoro-N-(1-{2-[3-fluoro-6-(methoxy)-1,5-naphthyridin-4-yl]ethyl}-4-piperidinyl)-3-oxo-3,4-dihydro-2H-pyrido[3,2-b][1,4]thiazine-6-carboxamide dihydrochloride

### (a) 6-Amino-5-bromo-3-fluoro-pyridine-2-carboxylic acid methyl ester

A mixture of 6-amino-5-bromo-pyridine-2-carboxylic acid methyl ester (19.8 g) (T. R. Kelly and F. Lang, J. Org. Chem. 61, 1996, 4623-4633) and 1-chloromethyl-4-fluoro-1,4-diazoniabicyclo[2.2.2]octane bis(tetrafluoroborate) (Selectfluor™), (34.3 g) in acetonitrile (340 ml) under argon was heated to 40° C. for 1 hour, 60° C. for 1 hour and then 80° C. overnight. After partitioning between EtOAc and water (500 ml each) the aqueous fraction was re-extracted with EtOAc (300 ml) and the combined organic solution dried with MgSO₄ and evaporated. Chromatography (20% then 30% EtOAc in hexane) afforded the product (2.09 g).

MS (+ve ion electrospray) m/z 249 and 251 (MH⁺).

### (b) 6-Amino-5-ethoxycarbonylmethylthio-3-fluoropyridine-2-carboxylic acid methyl ester

A solution of ethyl mercaptoacetate (1.15 ml) in DMF (40 ml) was ice-cooled under argon, treated with sodium hydride (420 mg of a 60% dispersion in oil) and stirred until all was in solution (about 1 hour). The ester (a) (2.48 g) was added, the mixture allowed to warm to room temp. and stirred overnight. EtOAc (150 ml) was added, the solution washed with water (3×150 ml), dried and evaporated. Chromatography of the residue (40% EtOAc in hexane) gave an oil (1.7 g). MS (+ve ion electrospray) m/z 289 (MH⁺)

### (c) Methyl 7-fluoro-3-oxo-3,4-dihydro-2H-pyrido[3,2-b][1,4]thiazine-6-carboxylate

A solution of the fluoropyridine (b) (1.7 g) in acetic acid (100 ml) was heated at 110° C. overnight, evaporated and dried under vacuum to give the product as a white solid (1.5 g). MS (+ve ion electrospray) m/z 243 (MH⁺).

### (d) 7-Fluoro-3-oxo-3,4-dihydro-2H-pyrido[3,2-b][1,4]thiazine-6-carboxylic acid

This compound was prepared from the ester (c) by the method of Example (7b) (86%).

### (e) Title Compound

A solution of carboxylic acid (d) (102 mg, 0.44 mmol) in THF (4 mL), at −15° C., under argon atmosphere, was treated with triethylamine (0.07 mL, 0.53 mmol) then iso-butylchloroformate (0.06 mL, 0.49 mmol). The mixture was stirred at −15° C. for 15 minutes and filtered through kieselguhr into a ice-cooled solution of amine (53i). The new reaction mixture was stirred for a further hour. Solvents were evaporated under vacuum and the residue was triturated under chloroform. The solid was filtered to afford the free base of the title compound as a solid (192 mg, 84%)

1H NMR δH (d6-DMSO) 11.08 (1H, s), 8.76 (1H, s), 8.26 (1H, d), 8.19 (1H, d), 7.96 (1H, d), 7.23 (1H, d), 4.03 (3H, s), 3.65-3.75 (1H, m), 3.61 (2H, s), 3.25-3.35 (2H, m, partly obscured by water), 2.93 (2H, bd), 2.68 (2H, bt), 2.17 (2H, bt), 1.77 (2H, bd), 1.40 (2H, bq). MS (+ve ion electrospray) m/z 515 (MH+).

This material was dissolved in chloroform/methanol and treated with an excess of 1M HCl in ether then evaporated to dryness. The solid was triturated with ether, filtered and dried under vacuum to provide the title compound.

The following examples were prepared by analogous methods to Example 62 using the acids shown:

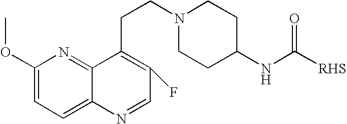

| Example | |
|---|---|
| 63 | N-(1-{2-[3-fluoro-6-(methoxy)-1,5-naphthyridin-4- |
| | yl]ethyl}-4-piperidinyl)-2-oxo-2,3-dihydro-1H- |
| | pyrido[2,3-b][1,4]thiazine-7-carboxamide |
| | dihydrochloride |
| | 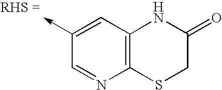 |
| | Acid is 2-oxo-2,3-dihydro-1H-pyrido[2,3-b][1,4]thiazine-7-carboxylic |
| | acid as in example (48c) |
| 64 | N-(1-{2-[3-fluoro-6-(methoxy)-1,5-naphthyridin-4- |
| | yl]ethyl}-4-piperidinyl)-3-oxo-3,4-dihydro-2H- |
| | pyrido[3,2-b][1,4]thiazine-6-carboxamide |
| | 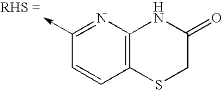 |
| | Acid is 3-oxo-3,4-dihydro-2H-pyrido[3,2-b][1,4]thiazine-6-carboxylic |
| | acid as in example (7b) |
| 65 | N-(1-{2-[3-fluoro-6-(methoxy)-1,5-naphthyridin-4-yl]ethyl}-4- |
| | piperidinyl)-3-oxo-3,4-dihydro-2H-pyrido[3,2-b][1,4]oxazine-6- |
| | carboxamide |
| | 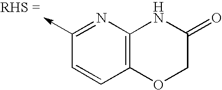 |
| | Preparation of 3-oxo-3,4-dihydro-2H-pyrido[3,2-b][1,4]oxazine-6- |
| | carboxylic acid |
| | 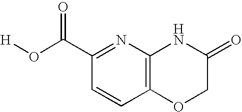 |
| | This acid was prepared from aldehyde (1l) (890 mg) by oxidation |
| | with Oxone (potassium peroxymonosulphate) (3.1 g) in a DMF |
| | solution (50 mL). After 1.5 hours at room temperature, dilution with |
| | water (50 mL) filtration and drying in vacuo afforded the acid as a |
| | white solid (750 mg, 77%). For this particular example, amide |
| | formation was accomplished by dissolving the acid (26 mg) and |
| | amine (53i) (41 mg) in DMF (0.5 ml) then treating with triethylamine |
| | (27 mg) and HATU (O-(7-azabenzotriazol-1-yl)N,N,N′,N′,- |
| | tetramethyluronium hexafluorophosphate) (56 mg). After 16 hours, |
| | dilution with water, filtration and drying in vacuc afforded the free |
| | base of the title compound (51 mg). |

### Example 66

### (3R,4S)-4-[(2,3-dihydro[1,4]dioxino[2,3-c]pyridin-7ylmethyl)amino]-1-{2-[3-fluoro-6-(methoxy)-1,5-naphthyridin-4-yl]ethyl}-3-piperidinol dihydrochloride Enantiomer 1

### (a) 1,1-dimethylethyl ((3R,4S)-1-{2-[3-fluoro-6-(methoxy)-1,5-naphthyridin-4-yl]ethyl}-3-hydroxy-4-piperidinyl)carbamate

This was prepared by reaction of vinyl naphthyridine (53h) (1.42 g) and piperidine (5c, Enantiomer1) (1.5 g) by heating in DMF (10 mL) with 1,1,3,3-tetramethylguanidine (0.5 mL) at 90° C. for 32 hours. Evaporation and chromatography on silica eluting with 5% methanol in dichloromethane afforded an oil (2.5 g). MS (ES) m/z 421 (M+H)⁺.

### (b) (3R,4S)-4-amino-1-{2-[3-fluoro-6-(methoxy)-1,5-naphthyridin-4-yl]ethyl}-3-piperidinol

A solution of carbamate (a) (2.5 g) in dichloromethane (30 mL) was treated with trifluoroacetic acid (25 mL) for 2 hours then evaporated to dryness and triturated with ether. The resulting solid was partitioned between saturated aqueous potassium carbonate solution and 10% methanol/chloroform. The aqueous phase was extracted a further 6 times with 10% methanol/chloroform and the combined organic extracts were dried and evaporated to afford an oil (1.72 g). MS (ES) m/z 321 (M+H)⁺.

### (c) Title Compound

The amine (b) (500 mg) and aldehyde (2c) (258 mg) were reacted together with sodium triacetoxyborohydride as in example (53j) to afford the free base of the title compound as a solid (420 mg, 55%).

1H NMR δH (CDCl₃) 8.61 (1H, s), 8.17 (1H, d), 8.10 (1H, s), 7.07 (1H, d), 6.84 (1H, s), 4.20-4.35 (4H, m), 4.08 (3H, s), 3.87 (1H, s), 3.83 (2H, s), 3.39 (2H, bt), 3.10 (1H, bd), 2.95 (1H, bd), 2.78 (2H, bt), 2.50-2.60 (1H, m), 2.34 (1H, d), 2.22 (1H, bt), 1.6-1.9 (m, including water). MS (+ve ion electrospray) m/z 470 (MH+).

This material, as a solution in chloroform/methanol, was treated with an excess of 1M HCl in ether and evaporated to dryness. The solid was triturated under ether, filtered and dried under vacuum to provide the title compound.

The following examples were prepared by analogous method to Example 66 using the aldehydes shown:

| | |
|---|---|
| | Isomer E1 |
| 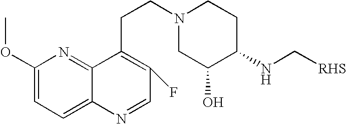 | |

| Example | |
|---|---|
| 67 | 6-{[((3R,4S)-1-{2-[3-fluoro-6-(methoxy)-1,5- |
| | naphthyridin-4-yl]ethyl}-3-hydroxy-4- |
| | piperidinyl)amino]methyl}-2H-pyrido[3,2- |
| | b][1,4]thiazin-3(4H)-one dihydrochloride |
| | 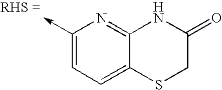 |
| | Aldehyde is 3-Oxo-3,4-dihydro-2H-pyrido[3,2-b][1,4]thiazine-6- |
| | carboxaldehyde as in example (7d) |
| 68 | 6-{[((3R,4S)-1-{2-[3-fluoro-6-(methoxy)-1,5- |
| | naphthyridin-4-yl]ethyl}-3-hydroxy-4- |
| | piperidinyl)amino]methyl}-2H-pyrido[3,2- |
| | b][1,4]oxazin-3(4H)-one dihydrochloride |
| | 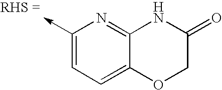 |
| | Aldehyde is 3-Oxo-3,4-dihydro-2H-pyrido[3,2-b][1,4]oxazine-6- |
| | carboxaldehyde as in example (1l)- |
| 69 | (3R,4S)-4-[(2,3-dihydro[1,4]dioxino[2,3-b]pyridin-7-ylmethyl)amino]- |
| | 1-{2-[3-fluoro-6-(methoxy)-1,5-naphthyridin-4-yl]ethyl)-3-piperidinol |
| | dihydrochloride |
| | 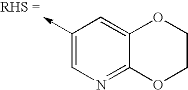 |
| | Aldehyde 2,3-dihydro[1,4]dioxino[2,3-b]pyridine-7-carbaldehyde |
| | was prepared as described in Example (40e) of WO02056882. |

### Example 70

### 6-{[((3S,4R)-1-{2-[3-fluoro-6-(methoxy)-1,5-naphthyridin-4-yl]ethyl}-3-hydroxy-4-piperidinyl)amino]methyl}-2H-pyrido[3,2-b][1,4]thiazin-3(4H)-one dihydrochloride Enantiomer 2—

### (a) (3S,4R)-4-amino-1-{2-[3-fluoro-6-(methoxy)-1,5-naphthyridin-4-yl]ethyl}-3-piperidinol

Vinyl-naphthyridine (53h) and piperidine (5c, Enantiomer 2) were reacted together and the adduct deprotected with trifluoroacetic acid as in Example (66a,b) to give an oil. MS (ES) m/z 321 (M+H)⁺.

### (b) Title Compound

The amine (a) and aldehyde (7d) were treated as in example (66c) to afford the free base of the title compound in 64% yield.

1H NMR δH (CDCl₃) 8.61 (1H, s), 8.18 (1H, d), 7.55 (1H, d), 7.06 (1H, d), 6.99 (1H, d), 4.07 (3H, s), 3.92 (1H, bs), 3.87 (2H, ABq), 3.43 (2H, s), 3.37 (2H, t), 3.14 (1H, bd), 2.98 (1H, bd), 2.7-2.9 (2H, m), 2.50-2.60 (1H, m), 2.34 (1H, d), 2.21 (1H, bt), 1.6-1.8 (2H, m). MS (+ve ion electrospray) m/z 499 (MH+).

This material, as a solution in chloroform/methanol, was treated with an excess of 1M HCl in ether and evaporated to dryness. The solid was triturated under ether, filtered and dried under vacuum to provide the title compound (85 mg).

### Example 71

### N-((3S,4R)-1-{2-[3-fluoro-6-(methoxy)-1,5-naphthyridin-4-yl]ethyl}-3-hydroxy-4-piperidinyl)-3-oxo-3,4-dihydro-2H-pyrido[3,2-b][1,4]thiazine-6-carboxamide hydrochloride Enantiomer 2—

Carboxylic acid (7b) and amine (70a) were treated as in Example (62) to afford the desired amide in 51% yield.

1H NMR δH (CDCl₃) 8.64 (1H, s), 8.20 (2H, d), 7.98 (1H, d), 7.83 (1H, d), 7.76 (1H, d), 7.09 (1H, d), 4.09 (3H, s), 3.95-4.05 (1H, m), 3.82 (1H, bs), 3.53 (2H, s), 3.39 (2H, t), 3.17 (1H, bd), 3.01 (1H, bd), 2.7-2.9 (3H, m), 2.44 (1H, d), 2.29 (1H, bt), 1.8-1.9 (1H, m), 1.6-1.8 (m, including water). MS (+ve ion electrospray) m/z 513 (MH+).

This material, as a solution in chloroform/methanol, was treated with an excess of 1M HCl in ether and evaporated to dryness. The residue was triturated with ether, filtered and dried under vacuum to provide the title compound as a pale yellow solid (55 mg).

### Example 72

### 7-{[((3R,4S)-1-{2-[3,8-difluoro-6-(methoxy)-4-quinolinyl]ethyl}-3-hydroxy-4-piperidinyl)amino]methyl}-1H-pyrido[2,3-b][1,4]thiazin-2(3H)-one dihydrochloride Enantiomer 1

### (a) (3S,4R)-4-amino-1-{2-[3-fluoro-6-(methoxy)-1,5-naphthyridin-4-yl]ethyl}-3-piperidinol

A mixture of vinyl-naphthyridine (47j) and piperidine (5c, Enantiomer 1) were reacted together and the adduct deprotected with trifluoroacetic acid as in Example (66a,b) to give an oil. MS (ES) m/z 338 (M+H)⁺.

### (a) Title Compound

The amine (a) and the aldehyde from Example (48) were treated as in Example (66c) to afford the free base of the title compound in 19% yield.

1H NMR δH (CDCl3) 8.63 (1H, s), 8.15 (1H, d), 8.00 (1H, bs), 7.17 (1H, d), 7.05 (1H, dd), 6.96 (1H, d), 3.95 (3H, s), 3.90 (1H, m), 3.85 (1H, d), 3.78 (1H, d), 3.58 (2H, s), 3.20 (2H, m), 3.12 (1H, m), 2.95 (1H, m), 2.70 (2H, m), 2.50 (1H, m), 2.30 (1H, m), 2.20 (1H, m), 1.75 (2H, m). MS (+ve ion electrospray) m/z 516 (MH+).

This material, as a solution in chloroform/methanol, was treated with an excess of 1M HCl in ether and evaporated to dryness. The solid was triturated under ether, filtered and dried under vacuum to provide the title compound as a white solid (80 mg).

The following examples were prepared by analogous methods to Example 70 using the aldehydes shown:

| | |
|---|---|
| | Isomer E1 |
| 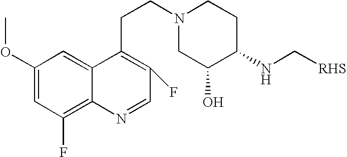 | |

| Exam- | |
|---|---|
| ple | |
| 73 | 6-{[((3R,4S)-1-{2-[3,8-difluoro-6-(methoxy)-4- |
| | quinolinyl]ethyl}-3-hydroxy-4- |
| | piperidinyl)amino]methyl}-2H-pyrido[3,2- |
| | b][1,4]thiazin-3(4H)-one dihydrochloride Enantiomer 1 |
| | 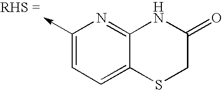 |
| | Aldehyde is 3-Oxo-3,4-dihydro-2H-pyrido[3,2-b][1,4]thiazine-6- |
| | carboxaldehyde as in example (7d) |
| 74 | (3R,4S)-1-{2-[3,8-difluoro-6-(methoxy)-4-quinolinyl]ethyl}-4- |
| | [(2,3-dihydro[1,4]dioxino[2,3-c]pyridin-7-ylmethyl)amino]-3- |
| | piperidinol dihydrochloride dihydrochloride Enantiomer 1 |
| | 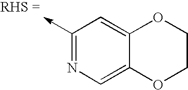 |
| | Aldehyde is 2,3-dihydro[1,4]dioxino[2,3-c]pyridine-7- |
| | carbaldehyde as in example(2c) |
| 75 | 6-{[((3R,4S)-1-{2-[3,8-difluoro-6-(methoxy)-4- |
| | quinolinyl]ethyl}-3-hydroxy-4-piperidinyl)amino]methyl}- |
| | 2H-pyrido[3,2-b][1,4]oxazin-3(4H)-one dihydrochloride |
| | 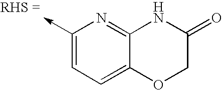 |
| | Aldehyde is 3-Oxo-3,4-dihydro-2H-pyrido[3,2-b][1,4]oxazine-6- |
| | carboxaldehyde as in example (1l) |

### Example 76

### N-[(4-fluoro-1 benzimidazol-2-yl)methyl]-1-{2-[3-fluoro-6-(methoxy)-4-quinolinyl]ethyl}-4-piperidinamine

### (a) 4-fluoro-1H-benzimidazole-2-carbaldehyde

Prepared from 4-fluoro-1H-benzoimidazole-2-ylmethanol, itself prepared from 3-fluoro-benzene-1,2-diamine by reaction with glycolic acid. MS (+ve ion electrospray) m/z 165 (MH+).

### (b) Title Compound

Amine (31g) and the aldehyde (a) were reacted together with sodium triacetoxyborhydride as in Example (53j) to afford the free base of the title compound in 56% yield.

1H NMR δH (CDCl₃) 8.55 (1H, s), 7.98 (1H, d), 7.33 (1H, dd), 7.31 (1H, m), 7.23 (1H, d), 7.18 (1H, td), 6.95 (1H, dd), 4.10 (2H, s), 3.97 (3H, s), 3.25 (2H, m), 3.08 (2H, m), 2.62 (3H, m), 2.18 (2H, t), 1.99 (2H, br d), 1.50 (2H, qd). MS (+ve ion electrospray) m/z 513 (MH+).

This material, as a solution in chloroform/methanol, was treated with an excess of 1M HCl in ether and evaporated to dryness. The solid was triturated with ether, filtered and dried under vacuum to provide the title compound.

The following examples were prepared by analogous methods to Example 76 using the aldehydes shown:

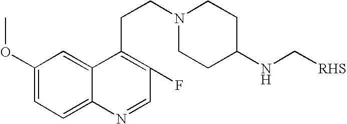

| Example | |
|---|---|
| 77 | 1-{2-[3-fluoro-6-(methoxy)-4-quinolinyl]ethyl}-N-(1,5,6,7- |
| | tetrahydro-1,8-naphthyridin-2-ylmethyl)-4-piperidinamine |
| | dihydrochloride |
| | 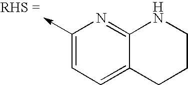 |
| | 1,5,6,7-tetrahydro-1,8-naphthyridine-2-carbaldehyde was prepared according to |
| | the procedure of WO 98/08840. |
| 78 | N-(3-cinnolinylmethyl)-1-{2-[3-fluoro-6-(methoxy)-4-quinolinyl]ethyl}-4- |
| | piperidinamine dihydrochloride |
| | 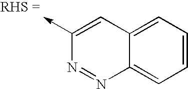 |
| | Preparation of 3-cinnolinecarbaldehyde |
| | 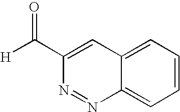 |
| | (a) 1-(3-cinnolinyl)-1,2,3,4-butanetetrol |
| | Anhydrous D-glucose (7.27 g, 40.4 mmol) was added to a warm, stirred |
| | solution of phenylhydrazine (26.0 g, 240.7 mmol) in HCl/water. The mixture was |
| | heated to reflux. A heavy yellow precipitate was formed and filtered after 2 |
| | hours then washed with warm water. The filtrate was cooled down to room |
| | temperature and further yellow precipitate was formed, filtered off and |
| | combined with the first one. The filtrate was basified to pH 9 by addition of |
| | diluted sodium hydroxide. The aqueous layer was extracted several times with |
| | chloroform. Some precipitate was formed in the aqueous layer, filtered and |
| | washed with water then dried under vacuum. The filtrate was heated at 80° C. |
| | with charcoal for 30 minutes, filtered and evaporated until more precipitate was |
| | formed. The mixture was cooled in an ice-bath and the precipitate was |
| | collected, washed with chilled water and dried under vacuum. The combined |
| | precipitates obtained after work-up afford the desired product (1.94 g, 19%). |
| | MS (+ve ion electrospray) m/z 250 (MH+). |
| | (b) 3-cinnolinecarbaldehyde |
| | A solution of (a) in hot water (200 mL) was added to dioxan (150 mL). The |
| | solution was cooled to 20° C. then a solution of sodium periodate (6.46 g) in |
| | water (400 mL) was added. The mixture was stirred in the dark for 80 minutes. |
| | The aqueous was extracted several times with diethyl ether. The aqueous was |
| | then salted by addition of sodium chloride, extracted several times with diethyl |
| | ether then several times with ethyl acetate. The combined extracts were dried |
| | over magnesium sulfate and evaporated under vacuum to afford the aldehyde |
| | (1.29 g, 100%). |
| | MS (+ve ion electrospray) m/z 158 (MH+). |
| 79 | N-(2,1,3-benzothiadiazol-5-ylmethyl)-1-{2-[3-fluoro-6-(methoxy)-4- |
| | quinolinyl]ethyl}-4-piperidinamine dihydrochloride |
| | 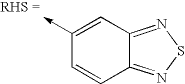 |
| | Preparation of 2,1,3-benzothiadiazole-5-carbaldehyde |
| | 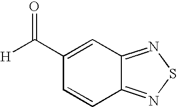 |
| | (a) Benzo[1,2,5]thiadiazol-5-yl-methanol |
| | Benzo[1,2,5]thiadiazole-5-carboxylic acid (2.00 g, 11.11 mmol) was |
| | dissolved in tetrahydrofuran (50 mL) and cooled to 0° C. To this was added |
| | triethylamine (1.80 mL, 12.87 mmol) followed by isobutylchloroformate (1.62 mL, |
| | 12.40 mmol) in a dropwise manner. The resulting slurry was stirred for a further |
| | 30 minutes at 0° C. and then filtered into a mixture of sodium borohydride |
| | (0.83 g, 21.84 mol) in ice water (20 mL). The resulting mixture was stirred at 0° C. |
| | for 30 minutes, evaporated to one quarter of its volume and then extracted with |
| | dichloromethane (3 × 50 mL). The organic phases were combined and then |
| | dried over sodium sulfate. This was followed by concentration under reduced |
| | pressure to provide the desired product as a white solid which was used |
| | without further purification (1.50 g, 81%). |
| | MS (+ve ion electrospray) m/z 167 (MH+). |
| | (b) 2,1,3-benzothiadiazole-5-carbaldehyde |
| | A stirred solution of alcohol (a) (3.5 g) in chloroform (150 mL) and |
| | tetrahydrofuran (300 mL) was treated with manganese dioxide (7.8 g) for 18 |
| | hours and was filtered and evaporated to give the aldehyde as a white solid |
| | (2.5 g). |
| | MS (+ve ion electrospray) m/z 165 (MH+). |
| 80 | 1-{2-[3-fluoro-6-(methoxy)-4-quinolinyl]ethyl}-N-([1,3]thiazolo[5,4- |
| | b]pyridin-6-ylmethyl)-4-piperidinamine dihydrochloride |
| | 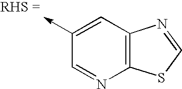 |
| | Preparation of [1,3]thiazolo[5,4-b]pyridine-6-carbaldehyde |
| | 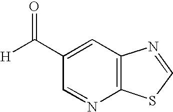 |
| | (a) 5-Amino-6-thioxo-1,6-dihydro-pyridine-3-carboxylic acid methyl ester |
| | A mixture of sodium sulfide nonahydrate (2.17 g) and sulfur (0.29 g) |
| | was heated in boiling water (20 mL) until the solution was homogeneous and |
| | added to a solution of 6-chloro-5-nitro-nicotinic acid methyl ester (3.1 Og) in |
| | methanol (50 mL). The mixture was boiled for 15 minutes and cooled. The |
| | resulting disulfide was collected and washed with water to give a yellow solid |
| | (2.46 g). The solid (5 g) in acetic acid (100 mL) and 4 M HCl in dioxan (50 mL) |
| | was treated with zinc dust (12 g) and the mixture was stirred at room |
| | temperature for 30 minutes, filtered and evaporated to dryness. Sodium |
| | acetate and sodium sulfate were added and the mixture was extracted with |
| | warm chloroform and chromatographed on silica gel, eluting with chloroform |
| | then methanol-chloroform to afford a yellow solid (2.3 g). |
| | MS (+ve ion electrospray) m/z 185 (MH+) |
| | (b) Thiazolo[5,4-b]pyridine-6-carboxylic acid methyl ester |
| | The amine (a) (0.7 g) was heated in formic acid (30 mL) under reflux for |
| | 30 minutes and was evaporated and chromatographed on silica gel |
| | (chloroform) to give a solid (0.65 g). |
| | MS (+ve ion electrospray) m/z 195 (MH+) |
| | (c) Thiazolo[5,4-b]pyridin-6-yl-methanol |
| | A solution of ester (b) (200 mg) in dry tetrahydrofuran (15 mL) and dry diethyl |
| | ether (15 mL), cooled to −45° C., was treated with a 1 M solution of lithium |
| | aluminium hydride in diethyl ether (1.55 mL) and the mixture was heated under |
| | reflux for 18 hours. It was cooled and an aqueous solution of saturated |
| | sodium carbonate was added cautiously. Dichloromethane and anhydrous |
| | sodium sulfate were added and the mixture was stirred for 15 minutes and |
| | filtered. The filtrate was evaporated to afford a white solid (95 mg). |
| | MS (+ve ion electrospray) m/z 167 (MH+) |
| | (d) [1,3]thiazolo[5,4-b]pyridine-6-carbaldehyde |
| | The alcohol (c) (65 mg) in chloroform (10 mL) was stirred with |
| | manganese dioxide (200 mg) for 5 hours, filtered and evaporated and |
| | chromatographed on silica gel, eluting with dichloromethane then chloroform, |
| | to give a solid (65 mg). |
| | MS (+ve ion electrospray) m/z 165 (MH+). |
| 81 | N-(3,4-dihydro-2H-pyrido[3,2-b][1,4]thiazin-6-ylmethyl)-1-{2-[3-fluoro-6- |
| | (methoxy)-4-quinolinyl]ethyl}-4-piperidinamine dihydrochloride |
| | 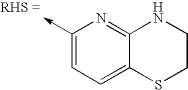 |
| | Preparation of 3,4-dihydro-2H-pyrido[3,2-b][1,4]thiazine-6- |
| | carbaldehyde |
| | 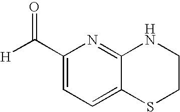 |
| | Prepared by reacting methyl 3-oxo-3,4-dihydro-2H-pyrido[3,2- |
| | b][1,4]thiazine-6-carboxylate with lithium aluminium hydride followed by |
| | oxidation with manganese dioxide to give the carboxaldehyde. |
| | MS (+ve ion electrospray) m/z 181 (MH+). |
| 82 | N-(1,3-benzothiazol-5-ylmethyl)-1-{2-[3-fluoro-6-(methoxy)-4- |
| | quinolinyl]ethyl}-4-piperidinamine dihydrochloride |
| | 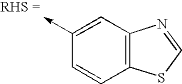 |
| | Preparation of 1,3-benzothiazole-5-carbaldehyde |
| | 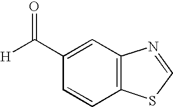 |
| | (a) Benzothiazol-5-ylcarboxylic acid |
| | 4-Chloro-3-nitrobenzoic acid (22 g, 0.11 mol) was suspended in water, |
| | sodium hydroxide (4.33 g, 0.11 mol) and sodium sulfide hydrate (32 g) were |
| | added, and the mixture heated at reflux for 24 hours. After acidification with 5 M |
| | hydrochloric acid the mixture was extracted with ethyl acetate. The extracts |
| | were dried over magnesium sulfate and evaporated under reduced pressure. |
| | The product from this reaction (1 g, 5.9 mmol) was dissolved in formic acid and |
| | heated at reflux in the presence of zinc (0.1 g) for 6 hours. The mixture was |
| | allowed to cool and was concentrated under reduced pressure. The residue |
| | was diluted with water and neutralised with saturated aqueous sodium |
| | hydrogen carbonate. Extraction with tetrahydrofuran and ethyl acetate (1:1) |
| | gave a pale yellow solid (0.48 g) that was purified on silica gel using a methanol |
| | dichloromethane gradient. |
| | MS (+ve ion electrospray) m/z 180 (MH+) |
| | (b) 1,3-benzothiazol-5-ylmethanol |
| | Acid (b) in tetrahydrofuran and triethylamine was cooled to 0° C. and |
| | isobutylchloroformate was added dropwise and the solution was stirred at 0° C. |
| | for 2 hours, when it was filtered into a stirred solution of sodium borohydride in |
| | ice/water. The mixture was stirred at 0° C. for 1 hour and allowed to warm to |
| | room temperature. It was acidified with 2 M hydrochloric acid, evaporated to |
| | half volume, and the resulting product was collected, washed with water and |
| | dried in vacuo, to give a white solid. |
| | MS (+ve ion electrospray) m/z 166 (MH+). |
| | (c) 1,3-benzothiazole-5-carbaldehyde |
| | Alcohol (b) was oxidised as in example (2c) to afford the product as a |
| | solid. |
| | MS (+ve ion electrospray) m/z 164 (MH+). |
| 83 | 1-{2-[3-fluoro-6-(methoxy)-4-quinolinyl]ethyl}-N-([1,2,3]thiadiazolo[5,4-b]pyridin- |
| | 6-ylmethyl)-4-piperidinamine dihydrochloride |
| | 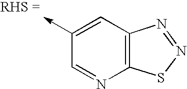 |
| | Preparation of [1,2,3]thiadiazolo[5,4-b]pyridin-6-ylmethyl methanesulfonate |
| | 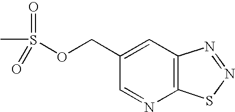 |
| | This intermediate was prepared from [1,2,3]thiadiazolo[5,4-b]pyridin-6- |
| | ylmethanol (prepared as in WO 2003064431) by reacting a THF solution of this |
| | alcohol with 1 equivalent each of triethylamine and methanesulfonyl |
| | chloride. The solution of the resulting methanesulfonate was added to a DMF |
| | solution containing 1 equivalent of of amine (31 g) and potassium carbonate. |
| | Workup and chromatography afforded the free base of the title compound in |
| | 40% yield |
| 84 | 7-{[(1-{2-[3-fluoro-6-(methoxy)-4-quinolinyl]ethyl}-4-piperidinyl)amino]methyl} |
| | 1H-pyrido[2,3-b][1,4]thiazin-2(3H)-one dihydrochloride |
| | 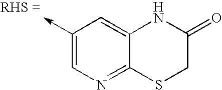 |
| | Aldehyde 2-oxo-2,3-dihydro-1H-pyrido[2,3-b][1,4]thiazine-7-carbaldehyde is |
| | from example (48)- |
| 85 | N-(2,3-dihydro[1,4]dioxino[2,3-b]pyridin-7-ylmethyl)-1-(2- |
| | [3-fluoro-6-(methoxy)-4-quinolinyl]ethyl}-4-piperidinamine |
| | dihydrochloride |
| | 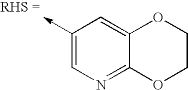 |
| | 2,3-dihydro[1,4]dioxino[2,3-b]pyridine-7-carbaldehyde was prepared as |
| | described in Example (40e) of WO02056882 |
| 86 | N-(2,3-dihydro[1,4]oxathiino[2,3-c]pyridin-7-ylmethyl)-1-{2-[3-fluoro-6- |
| | (methoxy)-4-quinolinyl]ethyl}-4-piperidinamine dihydrochloride |
| | 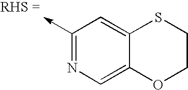 |
| | The aldehyde 2,3-dihydro[1,4]oxathiino[2,3-c]pyridine-7-carbaldehyde was |
| | prepared as in Example (60) |

### Example 87

### 4-[(2,3-dihydro[1,4]dioxino[2,3-c]pyridin-7-ylmethyl)amino]-1-{2-[3-fluoro-6-(methoxy)-4-quinolinyl]ethyl}-N-methyl-4-piperidinecarboxamide dihydrochloride

### (a) 1-(1,1-dimethylethyl) 4-methyl 4-amino-1,4-piperidinedicarboxylate

A suspension of 4-amino-1-{[(1,1-dimethylethyl)oxy]carbonyl}-4 piperidinecarboxylic acid (4.3 g, 17.7 mmol) in acetonitrile/MeOH (20 mL/2 mL) was treated with N-ethyl-N-(1-methylethyl)-2-propanamine (3.1 mL, 18 mmol) followed by trimethylsilyidiazomethane (2M in hexane), (10.6 mL, 21.1 mmol). The reaction mixture was stirred at room temperature for 24 hours. A further 2 mL of trimethylsilyidiazomethane was added and the mixture was stirred for a further 18 hours. Solvents were evaporated under vacuum. The residue was chromatographed on silica gel eluting with diethyl ether then ethyl acetate and 10% methanol in ethyl acetate to afford the product as a white solid (2.95 g, 65%). MS (ES) m/z 259 (M+H)⁺.

### (b) 1-(1,1-dimethylethyl) 4-methyl 4-[(2,3-dihydro[1,4]dioxino[2,3-c]pyridin-7-ylmethyl)amino]-1,4-piperidinedicarboxylate

A mixture of amine (a) (2.44 g, 9.47 mmol), aldehyde (2c) (1.56 g, 9.52 mmol), sodium triacetoxyborohydride (6.0 g, 28.5 mmol) and DMF (100 mL) was heated at 60° C. overnight. A further 0.8 g of aldehyde and 6.05 g of sodium triacetoxyborohydride were added and the stirring and heating were continued for a further 24 hours. DMF was evaporated under vacuum. The residue was dissolved in aqueous sodium bicarbonate and extracted several times with 10% MeOH in dichloromethane. The combined organic extracts were dried over magnesium sulfate and evaporated in vacuo. The crude was chromatographed on silica gel eluting with 2-5% MeOH in dichloromethane to afford the product as an oil (4.4 g, 100%). MS (ES) m/z 408 (M+H)⁺.

### (c) 4-[(2,3-dihydro[1,4]dioxino[2,3-c]pyridin-7-ylmethyl)amino]-1-{[(1,1-dimethylethyl)oxy]carbonyl)-4-piperidinecarboxylic acid

A mixture of ester (b) (4 g, 9.8 mmol), 2M sodium hydroxide (10 mL, 20 mmol), water (20 mL) and dioxan (100 mL) were heated under reflux for 3 days. The mixture was filtered and evaporated under vacuum. The residue was dissolved in a minimal amount of water and neutralised by dropwise addition of 5M HCl. A white precipitate was filtered off, washed with water and dried in vacuo to afford the product (3.29 g, 76%). MS (ES) m/z 294 (M+H)⁺.

### (d) 1,1-dimethylethyl 4-[(2,3-dihydro[1,4]dioxino[2,3-c]pyridin-7-ylmethyl)amino]-4-[(methylamino)carbonyl]-1-piperidinecarboxylate

A suspension of carboxylic acid (c) (0.98 g, 2.48 mmol) in DMF (35 mL) was treated with triethylamine (1.03 mL, 7.45 mmol), 1-hydroxybenzotriazole (0.38 g, 2.53 mmol) and EDC (0.53 g, 2.7 mmol) and stirred at room temperature for 45 minutes. Methylamine (0.17 g, 2.5 mmol) was added. The reaction mixture was stirred at room temperature overnight. More triethylamine (0.21 mL), 1-hydroxybenzotriazole (0.08 g) and EDC (0.11 g) were added. The reaction mixture was stirred for 18 hours. DMF was evaporated under vacuum. The residue was dissolved in water and basified by addition of aqueous sodium carbonate. The aqueous layer was extracted several times with dichloromethane/methanol. The combined organic were dried over magnesium sulfate and the residue was chromatographed on silica gel eluting with 2-5% MeOH in dichloromethane to afford the product as an oil (0.9 g, 89%). MS (ES) m/z 407 (M+H)⁺.

### (e) 4-[(2,3-dihydro[1,4]dioxino[2,3-c]pyridin-7-ylmethyl)amino]-N-methyl-4-piperidinecarboxamide

A solution of protected piperidine (d) (89 mg, 2.19 mmol) in dichloromethane (10 mL) was treated with trifluoroacetic acid (10 mL). The mixture was stirred for 1.45 hours and evaporated under vacuum. The residue was triturated with diethyl ether, dissolved in 10% methanol in dichloromethane and stirred with an excess of MP-carbonate resin (Argonaut Technologies, 2.54 mmol/g) for 3 hours. The resin was filtered off and washed with methanol/dichloromethane then methanol alternately. The filtrate was evaporated under vacuum to afford the product as an oil (810 mg, quantitative). MS (ES) m/z 307 (M+H)⁺.

### (f) Title Compound

A mixture of vinyl-quinoline (31e) and piperidine (e) was treated as in example (52h) to afford the desired product in 49% yield.

1H NMR δH (CDCl₃) 8.59 (1H, s), 8.15 (1H, s), 7.98 (1H, d), 7.92 (1H, m), 7.30 (1H, dd), 7.22 (1H, d), 6.75 (1H, s), 4.33 (2H, m), 4.29 (2H, m), 3.96 (3H, s), 3.61 (2H, s), 3.26 (2H, m), 2.92 (2H, m), 2.81 (3H, d), 2.67 (2H, m), 2.34 (4H, m). MS (ES) m/z 510 (M+H)⁺.

This material, as a solution in chloroform/methanol, was treated with an excess of 1M HCl in ether and evaporated to dryness. The solid was triturated under ether, filtered and dried under vacuum to provide the title compound as a white solid (72 mg).

### Example 88

### 4-[(2,3-dihydro[1,4]dioxino[2,3-c]pyridin-7-ylmethyl)amino]-1-{2-[3-fluoro-6-(methoxy)-4-quinolinyl]ethyl}-4-piperidinecarboxamide dihydrochloride

### (a) 1,1-dimethylethyl 4-(aminocarbonyl)-4-[(2,3-dihydro[1,4]dioxino[2,3-c]pyridin-7-ylmethyl)amino]-1-piperidinecarboxylate

A suspension of carboxylic acid (87c) (0.3 g, 0.76 mmol) in DMF (10 mL) was treated with triethylamine (0.21 mL, 1.52 mmol), 1-hydroxybenzotriazole (0.1 g, 0.76 mmol) and EDC (0.16 g, 0.84 mmol) and stirred at room temperature for 30 minutes. Ammonia was bubbled through for a few minutes until all solid was dissolved. The reaction mixture was stirred at room temperature overnight. As the reaction had not gone to completion, more ammonia was bubbled through and the reaction mixture was stirred for a further 36 hours. The residual ammonia was removed under vacuum and more triethylamine (0.21 mL, 1.52 mmol), 1-hydroxybenzotriazole (0.1 g, 0.76 mmol) and EDC (0.16 g, 0.84 mmol) were added. The reaction mixture was stirred for 2 hours and ammonia was bubbled through for 10 minutes. The reaction mixture was stirred overnight. DMF was evaporated under vacuum. The residue was partitioned between diluted sodium hydroxide and dichloromethane/methanol. The aqueous layer was reextracted with dichloromethane/methanol. The combined organic extracts were washed with diluted sodium hydroxide, dried over magnesium sulfate and the residue was chromatographed on silica gel eluting with 0-5% MeOH in ethyl acetate to afford the product as an oil (54 mg, 18%). MS (ES) m/z 393 (M+H)⁺.

### (b) 4-[(2,3-dihydro[1,4]dioxino[2,3-c]pyridin-7-ylmethyl)amino]-4-piperidinecarboxamide

A solution of protected piperidine (a) (54 mg, 0.14 mmol) in dichloromethane (1 mL) was treated with trifluoroacetic acid (1 mL). The mixture was stirred for 1.5 hours and evaporated under vacuum. The residue was triturated with diethyl ether, dissolved in 10% methanol in dichloromethane and stirred with 0.2 g of MP-carbonate resin (2.75 mmol/g) for 3 hours. The resin was filtered off and washed with methanol/dichloromethane then methanol alternately. The filtrate was evaporated under vacuum to afford the product as an oil (501 mg, quantitative). MS (ES) m/z 293 (M+H)⁺.

### (c) Title Compound

A mixture of vinyl-quinoline (31e) and piperidine (b) was treated as in example (52h) to afford the desired product in 17% yield.

1H NMR δH (CDCl₃) 8.59 (1H, s), 8.12 (1H, s) 7.99 (1H, d), 7.72 (1H, br s), 7.30 (1H, dd), 7.22 (1H, d), 6.76 (1H, s), 5.38 (1H, br s), 4.32 (2H, m), 4.28 (2H, m), 3.96 (3H, s), 3.67 (2H, s), 3.23 (2H, t), 2.89 (2H, m), 2.68 (2H, m), 2.43 (2H, t), 2.27 (2H, td), 1.74 (2H, br d). MS (ES) m/z 496 (M+H)⁺.

This material, as a solution in chloroform/methanol, was treated with an excess of 1M HCl in ether and evaporated to dryness. The solid was triturated under ether, filtered and dried under vacuum to provide the title compound as a white solid (70 mg).

### Example 89

### 4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-N-methyl-4-piperidinecarboxamide dihydrochloride

A mixture of vinyl-naphthyridine (53h) and piperidine (87e) was treated as in example (52h) to afford the desired product in 17% yield.

1H NMR δH (CDCl₃) 8.60 (1H, s), 8.17 (1H, d), 8.14 (1H, s), 7.88 (1H, m), 7.06 (1H, d), 6.74 (1H, s), 4.33 (2H, m), 4.29 (2H, m), 4.07 (3H, s), 3.60 (2H, s), 3.42 (2H, m), 2.94 (2H, m), 2.80 (3H, d), 2.75 (2H, m), 2.38 (2H, m), 2.25 (2H, m). MS (ES) m/z 511 (M+H)⁺.

This material, as a solution in chloroform/methanol, was treated with an excess of 1M HCl in ether and evaporated to dryness. The solid was triturated under ether, filtered and dried under vacuum to provide the title compound as a white solid (55 mg).

### Example 90

### 4-[(2,3-dihydro[1,4]dioxino[2,3-c]pyridin-7-ylmethyl)amino]-1-(2-[3-fluoro-6-(methoxy)-1,5-naphthyridin-4-yl]ethyl}-4-piperidinecarboxamide dihydrochloride

A mixture of vinyl-naphthyridine (53h) and piperidine (88b) was treated as in example (52h) to afford the desired product in 41% yield.

1H NMR δH (CDCl₃) 8.61 (1H, s), 8.17 (1H, d), 8.12 (1H, s), 7.70 (1H, br s), 7.06 (1H, d), 6.76 (1H, s), 5.28 (1H, br s), 4.33 (2H, m), 4.28 (2H, m), 4.08 (3H, s), 3.65 (2H, s), 3.41 (2H, t), 2.92 (2H, m), 2.78 (2H, m), 2.44 (2H, br), 2.25 (2H, m), 1.72 (2H, m). MS (ES) m/z 497 (M+H)⁺.

This material, as a solution in chloroform/methanol, was treated with an excess of 1M HCl in ether and evaporated to dryness. The solid was triturated under ether, filtered and dried under vacuum to provide the title compound as a white solid (62 mg).

### Example 91

### 1-{2-[3-chloro-6-(methoxy)-1,5-naphthyridin-4-yl]ethyl}-4-[(2,3-dihydro[1,4]dioxino[2,3-c]pyridin-7-ylmethyl)amino]-4-piperidinecarboxamide dihydrochloride

A mixture of vinyl-naphthyridine (3a) and piperidine (88b) was treated as in example (52h) to afford the desired product in 53% yield.

1H NMR δH (CDCl₃) 8.65 (1H, s), 8.17 (1H, d), 8.14 (1H, s), 7.68 (1H, br s), 7.09 (1H, d), 6.77 (1H, s), 5.30 (1H, br s), 4.31 (2H, m), 4.26 (2H, m), 4.07 (3H, s), 3.66 (2H, s), 3.55 (2H, m), 2.94 (2H, m), 2.73 (2H, m), 2.49 (2H, m), 2.28 (2H, m). MS (ES) m/z 513 (M+H)⁺.

This material, as a solution in chloroform/methanol, was treated with an excess of 1M HCl in ether and evaporated to dryness. The solid was triturated under ether, filtered and dried under vacuum to provide the title compound (58 mg).

### Example 92

### (4[(2,3-dihydro[1,4]dioxino[2,3-c]pyridin-7-ylmethyl)amino]-1-{2-[3-fluoro-6-(methoxy)-4-quinolinyl]ethyl}-4-piperidinyl)methanol dihydrochloride

### (a) 1-{[(1,1-dimethylethyl)oxy]carbonyl}-4-({[(phenylmethyl)oxy]carbonyl}amino)-4-piperidinecarboxylic acid

A solution of 4-amino-1-{[(1,1-dimethylethyl)oxy]carbonyl}-4 piperidinecarboxylic acid (10 g, 43.5 mmol) in water (400 mL), dimethoxyethane (50 mL) and 2% aqueous sodium hydroxide solution (50 mL) was treated at 0° C. with a solution of N-(benzyloxycarbonyloxy)succinimide (16 g, 65 mmol) in dimethoxyethane (50 mL). The mixture was stirred at room temperature overnight, filtered, concentrated, and extracted with ether. The aqueous phase (pH10) was taken to pH4 with aqueous HCl and extracted with ethyl acetate. Drying and evaporation afforded a solid which was triturated with ether, filtered and dried in vacuo (7.3 g, 44%). MS (ES) m/z 379 (M+H)⁺.

### (b) 1-(1,1-dimethylethyl) 4-methyl 4-({[(phenylmethyl)oxy]carbonyl}amino)-1,4-piperidinedicarboxylate

A mixture of acid (a) (70.3 g, 19.3 mmol), methyl iodide (1.2 mL) and potassium carbonate (5.3 g) in acetone (70 mL) was stirred for 3 days then filtered and evaporated. The residue was partitioned between ethyl acetate and water. The organic phase was dried and evaporated affording an oil (7 g, 92%). MS (ES) m/z 393 (M+H)⁺.

### (c) methyl 4-({[(phenylmethyl)oxy]carbonyl}amino)-4-piperidinecarboxylate

A solution of carbamate (b) (7 g, 17.8 mmol) in dichloromethane (35 mL) was treated with TFA (35 mL). After 1.5 hours the mixture was evaporated. The residue was partitioned between 10% methanol in dichloromethane and saturated aqueous sodium bicarbonate solution. The organic extract was dried and evaporated to give an oil (5.6 g, 100%). MS (ES) mm/z 293 (M+H)⁺.

### (d) Methyl 1-{2-[3-fluoro-6-(methoxy)-4-quinolinyl]ethyl}-4-({[(phenylmethyl)oxy]carbonyl}amino)-4-piperidinecarboxylate

A mixture of vinyl-quinoline (31e) and piperidine (c) was treated as in example (52h) to afford the desired product in 87% yield. MS (ES) m/z 496 (M+H)⁺.

### (e) Methyl 4-amino-1-{2-[3-fluoro-6-(methoxy)-4-quinolinyl]ethyl}-4-piperidinecarboxylate

A solution of protected amine (d) in ethanol was hydrogenated with palladium on charcoal to afford the product as an oil in a 90% yield. MS (ES) m/z 362 (M+H)⁺.

### (f) methyl 4-[(2,3-dihydro[1,4]dioxino[2,3-c]pyridin-7-ylmethyl)amino]-1-{2-[3-fluoro-6-(methoxy)-4-quinolinyl]ethyl}-4-piperidinecarboxylate

The amine (e) and aldehyde (2c) were treated as in example (2d) (except that 1.4 equivalent of aldehyde and 11.8 equivalent of sodium triacetoxyborohydride were needed) to afford the desired product in 62% yield.

MS (+ve ion electrospray) m/z 511 (MH+).

### (g) Title Compound

A solution of ester (f) (68 mg, 0.13 mmol) in anhydrous tetrahydrofuran (5 mL) was cooled in an ice-bath for 30 minutes. A 1M solution of lithium aluminium hydride (0.14 mL, 0.14 mmol) in diethyl ether was added dropwise and the mixture was stirred for 1 hour at 0° C. then allowed to warm to room temperature. A few drops of diluted sodium hydroxide were added, the mixture was filtered through Kieselguhr and washed through with ethyl acetate. The filtrate was evaporated under vacuum. The residue was chromatographed eluting with 5-10% methnaol in dichloromethane to afford the desired product as an oil (44 mg, 69%).

1H NMR δH (CDCl₃) 8.59 (1H, s), 8.09 (1H, s), 7.99 (1H, d), 7.31 (1H, dd), 7.23 (1H, d), 6.76 (1H, s), 4.32 (2H, m), 4.26 (2H, m), 3.95 (3H, s), 3.71 (2H, s), 3.40 (2H, s), 3.27 (2H, t), 2.79-2.53 (6H, m), 1.79-1.58 (4H, m). MS (ES) m/z 483 (M+H)⁺.

This material, as a solution in chloroform/methanol, was treated with an excess of 1M HCl in ether and evaporated to dryness. The solid was triturated under ether, filtered and dried under vacuum to provide the dihydrochloride salt of the title compound.

### Example 93

### N-[1-{2-[3-fluoro-6-(methoxy)-1,5-naphthyridin-4-yl]ethyl}-4-(hydroxymethyl)-4-piperidinyl]-3-oxo-3,4-dihydro-2H-pyrido[3,2-b][1,4]thiazine-6-carboxamide hydrochloride

### (a) 1,1-dimethylethyl 4-amino-4-(hydroxymethyl)-1-piperidinecarboxylate

A solution of ester (87a) (1 g, 3.88 mmol) in anhydrous tetrahydrofuran (10 mL) was cooled in an ice-bath. A 1M solution of lithium aluminium hydride in tetrahydrofuran (7.76 mL, 7.76 mmol) was added dropwise and the reaction mixture was stirred at 0° C. for 1.5 hours. Several drops of diluted sodium hydroxide were added cautiously. The mixture was filtered through Kieselguhr, washed through with ethyl acetate and evaporated under vacuum. The residue was chromatographed on silica gel eluting with 5-20% methanol in ethyl acetate to afford the product as an oil (0.37 g, 41%). MS (ES) m/z 231 (M+H)⁺.

### (b) 1,1-dimethylethyl 4-(hydroxymethyl)-4-{[(3-oxo-3,4-dihydro-2H-pyrido[3,2-b][1,4]thiazin-6-yl)carbonyl]amino}-1-piperidinecarboxylate

A solution of acid (7b) (0.34 g, 1.61 mmol) in DMF (10 mL) was treated with triethylamine (0.45 mL, 3.3 mmol) and O-(7-azabenzotriazol-1-yl)-N,N,N′,N′-tetramethyluronium hexafluorophosphate (0.63 g, 1.65 mmol). The mixture was stirred for 45 minutes and was added to aminoalcohol (a) (0.37 g, 1.61 mmol). The reaction mixture was stirred at room temperature for 18 hours and evaporated under vacuum. The residue was slurried with water. A precipitate was formed, filtered, washed with water and dried in vacuo to afford the product (0.4 g, 59%).

MS (ES) m/z 423 (M+H)⁺.

### (c) N-[4-(hydroxymethyl)-4-piperidinyl]-3-oxo-3,4-dihydro-2H-pyrido[3,2-b][1,4]thiazine-6-carboxamide

A solution of protected amine (b) (0.4 g, 0.95 mmol) in dichloromethane (10 mL) was treated with trifluoroacetic acid (10 mL). The reaction mixture was stirred at room temperature for 1.5 hours and evaporated under vacuum. The residue was dissolved in a minimum volume of water and basified by addition of sodium bicarbonate. The aqueous layer was extracted several times with 10% methanol in dichloromethane (with addition of sodium chloride). As the extraction was incomplete, the aqueous layer was acidified with a 2M solution of HCl and evaporated to dryness. The residue was extracted with 10% methanol in dichloromethane several more times. The combined organic extracts were dried over magnesium sulfate, filtered and evaporated to afford the product as an oil (0.3 g, 98%). MS (ES) m/z 323 (M+H)⁺.

### (d) Title Compound

A mixture of vinyl-naphthyridine (53h) and piperidine (c) was treated as in example (52h) to afford the desired product in 45% yield.

1H NMR δH (CDCl₃) 8.61 (1H, s), 8.25 (1H, br s), 8.18 (1H, d), 7.84 (1H, d), 7.79 (1H, d), 7.07 (1H, d), 4.06 (3H, s), 3.81 (2H, s), 3.54 (2H, s), 3.41 (2H, m), 2.84 (2H, m), 2.78 (2H, m), 2.43 (2H, t), 2.10 (2H, br d), 1.84 (2H, m). MS (ES) m/z 527 (M+H)⁺.

This material, as a solution in chloroform/methanol, was treated with an excess of 1M HCl in ether and evaporated to dryness. The solid was triturated under ether, filtered and dried under vacuum to provide the title compound.

### Example 94

### N-(1-{2-[3-fluoro-6-(methoxy)-4-quinolinyl]ethyl}-4-piperidinyl)-3-oxo-3,4-dihydro-2H-pyrido[3,2-b][1,4]oxazine-6-carboxamide hydrochloride

Acid (see Example 65) and amine (31g) were treated as in example (93b) to afford the free base of the title compound in 81% yield.

1H NMR δH (d6-DMSO) 8.78 (1H, s), 8.16 (1H, brs), 8.02 (1H, d), 7.62 (1H, d), 7.48 (2H, 2×d), 7.39 (1H, d), 4.76 (2H, s), 4.01 (3H, s), 3.78 (1H, br), 3.57-3.17 (6H, m), 2.13 (2H, br m), 1.85 (2H, br m). MS (ES) m/z 480 (M+H)⁺.

This material, as a solution in chloroform/methanol, was treated with an excess of 1M HCl in ether and evaporated to dryness. The solid was triturated under ether, filtered and dried under vacuum to provide the title compound.

### Example 95

### N-(1-{2-[3-fluoro-6-(methoxy)-4-quinolinyl]ethyl}-4-piperidinyl)₃-oxo-3,4-dihydro-2H-pyrido[3,2-b][1,4]thiazine-6-carboxamide hydrochloride

Acid (7b) and amine (31g) were treated as in example (93b) to afford the free base of the title compound in 66% yield.

1H NMR δH (CDCl₃/CD₃OD) 8.59 (1H, s), 8.01 (1H, d), 7.83 (1H, d), 7.78 (1H, d), 7.74 (1H, br), 7.35 (1H, dd), 7.25 (1H, d), 4.04 (1H, m), 3.99 (3H, s), 3.54 (2H, s), 3.31 (2H, m), 3.12 (2H, m), 2.74 (2H, m), 2.40 (2H, t), 2.09 (2H, br d). MS (ES) m/z 496 (M+H)⁺.

This material, as a solution in chloroform/methanol, was treated with an excess of 1M HCl in ether and evaporated to dryness. The solid was triturated under ether, filtered and dried under vacuum to provide the title compound.

### Example 96

### 7-{[((3R,4S)-1-{2-[3-fluoro-6-(methoxy)-4-quinolinyl]ethyl}-3-hydroxy-4-piperidinyl)amino]methyl}-1H-pyrido[2,3-b][1,4]thiazin-2(3H)-one dihydrochloride Enantiomer 1

Amine (34b) and aldehyde (see Example 48) were treated as in example (47m) to afford the free base of the title compound in 56% yield.

1H NMR δH (CDCl₃) 8.90 (1H, bs), 8.14 (1H, d), 8.01 (1H, d), 7.32 (1H, dd), 7.22 (1H, d), 7.17 (1H, d), 3.96 (3H, s), 3.90 (1H, s), 3.81 (2H, q), 3.57 (2H, s), 3.21 (2H, t), 3.11 (1H, d), 2.95 (1H, d), 2.73 (2H, m), 2.52 (1H, m), 2.30 (1H, d), 2.18 (1H, m), 1.77 (2H, m), 1.66 (2H, m). MS (ES) m/z 498 (M+H)⁺.

This material, as a solution in chloroform/methanol, was treated with an excess of 1M HCl in ether and evaporated to dryness. The solid was triturated under ether, filtered and dried under vacuum to provide the title compound.

### Example 97

### 6-{[((3R,4S)-1-{2-[3-chloro-8-fluoro-6-(methoxy)-4-quinolinyl]ethyl}-3-hydroxy-4-piperidinyl)amino]methyl}-2H-pyrido[3,2-b][1,4]thiazin-3(4H)-one dihydrochloride Enantiomer 1

### (a) 8-fluoro-6-(methoxy)-4(1H)-quinolinone

A mixture of 2-fluoro-4-methoxy-phenylamine (3.80 g; 26.7 mmol) and methyl propiolate (2.4 mL, 0.267 mol) in methanol (100 mL) was stirred for 72 hours at room temperature, then heated at 50° C. for 24 hours. It was evaporated and the product purified by chromatography on silica gel (dichloromethane) to give a solid (1.66 g), a portion of which was recrystallised from dichloromethane-hexane.

This solid (0.96 g) in warm Dowtherm A (5 mL) was added over 3 minutes to refluxing Dowtherm A (15 mL), and after a further 20 minutes at reflux the mixture was cooled and poured into ether. The precipate was filtered to give a solid (0.50 g, 61%). MS (ES) m/z 194 (M+H)⁺.

### (b) 3-chloro-8-fluoro-6-(methoxy)-4(1H)-quinolinone

Quinolone (a) (14.8 g, 76.7 mmol) in acetic acid (150 mL) was treated with N-chlorosuccinimide (11.3 g, 84.4 mmol) and the mixture was heated at 40° C. for 18 hours, cooled, the precipitate was filtered and dried under vacuum to afford the product as a solid (8.5 g, 49%). MS (ES) m/z 227/229 (M+H)⁺.

### (c) 3-chloro-8-fluoro-6-(methoxy)-4-quinolinyl trifluoromethanesulfonate

A suspension of 60% sodium hydride in oil (2.24 g, 56.04 mmol) was washed with hexane, the hexane solution decanted, and dry DMF (100 mL) added followed by quinolone (b) (8.5 g, 37.36 mmol). The mixture was stirred at room temperature for 15 minutes, cooled in ice and N-phenyltrifluoromethanesulphonimide (14.7 g, 41.09 mmol) added and the mixture was allowed to stir at room temperature overnight. It was evaporated under vacuum and the residue was chromatographed on silica gel eluting with hexane/dichloromethane to afford the product as a solid (13.9 g, 100%). MS (+ve ion electrospray) m/z 357/359 (MH+).

### (d) 3-chloro-4-ethenyl-8-fluoro-6-(methoxy)quinoline

Triflate (c) was treated as in example (23f) to afford the product in 72% yield. MS (+ve ion electrospray) m/z 239/241 (MH+).

### (e) 1,1-dimethylethyl ((3R,4S)-1-{2-[3-chloro-8-fluoro-6-(methoxy)-4-quinolinyl]ethyl}-3-hydroxy-4-piperidinyl)carbamate

Vinyl-quinoline (d) and piperidine (5c, Enantiomer 1) were treated as in Example (52h) to afford the adduct in 55% yield. MS (+ve ion electrospray) m/z 454/456 (MH+).

### (f) (3R,4S)-4-amino-1-{2-[3-chloro-8-fluoro-6-(methoxy)-4-quinolinyl]ethyl}-3-piperidinol

Carbamate (e) was treated as in example (66b) to afford the amine in 86% yield.

MS (+ve ion electrospray) m/z 354/356 (MH+).

### (g) Title Compound

Amine (f) and aldehyde (7d) were treated as in example (47m) to afford free base of the title compound in 60% yield.

1H NMR δH (CDCl₃) 9.20 (1H, bs), 8.66 (1H, s), 7.57 (1H, d), 7.09 (1H, dd), 7.04 (1H, d), 6.98 (1H, d), 3.94 (3H, s), 3.93 (1H, s), 3.89 (2H, q), 3.44 (2H, s), 3.55 (2H, m), 3.16 (1H, d), 3.00 (1H, d), 2.67 (3H, m), 2.37 (1H, d), 2.26 (1H, m), 1.79 (2H, m).

MS (+ve ion electrospray) m/z 533/535 (MH+).

This material, as a solution in chloroform/methanol, was treated with an excess of 1M HCl in ether and evaporated to dryness. The solid was triturated under ether, filtered and dried under vacuum to provide the title compound.

The following example was prepared by analogous method to Example 97 using the aldehydes shown:

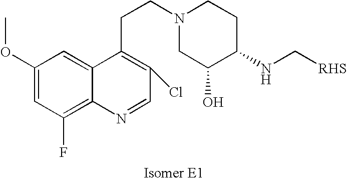

| Example | |
|---|---|
| 98 | (3R,4S)-1-{2-[3-chloro-8-fluoro-6-(methoxy)-4- |
| | quinolinyl]ethyl}-4-[(2,3-dihydro[1,4]dioxino[2,3- |
| | c]pyridin-7-ylmethyl)amino]-3-piperidinol |
| | dihydrochloride |
| | 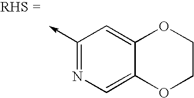 |
| | Aldehyde is 2,3-dihydro[1,4]dioxino[2,3-c]pyridine-7- |
| | carbaldehyde as in example (2c) |

### Example 99

### 2-{4-[(2,3-Dihydro[1,4]dioxino[2,3-c]pyridin-7-ylmethyl)amino]-1-piperidinyl}-1-[3-fluoro-6-(methoxy)-1,5-naphthyridin-4-yl]ethanol Dihydrochloride Hydrate Enantiomer 1

### (a) 1-[3-fluoro-6-(methoxy)-1,5-naphthyridin-4-yl]-1,2-ethanediol

To a solution of AD-mixβ (50 g) in tert-butanol/water (200 mL/200 mL), cooled in an ice-bath for 30 minutes, vinyl-naphthyridine (53h) (8 g, 39.2 mmol) was added and the reaction mixture was stirred at room temperature for 48 hours. Sodium sulfite (75 g) was added and the mixture was stirred for a further 30 minutes. It was extracted with diethyl ether then several times with 10% methanol in chloroform. The organic extract was evaporated under vacuum to afford the desired product as an oil (8.93 g, 96%). MS (+ve ion electrospray) m/z 239 (MH+).

enantiomeric excess=44%, as determined by chiral analytical hplc

### (b) 2-[3-fluoro-6-(methoxy)-1,5-naphthyridin-4-yl]-2-hydroxyethyl 4-methylbenzenesulfonate

To a solution of diol (a) (16.5 g) in dichloromethane (200 mL), triethylamine (10 mL) and dibutyltin oxide (350 mg) was added tosyl chloride (13.2 g). After 3 hours, the mixture was diluted with water/sodium bicarbonate and extracted several times with chloroform. The combined organic extracts were dried over magnesium sulfate and evaporated under vacuum. The residue was chromatographed on silica gel eluting with 20-30% ethyl acetate in chloroform to afford the desired product (20.3 g, 75%). MS (+ve ion electrospray) m/z 393 (MH+).

### (c) 7-fluoro-2-(methoxy)-8-(2-oxiranyl)-1,5-naphthyridine

To a suspension of tosylate (b) (10.5 g, 26.7 mmol) in anhydrous methanol (160 mL), cooled in an ice-bath, potassium carbonate (7.03 g, 50.9 mmol) was added. After 15 minutes with cooling, the mixture was stirred at room temperature for a further 1.75 hours. It was then diluted with water, extracted several times with dichloromethane, dried over magnesium sulfate and evaporated under vacuum. The residue was chromatographed on silica gel eluting with dichloromethane, chloroform then 20% ethyl acetate in chloroform to afford the product as an oil (5.55 g, 94%). MS (+ve ion electrospray) m/z 221 (MH+).

### (d) phenylmethyl 4-({[(1,1-dimethylethyl)oxy]carbonyl}amino)-1-piperidinecarboxylate

Piperidin-4-yl-carbamic acid tert-butyl ester (21 g, 0.10 mol) was added to a well stirred mixture of ethyl acetate (640 mL) and saturated sodium bicarbonate (533 mL). After 5 minutes, phenylmethyl chloridocarbonate was added dropwise over 10 minutes. The mixture was stirred at room temperature for 18 hours. The phases were separated. The organic layer was washed with diluted HCl and bicarbonate, dried over magnesium sulfate and evaporated under vacuum to afford the product as a white solid (29.3 g, 83%). MS (+ve ion electrospray) m/z 336 (MH+).

### (e) phenylmethyl 4-amino-1-piperidinecarboxylate

Carbamate (d) (19 g, 57 mmol) was dissolved in dichloromethane (200 mL) and treated with trifluoroacetic acid (120 mL). After 1 hour the mixture was evaporated and the residue partitioned between ethyl acetate and saturated aqueous sodium bicarbonate solution. The ethyl acetate extract was dried and evaporated affording an oil in quantitative yield (13.3 g).

MS (+ve ion electrospray) m/z 236 (MH+).

### (f) phenylmethyl 4-[(2,3-dihydro[1,4]dioxino[2,3-c]pyridin-7-ylmethyl)amino]-1-piperidinecarboxylate

Amine (e) (5.5 g) and aldehyde (2c) (3.3 g) were dissolved in dichloromethane/methanol (100 mL/5 mL) and treated with sodium triacetoxyborohydride (6.5 g, ˜1.5 equivalents). After 16 hours the mixture was partitioned between dichloromethane and saturated aqueous sodium bicarbonate. The organic extract was dried and evaporated to give an oil. Chromatography on silica eluting with 0-15% methanol in dichloromethane afforded an oil (6.4 g, 83%).

MS (+ve ion electrospray) m/z 384 (MH+).

### (g) phenylmethyl 4-((2,3-dihydro[1,4]dioxino[2,3-c]pyridin-7-ylmethyl){[(1,1-dimethylethyl)oxy]carbonyl}amino)-1-piperidinecarboxylate

A solution of amine (f) (14.4 g, 37 mmol) in anhydrous methanol (150 mL) was treated with sodium bicarbonate (9.02 g, 107 mmol) and bis(1,1-dimethylethyl) dicarbonate (15.6 g, 71 mmol). The mixture was stirred at room temperature for 18 hours. The mixture was filtered, evaporated under vacuum and the residue was chromatographed on silica gel eluting with 0-50% ethyl acetate in hexane to afford the product as an oil (13.5 g, 100%).

MS (+ve ion electrospray) m/z 484 (MH+).

### (h) 1,1-dimethylethyl (2,3-dihydro[1,4]dioxino[2,3-c]pyridin-7-ylmethyl)₄-piperidinylcarbamate

A solution of piperidine (g) (13.5 g, 27.9 mmol) in ethanol (200 mL) was hydrogenated with 10% palladium on charcoal at room temperature for 18 hours. The reaction mixture was filtered through Kieselguhr and evaporated under vacuum to afford the product as an oil (9.7 g, 99%).

MS (+ve ion electrospray) m/z 349 (MH+).

### (i) 1,1-dimethylethyl (2,3-dihydro[1,4]dioxino[2,3-c]pyridin-7-ylmethyl)(1-(2-(3-fluoro-6-(methoxy)-1,5-naphthyridin-4-yl]-2-hydroxyethyl)-4-piperidinyl)carbamate

A mixture of epoxide (c) (1.83 g, 8.3 mmol), amine (h) (3.2 g, 9.1 mmol) and lithium perchlorate (0.88 g, 8.3 mmol) in acetonitrile (50 mL) was stirred at room temperature for 48 hours. The mixture was diluted with water/sodium carbonate and extracted several times with dichloromethane. The extracts were dried over magnesium sulfate and evaporated under vacuum. The residue was chromatographed on silica gel eluting with 0-3% methanol in dichloromethane to afford the product as an oil (3.72 g, 78%).

MS (+ve ion electrospray) m/z 570 (MH+).

### (j) Title Compound

Carbamate (i) (3.72 g, 6.5 mmol) was dissolved in dichloromethane (70 mL) and treated with trifluoroacetic acid (10 mL). After 3 hours the mixture was evaporated and the residue partitioned between 10% methanol/dichloromethane and aqueous sodium carbonate solution. The aqueous phase was further extracted with 10% methanol/dichloromethane to afford a white foam (2.85 g, 93%).

This material was subjected to preparative hplc using a Kromasil C18 (4 inch column) to remove unwanted regioisomers then further purified by Chiralpak AD (3 inch column) to separate the enantiomers. This process afforded the free base of the title compound as the major, first eluted, isomer, as a white foam, (820 mg) which had >99% chemical and enantiomeric purity, [α]_{D} (25° C.)=−6.1 degrees (c=1%, methanol).

1H NMR δH (400 mHz, CD₃OD) 8.68 (1H, s), 8.25 (1H, d), 8.01 (1H, s), 7.21 (1H, d), 6.97 (1H, s), 6.03 (1H, m), 4.25-4.45 (4H, m), 4.11 (3H, s), 3.78 (2H, s), 2.90-3.20 (4H, m), 2.85 (1H, m), 2.50 (1H, m), 2.25 (2H, m), 1.89 (2H, m), 1.30-1.50 (2H, m)

MS (ES) m/z 470 (M+H)⁺.

This material was dissolved in ethanol and treated with 2.2 equivalents of 6M aqueous HCl. Crystallisation was aided by the addition of isopropanol affording after filtration and drying the title compound as a white solid, m.p. 198-200° C. In general, either enantiomer was obtained with moderate to good selectivity using chiral agents (AD-mixα or AD-mixβ) for the dihydroxylation step. Purification to >99% optical purity was accomplished by chiral preparative hplc in a manner analogous to that of Example 99.

### Example 100

### 2-{4-[(2,3-Dihydro[1,4]dioxino[2,3-c]pyridin-7-ylmethyl)amino]-1-piperidinyl}-1-[3-fluoro-6-(methoxy)-1,5-naphthyridin-4-yl]ethanol Dihydrochloride Hydrate Enantiomer 2

This Example was prepared exactly as described for Example 99, but using AD-mix a in the dihydroxylation step (98a). The compound was eluted from the HPLC Chiralpak AD column as the major, second eluting, isomer.

[α]_{D} (25° C.)=+6.3 degrees (c=1%, methanol).

It was converted to the hydrochloride by the method of Example 99.

### Example 101

### racemic,cis 4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinyl)methanol dihydrochloride

### (a) 4-Benzylamino-1-tert-butoxycarbonyl-3-ethoxycarbonyl-1,2,5,6-tetrahydropyridine

A solution of 1-tert-butoxycarbonyl-3-ethoxycarbonylpiperidin-4-one (prepared from 3-ethoxycarbonylpiperidin-4-one and di-tert-butyl-dicarbonate in dichloromethane and triethylamine) (25g) and benzylamine (10.85 g) in toluene was heated under reflux in a Dean and Stark apparatus for 18 hours and then evaporated to dryness to give an oil.

### (b) racemic,cis-4-Benzylamino-1-tert-butoxycarbonyl-3-ethoxycarbonylpiperidine

The enamine (a) (25g) in ethanol (300 ml) was hydrogenated over platinum oxide (1.5 g) for 4 days, filtered, and evaporated to dryness. It was chromatographed on silica gel (ethyl acetate-hexane) to afford the title compound as an oil.

MS (+ve ion electrospray) m/z 363 (MH+).

### (c) racemic, cis-4-Amino-1-tert-butoxycarbonyl-3-ethoxycarbonylpiperidine

The amine (b) (4g) in ethanol (80 ml) containing acetic acid (0.73 g) was hydrogenated at 50 psi (Parr reaction vessel) over 10% palladium-carbon (1g) for 18 hours, filtered and evaporated to dryness to afford the acetate salt of the title compound as a white solid (3g).

MS (+ve ion electrospray) m/z 273 (MH+).

### (d) racemic,cis 1-(1,1-dimethylethyl) 3-ethyl-4-[(2,3-dihydro[1,4]dioxino[2,3-c]pyridin-7-ylmethyl)amino]-1,3-piperidinedicarboxylate

To the acetate salt (c) (2.2 g, 8 mmol) in chloroform, sodium carbonate was added. The mixture was extracted several times with 10% ethanol in chloroform. The organic extracts were dried over sodium sulfate, filtered and evaporated under vacuum to afford an oil.

The oil (2.2 g) in ethanol/chloroform (5 mL/5 mL) was heated with aldehyde (2c) (1.33 g, 8 mmol) at 70° C. for 3 hours. The reaction mixture was cooled and sodium triacetoxyborohydride (5.14 gm 24 mmol) was added. The reaction mixture was stirred at room temperature for 18 hours. It was filtered. Chloroform and sodium carbonate were added. The solution was extracted several times with chloroform. The combined organic extracts were dried over sodium sulfate and evaporated under vacuum. The residue was chromatographed on silica gel eluting with dichloromethane then 2% methanol in dichloromethane to afford the product as an oil (2 g, 59%) MS (+ve ion electrospray) m/z 422 (MH+).

### (e) racemic,cis ethyl-4-[(2,3-dihydro[1,4]dioxino[2,3-c]pyridin-7-ylmethyl)amino]-3-piperidinecarboxylate

Protected piperidine (d) was treated as in example (66b) to afford the product as an oil in a quantitative yield. MS (+ve ion electrospray) m/z 322 (MH+).

### (f) racemic,cis ethyl-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinecarboxylate

A mixture of vinyl-naphthyridine (53h) and piperidine (e) was treated as in example (52h) to afford the product in a 26% yield. MS (+ve ion electrospray) m/z 526 (MH+).

### (g) Title Compound

A solution of ester (f) (0.1 g, 0.19 mmol) in anhydrous diethyl ether/tetrahydrofuran (10 mL/0.4 mL) was cooled to −5° C. in an ethanol-ice bath. A 1M solution of lithium aluminium hydride (0.4 mL, 0.4 mmol) in diethyl ether was added and the reaction mixture was stirred for 1.5 hour at −5° C. The reaction mixture was evaporated under vacuum. Chloroform and an aqueous solution of sodium carbonate were added. The aqueous was extracted several times with chloroform, dried over sodium sulfate and evaporated. The residue was chromatographed and silica gel, eluting with 2-10% methanol in dichloromethane to afford the free base of the title compound as an oil (45 mg).

1H NMR δH (400 mHz, CDCl₃) 8.60 (1H, s), 8.18 (1H, d), 8.09 (1H, s), 7.07 (1H, d), 6.80 (1H, s), 4.25-4.40 (4H, m), 4.10 (3H, s), 3.98 (1H, m), 3.70-3.95 (3H, m), 3.40 (2H, m), 2.88 (2H, m), 2.70 (2H, m), 2.40 (1H, br.d), 2.28 (1H, br. t), 2.05 (1H, m), 1.92 (1H, m), 1.70 (1H, m).

MS (ES) m/z 484 (M+H)⁺.

This material, as a solution in chloroform/methanol, was treated with an excess of 1M HCl in ether and evaporated to dryness. The solid was triturated under ether, filtered and dried under vacuum to provide the title compound (55 mg).

### Example 102

### racemic,cis-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinecarboxylic acid dihydrochloride

Ester (101f) (0.27 g, 0.5 mmol) was treated with a 2M solution of HCl. The reaction mixture was heated at 90° C. for 5 hours. It was evaporated under vacuum and taken to pH 5-6 by addition of a solution of sodium bicarbonate. The aqueous was extracted several times with 5% methanol in chloroform, dried over sodium sulfate and evaporated under vacuum. The residue was chromatographed on a silica gel column eluting with 2-30% methanol in chloroform to afford the free base of the title compound as an oil (30 mg)

1H NMR δH (400 mHz, CD₃OD) 8.60 (1H, s), 8.19 (1H, d), 8.08 (1H, s), 7.15 (1H, d), 7.00 (1H, s), 4.25-4.42 (4H, m), 4.18 (2H, m), 4.10 (3H, s), 3.40-3.70 (3H, m), 3.30 (m (under MeOD)), 3.13 (1H, m), 2.85 (3H, m), 2.50 (2H, m), 1.90-2.18 (2H, m). MS (ES) m/z 498 (M+H)⁺.

This material, as a solution in chloroform/methanol, was treated with an excess of 1M HCl in ether and evaporated to dryness. The solid was triturated under ether, filtered and dried under vacuum to provide the title compound (26 mg).

### Example 103

### racemic,cis-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinecarboxamide dihydrochloride

### (a) 1,1-dimethylethyl-3-(aminocarbonyl)-4-[(2,3-dihydro[1,4]dioxino[2,3-c]pyridin-7-ylmethyl)amino]-1-piperidinecarboxylate

A solution ester (101d) (1 g, 2.3 mmol) in anhydrous methanol (20 mL) and sodium cyanide (50 mg) was treated with liquid ammonia (30 mL). The reaction mixture was sealed in a 500 mL Berghoff bomb and heated at 55° C. for 72 hours. The mixture was evaporated to dryness and chromatographed on silica gel eluting with dichloromethane and 1-10% methanol in dichloromethane to afford the product as an oil (40 mg, 43%).

MS (ES) m/z 393 (M+H)⁺.

### (b) (3R,4S)-4-[(2,3-dihydro[1,4]dioxino[2,3-c]pyridin-7-ylmethyl)amino]-3-piperidinecarboxamide

Protected piperidine (a) was treated as in example (66b) to afford the product as an oil in a quantitative yield.

MS (+ve ion electrospray) m/z 392 (MH+).

### (c) Title Compound

A mixture of vinyl-naphthyridine (53h) and piperidine (b) was treated as in example (52h) to afford the free base of the title compound in 88% yield.

MS (+ve ion electrospray) m/z 526 (MH+).

1H NMR δH (400 mHz, CDCl₃) 8.60 (1H, s), 8.19 (1H, m), 8.17 (1H, d), 8.09 (1H, s), 7.10 (1H, d), 6.86 (1H, s), 5.10 (1H, m), 4.25-4.42 (4H, m), 4.09 (3H, s), 3.98 (1H, d), 3.78 (1H, d), 3.48 (1H, m), 3.34 (1H, m), 3.21 (2H, br.d), 2.80 (4H, m), 2.30 (1H, br.d), 2.17 (1H, br.t), 1.60-1.90 (4H, m).

MS (ES) m/z 496 (M+H)⁺.

This material, as a solution in chloroform/methanol, was treated with an excess of 1M HCl in ether and evaporated to dryness. The solid was triturated under ether, filtered and dried under vacuum to provide the title compound (35 mg).

### Example 104

### 1-{2-[3-chloro-6-(methoxy)-1,5-naphthyridin-4-yl]ethyl}-N-[(6-oxido-2,3-dihydro[1,4]dioxino[2,3-c]pyridin-7-yl)methyl]-4-piperidinamine dihydrochloride

### (a) (6-oxido-2,3-dihydro[1,4]dioxino[2,3-c]pyridin-7-yl)methanol

A solution of alcohol (2b) (0.5 g, 2.9 mmol) in chloroform (30 mL) was treated with m-chloroperbenzoic acid (2 g). The mixture was stirred at room temperature for 18 hours. The desired product precipitated out as a solid and was isloated by filtration. Sodium carbonate and water were added to the filtrate whereupon further solid precipitated out. This was also filtered off, dried and combined with the first solid (in total, 0.25 g, 46%). MS (+ve ion electrospray) m/z 184(MH+).

### (b) 2,3-dihydro[1,4]dioxino[2,3-c]pyridine-7-carbaldehyde 6-oxide

N-oxide (a) (0.25 g, 1.3 mmol) in chloroform (120 mL) was warmed and sonicated. Manganese dioxide (0.5 g) as added and the mixture was stirred at room temperature for 18 hours. The reaction mixture was filtered through celite and evaporated under vacuum to afford the product as a yellow solid (100 mg, 40%).

MS (+ve ion electrospray) m/z 182(MH+).

### (c) Title Compound

Amine (3c) (45 mg, 0.14 mmol) and aldehyde (b) were treated as in example (53j) to afford the free base of the title compound as an oil in a 51% yield.

1H NMR δH (400 mHz, CDCl₃) 8.68 (1H, s), 8.16 (1H, d), 7.99 (1H, s), 7.10 (1H, d), 6.98 (1H, s), 4.25-4.42 (4H, m), 4.09 (3H, s), 3.97 (2H, s), 3.57 (2H, m), 3.08 (2H, br.d), 2.70 (2H, m), 2.50 (1H, m), 2.20 (4H, m), 1.95 (1H, br.d), 1.50 (1H, m). MS (ES) m/z 486/488 (M+H)⁺.

This material, as a solution in chloroform/methanol, was treated with an excess of 1M HCl in ether and evaporated to dryness. The solid was triturated under ether, filtered and dried under vacuum to provide the title compound (40 mg).

### Example 105

### 6-{[(1-{2-[3-chloro-6-(methoxy)-1,5-naphthyridin-4-yl]-3-hydroxypropyl}-4-piperidinyl)amino]methyl}-2H-pyrido[3,2-b][1,4]thiazin-3(4H)-one dihydrochloride

### (a) methyl 2-(tributylstannanyl)propenoate

To a solution of methyl propiolate (2 mL, 22.48 mmol) and bis(triphenylphosphine)palladium(II) chloride (316 mg, 0.45 mmol) in tetrahydrofuran, tri-N-butyltin hydride was added dropwise and the reaction mixture was stirred at room temperature for 30 minutes. It was then evaporated under vacuum. The residue was chromatographed on silica gel eluting with petroleum ether to afford the product as a colourless oil in a quantitative yield.

MS (ES) m/z 375 (M+H)⁺.

### (b) methyl 2-[3-chloro-6-(methoxy)-1,5-naphthyridin-4-yl]-2-propenoate

To a solution of naphthyridine-triflate (1b, 4.88 mmol) in DMF (30 mL) was added stannane (a) (2.75 g, 7.33 mmol), tetrakis(triphenylphosphine)palladium(0) (564 mg, 0.49 mmol), lithium chloride (207 mg, 4.88 mmol) and cupper iodide (697 mg, 3.66 mmol). The reaction mixture was stirred at room temperature for 24 hours, then at 70° C. for a further 2 hours and at 100° C. for an other 18 hours. The reaction mixture was filtered and worked-up to afford the desired product in a 52% yield.

MS (ES) m/z 278/280 (M+H)⁺.

### (c) methyl 2-[3-chloro-6-(methoxy)-1,5-naphthyridin-4-yl]-3-[4-({[(1,1-dimethylethyl)oxy]carbonyl}amino)-1-piperidinyl]propanoate

Propenoate (b) and piperidin-4-yl-carbamic acid tert-butyl ester were treated as in example (52h) to afford the adduct in 90% yield. MS (ES) m/z 477/479 (M+H)⁺.

### (d) 1,1-dimethylethyl (1-{2-[3-chloro-6-(methoxy)-1,5-naphthyridin-4-yl]-3-hydroxypropyl}-4-piperidinyl)carbamate

Ester (c) was reduced with lithium aluminium hydride as in example (92g) to afford the alcohol in 16% yield.

MS (ES) m/z 449/451 (M+H)⁺.

### (e) 3-(4-amino-1-piperidinyl)-2-[3-chloro-6-(methoxy)-1,5-naphthyridin-4-yl]-1-propanol

Carbamate (d) was dissolved in dichloromethane and treated with excess HCl in dioxan. After stirring for 2 hours, the reaction mixture was evaporated under vacuum. The crude HCl salt was neutralised and extracted by the workup procedure of Example (66b) affording the free amine in quantitative yield.

MS (ES) m/z 349/351 (M+H)⁺.

### (f) Title Compound

Piperidine (e) and aldehyde (7d) were treated as in Example (53j) to afford the free base of the title compound in 60% yield.

¹H NMR δH (d4-MeOH) 8.66 (1H, s), 8.18 (1H, d), 7.64 (1H, d), 7.18 (1H, d), 4.27 (1H, m), 4.10 (1H, m), 4.06 (2H, s), 3.77(2H, s), 3.48 (2H, s), 3.35 (4H, m), 3.20-3.15 (1H, m), 3.06 (1H, d), 2.91 (1H, d), 2.47 (1H, m), 2.06 (2H, t), 1.90-1.83 (2H, m), 1.36-1.27 (2H, m). MS (ES) m/z 531/533 (M+H)⁺.

This material, as a solution in chloroform/methanol, was treated with an excess of 1M HCl in ether and evaporated to dryness. The solid was triturated under ether, filtered and dried under vacuum to provide the title compound.

### Example 106

### 6-[({1-[2-(3,6-difluoro-4-quinolinyl)ethyl]-4-piperidinyl}amino)methyl]-2H-pyrido[3,2-b][1,4]thiazin-3(4H)-one dihydrochloride

### (a) 4-ethenyl-3,6-difluoroquinoline

4-Fluoroaniline was converted through the same series of reactions as outlined in Example (47 c-j) to afford the desired vinyl-quinoline as an oil. MS (ES) m/z 192 (M+H)⁺.

### (b) 1,1-dimethylethyl {1-[2-(3,6-difluoro-4-quinolinyl)ethyl]-4-piperidinyl}carbamate

Vinyl quinoline (a) (0.1 g, 0.5 mmol) and piperidin-4-yl-carbamic acid tert-butyl ester (0.1 g, 0.5 mmol) in DMF (0.2 mL/mmol) were treated as in Example (47k) to afford the product as a solid (0.17 g, 86%). MS (ES) m/z 391 (M+H)⁺.

### (c) 1-[2-(3,6-difluoro-4-quinolinyl)ethyl]-4-piperidinamine

Carbamate (b) was treated with trifluoroacetic acid as in Example (471) to afford the product as a solid (0.13 g, 97%). MS (ES) m/z 291 (M+H)⁺.

### (d) Title Compound

Amine (c) and aldehyde (7d) were treated as in example (47m) to afford the free base of the title compound as a solid (0.13 g, 65%).

¹H NMR δH (d4-MeOH) 8.71 (s, 1H), 8.1 (dd, 1H), 7.8 (dd, 1H), 7.75 (d, 1H), 7.53-7.59 (m, 1H), 7.08 (s, 1H), 4.13 (s, 2H), 3.53 (s, 2H), 3.36 (m, 2H), 3.29-3.35 (m, 3H), 3.17-3.22 (m, 2H), 2.91-3.09 (m, 1H), 2.75-2.78 (m, 2H), 2.23-2.33 (m, 2H), 2.11-2.15 (m, 2H), 1.61-1.71 (m, 2H). MS (ES) m/z 469 (M+H)⁺.

This material, as a solution in chloroform/methanol, was treated with an excess of 1M HCl in ether and evaporated to dryness. The solid was triturated under ether, filtered and dried under vacuum to provide the title compound (155 mg).

The following examples were prepared by analogous method to Example 106:

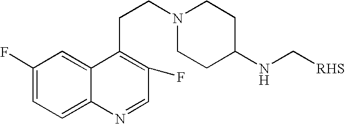

| Example | |
|---|---|
| 107 | 1-[2-(3,6-difluoro-4-quinolinyl)ethyl]-N-(2,3- |
| | dihydro[1,4]dioxino[2,3-c]pyridin-7-ylmethyl)-4- |
| | piperidinamine hydrochloride dihydrochloride |
| | 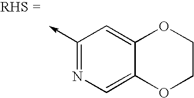 |
| | Aldehyde is 2,3-dihydro[1,4]dioxino[2,3-c]pyridine-7- |
| | carbaldehyde as in example (2c) |
| 108 | 6-[({1-[2-(3,6-difluoro-4-quinolinyl)ethyl]-4- |
| | piperidinyl}amino)methyl]-2H-pyrido[3,2- |
| | b][1,4]oxazin-3(4H)-one dihydrochloride |
| | 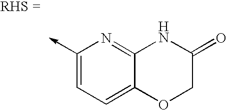 |
| | Aldehyde is 3-oxo-3,4-dihydro-2H-pyrido[3,2-b][1,4]oxazine- |
| | 6-carboxaldehyde as in example (1l) |

### Example 109

### 6-{[(1-{2-[3-chloro-6-fluoro-5-(methoxy)-4-quinolinyl]-1-methylethyl}-4-piperidinyl)amino]methyl}-2H-pyrido[3,2-b][1,4]oxazin-3(4H)-one dihydrochloride

Amine (22l) and aldehyde (1l) were treated as in example (22m) to afford the free base of the title compound as a solid in 65% yield.

¹H NMR δH (d4-MeOH) 8.72 (s, 1H), 7.81 (dd, 1H), 7.65 (dd, 1H), 7.34 (d, 1H), 7.03 (d, 1H), 4.76 (s, 2H), 4.12 (s, 3H), 4.10-4.12 (m, 1H), 3.70-4.12 (m, 2H), 3.35 (m, 3H), 3.30-3.31 (m, 2H) 3.20-3.25 (m, 2H), 3.00 (m, 1H), 2.73-2.80 (m, 2H), 2.32-2.42 (m, 2H), 2.12-2.17 (m, 2H), 1.68-1.74 (m, 2H). MS (ES) m/z 499 (M+H)⁺.

This material, as a solution in chloroform/methanol, was treated with an excess of 1M HCl in ether and evaporated to dryness. The solid was triturated under ether, filtered and dried under vacuum to provide the title compound (34 mg).

The following example was prepared by analogous methods to Example 109:

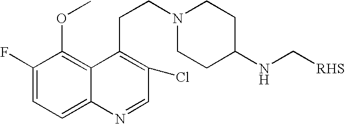

| Example | |
|---|---|
| 110 | 1-{2-[3-chloro-6-fluoro-5-(methoxy)-4-quinolinyl]ethyl}-N- |
| | (2,3-dihydro[1,4]dioxino[2,3-c]pyridin-7-ylmethyl)-4- |
| | piperidinamine dihydrochloride |
| | 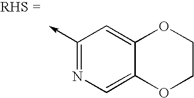 |
| | Aldehyde is 2,3-dihydro[1,4]dioxino[2,3-c]pyridine-7- |
| | carbaldehyde as in example (2c) |

### Example 111

### 1-[2-(6-chloro-3-fluoro-4-quinolinyl)ethyl]-4-[(2,3-dihydro[1,4]dioxino[2,3-c]pyridin-7-ylmethyl)amino]-N-methyl-4-piperidinecarboxamide dihydrochloride

### (a) 6-chloro-4-ethenyl-3-fluoroquinoline

4-Chloroaniline converted through the same series of reactions as outlined in Example (47 a-j) to afford the desired vinyl-quinoline as an oil.

MS (ES) m/z 208 (M+H)⁺.

### (b) Title Compound

Vinyl-quinoline (a) and piperidine (87e) were treated as in Example (52h) to afford the free base of the title compound as an oil.

1H NMR δH (CDCl₃) 8.72 (1H, s), 8.13 (1H, s), 8.02 (1H, d), 7.98 (1H, d), 7.87 (1H, m), 7.59 (1H, dd), 6.74 (1H, s), 4.31 (2H, m), 4.27 (2H, m), 3.59 (2H, s), 3.23 (2H, br t), 2.89 (2H, m), 2.78 (3H, d), 2.66 (2H, m), 2.42 (2H, t), 2.28 (2H, td), 1.69 (2H, br d).

MS (ES) m/z 515 (M+H)⁺.

This material, as a solution in chloroform/methanol, was treated with an excess of 1M HCl in ether and evaporated to dryness. The solid was triturated under ether, filtered and dried under vacuum to provide the title compound.

### Example 112

### 2-{4-[(2,3-dihydro[1,4]dioxino[2,3-c]pyridin-7-ylmethyl)amino]-1-piperidinyl}-1-[3-fluoro-6-(methoxy)-4-quinolinyl]ethanol dihydrochloride Enantiomer 2

Vinyl-quinoline (31e) was taken through the sequence outlined in Example (99), using AD-mixα as a chiral agent for the dihydroxylation step. Final purification was by chiral preparative hplc, again in a manner analogous to that described in Example (100), affording the free base of the title compound as a foam, as the major, second eluting enantiomer.

1H NMR δH (400 mHz, CDCl₃) 8.56 (1H, s), 8.10 (1H, s), 7.95 (1H, d), 7.92 (1H, d), 7.29 (1H, dd), 6.83 (1H, s), 5.58 (1H, dd), 4.25-4.35 (4H, m), 3.93 (3H, s), 3.81 (2H, s), 3.18 (1H, m), 3.03 (1H, m), 2.90 (1H, m), 2.60 (2H, m), 2.49 (1H, br.t), 2.18 (1H, br.t), 1.90 (2H, m), 1.80 (2H, m), 1.40-1.65 (2H, m)

MS (ES) m/z 469 (M+H)⁺.

This material, as a solution in chloroform/methanol, was treated with an excess of 1M HCl in ether and evaporated to dryness. The solid was triturated under ether, filtered and dried under vacuum to provide the title compound as a white solid (530 mg)

### Example 113

### 6-{[trans-1-{2-[3-fluoro-6-(methoxy)-1,5-naphthyridin-4-yl]ethyl}-3-hydroxy-4-piperidinyl)amino]methyl}-2H-pyrido[3,2-b][1,4]thiazin-3(4H)-one dihydrochloride Enantiomer E2

### (a) 1,1-dimethylethyl ((trans-1-{2-[3-fluoro-6-(methoxy)-1,5-naphthyridin-4-yl]ethyl}-3-hydroxy-4-piperidinyl)carbamate, Isomer E2

Vinyl naphthyridine (53h) (1.25 g, 6.1 mmole) was heated to 100° C. together with trans-1,1-dimethylethyl (3-hydroxy-4-piperidinyl)carbamate (prepared by hydrogenation of Example 17f, Isomer E2) (1.32 g, 6.1 mmole) in DMF (5 mL). After 24 hours, the mixture was concentrated in vacuo and purified on silica (CHCl₃/MeOH with 5% NH₄OH, 9:1) to give the product as an oil (1.9 g, 75%).

MS (ES) m/z 421 (M+H)⁺.

### (b) Title Compound

To a solution of carbamate (a) (1.9 g, 4.57 mmole) in dichloromethane (100 mL) was added 4M HCl in dioxane (20 mL). After stirring for 3 h, the reaction contents were concentrated under vacuum to give a white solid which was used without further purification (98%). MS (ES) m/z 321 (M+H)⁺.

To a solution of the above piperidinol hydrochloride salt (ca. 1.0 mmole) in ethanol (20 mL) and dichloromethane (20 mL) was added triethyl amine (0.56 mL, 4.0 mmole) and aldehyde (7d) (0.19 g, 1.0 mmole). After 24 hours at room temperature, sodium borohydride (42 mg, 1.1 mmole) was added and the reaction mixture stirred for 5 hours. Silica gel (˜2 g) was added to the mixture and the reaction contents stirred for an additional 2 hours. The reaction slurry was concentrated to dryness in vacuo and loaded onto a silica gel column (eluting with CHCl₃/MeOH with 5% NH₄OH, 9:1) to afford the title compound as a white foam.

This material, as a solution in chloroform/methanol, was treated with an excess of 2M HCl in ether and evaporated to dryness. The solid was triturated with ether, filtered and dried under vacuum to provide the title compound (71%) as a white solid.

¹H NMR of the dihydrochloride salt δH (CD₃OD) 8.67 (1H, s), 8.31 (1H, d), 7.85 (1H, d), 7.32 (1H, d), 7.17 (1H, d), 4.76 (4H, m), 4.51 (2H, m), 4.43 (1H, m), 4.18 (3H, s), 3.93 (2H, m), 3.87 (2H, m), 3.71 (2H, m), 3.15 (1H, m), 2.59 (1H, s), 2.23 (1H, m). MS (+ve ion electrospray) m/z 499 (M+H)⁺.

### Example 114

### 6-{[trans-1-{2-[3-fluoro-6-(methoxy)-1,5-naphthyridin-4-yl]ethyl}-3-hydroxy-4-piperidinyl)amino]methyl}-2H-pyrido[3,2-b][1,4]oxazin-3(4H)-one dihydrochloride Enantiomer E2

This was prepared by the analogous process to Example (113) with the exception that aldehyde (1l) was used in the reductive alkylation step.

¹H NMR (of the dihydrochloride salt) δH (CD₃OD) 8.93 (1H, s), 8.35 (1H, d), 7.58 (1H, d), 7.37 (1H, d), 7.12 (1H, d), 4.73 (3H, m), 4.44 (2H, m), 4.39 (1H, m), 4.21 (3H, s), 3.85 (3H, m), 3.77 (2H, m), 3.71 (2H, m), 3.18 (1H, m), 2.60 (1H, s), 2.22 (1H, m). MS (+ve ion electrospray) m/z 483 (M+H)⁺.

### Example 115

### trans-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinol dihydrochloride Enantiomer E2

This was prepared by the analogous process to Example (113) with the exception that aldehyde (2c) was used in the reductive alkylation step.

¹H NMR of the dihydrochloride salt δH (CD₃OD) 8.82 (1H, s), 8.48(1H, s), 8.31 (1H, d), 7.59 (1H, s), 7.29 (1H, d), 4.65 (4H, m), 4.51 (2H, m), 4.40 (1H, m), 4.21 (3H, s), 3.97 (1H, m), 3.89 (1H, m), 3.80 (2H, m), 3.63 (4H, m), 3.19 (1H, m), 2.64 (1H, s), 2.30 (1H, m). MS (+ve ion electrospray) m/z 470 (M+H)⁺.

### Example 116

### 6-{[trans-1-{2-[3-fluoro-6-(methoxy)-4-quinolinyl]ethyl}-3-hydroxy-4-piperidinyl)amino]methyl}-2H-pyrido[3,2-b][1,4]thiazin-3(4H)-one dihydrochloride Enantiomer E2

This was prepared by the analogous process to Example (113) with the exception that vinyl quinoline (31e) was used in place of vinyl naphthyridine (53h).

¹H NMR of the dihydrochloride salt δH (CDCl₃) 8.60 (1H, s), 8.01 (1H, d), 7.57 (1H, d), 7.32 (1H, d), 7.20 (1H, s), 6.94 (1H, d), 4.07 (1H, d), 3.96 (3H, s), 3.87 (1H, d), 3.62 (1H, m), 3.47 (2H, s), 3.25 (3H, m), 3.02 (1H, m), 2.73 (2H, m), 2.49 (1H, m), 2.13 (3H, m), 1.52 (1H, m).

MS (+ve ion electrospray) m/z 498 (M+H)⁺.

The following example was prepared by analogous methods to Examples 115/116

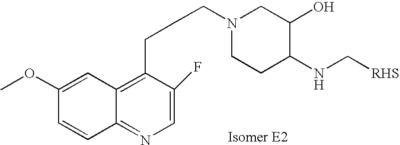

| Example | |
|---|---|
| 117 | trans-4-[(2,3-dihydro[1,4]dioxino[2,3-c]pyridin-7-ylmethyl) |
| | amino]-1-{2-[3-fluoro-6-(methoxy)-4-quinolinyl]ethyl}-3- |
| | piperidinol dihydrochloride |
| | 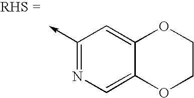 |
| | Aldehyde is 2,3-dihydro[1,4]dioxino[2,3-c]pyridine-7- |
| | carbaldehyde as in example (2c) |

### Example 118

### N-trans-1-{2-[3-fluoro-6-(methoxy)-1,5-naphthyridin-4-yl]ethyl}-3-hydroxy-4-piperidinyl)-3-oxo-3,4-dihydro-2H-pyrido[3,2-b][1,4]thiazine-6-carboxamide hydrochloride Enantiomer E2

To a solution of trans-4-amino-1-{2-[3-fluoro-6-(methoxy)-1,5-naphthyridin-4-yl]ethyl}-3-piperidinol hydrochloride salt Isomer E2) (see Example 113b for the crude preparation of this intermediate), (0.62 mmole) in DMF (20 mL) was added hydroxy benzotriazole hydrate (0.92 g, 0.68 mmole), 1-(3-dimethylaminopropyl)-3-ethylcarbodiimide hydrochloride (0.13 g, 0.68 mmole), diisopropylethyl amine (0.43 mL, 2.48 mmole) and carboxylic acid (7b) (0.13 g, 0.62 mmole). After stirring for 24 hours, the reaction contents were concentrated in vacuo and purified on silica (CHCl₃/MeOH with 5% NH₄OH, 9:1) to afford the title compound as an off-white solid.

This material, as a solution in chloroform/methanol, was treated with an excess of 2M HCl in ether and evaporated to dryness. The solid was triturated with ether, filtered and dried under vacuum to provide the title compound (85%) as a white solid.

¹H NMR of the dihydrochloride salt δH (CDCl₃) 8.61 (1H, s), 8.19 (1H, d), 7.79 (2H, m), 7.31 (1H, d), 7.10 (1H, d), 4.50 (1H, m), 4.15 (3H, s), 3.65-3.89 (4H, m), 3.42 (3H, m), 3.09 (2H, s), 2.92 (2H, m), 2.47 (1H, m), 2.11 (1H, m).

MS (+ve ion electrospray) m/z 513 (M+H)⁺.

### Example 119

### N-trans-1-{2-[3-fluoro-6-(methoxy)-1,5-naphthyridin-4-.yl]ethyl}-3-hydroxy-4-piperidinyl)-2,3-dihydro[1,4]dioxino[2,3-c]pyridine-7-carboxamide hydrochloride Enantiomer E2

### (a) 2,3-dihydro[1,4]dioxino[2,3-c]pyridine-7-carboxylic acid

Aldehyde (2c) (1.65 g, 10 mmol) was dissolved in water/acetone (150 mL/50 mL) the treated with sulfamic acid (1.3 g) and sodium chlorite (1.2 g) at 0° C. The mixture was allowed to warm to room temperature over 1 hour, then evaporated to dryness. Chromatography on silica eluting with 10% methanol/dichloromethane afforded the acid (1.6 g). MS (APCl⁻) m/z 180 ([M−H]⁻,

### (b) Title Compound

This was prepared using the sampe procedure as for Example (118) except using carboxylic acid (a) to afford the product as a white solid (79%).

¹H NMR of the dihydrochloride salt δH (CD₃OD) 8.81 (1H, s), 8.54 (1H, s), 8.30 (1H, d), 8.13 (1H, s), 7.28 (1H, d), 4.67 (2H, m), 4.56 (2H, m), 4.19 (3H, s), 3.90 (2H, m), 3.81 (4H, m), 3.65 (2H, m), 3.12 (2H, m), 2.31 (1H, m), 2.17 (1H, m).

MS (+ve ion electrospray) m/z 484 (M+H)⁺.

### Example 120

### racemic, trans-6{[(1-{2-[3-fluoro-6-(methoxy)-1,5-naphthyridin-4-yl]ethyl}3-hydroxy-3-methyl-4-piperidinyl)amino]methyl}-2H-pyrido[3,2-b][1,4]thiazin-3(4H)-one dihydrochloride

### (a) 5-methyl-1-(phenylmethyl)-1,2,3,6-tetrahydropyridine

3-Methylpyridine (20 g, 0.215 mmol) and benzyl chloride (25 mL, 0.215 mmol) were combined at 25° C. and stirred 24 h. The resulting salt was washed several times with Et₂O and used without further purification.

MS (+ve ion electrospray) m/z 184 (M+H)⁺.

The above salt (27 g, 0.123 mmol) in EtOH (150 mL) was added dropwise to a solution of NaBH₄ (18.6 g, 0.492 mol) in EtOH (423 mL) at 0° C. The resulting suspension gradually warmed to 25° C. over 12 hours, was concentrated and partitioned between water-dichloromethane. The aqueous phase was washed several times with dichloromethane and the combined organic fractions were dried (Na₂SO₄), concentrated and chromatographed on silica gel to afford the product as an orange oil (10 g, 43%).

MS (+ve ion electrospray) m/z 188 (M+H)⁺.

### (b) 2-(trimethylsilyl)ethyl 5-methyl-3,6-dihydro-1 (2h)-pyridinecarboxylate

To a solution of piperidine (a) (8 g, 42.75 mmol) in dry toluene (43 mL) at 25° C. was added dropwise a solution of 2-(trimethylsilyl)ethyl chloridocarbonate (55 mL, 51.3 mmol) [freshly prepared by the procedure of Shute and Rich Synthesis 1987, 346.] and the resulting solution stirred at 80° C. After 12 hours the solution was concentrated and chromatographed on silica gel eluting with 0-5% MeOH in DCM affording the product as an orange oil that was used without further purification (10.39, >quant. contaminated with residual benzyl chloride).

MS (+ve ion electrospray) m/z 242 (M+H)⁺.

### (c) 2-(trimethylsilyl)ethyl 1-methyl-7-oxa-3-azabicyclo[4.1.0]heptane-3-carboxylate

To a solution of piperidine (b) (10 g, 41.4 mmol) in dry dichloromethane (138 mL) at 0° C. was added m-chloroperbenzoic acid (8.58 g, 49.7 mmol) batchwise. After stirring 12 hours at 25° C., the solution was partitioned between a 1N aqueous solution of sodium hydroxide and dichloromethane and the aqueous phase was back extracted several times with dichloromethane. The combined organic fractions were combined, dried (Na₂SO₄), concentrated and chromatographed on silica gel eluting with 2% methanol in dichloromethane to afford the product as a clear oil (6 g, 56%). MS (+ve ion electrospray) m/z 258 (M+H)⁺.

### (d) 2-(trimethylsilyl)ethyl (3S,4S)-4-amino-3-hydroxy-3-methyl-1-piperidinecarboxylate and 2-(trimethylsilyl)ethyl (3R,4R)-4-amino-3-hydroxy-3-methyl-1-piperidinecarboxylate

A solution of epoxide (c) (6 g, 23.3 mmol) in NH₄OH (50 mL) was heated to 90° C. in a sealed tube. After 12 hours, the resulting solution was concentrated and used directly without further purification. MS (+ve ion electrospray) m/z 275 (M+H)⁺.

### (e) 2-(trimethylsilyl)ethyl (3S,4S)-4-({[(1,1-dimethylethyl)oxy]carbonyl}amino)-3-hydroxy-3-methyl-1-piperidinecarboxylate and 2-(trimethylsilyl)ethyl (3R,4R)-4-({[(1,1-dimethylethyl)oxy]carbonyl}amino)-3-hydroxy-3-methyl-1-piperidinecarboxylate

To a solution of aminopiperidine (d) (6 g, 21.86 mmol) in dry acetonitrile (109 mL) at 25° C. were added N,N-diisopropylethylamine (5.7 mL, 32.8 mmol) and bis(1,1-dimethylethyl) dicarbonate (7.5 mL, 32.8 mmol). After 1 hour the solution was concentrated and chromatographed on silica gel eluting with 1% MeOH in DCM containing 1% NH₄OH affording the product as a white solid (6.7 g, 82%).

MS (+ve ion electrospray) m/z 397 (M+H)⁺.

### (f) 1,1-dimethylethyl [(3S,4S)-3-hydroxy-3-methyl-4-piperidinyl]carbamate and 1,1-dimethylethyl [(3R,4R)-3-hydroxy-3-methyl-4-piperidinyl]carbamate

To a solution of protected piperidine (e) (1 g, 2.67 mmol) in dry acetonitrile (27 mL) at 25° C. was added tetrabutylammonium fluoride (1M in THF, 3.2 mL, 3.20 mmol). After stirring at 50° C. for 12 hours, the solution was concentrated and used without further purification. MS (+ve ion electrospray) m/z 231 (M+H)⁺.

### (g) Rac-1,1-dimethylethyl (3-hydroxy-3-methyl-1-{2-[6-(methoxy)-1,5-naphthyridin-4-yl]ethyl}-4-piperidinyl)carbamate

A solution of (racemic) piperidine (f) (614 mg, 2.66 mmol) and vinyl quinoline (53h) (500 mg, 2.42 mmol) in dry DMF (5.0 mL) was stirred at 90° C. After 48 hours, the solution was concentrated and chromatographed on silica gel eluting with 3% MeOH in DCM with 1% NH₄OH to afford the product as a yellow oil (560 mg, 50%).

MS (+ve ion electrospray) m/z 417 (M+H)⁺.

### (h) Title Compound

Carbamate (g) (175 mg, 0.42 mmol) was deprotected giving the hydrochloride salt using the same procedure as used in Example (119)

MS (+ve ion electrospray) m/z 316 (M+H)⁺.

A solution of the above salt in dichloromethane and ethanol (6 mL) at 25° C. were added N,N-diisopropylethylamine (731 μL, 4.20 mmol), Na₂SO₄ (94 mg, 0.662 mmol) and aldehyde (7d) (89 mg, 0.42 mmol). After 12 hours, sodium borohydride (25 mg, 0.504 mmol) was added and the reaction stirred an additional 2 hours, was concentrated and chromatographed on silica gel eluting with 3% MeOH in DCM with 1% NH₄OH to afford the free base of the title compound as a yellow solid (100 mg, 37%).

¹H NMR (CD₃OD, 500 MHz) δ 8.64 (s, 1H), 8.21 (d), 7.68 (d, 1H), 7.19 (d, 1H), 7.03 (d, 1H), 4.12 (s, 3H), 3.82 (m, 2H), 3.53 (s, 2H), 3.33-3.45 (m, 2H), 3.00-3.03 (m, 1H), 2.78-2.81 (m, 3H), 2.32-2.39 (m, 1H), 2.12-2.13 (m, 1H), 2.01-2.04 (m, 1H), 1.93-1.96 (m, 1H), 1.22-1.26 (m, 1H), 1.08 (s, 3H). MS (+ve ion electrospray) m/z 513 (M+H)⁺.

This material, as a solution in MeOH, was treated with an excess of 4M HCl in dioxane and evaporated to dryness to provide the title compound.

Subsequent to this work, Example (120e) has been resolved:—

### Enantiomeric resolution of (+/−)-trans-2-(trimethylsilyl)ethyl-4-({[(1,1-dimethylethyl)oxy]carbonyl}amino)-3-hydroxy-3-methyl-1-piperidinecarboxylate by chiral HPLC

(+/−)-trans-2-(trimethylsilyl)ethyl-4-({[(1,1-dimethylethyl)oxy]carbonyl}amino)-3-hydroxy-3-methyl-1-piperidinecarboxylate (1.8 g) was dissolved in 50 mL of acetonitrile and applied to a column of ChiralPak AD (77×240 mm, 20 u). Elution with acetonitrile: isopropyl alcohol (95:5) was carried out at a flowrate of 300 mL/min, and uv detection at 220 nm to yield the separate enantiomers:

Isomer E1 (0.77 g) alpha D +13.6° (c=1, CH₃OH); chiral purity >98% ee with retention time 2.4 min on analytical HPLC [Chiralpak AD 4.6×150 mm, 10 u, acetonitrile: isopropyl alcohol (95:5), 1.0 mL/min, uv 205 nm].

Isomer E2 (0.78 g) alpha D −13.30 (c=1, CH₃OH); chiral purity >98% ee with retention time 3.1 min on analytical HPLC [Chiralpak AD 4.6×150 mm, 10 u, acetonitrile: isopropyl alcohol (95:5), 1.0 mL/min, uv 205 nm].

The following racemic example was prepared by analogous methods to Example 120 using the aldehyde shown below:

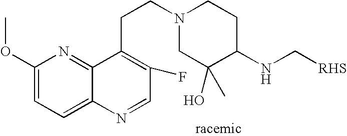

| Example | |
|---|---|
| 121 | Trans-4-[(2,3-dihydro[1,4]dioxino[2,3-c]pyridin-7-ylmethyl) |
| | amino]-1-{2-[3-fluoro-6-(methoxy)-1,5-naphthyridin-4- |
| | yl]ethyl}-3-methyl-3-piperidinol dihydrochloride |
| | 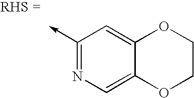 |
| | Aldehyde is 2,3-dihydro[1,4]dioxino[2,3-c]pyridine-7- |
| | carbaldehyde as in example (2c) |

### Example 122

### 6-{[trans-1-{2-[3-fluoro-6-(methoxy)-1,5-naphthyridin-4-yl]ethyl}-3-hydroxy-4-methyl-4-piperidinyl)amino]methyl}-2H-pyrido[3,2-b][1,4]thiazin-3(4H)-one dihydrochloride Enantiomer E1

### (a) 4-methyl-1-(phenylmethyl)-1,2,3,6-tetrahydropyridine

4-methylpyridine (10 g, 0.107 mmol) and benzyl chloride (12 mL, 0.107 mmol) were combined at 25° C. and stirred 24 hours The resulting salt was washed several times with Et₂O and used without further purification.

MS (+ve ion electrospray) m/z 184 (M+H)⁺.

The above salt (15 g, 68.3 mmol) in EtOH (100 mL) was added dropwise to a solution of sodium borohydride (10 g, 0.273 mol) in ethanol (235 mL) at 0° C. The resulting suspension gradually warmed to 25° C. over 12 hours, was concentrated and partitioned between water and dichloromethane. The aqueous phase was washed several times with DCM and the combined organic fractions were dried (Na₂SO₄), concentrated and chromatographed on silica gel yielding the product as an orange oil (12.8 g, quant.).

MS (+ve ion electrospray) m/z 188 (M+H)⁺.

### (b) Methyl 4-methyl-3,6-dihydro-1 (2H)-pyridinecarboxylate

To a solution of tetrahydropyridine (a) (6 g, 32.1 mmol) in dry toluene (32 mL) at 25° C. was added dropwise a solution of methyl chloroformate (5 mL, 64.1 mmol). After 12 hours at 80° C. the resulting solution was concentrated and chromatographed on silica gel eluting with 10% MeOH in DCM to afford the product as an orange oil (5.8 g, >quant. contaminated with residual benzyl chloride) that was used without further purification.

MS (+ve ion electrospray) m/z 156 (M+H)⁺.

### (c) Methyl 6-methyl-7-oxa-3-azabicyclo[4.1.0]heptane-3-carboxylate

To a solution of tetrahydropyridine (b) (1.0 g, 6.4 mmol) in dry dichloromethane (21 mL) at 0° C. was added m-chloroperbenzoic acid (1.3 g, 7.7 mmol) batchwise. After stirring 12 hours at 25° C., the solution was partitioned between 1N NaOH-DCM and the aqueous phase was back extracted several times with dichloromethane. The combined organic fractions were dried (Na₂SO₄), concentrated and chromatographed on silica gel eluting with 1% MeOH in DCM yielding the product as a clear oil (1.0 g, 91%).

MS (+ve ion electrospray) m/z 172 (M+H)⁺.

### (d) (+/−)-trans-methyl-4-amino-3-hydroxy-4-methyl-1-piperidinecarboxylate

To a refluxing solution of trimethylsilyl cyanide (4.6 mL, 34.3 mmol), ZnI₂ (273 mg, 0.857 mmol) in dry dichloromethane (171 mL) was added epoxide (96c) (3.0 g, 17.13 mmol). After 12 hours the solution was cooled, concentrated and chromatographed on silica gel eluting with dichloromethane yielding the isonitrile as a yellow oil which was used without further purification.

MS (+ve ion electrospray) m/z 273 (M+H)⁺.

To the above isonitrile in dry MeOH (100 mL) was added excess 4M HCl in dioxane (18 mL, 71.6 mmol). After 1 hour, the solution was concentrated, the residue was taken up in MeOH and excess N,N-diisopropylethyl amine was added to neutralise the salt. The solution was concentrated and chromatographed on silica gel eluting with 9% MeOH-1% NH₄OH in DCM yielding the product as a white solid (2.0 g, 74%).

MS (+ve ion electrospray) m/z 189 (M+H)⁺.

### Enantiomeric resolution of (+/−)-trans-methyl-4-amino-3-hydroxy-4-methyl-1-piperidinecarboxylate by chiral HPLC

(+/−)-trans-methyl-4-amino-3-hydroxy-4-methyl-1-piperidinecarboxylate (1.0 g) was dissolved in 100 mL of acetonitrile:isopropyl alcohol:isopropylamine (85:15:0.1) and applied to a column of ChiralPak AD (77×240 mm, 20 u). Elution with acetonitrile: isopropyl alcohol:isopropylamine (85:15:0.1) was carried out at a flowrate of 300 mL/min, and uv detection at 220 nm to yield the separate enantiomers:

Isomer E1 (0.41 g) alpha D −8.8° (c=1, CH₃OH); chiral purity >99% ee with retention time 2.8 min on analytical HPLC (Chiralpak AD 4.6×150 mm, 10 u, acetonitrile: isopropyl alcohol:isopropylamine (85:15:0.1), 1.0 mL/min, uv 205 nm].

Isomer E2 (0.40 g) alpha D +9.1° (c=1, CH₃OH); chiral purity >99% ee with retention time 3.7 min on analytical HPLC [Chiralpak AD 4.6×150 mm, 10 u, acetonitrile: isopropyl alcohol:isopropylamine (85:15:0.1), 1.0 mL/min, uv 205 nm].

### (e) trans-4-amino-4-methyl-3-piperidinol

A solution of piperidinecarboxylate (d, Isomer E1) (1.0 g, 5.32 mmol) in ethanol (12 mL) and 1N NaOH (16 mL) was stirred at reflux. After 12 hours, the solution was concentrated and the residue was extracted with MeOH. The organic fractions were concentrated and chromatographed on silica gel eluting with 9% MeOH, 1% NH₄OH in DCM affording the product as a clear oil (691 mg, quant.).

MS (+ve ion electrospray) m/z 131 (M+H)⁺.

### (f) trans-4-amino-4-methyl-1-{3-fluoro-2-[6-(methoxy)-1,5-naphthyridin-4-yl]ethyl}-3-piperidinol

A solution of piperidinol (e, Isomer E1) (384 mg, 2.95 mmol) and vinyl-naphthyridine (53h) (450 mg, 2.21 mmol) in dry DMF (5.0 mL) was stirred at 90° C. After 12 hours, the solution was concentrated and chromatographed on silica gel eluting with 6% MeOH in DCM with 1% NH₄OH affording the product as a yellow oil (425 mg, 50%).

MS (+ve ion electrospray) m/z 317 (M+H)⁺.

### (g) Title Compound

A solution of amine (f, Isomer E1) (175 mg, 0.552 mmol), aldehyde (7d) (89 mg, 0.552 mmol) and Na₂SO₄ (94 mg, 0.662 mmol) in DCM-EtOH (1:1, 6 mL) was stirred for 12 hours. Sodium borohydride (25 mg, 0.662 mmol) was added and the solution stirred an additional 2 hours. The reaction mixture was concentrated and chromatographed on silica gel eluting with 5% MeOH in DCM with 1% NH₄OH affording the free base of the title compound as a yellow solid (100 mg, 37%)

¹H NMR (CD₃OD, 400 MHz), δ 8.66 (s, 1H), 8.22 (d, 1H), 7.69 (d, 1H), 7.20 (d, 1H), 7.05 (d, 1H), 4.14 (s, 3H), 3.83 (AB quartet, 2H), 3.73-3.75 (m, 1H), 3.48-3.52 (m, 4H), 3.04-3.06 (m, 1H), 2.90-2.93 (m, 1H), 2.85-2.87 (m, 2H), 2.35-2.49 (m, 2H), 1.70-1.73 (m, 2H), 1.18 (s, 3H).

MS (+ve ion electrospray) m/z 513 (M+H)⁺.

This material, as a solution in MeOH, was treated with an excess of 4M HCl in dioxane and evaporated to dryness to provide the title compound.

The following example was prepared by analogous methods to Example 122:

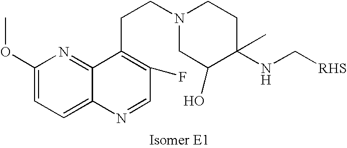

| Example | RHS |
|---|---|
| 123 | Trans-4-[(2,3-dihydro[1,4]dioxino[2,3-c]pyridin-7-ylmethyl) |
| | amino]-1-{2-[3-fluoro-6-(methoxy)-1,5-naphthyridin-4- |
| | yl]ethyl}-4-methyl-3-piperidinol dihydrochloride |
| | 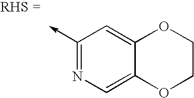 |
| | Aldehyde is 2,3-dihydro[1,4]dioxino[2,3-c]pyridine-7- |
| | carbaldehyde as in example (2c) |

### Example 124

### 6-{[trans-1-{2-[3-fluoro-6-(methoxy)-1,5-naphthyridin-4-yl]ethyl}-3-hydroxy-4-methyl-4-piperidinyl)amino]methyl}-2H-pyrido[3,2-b][1,4]thiazin-3(4H)-one dihydrochloride Enantiomer E2

### (a) trans-4-amino-4-methyl-3-piperidinol

A solution of piperidinecarboxylate (122d—Isomer E2) (1.0 g, 5.32 mmol) in ethanol (12 mL) and 1N NaOH (16 mL) was stirred at reflux. After 12 hours, the solution was concentrated and the residue was extracted with MeOH. The organic fractions were concentrated and chromatographed on silica gel eluting with 9% MeOH, 1% NH₄OH in DCM affording the product as a clear oil (691 mg, quant.).

MS (+ve ion electrospray) m/z 131 (M+H)⁺.

### (b) trans-4-amino-4-methyl-1-{2-[6-(methoxy)-1,5-naphthyridin-4-yl]ethyl}-3-piperidinol

A solution of piperidinol (e, Isomer E2) (384 mg, 2.95 mmol) and vinyl-naphthyridine (53h) (450 mg, 2.21 mmol) in dry DMF (5.0 mL) was stirred at 90° C. After 12 hours, the solution was concentrated and chromatographed on silica gel eluting with 6% MeOH in DCM with 1% NH₄OH affording the product as a yellow oil (425 mg, 50%).

MS (+ve ion electrospray) m/z 317 (M+H)⁺.

### (c) Title Compound

A solution of amine (b, Isomer E2) (175 mg, 0.552 mmol), aldehyde (7d) (89 mg, 0.552 mmol) were treated as in example (123g) to afford the free base of the title compound in a 60% yield.

¹H NMR (CD₃OD, 400 MHz), δ 8.66 (s, 1H), 8.22 (d, 1H), 7.69 (d, 1H), 7.20 (d, 1H), 7.05 (d, 1H), 4.14 (s, 3H), 3.83 (AB quartet, 2H), 3.73-3.75 (m, 1H), 3.48-3.52 (m, 4H), 3.04-3.06 (m, 1H), 2.90-2.93 (m, 1H), 2.85-2.87 (m, 2H), 2.35-2.49 (m, 2H), 1.70-1.73 (m, 2H), 1.18 (s, 3H).

MS (+ve ion electrospray) m/z 513 (M+H)⁺.

This material, as a solution in MeOH, was treated with an excess of 4M HCl in dioxane and evaporated to dryness to provide the title compound.

The following example was prepared by analogous methods to Example 124, using the aldehydes shown below:

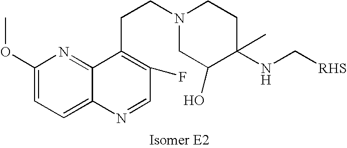

| Example | |
|---|---|
| 125 | Trans-4-[(2,3-dihydro[1,4]dioxino[2,3-c]pyridin-7- |
| | ylmethyl)amino]-1-{2-[3-fluoro-6-(methoxy)-1,5- |
| | naphthyridin-4-yl]ethyl}-4-methyl-3-piperidinol |
| | dihydrochloride |
| | 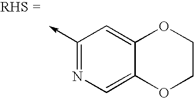 |
| | Aldehyde is 2,3-dihydro[1,4]dioxino[2,3-c]pyridine-7- |
| | carbaldehyde as in example (2c) |

### Example 126

### N-(3,4-dihydro-2H-pyrano[2,3-c]pyridin-6-ylmethyl)-1-{2-[3-fluoro-6-(methoxy)-1,5-naphthyridin-4-yl]ethyl}-4-piperidinamine dihydrochloride

### (a) [4-(3-hydroxy-1-propyn-1-yl)-5-({[4-(methoxy)phenyl]methyl)oxy)-2-pyridinyl]methyl acetate

A mixture of triflate (60d) (1.0 g, 2.3 mmol), propynol (0.15 mL, 2.5 mmol), copper diodide (22 mg, 0.125 mmol), palladium dichloro-bis-triphenylphosphine (II) (32 mg, 0.046 mmol), triethylamine (5.7 mL, 41.4 mmol) in acetonitrile (30 mL) was stirred at 50° C. for one hour. A further equivalent of propynol was added and the reaction mixture was stirred at 50° C. for a further 18 hours. The reaction mixture was evaporated under vacuum to dryness. The residue was partionned between ethyl acetate and a 0.1 M solution of sodium ethylenediamineacetate. The organic layer was washed with water and dried over sodium sulfate. The residue was chromatographed on silica gel eluting with 25-100% ethyl acetate in 40-60 petroleum ether to afford the product as on oil (0.48 g, 61%).

MS (+ve ion electrospray) m/z 342(MH+).

### (b) [5-hydroxy-4-(3-hydroxypropyl)-2-pyridinyl]methyl acetate

A solution of alkyne (a) (3.3 g, 7.7 mmol) in ethanol (100 mL) was hydrogenated in a Parr under 3 atmospheres of hydrogen with palladium on charcoal for 6 hours. The reaction mixture was filtered through Kieselguhr and washed several times with ethanol then evaporated to dryness under vacuum to afford the product as a white solid (2.17 g, 100%).

MS (+ve ion electrospray) m/z 226(MH+).

### (c) 3,4-Dihydro-2H-pyrano[2,3-c]pyridin-6-ylmethyl acetate

A mixture of triphenylphosphine (4.92 g, 18.8 mol) and diisopropylazidicarboxylate (3.74 mL, 18.8 mol) in tetrahydrofuran (100 mL) was stirred under argon for 1 hour. A solution of diol (b) (2.12 g, 9.38 mmol) in tetrahydrofuran was added and the reaction mixture was stirred at room temperature for 2 hours. It was evaporated under vacuum. The residue was chromatographed on silica gel eluting with 25-50% ethyl acetate in petroleum ether then with 50-75% ethyl acetate to afford the product as a yellow oil (1.42 g, 60%).

MS (+ve ion electrospray) m/z 208 (MH+).

### (d) 3,4-Dihydro-2H-pyrano[2,3-c]pyridin-6-ylmethanol

A solution of acetate (c) (1.52 g, 5.85 mmol) in tetrahydrofuran/water 1/1 (40 mL) was treated with a 2N solution of sodium hydroxide (5.9 mL, 11.7 mmol). The reaction mixture was stirred at room temperature for 2 hours. The reaction mixture was saturated with potassium carbonate and extracted several times with ethyl acetate. The combined organic extracts were dried over magnesium sulfate and evaporated under vacuum to afford the product as an oil (1.22 g, 100%).

MS (+ve ion electrospray) m/z 166 (MH+).

### (e) 3,4-dihydro-2H-pyrano[2,3-c]pyridine-6-carbaldehyde

Alcohol (d) (1.22 g) was oxidised with manganese(II)oxide as in Example (2c) to afford the aldehyde as a white solid (0.532 g, 60%).

MS (+ve ion electrospray) m/z 164 (MH+).

### (f) Title Compound

A mixture of the hydrochloride salt of amine (53i) (prepared from deprotection with HCl instead of TFA, according to the procedure of Example 113b) (130 mg, 0.35 mmol) and aldehyde (e) (57 mg, 0.35 mmol) in methanol (8 mL) was treated with sodium bicarbonate (319 mg, 1.73 mmol) at room temperature. The reaction was allowed to stir at room temperature for 18 hours. Sodium borohydride (13 mg, 0.35 mmol) was added, the mixture was continuously stirred for 1 hour at room temperature. Methanol was removed under reduced pressure and the residue was partitioned between ethyl acetate and water. The organic layer was separated, washed with aqueous sodium chloride, dried over magnesium sulfate and concentrated under reduced pressure. The residue was chromatographed on silica gel eluting with 0-10% methanol in dichloromethane to afford the free base of the product as a solid. (97 mg, 64%).

¹H NMR (CD₃OD, 400 MHz): 8.48 (s, 1H), 8.1 (d, 1H), 7.8 (s, 1H), 7.05 (dd,s, 2H), 4.1 (m, 2H), 4.0 (s, 3H), 3.6(s, 2H), 3.3(m, 2H), 3.0 (m, 2H), 2.7-2.8(m, 4H), 2.4(m, 1H), 2.1(m,3H), 1.8-1.9(m, 3H), 1.3(m, 2H)

MS (+ve ion electrospray) m/z 452 (MH+).

### Example 127

### {[(1-{2-[3-Fluoro-6-(methoxy-5-naphthyridin-4-yl]ethyl}-4-piperidinyl)amino]methyl}-3,4-dihydro-1,8-naphthyridin-2-(1H)-one

### (a) Methyl 6-amino-5-[(1E)-3-(ethyloxy)-3-oxo-1-propen-1-yl]-2-pyridinecarboxylate

A mixture of palladium acetate (211 mg, 0.23 mmol), tri-tolylphosphite (280 mg, 0.92 mmol) and triethylamine (3.18 mL, 23 mmol) were stirred at room temperature for 30 minutes in degassed DMF. Methyl 6-amino-5-bromopyridine-2-carboxylate (T. R. Kelly and F. Lang, J. Org. Chem. 61, 1996, 4623-4633) (58 mg, 4.60 mmol) was added followed by ethyl acrylate (2.49 mL, 23 mmol). The resultant solution was stirred at 100° C. for 18 hours. The reaction mixture was cooled down to room temperature and filtered through Kieselguhr. DMF was evaporated under vacuum and the residue was chromatographed on silica gel eluting with 25-50% petroleum ether in ethyl acetate to afford the product as an oil (360 mg, 31%).

MS (+ve ion electrospray) m/z 251 (MH+).

### (b) methyl 6-amino-5-[3-(ethyloxy)-3-oxopropyl]-2-pyridinecarboxylate

A solution of acrylate ester (a) (350 mg, 1.41 mmol) in methanol (50 mL) was hydrogenated with palladium on charcoal for 18 hours. The reaction mixture was filtered through Kieselguhr and evaporated under vacuum to afford the product as an oil (345 mg, 97%).

MS (+ve ion electrospray) m/z 253 (MH+).

### (c) methyl 7-oxo-1,5,6,7-tetrahydro-1,8-naphthyridine-2-carboxylate

A solution of amino ester (b) (360 mg, 1.4 mmol) in acetic acid (20 mL) was heated to 100° C. for 1 hour. Acetic acid was evaporated in vacuo and the residue dried under high vacuum for 18 hours to afford a yellow solid (361 mg, 100%).

MS (+ve ion electrospray) m/z 207 (MH+).

### (d) 7-oxo-1,5,6,7-tetrahydro-1,8-naphthyridine-2-carboxylic acid

A solution of carboxylate (c) (355 mg, 1.72 mmol) in dioxan (5 mL)/water (1 mL) was treated dropwise with 2M NaOH solution (1 mL) and stirred for 1 hour. After evaporation to approx. 2 mL, water was added and 2N HCl to pH4. The precipitated solid was filtered off, washed with a small volume of water and dried under vacuum to give the product as a solid (263 mg, 79%).

MS (+ve ion electrospray) m/z 193 (MH+).

### (e) 7-(hydroxymethyl)-3,4-dihydro-1,8-naphthyridin-2(1H)-one

A solution of carboxylic acid (d) (293 mg, 1.53 mmol) in dichloromethane (5 mL)/tetrahydrofuran (5 mL) with triethylamine (466 mg, 3.36 mmol) was cooled to −10° C. and isobutyl chloroformate (0.218 mL, 1.68 mmol) added. After 20 minutes the suspension was filtered through Kieselguhr into an ice-cooled solution of sodium borohydride (110 mg, 4.59 mmol) in water (1 mL), the mixture was stirred 30 minutes and the pH reduced to 7 with dilute hydrochloric acid. The solvent was evaporated and the residue triturated under water. The residue was filtered and dried under vacuum to afford the product as a white solid (262 mg, 96%).

MS (+ve ion electrospray) m/z 179 (MH+).

### (f) 7-oxo-1,5,6,7-tetrahydro-1,8-naphthyridine-2-carbaldehyde

Alcohol (e) was treated as in example (2c) to afford the product as a white solid (72.2 mg, 28%).

MS (+ve ion electrospray) m/z 177 (MH+).

### (g) Title Compound

A solution of amine (53i) (0.257 mg, 0.624 mmol) and solid sodium bicarbonate (262.1 mg, 3.12 mmol) in methanol (2.8 mL) was stirred at room temperature for 5 minutes. Dichloromethane (2.8 mL), aldehyde (f) (116 mg, 0.661 mmol) and sodium sulfate (710 mg, 5.0 mmol) were added and the reaction mixture was stirred at room temperature for 24 hours. The intermediate imine was treated with sodium triacetoxyborohydride (0.263.3 mg, 2.05 mmol) and stirred for an additional 48 hours. The reaction was acidified to pH 3 with 6N HCl, then stirred for 10 minutes. The solvents were removed under reduced pressure and the residue was partitioned between dichloromethane and aqueous sodium bicarbonate. The organic layer was dried over sodium sulfate and evaporated under vacuum. The residue was chromatographed on silica gel eluting with 1-5% methanol in dichloromethane to afford the title compound as an amorphous yellow solid (92.1 mg, 32%).

1H NMR δ (CDCl₃) 8.62 (1H, s), 8.22 (1H, bs), 8.17 (1H, d), 7.42 (1H, d), 7.06, (1H, d), 6.94 (1H, d), 4.08 (3H, s), 3.84 (2H, s) 3.41 (2H, t), 3.06 (2H, bd), 2.93 (2H, t), 2.75 (2H, t), 2.65 (2H, t), 2.55 (1H, m), 2.20 (2H, bt), 2.05 (1H, bs) 1.94, (2H, bd), 1.51 (2H, m)

MS (ES) m/z 465.4 (M+H)⁺.

### Example 128

### 7-{[(1-{2-[3-fluoro-6-(methoxy)-1,5-naphthyridin-4-yl]ethyl}-4-piperidinyl)amino]methyl}-2,3-dihydro-1,5-benzothiazepin-4(5H)-one

### (a) Methyl 4-([3-(methoxy)-3-oxopropyl]thio}-3-nitrobenzoate

To a solution of methyl 4-chloro-3-nitrobenzoate (4.53 g, 0.021 mol) and methyl 3-mercaptopropionate (2.78 g, 0.023 mol) in dimethylformamide (15 mL) was added anhydrous potassium carbonate (0.023 mol, 3.17 g). After stirring at ambient temperature for 16 hours, the reaction was quenched with ice water. The precipitated product was filtered, washed well with water and dried under vacuum to give a bright yellow solid (6.11 g, 97%).

MS (ES) m/z 300.2. (M+H)⁺.

### (b) Methyl 3-amino-4-{[3-(methoxy)-3-oxopropyl]thiobenzoate

To a solution of nitrobenzoate (a) (7.58 g, 0.025 mol) in glacial acetic acid (186 mL) was added iron powder (14.0 g, 0.250 mol). After heating at 75° C. for 6 hours, the warm mixture was filtered and the filtrate concentrated under reduced pressure. The residue was partitioned between ethyl acetate and aqueous sodium chloride, and the organic layer was dried over magnesium sulfate. Filtration and evaporation afforded the product (7.03 g, 100%) which was used without further purification.

MS (ES) m/z 270.2. (M+H)⁺.

### (c) Methyl 4-oxo-2,3,4,5-tetrahydro-1,5-benzothiazepine-7-carboxylate

A suspension of ester (b) (3.00 g, 0.011 mol) in Decalin™ (120 ml) was heated at 160° C. for 40 hours. The reaction was allowed to cool and the precipitate was collected by filtration. The solid was dissolved in 1:1 acetone: methanol mixture and treated with decolorizing carbon. The solvent was evaporated in vacuo to afford a tan solid (1.67 g, 73%)

MS (ES) m/z 238.0. (M+H)⁺.

### (d) 7-(Hydroxymethyl)-2,3-dihydro-1,5-benzothiazepin-4(5H)-one

To a solution of the ester (c) (300 mg, 1.27 mmol) in tetrahydrofuran (7 mL) was added lithium borohydride (55.2 mg, 2.52 mmol) at 0° C. The reaction was stirred at room temperature for 16 hours, then quenched with methanol. The reaction was stirred for 20 minutes, then the solvents removed under reduced pressure. The residue was partitioned between ethyl acetate and aqueous sodium chloride, and the organic layer was dried over magnesium sulfate. The solvent was evaporated in vacuo to afford a semisolid mass which was triturated with cold acetonitrile to give the product as an off-white solid (95 mg, 35%).

MS (ES) m/z 210.0. (M+H)⁺.

### (e) 4-Oxo-2,3,4,5-tetrahydrobenzo[b] [1,4]thiazepine-7-carboxaldehyde

To a solution of alcohol (d) (92 mg, 0.44 mmol) in 1:6 dichloromethane: ethyl acetate (35 mL) was added Dess-Martin periodinane (242 mg, 0.57 mmol). The reaction was stirred at room temperature for 1.5 hours, then quenched with a cold aqueous 1N solution of sodium hydroxide. The layers were separated and the organic layer was washed with a 0.5 N solution of sodium hydroxide, brine and dried over sodium sulfate. The solvent was evaporated in vacuo to afford the product as an off-white solid (72 mg, 79%).

MS (ES) m/z 208.0 (M+H)⁺.

### (f) Title Compound

Amine (53i) and aldehyde (e) were treated as in Example (128) to afford the product as an amorphous light yellow solid in a 20% yield

1H NMR δ (CDCl₃) ¹H NMR δ (CDCl₃) 8.61 (1H, s), 8.17 (1H, d), 7.52 (1H, d), 7.43, (1H, bs), 7.13, (1H, d), 7.08 (1H, s), 7.07 (1H, d), 4.08 (3H, s), 3.82 (2H, s) 3.42 (4H, apparent q), 3.06 (2H, bd), 2.7 (2H, m), 2.62 (2H, t), 2.52 (1H, m), 2.18 (2H, bt), 1.93, (2H, bd), 1.50 (1H, bs), 1.45 (2H, m).

MS (ES) m/z 496.4 (M+H)⁺.

### Example 129

### trans-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinol dihydrochloride Enantiomer E1

### (a) 1,1-dimethylethyl ((trans-1-{2-[3-fluoro-6-(methoxy)-1,5-naphthyridin-4-yl]ethyl}-3-hydroxy-4-piperidinyl)carbamate

The vinyl naphthyridine (53h) (1.25 g, 6.1 mmole) was heated to 100° C. together with trans-1,1-dimethylethyl (3-hydroxy-4-piperidinyl)carbamate (prepared by hydrogenation of Example 17f, Isomer E1) (1.32 g, 6.1 mmole) in DMF (5 mL). After 24 hours, the mixture was concentrated in vacuo and purified on silica (CHCl₃/MeOH with 5% NH₄OH, 9:1) to give the product as an oil (1.9 g, 75%).

MS (ES) m/z 421 (M+H)⁺.

### (b) Title Compound

To a solution of carbamate (a) (1.9 g, 4.57 mmole) in dichloromethane (100 mL) was added 4M HCl in dioxane (20 mL). After stirring for 3 h, the reaction was evaporated to give a white solid which was used without further purification (98%).

MS (ES) m/z 321 (M+H)⁺.

To a solution of the above hydrochloride salt (ca 1.0 mmole) in ethanol (20 mL) and dichloromethane (20 mL) was added triethyl amine (0.56 mL, 4.0 mmole) and aldehyde (2c) (0.17 g, 1.0 mmole). After 24 hours at room temperature, sodium borohydride (42 mg, 1.1 mmole) was added and the reaction mixture stirred for 5 hours. Silica gel (−2 g) was added to the mixture and the reaction contents stirred for an additional 2 hours. The reaction slurry was concentrated to dryness in vacuo and loaded onto a silica gel column (eluting with CHCl₃/MeOH containing 5% NH₄OH, 9:1) to afford the free base of the title compound as a white foam.

This material, as a solution in chloroform/methanol, was treated with an excess of 2M HCl in ether and evaporated to dryness. The solid was triturated with ether, filtered and dried under vacuum to provide the title compound (71%) as a white solid.

¹H NMR of the dihydrochloride salt δH (CD₃OD) 8.82 (1H, s), 8.48(1H, s), 8.31 (1H, d), 7.59 (1H, s), 7.29 (1H, d), 4.65 (4H, m), 4.51 (2H, m), 4.40 (1H, m), 4.21 (3H, s), 3.97 (1H, m), 3.89 (1H, m), 3.80 (2H, m), 3.63 (4H, m), 3.19 (1H, m), 2.64 (1H, s), 2.30 (1H, m).

MS (+ve ion electrospray) m/z 470 (M+H)⁺.

The following example was prepared by analogous methods to Example 129 using the aldehyde shown below:

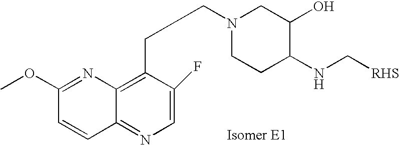

| Example | |
|---|---|
| 130 | 6-{[(-1-{2-[3-fluoro-6-(methoxy)-1,5-naphthyridin-4- |
| | yl]ethyl}-3-hydroxy-4-piperidinyl)amino]methyl}-2H- |
| | pyrido[3,2-b][1,4]oxazin-3(4H)-one dihydrochloride |
| | 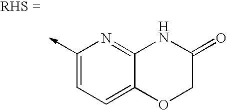 |
| | Aldehyde is 3-Oxo-3,4-dihydro-2H-pyrido[3,2-b][1,4]oxazine- |
| | 6-carboxaldehyde as in example (1l) |

### Example 131

### trans-6-{[(1-{2-[3-fluoro-6-(methoxy)-4-quinolinyl]ethyl}-3-hydroxy-4-piperidinyl)amino]methyl}-2H-pyrido[3,2-b][1,4]thiazin-3(4H)-one—Enantiomer E1

This was prepared from vinyl quinoline Example (31e) using the methodology of Examples 17 (enantiomer 1 series) affording the free base odf the title compound.

¹H NMR (400 MHz, CDCl3) δ 8.49 (s, 1H), 7.90 (d, 1H), 7.45 (d, 1H), 7.22 (dd, 1H), 7.10 (s, 1H), 6.81 (d, 1H), 3.95 (d, 1H), 3.85 (s, 3H), 3.77 (d, 1H), 3.59 (m, 1H), 3.31 (s, 2H), 3.21 (dd, 1H), 3.14 (t, 2H), 2.95 (d, 1H), 2.63 (m, 2H), 2.39 (m, 1H), 2.10 (m, 1H), 2.07 (m, 1H), 2.04 (m, 1H), 1.94 (m, 1H), 1.46 (m, 1H).

MS (ES) m/z 498 (M+H)⁺.

The title compound was then prepared by dissolving the product in chloroform and adding 2 equivalents of HCl/ether. The mixture was stirred for 15 minutes and the solvent removed under reduced pressure yielding an off white solid (0.191 g).

The following examples were prepared by analogous methods to Example 131:

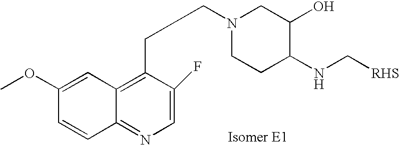

| Example | |
|---|---|
| 132 | trans-4-[(2,3-dihydro[1,4]dioxino[2,3-c]pyridin-7-ylmethyl) |
| | amino]-1-{2-[3-fluoro-6-(methoxy)-4-quinolinyl]ethyl}-3- |
| | piperidinol dihydrochloride |
| | 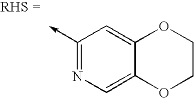 |
| | Aldehyde is 2,3-dihydro[1,4]dioxino[2,3-c]pyridine-7- |
| | carbaldehyde as in example (2c) |
| 133 | trans-6-{[(1-{2-[3-fluoro-6-(methoxy)-4-quinolinyl]ethyl}-3- |
| | hydroxy-4-piperidinyl)amino]methyl}-2H-pyrido[3,2- |
| | b][1,4]oxazin-3(4H)-one dihydrochloride |
| | 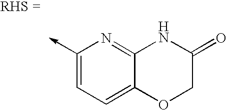 |
| | Aldehyde is 3-oxo-3,4-dihydro-2H-pyrido[3,2-b][1,4]oxazine- |
| | 6-carboxaldehyde as in example (1l) |

### Example 134

### trans-N-(1-{2-[3-fluoro-6-(methoxy)-1,5-naphthyridin-4-yl]ethyl}-3-hydroxy-4-piperidinyl)-3-oxo-3,4-dihydro-2H-pyrido[3,2-b][1,4]thiazine-6-carboxamide hydrochloride Enantiomer E1

Piperidinol hydrochloride salt Isomer E1 {prepared as in Example (129b)} and carboxylic acid (7b) were treated as in Example (118) to afford the free base of the title compound as a white solid.

This material, as a solution in chloroform/methanol, was treated with an excess of 2M HCl in ether and evaporated to dryness. The solid was triturated with ether, filtered and dried under vacuum to provide the title compound (85%) as a white solid.

¹H NMR of the dihydrochloride salt δH (CDCl₃) 8.61 (1H, s), 8.19 (1H, d), 7.79 (2H, m), 7.31 (1H, d), 7.10 (1H, d), 4.50 (1H, m), 4.15 (3H, s), 3.65-3.89 (4H, m), 3.42 (3H, m), 3.09 (2H, s), 2.92 (2H, m), 2.47 (1H, m), 2.11 (1H, m).

MS (+ve ion electrospray) m/z 513 (M+H)⁺.

The following examples were prepared by analogous method to Example 134 using the acids shown below:

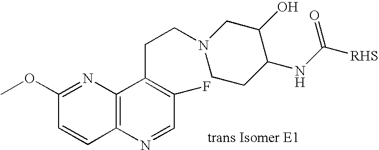

| Example | RHS |
|---|---|
| 135 | trans-N-((3R,4R)-1-{2-[3-fluoro-6-(methoxy)-1,5- |
| | naphthyridin-4-yl]ethyl}-3-hydroxy-4-piperidinyl)-3-oxo- |
| | 3,4-dihydro-2H-pyrido[3,2-b][1,4]oxazine-6-carboxamide |
| | Isomer E1 hydrochloride |
| | 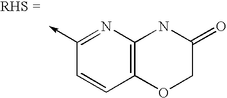 |
| | 3-oxo-3,4-dihydro-2H-pyrido[3,2-b][1,4]oxazine-6-carboxylic |
| | acid was prepared as in example (65) |
| 136 | trans-N-(1-{2-[3-fluoro-6-(methoxy)-1,5-naphthyridin-4- |
| | yl]ethyl}-3-hydroxy-4-piperidinyl)-2,3-dihydro[1,4]di- |
| | oxino[2,3-c]pyridine-7-carboxamide |
| | Isomer E1_hydrochloride |
| | 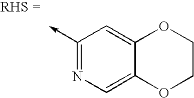 |
| | 2,3-dihydro[1,4]dioxino[2,3-c]pyridine-7-carboxylic acid is |
| | as in example (119a) |

### Example 137

### 6-{[trans-1-{2-[3-fluoro-6-(methoxy)-1,5-naphthyridin-4-yl]ethyl}-3-hydroxy-4-piperidinyl)amino]methyl}-2H-pyrido[3,2-b][1,4]thiazin-3(4H)-one Enantiomer E1

This compound was prepared by the same methodology and exhibited the same spectroscopic properties (NMR and MS) as the enantiomeric analogue (Example 113, Isomer E2).

### Example 138

### 6-{[(1-{2-[3-fluoro-6-(methoxy)-1,5-naphthyridin-4-yl]ethyl}-4-methyl-4-piperidinyl)amino]methyl}-2H-pyrido[3,2-b][1,4]oxazin-3(4H)-one dihydrochloride

### (a) 1-(1,1-dimethylethyl) 4-methyl 1,4-piperidinedicarboxylate

To a stirred solution of methyl-4-piperidinecarboxylate (10 g, 0.070 mol) in dioxane (140 mL) was added triethylamine (14.6 mL, 0.105 mol) and di-t-butyl-dicarbonate (19 g, 0.087 mol). The reaction mixture was stirred at ambient temperature for 96 hours. The solution was concentrated in vacuo. The residue was taken up in ethyl acetate (300 mL) and washed with brine solution (2×200 mL). The organic layer was obtained, dried over sodium sulfate, and concentrated to afford the title compound (17 g, 98%) as a yellow oil.

### (b) 1-(1,1-dimethylethyl) 4-methyl 4-methyl-1,4-piperidinedicarboxylate

An oven-dried flask equipped with a stirring bar and rubber septum was charged with anhydrous THF (100 mL) and placed under a stream of nitrogen. Diisopropylamine (6.34 mL, 0.0452 mol) was added and the solution cooled to −78° C. To the cooled solution was added n-butyllithium (1.6 M in hexanes, 28 mL, 0.0452 mol) over 5 minutes. The reaction mixture was stirred for 30 minutes then

### (a) (10 g, 0.0411 mol) was added and the mixture stirred for an additional hour.

After 1 hour methyl iodide (3.07 mL, 0.0493 mol) was added and stirred for 1.5 hours. The reaction mixture was quenched with brine and concentrated in vacuo. The residue was taken up in ethyl acetate (250 mL) and washed with saturated NaHCO₃ (2×150 mL) and brine (2×100 mL). The organic layer was dried over sodium sulfate and concentated in vacuo. The crude product was purified by silica gel column chromatography eluting with 20% ethyl acetate/hexanes to obtain the title compound (7.95 g, 95%) as a pale yellow oil.

LC-MS (ES) m/z 158.2 (M+H)ₜ (minus Boc).

### (c) 1-{[(1,1-dimethylethyl)oxy]carbonyl}-4-methyl-4-piperidinecarboxylic acid

To a round bottom flask was added (b) (7.6 g, 0.0295 mol) in 200 mL of methanol. To this solution was added a solution of 1N sodium hydroxide (29.5 mL, 0.0295 mol) and the mixture was heated to 45° C. for 18 hours. The reaction mixture was concentrated in vacuo. The residue was dissolved in water (100 mL) and the pH carefully adjusted to −3 by the addition of 1N HCl. The crude product was extracted into chloroform (3×300 mL), dried over sodium sulfate and concentrated in vacuo to obtain the title compound (5.86 g; 81%) as a light yellow oil which solidified upon standing.

### (d) 1,1-dimethylethyl 4-methyl-4-({[(phenylmethyl)oxy]carbonyl)amino)-1-piperidinecarboxylate

To an oven-dried round bottom flask equipped with a stirring bar and a rubber septum was added (c) (3.5 g, 0.0144 mol) in anhydrous toluene (100 mL). To this mixture was added triethylamine (4 mL, 0.0288 mol) and diphenylphosphoryl azide (6.2 mL, 0.0288 mol). The reaction was heated to 85° C. under nitrogen for 2 hours then benzyl alcohol (3 mL, 0.0288 mol) was added and the reaction mixture was stirred at 85° C. for 18 hours. The reaction mixture was concentrated in vacuo and chromatographed on silica gel chromatography eluting with 20% ethyl acetate/hexanes to provide the product as a colorless oil (3 g, 60%).

LC-MS (ES) m/z 249.4 (M+H)⁺ (minus BOC).

### (e) phenylmethyl (4-methyl-4-piperidinyl)carbamate

To a round bottom flask equipped with a stirring bar was added (d) (3 g, 0.0086 mol) in 50% trifluoroacetic acid in dichloromethane (100 mL). After 30 minutes, the reaction mixture was concentrated in vacuo and 100 mL of saturated NaHCO₃ was added and the product extracted into dichloromethane (2×100 mL). The organic layer was dried over sodium sulfate and concentrated to provide the product as a yellow oil (2 g, 95%).

LC-MS (ES) m/z 249.4 (M+H)⁺.

### (f) Phenylmethyl (1-{2-[3-fluoro-6-(methoxy)-1,5-naphthyridin-4-yl]ethyl}-4-methyl-4-piperidinyl)carbamate

A mixture of vinyl-naphthyridine (53h) (800 mg; 3.92 mmol), (e) (1.42 g; 3.92 mmol), and triethylamine (1.09 mL; 7.84 mmol) in DMF (2 mL) was heated to 100° C. for 18 hours then concentrated in vacuo. The residue was chromatographed on silica gel eluting with ethyl acetate to afford the product as a brown oil (600 mg, 34%).

MS (+ve ion electrospray) m/z 453 (M+H)⁺.

### (g) 1-{2-[3-fluoro-6-(methoxy)-1,5-naphthyridin-4-yl]ethyl}-4-methyl-4-piperidinamine

A solution of (f) (600 mg; 1.33 mmol) in ethanol (100 mL) was hydrogenated under 1 atmosphere with palladium hydroxide on charcoal (60 mg) for 18 hours. The reaction mixture was filtered through Kieselguhr and concentrated to afford the product as a yellow oil (380 mg, 90%)

MS (+ve ion electrospray) m/z 319 (M+H)⁺.

### (h) Title Compound

The amine (g) and aldehyde (1l) were reacted together in a manner similar to that of Example (129b), using sodium borohydride as reducing agent, affording the free base of the title compound in 40% yield.

¹H NMR (400 MHz, CDCl₃) δ 8.54 (s, 1H), 8.10 (d, 1H), 7.10 (d, 1H), 6.90 (d, 1H), 6.88 (d, 2H) 4.48 (s, 2H), 3.99 (s, 3H), 3.31 (s, 2H), 2.59 (m, 6H), 1.67 (m, 4H), and 1.10 (s, 3H).

MS (+ve ion electrospray) m/z 481 (M+H)⁺.

This material, as a solution in chloroform/methanol, was treated with an excess of 2M HCl in ether and evaporated to dryness. The solid was triturated with ether, filtered and dried under vacuum to provide the title compound as a white solid.

The following examples were prepared by analogous method to Example 138 using the aldehydes shown below:

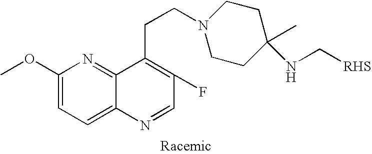

| Example | |
|---|---|
| 139 | 6-{[(1-{2-[3-fluoro-6-(methoxy)-1,5-naphthyridin-4-yl]ethyl}- |
| | 4-methyl-4-piperidinyl)amino]methyl}-2H-pyrido[3,2- |
| | b][1,4]thiazin-3(4H)-one dihydrochloride |
| | 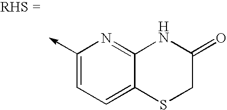 |
| | Aldehyde is 3-oxo-3,4-dihydro-2H-pyrido[3,2-b][1,4]thiazine- |
| | 6-carboxaldehyde as in example (7d) |
| 140 | N-(2,3-dihydro[1,4]dioxino[2,3-c]pyridin-7-ylmethyl)-1-{2- |
| | [3-fluoro-6-(methoxy)-1,5-naphthyridin-4-yl]ethyl}-4-methyl- |
| | 4-piperidinamine dihydrochloride |
| | 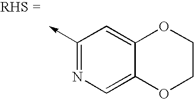 |
| | Aldehyde is 2,3-dihydro[1,4]dioxino[2,3-c]pyridine-7- |
| | carbaldehyde as in example (2c) |
| 141 | N-(1-{2-[3-fluoro-6-(methoxy)-1,5-naphthyridin-4- |
| | yl]ethyl}-4-methyl-4-piperidinyl)-2,3-dihydro-1,4- |
| | benzodioxin-6-sulfonamide |
| | 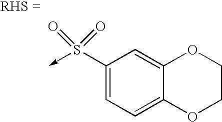 |
| | 2,3-dihydro-1,4-benzodioxin-6-sulfonyl chloride is |
| | commercially available and sulfonamide formation used |
| | triethylamine as amine base |

### Example 142

### cis-6-{[(1-{2-[3-chloro-8-fluoro-6-(methoxy)-4-quinolinyl]ethyl}-3-fluoro-4-piperidinyl)amino]methyl}-2H-pyrido[3,2-b][1,4]thiazin-3(4H)-one dihydrochloride Enantiomer 1

### (a) cis-4-Benzylamino-1-tert-butoxycarbonyl-3-fluoropiperidine

4-Benzyl-1-tert-butoxycarbonyl-3-fluoropiperidine was prepared according to the procedures of J. Med. Chem. 1999, 42, 2087-2104 as a mixture of isomers (approx 8:1 cis:trans, 29.8 g, 0.096 mole). The mixture was dissolved in DCM, extracted with 0.2M HCl, basified with Na₂CO₃ solution, extracted with DCM and chromatographed on silica gel to give the cis-isomer in the later fractions (15.6 g, 52%). Combined batches (32 g, 0.103 mole) were separated by preparative HPLC on a Chiralpak AD column eluting with hexane:ethanol (9:1) to give faster running enantiomer [Enantiomer 1] (15.09, 47%, 99% ee) [□]_{D} +40.5° and slower running enantiomer [Enantiomer 2] (15.0 g, 47%, 97% ee) [□]_{D} −39.5°.

### (b) cis-1,1-dimethylethyl 4-amino-3-fluoro-1-piperidinecarboxylate, Enantiomer 1

To a solution of cis-4-benzylamino-1-tert-butoxycarbonyl-3-fluoropiperidine (a, Enantiomer 1) (29 g, 94 mmole) in EtOH (300 mL) was added palladium hydroxide (8g). The reaction was hydrogenated for 6 hours, then was filtered through Kieselguhr. The filtrate was concentrated under reduced pressure to afford the title compound as a white solid (20.5 g, 100%).

MS (ES) m/z 219 (M+H)⁺.

### (c) cis-1,1-dimethylethyl 3-fluoro-4-({[(phenylmethyl)oxy]carbonyl}amino)-1-piperidinecarboxylate, Enantiomer 1

To a solution of amine (b, Enantiomer1) (23 g, 105 mmol) in ethyl acetate (200 mL) was added a saturated solution of sodium bicarbonate (200 mL) followed by benzyl chloroformate (16 mL, 116 mmol). The reaction mixture was stirred for 4.5 hours. The layers were separated and the aqueous extraacted with ethyl acetate. The combined organic extracts were dried over magnesium sulfate and evaporated under vacuum to afford the product as an oil (37.4 g, 100%).

MS (ES) m/z 353 (M+H)⁺.

### (d) cis-phenylmethyl (3-fluoro-4-piperidinyl)carbamate

The carbamate (c, Enantiomer 1) (37 g, 105 mmol) in dichloromethane (150 mL) was treated with trifluoroacetic acid (60 mL) at room temperature for 4 hours. The residue was basified with sodium carbonate and extracted with 10% methanol-dichloromethane. The combined organic extracts were dried over magnesium sulfate and evaporated under vacuum to afford the product as a white solid (26.8 g, 100%).

MS (ES) m/z 253 (M+H)⁺.

### (e) cis-phenylmethyl (1-{2-[3-chloro-8-fluoro-6-(methoxy)-4-quinolinyl]ethyl}-3-fluoro-4-piperidinyl)carbamate, Enantiomer 1

Vinyl-quinoline (97d) and fluoropiperidine (d, Enantiomer 1) were treated as in example (52h) to afford the product as an oil in 25% yield.

MS (ES) m/z 490 (M+H)⁺.

### (f) cis-1-{2-[3-chloro-8-fluoro-6-(methoxy)-4-quinolinyl]ethyl}-3-fluoro-4-piperidinamine, Enantiomer 1

The carbamate (d, Enantiomer 1) (0.103 g, 0.2 mmol) in ethanol was hydrogenated with 10% palladium on charcoal for 18 hours. The mixture was filtered through Kieselguhr and evaporated under vaccum to afford the product as an oil (26 mg, 35%).

MS (ES) m/356 (M+H)⁺.

### (g) Title Compound

Amine (f, Enantiomer 1) and aldehyde (7d) were treated as in Example (53j) to afford the free base of the title compound as an oil in a 46% yield.

1H NMR δH (CDCl₃) 8.67 (1H, s) 8.34 (1H, bs), 7.59 (1H, d), 7.08 (3H, m), 4.86 (1H, d), 3.94 (3H, s) 3.91 (2H, s), 3.47 (2H, s), 3.37 (3H, m), 3.07 (1H, d), 2.68 (3H, m), 2.43 (1H, dd), 2.29 (1H, m), 1.87 (3H, m).

MS (ES) m/z 534 (M+H)⁺.

This material, as a solution in chloroform/methanol, was treated with an excess of 2M HCl in ether and evaporated to dryness. The solid was triturated with ether, filtered and dried under vacuum to provide the title compound as a white solid.

### Example 143

### cis-1-{2-[3,8-difluoro-(methoxy)-4-quinolinyl]ethyl}-N-(2,3-dihydro[1,4]dioxino[2,3-c]pyridin-7-ylmethyl)-3-fluoro-4-piperidinamine dihydrochloride Enantiomer1

The free base of this compound was prepared by methods analogous to those of Example (142) with the exceptions that the vinyl quinoline used was Example (47j) and the aldehyde used in the last stage was Example (2c).

1H NMR δH (CDCl₃) 8.61 (1H, s) 8.11 (1H, s), 7.04 (2H, m), 6.76 (1H, s), 4.85 (1H, d), 4.31 (4H, m), 3.95 (3H, s) 3.87 (2H, s), 3.33 (1H, m), 3.23 (2H, t), 3.04 (1H, d), 2.68 (3H, m), 2.43 (1H, dd), 2.23 (1H, m), 1.86 (2H, m).

MS (ES) m/z 489 (M+H)⁺.

This material, as a solution in chloroform/methanol, was treated with an excess of 2M HCl in ether and evaporated to dryness. The solid was triturated with ether, filtered and dried under vacuum to provide the title compound as a white solid.

### Example 144

### cis-1-{2-[3,8-difluoro-6-(methoxy)-4-quinolinyl]ethyl}-N-(2,3-dihydro[1,4]dioxino[2,3-c]pyridin-7-ylmethyl)-3-fluoro-4-piperidinamine dihydrochloride Enantiomer 2

This was prepared in an analogous way to Example 143, with the exception that cis-4-benzylamino-1-tertbutoxycarbonyl-3-fluoropiperidine, Enantiomer 2 (Example 143a) was used as the starting material. Spectroscopic properties (NMR and MS) and salt formation was the same.

The following examples were prepared by analogous methods to Example 143 using the aldehydes shown below:

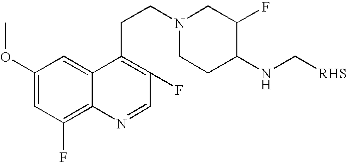

| Example | |
|---|---|
| 145 | cis-6-{[(1-{2-[3,8-difluoro-6-(methoxy)-4-quinolinyl]ethyl}-3- |
| | fluoro-4-piperidinyl)amino]methyl}-2H-pyrido[3,2- |
| | b][1,4]oxazin-3(4H)-one dihydrochloride, Enantiomer 1, |
| 146 | cis-6-{[(1-{2-[3,8-difluoro-6-(methoxy)-4- |
| | quinolinyl]ethyl}-3-fluoro-4- |
| | piperidinyl)amino]methyl}-2H-pyrido[3,2- |
| | b][1,4]oxazin-3(4H)-one dihydrochloride, |
| | Enantiomer 2, |
| | 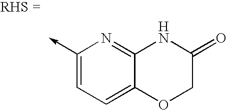 |
| | Aldehyde is 3-Oxo-3,4-dihydro-2H-pyrido[3,2-b][1,4]oxazine- |
| | 6-carboxaldehyde as in example (1l) |
| 147 | cis-1-{2-[3,8-difluoro-6-(methoxy)-4-quinolinyl]ethyl}- |
| | N-(2,3-dihydro-1,4-benzodioxin-6-ylmethyl)-3-fluoro-4- |
| | piperidinamine dihydrochloride, Enantiomer 1 |
| 148 | cis-1-{2-[3,8-difluoro-6-(methoxy)-4- |
| | quinolinyl]ethyl}-N-(2,3-dihydro-1,4-benzodioxin-6- |
| | ylmethyl)-3-fluoro-4-piperidinamine dihydrochloride, |
| | Enantiomer 2 |
| | 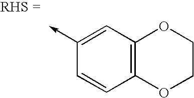 |
| | Aldehyde is 2,3-dihydro-1,4-benzodioxin-6-carbaldehyde- |
| | commercially available |
| 149 | cis-6-{[(-1-{2-[3,8-difluoro-6-(methoxy)-4-quinolinyl]ethyl}- |
| | 3-fluoro-4-piperidinyl)amino]methyl}-2H-pyrido[3,2- |
| | b][1,4]thiazin-3(4H)-one dihydrochloride, Enantiomer 1 |
| 150 | cis-6-{[(-1-{2-[3,8-difluoro-6-(methoxy)-4- |
| | quinolinyl]ethyl}-3-fluoro-4- |
| | piperidinyl)amino]methyl}-2H-pyrido[3,2- |
| | b][1,4]thiazin-3(4H)-one dihydrochloride, |
| | Enantiomer 2 |
| | 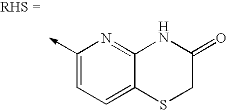 |
| | Aldehyde is 3-Oxo-3,4-dihydro-2H-pyrido[3,2- |
| | b][1,4]thiazine-6-carboxaldehyde as in example (7d) |

Aldehyde is 3-Oxo-3,4-dihydro-2H-pyrido[3,2-b][1,4]thiazine-6-carboxaldehyde as in example (7d)

### Example 151

### cis-N-(1-{2-[3,8-difluoro-6-(methoxy)-4-quinolinyl]ethyl}-3-fluoro-4-piperidinyl)-3-oxo-3,4-dihydro-2H-pyrido[3,2-b][1,4]thiazine-6-carboxamide hydrochloride Enantiomer 1

Amine (142f) and carboxylic acid (7b) were treated as in Example (118) to afford the free base of the title compound as an oil in almost 100% yield.

1H NMR δH (CDCl₃) 8.63 (1H, s), 8.29 (1H, s), 7.91 (1H, d), 7.85 (1H, d), 7.79 (1H, d), 7.07 (1H, dd), 7.03 (1H, d), 4.80 (1H, d), 4.20 (1H, m), 3.96 (3H, s), 3.48 (2H, s), 3.54 (2H, m), 3.40 (1H, m) 3.25 (2H, t), 3.14 (1H, d), 2.75 (2H, m), 2.49 (1H, dd), 2.38 (1H, t), 1.97 (1H, m), 1.92 (1H, m).

MS (ES) m/z 532 (M+H)⁺.

This material, as a solution in chloroform/methanol, was treated with an excess of 2M HCl in ether and evaporated to dryness. The solid was triturated with ether, filtered and dried under vacuum to provide the dihydrochloride salt of the title compound as a white solid.

### Example 152

### 6-{[((3S,4R)-1-{2-[3-chloro-8-fluoro-6-(methoxy)-4-quinolinyl]ethyl}-3-hydroxy-4-piperidinyl)amino]methyl}-2H-pyrido[3,2-b][1,4]thiazin-3(4H)-one dihydrochloride Enantiomer E2

### (a) 1,1-dimethylethyl ((3S,4R)-1-{2-[3-chloro-8-fluoro-6-(methoxy)-4-quinolinyl]ethyl}-3-hydroxy-4-piperidinyl)carbamate, Isomer E2

Vinyl-quinoline (97d) and 1,1-dimethylethyl [(3S,4R)-3-hydroxy-4-piperidinyl]carbamate (5c, Enantiomer 2) were treated as in Example (47k) to afford the product as an oil in 33% yield.

MS (ES) m/z 454/456 (M+H)⁺.

### (b) (3S,4R)-4-amino-1-{2-[3-chloro-8-fluoro-6-(methoxy)-4-quinolinyl]ethyl}-3-piperidinol, Enantiomer 1

Carbamate (a) was treated as in example (471) to afford the product as a solid in a 98% yield.

MS (ES) m/z 354/356 (M+H)⁺.

### (c) Title Compound

Amine (b) and aldehyde (2c) were treated as in example (52j) to afford the free base of the title compound as an oil in a 31% yield.

1H NMR δH (CDCl₃) 8.67 (1H, s), 8.10 (1H, s), 7.09 (1H, dd), 7.05 (1H, d), 6.81 (1H, s), 4.30 (4H, m), 3.94 (3H, s), 3.89 (1H, s), 3.83 (2H, s), 3.37 (2H, t), 3.14 (1H, d), 2.97 (1H, d), 2.65 (2H, m), 2.36 (1H, d), 2.25 (1H, m), 1.97 (1H, m), 1.75 (2H, m).

MS (ES) m/z 503/505 (M+H)⁺.

This material, as a solution in chloroform/methanol, was treated with an excess of 2M HCl in ether and evaporated to dryness. The solid was triturated with ether, filtered and dried under vacuum to provide the title compound as a white solid.

### Example 153

### trans-6-({1-[2-(3-Chloro-6-methoxy-[1,5]naphthyridin-4-yl)-ethyl]-3-hydroxy-piperidin-4-ylamino}-methyl)-4H-pyrido[3,2-b][1,4] oxazin-3-one trihydrochloride Enantiomer 1

A solution of amine (41a) and aldehyde (1l) were treated as in Example (40) to afford the title compound as a white solid.

¹H NMR (400 MHz, DMSO-d₆) δ 9.81 (s, 1H), 9.31 (s, 1H), 8.84 (s, 1H), 8.33 (d, 1H,), 7.46 (d, 1H), 7.34 (d, 1H), 7.23 (d, 1H), 4.70 (s, 2H), 4.38 (m, 7H), 4.12 (s, 3H), 3.81 (m, 3H), 3.56 (m, 1H), 3.43 (m, 3H), 3.18 (m, 1H), 2.99 (m, 1H), 2.56 (m, 1H), 2.18 (m, 1H).

LC-MS (ES) m/z 499.4 (M+H)⁺.

### Example 154

### trans-1-{2-[3-chloro-6-(methoxy)-1,5-naphthyridin-4-yl]ethyl}-4-[(2,3-dihydro[1,4]dioxino[2,3-c]pyridin-7-ylmethyl)amino]-3-piperidinol Enantiomer 1

A solution of amine (41a) and aldehyde (2c) were treated as in Example (40) to afford the product as a white solid.

MS (ES) m/z 486 (M+H)⁺.

### Example 155

### trans-1-{2-[3-chloro-6-(methoxy)-1,5-naphthyridin-4-yl]ethyl}-4-[(2,3-dihydro[1,4]dioxino[2,3-c]pyridin-7-ylmethyl)amino]-3-piperidinol Enantiomer 2

A solution of amine (see Example 46) and aldehyde (2c) were treated as in Example (40) to afford the product as a white solid.

MS (ES) m/z 486 (M+H)⁺.

### Example 156

### 2-{4-[(2,3-dihydro[1,4]dioxino[2,3-c]pyridin-7-ylmethyl)amino]-1-piperidinyl}-1-[3-fluoro-6-(methoxy)-4-quinolinyl]ethanol dihydrochloride Enantiomer 1

This is the alternative enantiomer to Example (112, Enantiomer 2) and was isolated by chiral preparative hplc as described in Example (99). The free base of the title compound was isolated as a white foam, as the major, first eluting enantiomer.

1H NMR δH (400 mHz, CDCl₃) 8.56 (1H, s), 8.10 (1H, s), 7.95 (1H, d), 7.92 (1H, d), 7.29 (1H, dd), 6.83 (1H, s), 5.58 (1H, dd), 4.25-4.35 (4H, m), 3.93 (3H, s), 3.81 (2H, s), 3.18 (1H, m), 3.03 (1H, m), 2.90 (1H, m), 2.60 (2H, m), 2.49 (1H, br.t), 2.18 (1H, br.t), 1.90 (2H, m), 1.80 (2H, m), 1.40-1.65 (2H, m)

MS (ES) m/z 469 (M+H)⁺.

This material, as a solution in chloroform/methanol, was treated with an excess of 1M HCl in ether and evaporated to dryness. The solid was triturated under ether, filtered and dried under vacuum to provide the title compound as a white solid (70 mg).

### Example 157

### N-(2,3-dihydro-1,4-benzodioxin-6-ylmethyl)-1-{2-[3-fluoro-6-(methoxy)-1,5-naphthyridin-4-yl]ethyl}-4-piperidinamine

Amine (53i) and 2,3-dihydro-1,4-benzodioxin-6-carbaldehyde as in Example (148) were treated as in Example (53j) to afford the free base of the compound.

¹H NMR (400 MHz, d₄-MeOH) 8.59 (s, 1H), 8.14 (d, 1H), 7.10 (d, 1H), 6.90 (s, 1H), 6.79-6.85 (m, 2H), 4.07 (s, 3H), 4.22 (s, 4H), 3.84 (s, 2H), 3.32-3.29 (m, 2H), 3.13-3.16 (m, 2H), 2.72-2.81 (m, 3H), 2.18-2.21 (m, 2H), 2.12-2.05 (m, 2H), 1.51-1.60 (m, 2H).

MS (ES) m/z 453 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 158

### (3S,4R)-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinol dihydrochloride Enantiomer 2

Amine (70a) and aldehyde (2c) were treated as in Example (53j) to afford the free base of the compound.

1H NMR δH (CDCl₃) 8.61 (1H, s), 8.17 (1H, d), 8.10 (1H, s), 7.07 (1H, d), 6.84 (1H, s), 4.20-4.35 (4H, m), 4.08 (3H, s), 3.87 (1H, s), 3.83 (2H, s), 3.39 (2H, bt), 3.10 (1H, bd), 2.95 (1H, bd), 2.78 (2H, bt), 2.50-2.60 (1H, m), 2.34 (1H, d), 2.22 (1H, bt), 1.6-1.9 (m, including water)

MS (ES) m/z 470 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 159

### (3R,4S) 1-{2-[3-fluoro-6-(methoxy)-1,5-naphthyridin-4-yl]ethyl}-4-[([1,3]oxathiolo[5,4-c]pyridin-6-ylmethyl)amino]-3-piperidinol dihydrochloride Enantiomer E1

Amine (66b) and aldehyde (61) were treated as in Example (53j) to afford the free base of the compound.

¹H NMR (400 MHz, CDCl3) 8.61 (s, 1H), 8.18-8.16 (d, 1H), 8.00 (s, 1H), 7.26-7.23 (d, 1H), 7.08-7.06 (d, 1H), 5.74-5.73 (s, 2H), 4.08-3.88 (s, 3H), 3.85 (s, 2H), 3.40-3.36 (m, 2H), 2.92-2.80 (m, 3H), 2.77-2.75 (m, 2H), 2.53-2.51 (m, 1H), 2.34-2.20 (m, 2H), 1.72-1.60 (m, 4H).

MS (ES) m/z 472 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 160

### 6-{[(1-{2-[3-chloro-8-fluoro-6-(methoxy)-4-quinolinyl]ethyl}-4-piperidinyl)amino]methyl}-2H-pyrido[3,2-b][1,4]thiazin-3(4H)-one

### (a) 1,1-dimethylethyl (1-{2-[3-chloro-8-fluoro-6-(methoxy)-4-quinolinyl]ethyl}-4-piperidinyl)carbamate

Vinyl-quinoline (98d) and piperidin-4-yl-carbamic acid tert-butyl ester were treated as in Example (52h) to afford the product in 73% yield.

MS (ES) m/z 438/440 (M+H)⁺.

### (b) 1-{2-[3-chloro-8-fluoro-6-(methoxy)-4-quinolinyl]ethyl}-4-piperidinamine

Carbamate (a) was treated as in Example (66b) to afford the amine in a quantitative yield.

MS (ES) m/z 338/340 (M+H)⁺.

### (c) Title Compound

Amine (b) and aldehyde (7d) were treated as in Example (53j) to afford the free base of the compound.

1H NMR δH (CDCl₃) 8.67 (1H, s), 8.06 (1H, bs), 7.57 (1H, d), 7.09 (2H, dd), 6.99 (1H, d), 3.95 (3H, s), 3.85 (2H, s), 3.48 (2H, s), 3.39 (2H, m) 3.06 (2H, m), 2.70-2.52 (3H, m), 2.21 (2H, m), 1.96 (2H, d), 1.55 (2H, m).

MS (ES) m/z 517 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 161

### 1-{2-[3-chloro-8-fluoro-6-(methoxy)-4-quinolinyl]ethyl}-N-(2,3-dihydro[1,4]dioxino[2,3-c]pyridin-7-ylmethyl)-4-piperidinamine

Amine (160b) and aldehyde (2c) were treated as in Example (53j) to afford the free base of the compound.

1H NMR δH (CDCl₃) 8.66 (1H, s) 8.11 (1H, s), 7.08 (2H, m), 6.83 (1H, s), 4.33 (2H, m), 4.27(2H, m), 3.94 (3H, s), 3.81 (2H, s), 3.37 (2H, m), 3.05 (2H, m), 2.68-2.51 (3H, m), 2.23 (2H, t), 2.20 (2H, d), 1.55 (2H, m).

MS (ES) m/z 487 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 162

### (3S,4R)-1-[2-(3,6-dichloro-4-quinolinyl)ethyl]-4-[(2,3-dihydro[1,4]dioxino[2,3-c]pyridin-7-ylmethyl)amino]-3-piperidinol dihydrochloride Enantiomer E2

### (a) 1,1-dimethylethyl ((3S,4R)-1-[2-(3,6-dichloro-4-quinolinyl)ethyl]-3-hydroxy-4-piperidinyl)carbamate

Vinyl-quinoline (27e) and piperidine (5c, enantiomer E2) were treated as in Example (23g) to afford the product as an oil.

MS (ES) m/z 440 (M+H)⁺.

### (b) (3S,4R)-4-amino-1-[2-(3,6-dichloro-4-quinolinyl)ethyl]-3-piperidinol

Carbamate (a) was treated as in Example (23h) to afford the product as an oil. MS (ES) m/z 340 (M+H)⁺.

### (c) Title Compound

Amine (b) and aldehyde (2c) were treated as in Example (23i) to afford the product as an oil. MS (ES) m/z 490 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 163

### 6-[({(3S,4R)-1-[2-(3,6-dichloro-4-quinolinyl)ethyl]-3-hydroxy-4-piperidinyl}amino)methyl]-2H-pyrido[3,2-b][1,4]thiazin-3(4H)-one dihydrochloride Enantiomer E2

Amine (162b) and aldehyde (7d) were treated as in Example (23i) to afford the product as an oil.

MS (ES) m/z 518 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 164

### (3S,4R)-1-[2-(3-chloro-6-fluoro-4-quinolinyl)ethyl]-4-[(2,3-dihydro[1,4]dioxino[2,3-c]pyridin-7-ylmethyl)amino]-3-piperidinol dihydrochloride Enantiomer E2

### (a) 1,1-dimethylethyl {(3S,4R)-1-[2-(3-chloro-6-fluoro-4-quinolinyl)ethyl]-3-hydroxy-4-piperidinyl}carbamate

Vinyl-quinoline (25e) and piperidine (5c, enantiomer E2) were treated as in Example (23g) to afford the product as an oil.

MS (ES) m/z 424 (M+H)⁺.

### (b) (3S,4R)-4-amino-1-[2-(3-chloro-6-fluoro-4-quinolinyl)ethyl]-3-piperidinol

Carbamate (a) was treated as in Example (23h) to afford the product as an oil. MS (ES) m/z 324 (M+H)⁺.

### (c) Title Compound

Amine (b) and aldehyde (2c) were treated as in Example (23i) to afford the product as an oil.

MS (ES) m/z 473 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 165

### 6-[({(3S,4R)-1-[2-(3-chloro-6-fluoro-4-quinolinyl)ethyl]-3-hydroxy-4-piperidinyl}amino)methyl]-2H-pyrido[3,2-b][1,4]thiazin-3(4H)-one dihydrochloride Enantiomer E2

Amine (164b) and aldehyde (7d) were treated as in Example (23i) to afford the product as an oil.

MS (ES) m/z 502 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 166

### N-(1-{2-[3-fluoro-6-(methoxy)-1,5-naphthyridin-4-yl]ethyl}-4-methyl-4-piperidinyl)-2,3-dihydro[1,4]dioxino[2,3-c]pyridine-7-carboxamide dihydrochloride

Amine (138g) and carboxylic acid (119a) were treated as in Example (118) to afford the title compound.

MS (ES) m/z 482 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

The following Examples were prepared by analogous method to Example 134 using the acids shown below:

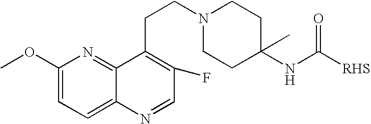

| Example | |
|---|---|
| 167 | N-(1-{2-[3-fluoro-6-(methoxy)-1,5-naphthyridin-4-yl]ethyl}- |
| | 4-methyl-4-piperidinyl)-3-oxo-3,4-dihydro-2H-pyrido[3,2- |
| | b][1,4]oxazine-6-carboxamide |
| | 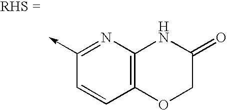 |
| | 3-oxo-3,4-dihydro-2H-pyrido[3,2-b][1,4]oxazine-6-carboxylic |
| | acid was prepared as in Example (65) |
| 168 | N-(1-{2-[3-fluoro-6-(methoxy)-1,5-naphthyridin-4-yl]ethyl}- |
| | 4-methyl-4-piperidinyl)-3-oxo-3,4-dihydro-2H-pyrido[3,2- |
| | b][1,4]thiazine-6-carboxamide |
| | 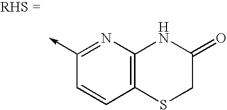 |
| | 3-Oxo-3,4-dihydro-2H-pyrido[3,2-b][1,4]thiazine-6-carboxylic |
| | acid was prepared as in Example (7b) |

### Example 169

### trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]oxazin-3(4H)-one dihydrochloride Enantiomer E1

Amine (120e, enantiomer E1) and aldehyde (1l) were treated as in Example (120h) to afford the title compound.

MS (ES) m/z 497 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 170

### trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]thiazin-3(4H)-one dihydrochloride Enantiomer E1

Amine (120e, enantiomer E1) and aldehyde (7d) were treated as in Example (120h) to afford the title compound.

MS (ES) m/z 513 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 171

### trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]oxazin-3(4H)-one dihydrochloride Enantiomer E2

Amine (120e, enantiomer E2) and aldehyde (1l) were treated as in Example (120h) to afford the title compound.

MS (ES) m/z 497 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 172

### trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]thiazin-3(4H)-one dihydrochloride Enantiomer E2

Amine (120e, enantiomer E2) and aldehyde (7d) were treated as in Example (120h) to afford the title compound.

MS (ES) m/z 513 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 173

### trans-4[(2,3-dihydro-1,4-benzodioxin-6-ylmethyl)amino]-1-{2-[3-fluoro-6-(methoxy)-4-quinolinyl]ethyl}-3-piperidinol hydrochloride Enantiomer E1

This was prepared by hydrogenation of piperidine (17f, enantiomer E1) over Pearlman's catalyst by the method of Example (5c), followed by reaction with the vinyl-quinoline (31e), removal of BOC protecting group and reaction with aldehyde (148) by the methods of Examples (5d-f) to afford the free base of the title compound.

MS (ES) m/z 468 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 174

### trans-4-[(2,3-dihydro-1,4-benzodioxin-6-ylmethyl)amino]-1-{2-[3-fluoro-6-(methoxy)-1,5-naphthyridin-4-yl]ethyl}-3-piperidinol dihydrochloride Enantiomer E2

This was prepared by the method of Example (113) using aldehyde (148) instead of aldehyde (7d) to afford the free base of the title compound.

MS (ES) m/z 469 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 175

### trans 4-[(2,3-dihydro-1,4-benzodioxin-6-ylmethyl)amino]-1-{2-[3-fluoro-6-(methoxy)-1,5-naphthyridin-4-yl]ethyl}-3-piperidinol dihydrochloride Enantiomer E1

This was prepared by the method of Example (129) using aldehyde (148) instead of aldehyde (2c) to afford the free base of the title compound.

MS (ES) m/z 469 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 176

### (3S,4R)-1-{2-[3,8-difluoro-6-(methoxy)-4-quinolinyl]ethyl}-4-[(2,3-dihydro[1,4]dioxino[2,3-c]pyridin-7-ylmethyl)amino]-3-piperidinol dihydrochloride Enantiomer E2

This was prepared by the methods of Example 74 using piperidine (5c, enantiomer E2) instead of piperidine (5c, enantiomer E1) to afford the free base of the title compound.

MS (ES) m/z 487 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 177

### (3S,4R)-1-{2-[3,8-difluoro-6-(methoxy)-4-quinolinyl]ethyl}-4-[(2,3-dihydro-1,4-benzodioxin-6-ylmethyl)amino]-3-piperidinol dihydrochloride Enantiomer E2

This was prepared by the method of Example 176 using aldehyde as in Example (148) instead of aldehyde (2c) to afford the free base of the title compound.

MS (ES) m/z 486 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 178

### N-(2,3-dihydro-1-benzofuran-5-ylmethyl)-1-{2-[3-fluoro-6-(methoxy)-1,5-naphthyridin-4-yl]ethyl}-4-piperidinamine dihydrochloride

This was prepared following the method of Example (53j) using 2,3-dihydro-1-benzofuran-5-carbaldehyde (commercially available) instead of aldehyde (2c) to afford the free base of the title compound.

MS (ES) m/z 437 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 179

### 6-{[(1-{2-[3-fluoro-6-(methoxy)-4-quinolinyl]-2-hydroxyethyl}-4-piperidinyl)amino]methyl}-2H-pyrido[3,2-b][1,4]oxazin-3(4H)-one dihydrochloride Enantiomer E1

This compound was prepared as described in Example 112 but using AD-mix β in the dihydroxylation step (99a) and aldehyde (1l) instead of aldehyde (2c). The compound was eluted from the HPLC Chiralpak AD column as the major, faster eluting, isomer.

[α]_{D} (25° C.)=+70.8 degrees (c=1%, methanol).

It was converted to the title compound by the method of Example (99).

### Example 180

### 6-{[(1-{2-[3-fluoro-6-(methoxy)-4-quinolinyl]-2-hydroxyethyl}-4-piperidinyl)amino]methyl}-2H-pyrido[3,2-b][1,4]oxazin-3(4H)-one dihydrochloride Enantiomer E2

This compound was prepared as described in Example 112 but using AD-mix β in the dihydroxylation step (99a) and aldehyde (1l) instead of aldehyde (2c) in step (99f). The compound was eluted from the HPLC Chiralpak AD column as the minor, slower eluting, isomer.

[α]_{D} (25° C.)=−71.4 degrees (c=1%, methanol).

It was converted to the title compound by the method of Example (99).

### Example 181

### 6-{[(1-{2-[3-fluoro-6-(methoxy)-1,5-naphthyridin-4-yl]-2-hydroxyethyl}-4-piperidinyl)amino]methyl}-2H-pyrido[3,2-b][1,4]oxazin-3(4H)-one dihydrochloride Enantiomer E2

This compound was prepared as described in Example (99) but using aldehyde (1l) instead of aldehyde (2c) in step (99f). The compound was eluted from the HPLC Chiralpak AD column as the minor, slower eluting, isomer.

[α]_{D} (25° C.)=+8.7 degrees (c=1%, methanol).

It was converted to the hydrochloride by the method of Example (99).

### Example 182

### 6-{[(1-{2-[3-fluoro-6-(methoxy)-1,5-naphthyridin-4-yl]-2-hydroxyethyl}-4-piperidinyl)amino]methyl}-2H-pyrido[3,2-b][1,4]oxazin-3(4H)-one dihydrochloride Enantiomer E1

This compound was prepared as described in Example 99 but using aldehyde (1l) instead of aldehyde (2c) in step (99f). The compound was eluted from the HPLC Chiralpak AD column as the major, faster elunting, isomer.

[α]_{D} (25° C.)=−8.3 degrees (c=1%, methanol).

It was converted to the title compound by the method of Example (99).

### Example 183

### 6-{[(1-{2-[3-chloro-8-fluoro-6-(methoxy)-4-quinolinyl]-2-hydroxyethyl}-4-piperidinyl)amino]methyl}-2H-pyrido[3,2-b][1,4]oxazin-3(4H)-one dihydrochloride Enantiomer E1

Vinyl-quinoline (97d) was taken through the sequence outlined in Example (99) using AD-mixβ:α (2:1) as a chiral agent for the dihydroxylation step and aldehyde (1l) instead of aldehyde (2c) in step (99f).

The compound was eluted from the HPLC Chiralpak AD column as the major, faster eluting, isomer.

[α]_{D} (25° C.)=+65.2 degrees (c=1%, methanol).

It was converted to the title compound by the method of Example (99).

### Example 184

### 6-{[(1-{2-[3-chloro-8-fluoro-6-(methoxy)-4-quinolinyl]-2-hydroxyethyl}-4-piperidinyl)amino]methyl}-2H-pyrido[3,2-b][1,4]oxazin-3(4H)-one dihydrochloride Enantiomer E2

Vinyl-quinoline (97d) was taken through the sequence outlined in Example (99) using AD-mixβ:α (2:1) as a chiral agent for the dihydroxylation step and aldehyde (1l) instead of aldehyde (2c) in step (100f).

The compound was eluted from the HPLC Chiralpak AD column as the minor, slower eluting, isomer.

[α]_{D} (25° C.)=−66.3 degrees (c=1%, methanol).

It was converted to the title compound by the method of Example (99).

### Example 185

### 6-[3-chloro-8-fluoro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E1

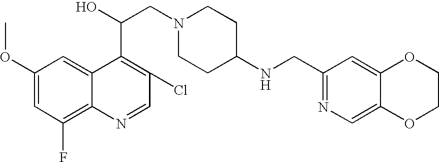

Vinyl-quinoline (98d) was taken through the sequence outlined in Example (99) using AD-mixβ:α (2:1) as a chiral agent for the dihydroxylation step.

The compound was eluted from the HPLC Chiralpak AD column as the major, faster eluting, isomer.

[α]_{D} (25° C.)=+16.4 degrees (c=1%, methanol).

It was converted to the title compound by the method of Example (99).

### Example 186

### 6-[3-chloro-8-fluoro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E2

Vinyl-quinoline (98d) was taken through the sequence outlined in Example (99) using AD-mixβ:α (2:1) as a chiral agent for the dihydroxylation step.

The compound was eluted from the HPLC Chiralpak AD column as the minor, slower eluting, isomer.

[α]_{D} (25° C.)=−16.0 degrees (c=1%, methanol).

It was converted to the title compound by the method of Example (99).

### Example 187

### 1-[3,8-difluoro-6-(methoxy)-4-quinolinyl]-2-(4[(2,3-dihydro[1,4]dioxino[2,3-c]pyridin-7-ylmethyl)amino]-1-piperidinyl}ethanol dihydrochloride Enantiomer E2

Vinyl-quinoline (47j) was taken through the sequence outlined in Example (99) using AD-mixα as a chiral agent for the dihydroxylation step.

The compound was eluted from the HPLC Chiralpak AD column as the major, slower eluting, isomer.

It was converted to the title compound by the method of Example (99).

### Example 188

### 1-[3,8-difluoro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E1

Vinyl-quinoline (47j) was taken through the sequence outlined in Example (99) using AD-mixα as a chiral agent for the dihydroxylation step.

The compound was eluted from the HPLC Chiralpak AD column as the minor, faster eluting, isomer.

It was converted to the title compound by the method of Example (99).

### Example 189

### 1-[3-chloro-6-(methoxy)-4-quinolinyl]-2-{4[(2,3-dihydro[1,4]dioxino[2,3-c]pyridin-7-ylmethyl)amino]-1-piperidinyl}ethanol dihydrochloride Enantiomer E2

Vinyl-quinoline (4c) was taken through the sequence outlined in Example (99) using AD-mixα as a chiral agent for the dihydroxylation step.

The compound was eluted from the HPLC Chiralpak AD column as the major, slower eluting, isomer.

[α]_{D} (25° C.)=−23.1 degrees (c=1%, methanol).

It was converted to the title compound by the method of Example (99).

### Example 190

### 1-[3-chloro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E1

Vinyl-quinoline (4c) was taken through the sequence outlined in Example (99).

The compound was eluted from the HPLC Chiralpak AD column as the major, faster eluting, isomer.

[α]_{D} (25° C.)=+23.6 degrees (c=1%, methanol).

It was converted to the title compound by the method of Example (99).

### Example 191

### 1-[3-chloro-6-(methoxy)-1,5-naphthyridin-4-yl]-2-{4-[(2,3-dihydro[1,4]dioxino[2,3-c]pyridin-7-ylmethyl)amino]-1-piperidinyl}ethanol dihydrochloride Enantiomer E2

Vinyl-naphthyridine (3a) was taken through the sequence outlined in Example (99).

The compound was eluted from the HPLC Chiralpak AD column as the minor, slower eluting, isomer.

[α]_{D} (25° C.)=−7.5 degrees (c=1%, methanol).

It was converted to the title compound by the method of Example (99).

### Example 192

### 2-{4-[(2,3-dihydro[1,4]dioxino[2,3-c]pyridin-7-ylmethyl)amino]-3-fluoro-1-piperidinyl}-1-[3-fluoro-6-(methoxy)-1,5-naphthyridin-4-yl]ethanol dihydrochloride Enantiomer E2

### (a) 7-fluoro-2-(methoxy)-8-(2-oxiranyl)-1,5-naphthyridine

Vinyl-naphthyridine (53h) was treated as in Example (99 a, b and c) but using AD-mix a in the dihydroxylation step (99a) to afford the product.

MS (ES) m/z 221 (M+H)⁺.

### (b) phenylmethyl (3-fluoro-1-{2-[3-fluoro-6-(methoxy)-1,5-naphthyridin-4-yl]-2-hydroxyethyl}-4-piperidinyl)

This was prepared as in Example (99i) using epoxide (a) and piperidine (142d, enantiomer E2).

MS (ES) m/z 473 (M+H)⁺.

### (c) 2-(4-amino-3-fluoro-1-piperidinyl}-1-[3-fluoro-6-(methoxy)-1,5-naphthyridin-4-yl]ethanol

Piperidine (b) was treated as in Example (99h) to afford the product.

MS (ES) m/z 339 (M+H)⁺.

### (d) Title Compound

Amine (d) and aldehyde (2c) were treated as in Example (99f) to afford the free base of the product (88% de).

The compound was eluted from the HPLC Chiralpak AD column as the minor, slower eluting, isomer.

[α]_{D} (25° C.)=+3.4 degrees (c=1%, methanol).

It was converted to the title compound by the method of Example (99).

### Example 193

### 2-{4-[(2,3-dihydro[1,4]dioxino[2,3-c]pyridin-7-ylmethyl)amino]-3-fluoro-1-piperidinyl}-1-[3-fluoro-6-(methoxy)-1,5-naphthyridin-4-yl]ethanol dihydrochloride Enantiomer E1

This was prepared as in Example (192), but piperidine (142d, enantiomer E1) in step (192b).

The compound (99.4% de) was eluted from the HPLC Chiralpak AD column as the major, faster eluting, isomer.

[α]_{D} (25° C.)=+16.3 degrees (c=1%, methanol).

It was converted to the hydrochloride by the method of Example (99).

### Example 194

### 7-{[(1-{2-[3,8-difluoro-6-(methoxy)-4-quinolinyl]ethyl}-3-fluoro-4-piperidinyl)amino]methyl}-1H-pyrido[2,3-b][1,4]thiazin-2(3H)-one dihydrochloride Enantiomer E2

Vinyl-quinoline (47j) and fluoropiperidine (142d, Enantiomer E2) and 2-oxo-2,3-dihydro-1H-pyrido[2,3-b][1,4]thiazine-7-carbaldehyde (aldehyde as in Example 56) were treated as in Example (142e, f and g) to afford the free base of the title compound.

MS (ES) m/z 518 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 195

### 1-{2-[3-fluoro-6-(methoxy)-1,5-naphthyridin-4-yl]ethyl}-N-{[8-(methoxy)-2,3-dihydro-1,4-benzodioxin-6-yl]methyl}-4-piperidinamine

### (a) 8-(methoxy)-2,3-dihydro-1,4-benzodioxin-6-carbaldehyde

To 3,4-dihydroxy-5-methoxy benzaldehyde (5.0 g, 29.7 mmol) was added acetone (100 mL), 1,2 dibromoethane (3.56 mL, 41.4 mL), and potassium carbonate (2.97 g, 21.5 mmol). The solution was heated to reflux and stirred for 3 days. The solution was then cooled to room temperature and the solvent removed under reduced pressure. Ethyl acetate was added and the solution was washed with water and brine. The organic layer was then dried over Na₂SO₄, filtered, and the solvent removed under reduced pressure yielding a crude solid. This was chromatographed on silica gel to yield a white solid (0.890 g, 15%).

MS (ES) m/z 195 (M+H)⁺.

### (b) Title Compound

Amine (53i) and aldehyde (a) were treated as in example (53j) to afford the free base of the compound.

MS (ES) m/z 483 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

### Example 196

### 1-{2-[3-fluoro-6-(methoxy)-1,5-naphthyridin-4-yl]ethyl}-N-[(7-methyl-2,3-dihydro-1,4-benzodioxin-6-yl)methyl]-4-piperidinamine

### (a) 2,3-dihydro-1,4-benzodioxin-6-ol

2,3-Dihydro-benzo{1,4]dioxine-6-carbaldehyde (1.78 g, 10.8 mmol) was dissolved in CH₂Cl₂ (10 mL). M-chloroperbenzoic acid (4.11 g, 23.9 mmol) was added and the solution heated to reflux for 5 hours. The solution was then allowed to cool to room temperature and further cooled in an ice bath. The remaining solid was filtered off (excess m-chloroperbenzoic acid) and the solution washed with saturated NaHCO₃ solution, water, and brine. This was chromatographed on silica gel to yield a white solid (1.65 g, 100%).

MS (ES) m/z 153 (M+H)⁺.

### (b) 6-(methoxy)-2,3-dihydro-1,4-benzodioxin

Alcohol (a) (1.55 g, 10.2 mmol) was dissolved in acetone (10 mL). Dimethyl sulfate (1.06 mL, 11.2 mmol) and potassium carbonate (3.71 g, 26.8 mmol) were added and the solution heated to reflux. The solution was stirred at reflux for 18 hours. It was then cooled to room temperature and concentrated under reduced pressure. The remaining material was diluted with water and extracted several times with EtOAc. The combined organic layers were dried over Na₂SO₄, and evaporated to yield a colorless oil. (0.86 g, 51%).

MS (ES) m/z 167 (M+H)⁺.

### (c) 7-(methoxy)-2,3-dihydro-1,4-benzodioxin-6-carbaldehyde

Methoxy-benzodioxine (b) (0.85 g, 5.11 mmol) was dissolved in DMF (0.60 mL, 7.66 mmol) and phosphorous trichloride (0.57 mL, 6.14 mmol) was added. The solution was heated to 100° C. and allowed to stir for 5 hours. The solution was poured into ice water and was brought to pH 14 with aqueous sodium hydroxide. A white solid precipitated out, was filtered and dried under vacuum to afford the product (0.91 g, 92%).

MS (ES) m/z 195 (M+H)⁺.

### (d) 7-hydroxy-2,3-dihydro-1,4-benzodioxin-6-carbaldehyde

Aldehyde (c) (0.840 g, 4.33 mmol) was dissolved in dichloromethane (10 mL) and boron tribromide (8.66 mL, 8.66 mmol) was added. The solution was allowed to stir at room temperature for 1 hour. The reaction was diluted with water and brought to pH=7 with a saturated potassium carbonate solution. It was then extracted several times with dichloromethane and the combined organic layers washed with brine, dried over Na₂SO₄, and evaporated to yield an off white solid (0.793 g, 100%).

MS (ES) m/z 181 (M+H)⁺.

### (e) 7-formyl-2,3-dihydro-1,4-benzodioxin-6-yl trifluoromethanesulfonate

Aldehyde (d) (0.500 g, 2.78 mmol) was dissolved in DMF (10 mL). Triethylamine (0.58 mL, 4.16 mmol) and N-phenyltrifluoromethanesulphonimide (1.09 g, 3.06 mmol) were added. The solution was allowed to stir at room temperature for 48 hours. The reaction was then diluted with dichloromethane and washed with a saturated potassium carbonate solution. The aqueous layer was extracted with dichloromethane and the combined organic layers washed with brine, dried over Na₂SO₄, and evaporated to yield an oil. This was chromatographed on silica gel to yield a colorless oil with some triflamide contamination (1.07 g, >100%).

MS (ES) m/z 313 (M+H)⁺.

### (f) 7-methyl-2,3-dihydro-1,4-benzodioxin-6-carbaldehyde

Triflate (e) (0.800 g, 2.56 mmol) was dissolved in DMF (10 mL).

Dichlorobis(triphenylphosphine)palladium (II) (0.09 g, 0.13 mmol), lithium trichloride (0.33 g, 7.68 mmol), and tetramethyltin (0.53 mL, 3.84 mmol) were added. The solution was heated to 100° C. and stirred for 1 hour. The solution was cooled to room temperature and diluted with ethyl acetate. It was then washed twice with water and brine. The organic layer was dried over Na₂SO₄ and evaporated to yield a crude solid. This was chromatographed on silica gel to yield an off-white solid (0.235 g, 52%).

MS (ES) m/z 179 (M+H)⁺.

### (g) Title Compound

Amine (53i) and aldehyde (f) were treated as in example (53j) to afford the free base of the compound.

MS (ES) m/z 467 (M+H)⁺.

This material, as a solution in chloroform/methanol, was treated with an excess of 1M HCl in ether and evaporated to dryness. The solid was triturated under ether, filtered and dried under vacuum to provide the title compound as a white solid.

Biological Activity

Antimicrobial Activity Assay:

Whole-cell antimicrobial activity was determined by broth microdilution using the National Committee for Clinical Laboratory Standards (NCCLS) recommended procedure, Document M7-A6, “Methods for Dilution Susceptibility Tests for Bacteria that Grow Aerobically”. The compounds were tested in serial two-fold dilutions ranging from 0.016 to 16 mcg/mL. Compounds were evaluated against a panel of Gram-positive organisms, including Staphylococcus aureus WCUH29, Streptococcus pneumoniae 1629, Streptococcus pyogenes CN 10, and Enterococcus faecalis 2. In addition, compounds were evaluated against a panel of Gram-negative strains including Haemophilus influenzae NEMC1, E. coli 7623, and Moraxella catarrhalis Ravasio. The minimum inhibitory concentration (MIC) was determined as the lowest concentration of compound that inhibited visible growth. A mirror reader was used to assist in determining the MIC endpoint.

One skilled in the art would consider any compound with a MIC of less than 20 μg/mL to be a potential lead compound. Compounds of the present invention have MIC's ≦20 μg/ml versus all the organisms named above.

Examples 1, 3-13, 15-23, 25-32, 34-37, 39-41, 43-45, 47-56, 58-62, 66, 68, 70, 72-77, 79, 81, 84-86, 91-100, 105-106, 109-110, 113, 116-118, 120, 122-128, 133-135, 138-140, 142-151, 153-165, 167-172, 174, 176-178, 181, 182, 187-188, 194 had MIC's ≦2 μg/ml versus all the organisms named above.

Rat Infection Model:

Certain compounds of this invention were tested in the rat infection model. Specific pathogen-free male Sprague-Dawley CD rats were used for all bacterial strains. Each therapy group consists of 5 animals. Infection was carried out by intrabronchial instillation of 100 ul bacterial suspension for H. influenzae H128, and 50 ul of bacterial suspension for S. pneumoniae 1629 via non-surgical intubation. All compounds were administered at 1, 7, 24 and 31 hr post infection via oral gavage. In each experiment, an additional group of animals was included and served as untreated infected controls. Approximately 17 hr after the end of therapy, the animals were killed and their lungs excised and enumeration of the viable bacteria was conducted by standard methods. The lower limit of detection was 1.7 log10 CFU/lungs.

In vivo, activity was observed in infection models in rats versus S. pneumoniae 1629 at doses ranging from 25-100 mg/Kg with oral dosing and for some compounds versus H. influenzae H128 at doses from 25-100 mg/Kg with oral dosing. Certain formula (I) compounds showed a greater than 2 log drop in viable counts in the lungs compared to non-treated controls versus S. pneumoniae 1629. Certain compounds of formula (I) showed greater than a 4 log drop in viable counts in the lungs compared to non-treated controls versus H. influenzae H 128. The compounds of this invention are particularly interesting due to their low toxicity with no toxicity being observed in rats with dosing twice daily for 2 days at 50 mg/Kg.

What is claimed is:

1. A compound according to formula (I): 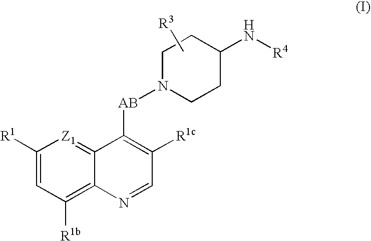
wherein:
Z₁ is Nor CR^{1a};
R¹ and R^{1a} are independently selected from H, nitro, halogen, (C₁₋₃)alkylthio, (C₁₋₃)alkyl, and (C₁₋₃)alkoxy optionally substituted by (C₁₋₃)alkoxy; or R¹ and R^{1a} are joined together to form ethylenedioxy;
R^{1b} is H or halogen;
with the proviso that when Z₁ is N, then R^{1b} is H and when Z₁ is CR^{1a} then R¹ is not H;
R^{1c} is halogen;
AB is CHR⁶—CO or CHR⁶—CH₂;
R⁶ is H, NH₂, —CH₂OH, or hydroxy;
R³ is up to two substituents selected from H, halogen, (C₁₋₃)alkyl, hydroxy(C₁₋₃)alkyl, CONH₂, COOH, —CH₂CONH₂, —CH₂COOH, —CONHCH₃, and hydroxy in the 3-position optionally substituted by (C₁₋₃)alkyl;
R⁴ is a group —U—R⁵ where R⁵ is a substituted or unsubstituted bicyclic carbocyclic or heterocyclic ring system (A): 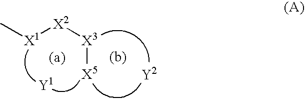
containing up to four heteroatoms in each ring in which
at least one of rings (a) and (b) is aromatic;
X¹ is C or N when part of an aromatic ring or CR¹⁴ when part of a non aromatic ring;
X² is N, NR¹³, O, S(O)ₓ, CO or CR¹⁴ when part of an aromatic or non-aromatic ring or may in addition be CR¹⁴R¹⁵ when part of a non aromatic ring;
X³ and X⁵ are independently N or C;
Y¹ is a 0 to 4 atom linker group each atom of which is independently selected from N, NR¹³, O, S(O)ₓ, CO and CR¹⁴ when part of an aromatic or non-aromatic ring or may additionally be CR¹⁴R¹⁵ when part of a non aromatic ring,
Y² is a 2 to 6 atom linker group, each atom of Y² being independently selected from N, NR¹³, O, S(O)ₓ, CO and CR¹⁴ when part of an aromatic or non-aromatic ring or may additionally be CR¹⁴R¹⁵ when part of a non aromatic ring;
each of R¹⁴ and R¹⁵ is independently selected from H; (C₁₋₄)alkylthio;
halo; (C₁₋₄)alkyl; (C₂₋₄)alkenyl; hydroxy; hydroxy(C₁₋₄)alkyl; mercapto(C₁₋₄)alkyl; (C₁₋₄)alkoxy; trifluoromethoxy; nitro; cyano; carboxy; amino or aminocarbonyl optionally substituted by (C₁₋₄)alkyl;
each R¹³ is independently H; trifluoromethyl; (C₁₋₄)alkyl optionally substituted by hydroxy, carboxy, (C₁₋₄)alkoxy, (C₁₋₆)alkylthio, halo or trifluoromethyl; (C₂₋₄)alkenyl; or aminocarbonyl wherein the amino group is optionally substituted (C₁₋₄)alkyl;
each x is independently 0, 1 or 2; and
U is CO, SO₂ or CH₂; or a pharmaceutically acceptable salt thereof.

2. A compound according to claim 1 wherein R¹ is F, Cl, OCH₃, methyl, or SCH₃.

3. A compound according to claim 1 R^{1a} is H, OCH₃, OCH₂CH₂OCH₃.

4. A compound according to claim 1 wherein R^{1b} is H or F.

5. A compound according to claim 1 wherein R^{1c} is Cl or F.

6. A compound according to claim 1 wherein R³ is H, OH, OCH₃, or CH₂OH.

7. A compound according to claim 1 wherein R⁶ is H or OH.

8. A compound according to claim 1 wherein the group —U— is —CH₂—.

9. A compound according to claim 1 wherein R⁵ is:
benzo[1,2,5]thiadiazol-5-yl;
4H-benzo[1,4] thiazin-3-one-6-yl;
2,3-dihydro-benzo[1,4]dioxin-6-yl;
benzo[1,2,3]thiadiazol-5-yl;
3-oxo-3,4-dihydro-2H-benzo[1,4]oxazin-6-yl;
7-fluoro-3-oxo-3,4-dihydro-2H-benzo[1,4] oxazin-6-yl;
2-oxo-2,3-dihydro-1H-pyrido[2,3-b][1,4]thiazin-7-yl;
2,3-Dihydro-[1,4]dioxino[2,3-c]pyridin-7-yl;
3-oxo-3,4-dihydro-2H-pyrido[3,2-b][1,4]oxazin-6-yl;
[1,2,3]thiadiazolo[5,4-b]pyridin-6-yl;
3-oxo-3,4-dihydro-2H-pyrido[3,2-b][1,4]thiazin-6-yl;
7-chloro-3-oxo-3,4-dihydro-2H-pyrido[3,2-b][1,4]thiazin-6-yl; or
7-fluoro-3-oxo-3,4-dihydro-2H-pyrido[3,2-b][1,4]thiazin-6-yl.

10. A compound according to claim 1 which is:
6-({1-[(Racemic)-2-(3-Chloro-6-methoxy-[1,5]naphthyridin-4-yl)-2-hydroxy-ethyl]-piperidin-4-ylamino}-methyl)-4H-pyrido[3,2-b][1,4]oxazin-3-one Dihydrochloride;
(Racemic)-1-(3-Chloro-6-methoxy-[1,5]naphthyridin-4-yl)-2-{4-[(2,3-dihydro-[1,4]dioxino[2,3-c]pyridin-7-ylmethyl)-amino]-piperidin-1-yl}-ethanol Dihydrochloride;
{1-[2-(3-Chloro-6-methoxy-[1,5]naphthyridin-4-yl)-ethyl]-piperidin-4-yl}-(2,3-dihydro-[1,4]dioxino[2,3-c]pyridin-7-ylmethyl)-amine Dihydrochloride;
{1-[2-(3-Chloro-6-methoxy-quinolin-4-yl)-ethyl]-piperidin-4-yl}-(2,3-dihydro-[1,4]dioxino[2,3-c]pyridin-7-ylmethyl)-amine Dihydrochloride;
6-({(cis)-1-[2-(3-Chloro-6-methoxy-quinolin-4-yl)-ethyl]-3-hydroxy-piperidin-4-ylamino}-methyl)-4H-pyrido[3,2-b][1,4]oxazin-3-one Dihydrochloride Enantiomer 1;
6-({(cis)-1-[2-(3-Chloro-6-methoxy-quinolin-4-yl)-ethyl]-3-hydroxy-piperidin-4-ylamino}-methyl)-4H-pyrido[3,2-b][1,4]oxazin-3-one Dihydrochloride Enantiomer 2;
6-({(cis)-1-[2-(3-Chloro-6-methoxy-quinolin-4-yl)-ethyl]-3-hydroxy-piperidin-4-ylamino}-methyl)-4H-pyrido[3,2-b][1,4]thiazin-3-one Dihydrochloride Enantiomer 1;
6-({(cis)-1-[2-(3-Chloro-6-methoxy-quinolin-4-yl)-ethyl]-3-hydroxy-piperidin-4-ylamino}-methyl)-4H-pyrido[3,2-b][1,4]thiazin-3-one Dihydrochloride Enantiomer 2;
6-({(cis)-1-[2-(3-Chloro-6-methoxy-[1,5]naphthyridin-4-yl)-ethyl]-3-hydroxy-piperidin-4-ylamino}-methyl)-4H-pyrido[3,2-b][1,4]oxazin-3-one Dihydrochloride Enantiomer 1;
6-({(cis)-1-[2-(3-Chloro-6-methoxy-[1,5]naphthyridin-4-yl)-ethyl]-3-hydroxy-piperidin-4-ylamino}-methyl)-4H-pyrido[3,2-b][1,4]oxazin-3-one Dihydrochloride Enantiomer 2;
6-({(cis)-1-[2-(3-Chloro-6-methoxy-[1,5]naphthyridin-4-yl)-ethyl]-3-hydroxy-piperidin-4-ylamino}-methyl)-4H-pyrido[3,2-b][1,4]thiazin-3-one Dihydrochloride Enantiomer 1;
6-({(cis)-1-[2-(3-Chloro-6-methoxy-[1,5]naphthyridin-4-yl)-ethyl]-3-hydroxy-piperidin-4-ylamino}-methyl)-4H-pyrido[3,2-b][1,4]thiazin-3-one Dihydrochloride Enantiomer 2;
6-({1-[2-(3-Chloro-6-methoxyquinolin-4-yl)ethyl]piperidin-4-yl amino}methyl)-4H-pyrido[3,2-b][1,4]thiazin-3-one Trihydrochloride;
6-({1-[2-(3-chloro-6-methoxyquinolin-4-yl)ethyl]piperidin-4-yl amino}methyl)-4H-pyrido[3,2-b][1,4]oxazin-3-one Trihydrochloride;
6-({1-[2-(3-Chloro-6-methoxynaphthyridin-4-yl)ethyl]piperidin-4-yl amino}methyl)-4H-pyrido[3,2-b][1,4]thiazin-3-one Dihydrochloride;
6-({1-[2-(3-chloro-6-methoxynaphthyridin-4-yl)ethyl]piperidin-4-yl amino}methyl)-4H-pyrido[3,2-b][1,4]oxazin-3-one Dihydrochloride;
6-({(trans)-1-[2-(3-Chloro-6-methoxyquinolin-4-yl)ethyl]3-hydroxypiperidin-4-yl amino}methyl)-4H-pyrido[3,2-b][1,4]thiazin-3-one Trihydrochloride enantiomer 2;
6-({(trans)-1-[2-(3-Chloro-6-methoxyquinolin-4-yl)ethyl]3-hydroxypiperidin-4-yl amino}methyl)-4H-pyrido[3,2-b][1,4]oxazin-3-one Trihydrochloride enantiomer 2;
6-(trans)-1-[2-(3-Chloro-6-methoxyquinolin-4-yl)ethyl]3-hydroxypiperidin-4-yl amino}methyl)-4H-pyrido[3,2-b][1,4]thiazin-3-one-Trihydrochloride enantiomer 1;
6-(trans)-1-[2-(3-Chloro-6-methoxyquinolin-4-yl)ethyl]3-hydroxypiperidin-4-yl amino}methyl)-4H-pyrido[3,2-b][1,4]oxazin-3-one Trihydrochloride enantiomer 1;
6-({1-[2-(3-Chloro-6-methoxyquinolin-4-yl)ethyl]4-hydroxymethylpiperidin-4-ylamino}methyl)-4H-pyrido[3,2-b][1,4]thiazin-3-one Dihydrochloride;
6-({1-[2-(3-Chloro-6-fluoro-5-methoxy-quinolin-4-yl)-ethyl]-piperidin-4-ylamino}-methyl)-4H-pyrido[3,2-b][1,4]thiazin-3-one Dihydrochloride;
6-({1-[2-(3-Chloro-6-methyl-[1,5]naphthyridin-4-yl)-ethyl]-piperidin-4-ylamino}-methyl)-4H-pyrido[3,2-b][1,4]thiazin-3-one Dihydrochloride;
{1-[2-(3-Chloro-6-methyl-[1,5]naphthyridin-4-yl)-ethyl]-piperidin-4-yl}-(2,3-dihydro[1,4]dioxino[2,3-c]pyridin-7-ylmethyl)-amine Dihydrochloride;
6-({1-[2-(3-Chloro-6-fluoro-quinolin-4-yl)-ethyl]-piperidin-4-ylamino}-methyl)-4H-pyrido[3,2-b][1,4]thiazin-3-one Dihydrochloride;
{1-[2-(3-Chloro-6-fluoro-quinolin-4-yl)-ethyl]-piperidin-4-yl}-(2,3-dihydro[1,4]dioxino[2,3-c]pyridin-7-ylmethyl)-amine Dihydrochloride;
6-({1-[2-(3,6-Dichloro-quinolin-4-yl)-ethyl]-piperidin-4-ylamino}-methyl)-4H-pyrido[3,2-b][1,4]thiazin-3-one Dihydrochloride;
{1-[2-(3,6-Dichloro-quinolin-4-yl)-ethyl]-piperidin-4-yl}-(2,3-dihydro[1,4]dioxino[2,3-c]pyridin-7-ylmethyl-amine Dihydrochloride;
(cis)-1-[2-(3-Chloro-6-methoxy-[1,5]naphthyridin-4-yl)-ethyl]-4-[(2,3-dihydro-[1,4]dioxino[2,3-c]pyridin-7-ylmethyl)-amino]-piperidin-3-ol Dihydrochloride Enantiomer 1;
(cis)-1-[2-(3-Chloro-6-methoxy-[1,5]naphthyridin-4-yl)-ethyl]-4-[(2,3-dihydro-[1,4]dioxino[2,3-c]pyridin-7-ylmethyl)-amino]-piperidin-3-ol Dihydrochloride Enantiomer 2;
6-({1-[2-(3-Fluoro-6-methoxyquinolin-4-yl)ethyl]piperidin-4-yl amino}methyl)-4H-pyrido[3,2-b][1,4]thiazin-3-one dihydrochloride;
{1-[2-(3-Fluoro-6-methoxy-quinolin-4-yl)-ethyl]-piperidin-4-yl}-(2,3-dihydro-[1,4]dioxino[2,3-c]pyridin-7-ylmethyl)-amine dihydrochloride;
cis-4-[(2,3-Dihydro-[1,4]dioxino[2,3-c]pyridin-7-ylmethyl)-amino]-1-[2-(3-fluoro-6-methoxy-quinolin-4-yl)-ethyl]-piperidin-3-ol Enantiomer 2 dihydrochloride;
cis-4-[(2,3-Dihydro-[1,4]dioxino[2,3-c]pyridin-7-ylmethyl)-amino]-1-[2-(3-fluoro-6-methoxy-quinolin-4-yl)-ethyl]-piperidin-3-ol dihydrochloride dihydrochloride Enantiomer 1;
{1-[2-(3-Chloro-6-methoxy-[1,5]naphthyridin-4-yl)-2-hydroxyethyl]-piperidin-4-yl}-(2,3-dihydro-[1,4]dioxino[2,3-c]pyridin-7-ylmethyl)-amine Dihydrochloride Enantiomer 1;
6-({1-[2-(3-Chloro-6-methoxy-[1,5]naphthyridin-4-yl)-2-hydroxy-ethyl]-piperidin-4-ylamino}-methyl)-4H-pyrido[3,2-b][1,4]thiazin-3-one Dihydrochloride Enantiomer 1;
6-({1-[2-(3-Chloro-6-methoxy-[1,5]naphthyridin-4-yl)-2-hydroxy-ethyl]-piperidin-4-ylamino}-methyl)-4H-pyrido[3,2-b][1,4]thiazin-3-one Dihydrochloride Enantiomer 2;
{6-(trans)-1-[2-(3-Chloro-6-methoxyquinolin-4-yl)ethyl]-3-hydroxypiperidin-4-yl}-(2,3-dihydro-[1,4]dioxino[2,3-c]pyridin-7-ylmethyl)amine Enantiomer 2;
(trans)-6-({1-[2-(3-Chloro-6-methoxy-[1,5]naphthyridin-4-yl)-ethyl]-3-hydroxy-piperidin-4-ylamino}-methyl)-4H-pyrido[3,2-b][1,4]-thiazin-3-one Dihydrochloride Enantiomer 2;
trans-6-({1-[2-(3-Chloro-6-methoxy-[1,5]naphthyridin-4-yl)-ethyl]-3-hydroxy-piperidin-4-ylamino}-methyl)-4H-pyrido[3,2-b][1,4] oxazin-3-one Trihydrochloride Enantiomer 2;
trans-6-({1-[2-(3-Chloro-6-methoxy-[1,5]naphthyridin-4-yl)-ethyl]-3-hydroxy-piperidin-4-ylamino}-methyl)-4H-pyrido[3,2-b][1,4] thiazin-3-one dihydrochloride Enantiomer 1;
6-({(3R,4r,5S)-1-[2-(3-Chloro-6-methoxy-quinolin-4-yl)-ethyl]-3,5-dihydroxy-piperidin-4-ylamino)}-methyl)-4H-pyrido[3,2-b][1,4]oxazin-3-one dihydrochloride;
6-({1-[2-(3-Fluoro-6-methoxyquinolin-4-yl)ethyl]piperidin-4-yl amino}methyl)-4H-pyrido[3,2-b][1,4]oxazin-3-one dihydrochloride;
{1-[2-(3-Bromo-6-methoxy-quinolin-4-yl)-ethyl]-piperidin-4-yl}-(2,3-dihydro-[1,4]dioxino[2,3-c]pyridin-7-ylmethyl)-amine Dihydrochloride;
cis-1-[2-(3-Chloro-6-methoxy-quinolin-4-yl)-ethyl]-4-[(2,3-dihydro-[1,4]dioxino[2,3-c]pyridin-7-ylmethyl)-amino]-piperidin-3-ol Dihydrochloride Enantiomer 1;
cis-1-[2-(3-Chloro-6-methoxy-quinolin-4-yl)-ethyl]-4-[(2,3-dihydro-[1,4]dioxino[2,3-c]pyridin-7-ylmethyl)-amino]-piperidin-3-ol Dihydrochloride Enantiomer 2;
1-{2-[3,8-difluoro-6-(methoxy)-4-quinolinyl]ethyl}-N-(2,3-dihydro[1,4]dioxino[2,3-c]pyridin-7-ylmethyl)-4-piperidinamine dihydrochloride;
7-{[(1-{2-[3,8-Difluoro-6-(methoxy)-4-quinolinyl]ethyl}-4-piperidinyl)amino]methyl}-1H-pyrido[2,3-b][1,4]thiazin-2(3H)-one dihydrochloride;
6-{[(1-{2-[3,8-Difluoro-6-(methoxy)-4-quinolinyl]ethyl}-4-piperidinyl)amino]methyl}-2H-pyrido[3,2-b][1,4]oxazin-3(4H)-one dinydrochloride;
6-{[(1-{2-[3,8-difluoro-6-(methoxy)-4-quinolinyl]ethyl}-4-piperidinyl)amino]methyl}-2H-pyrido[3,2-b][1,4]thiazin-3(4H)-one dihydrochloride;
1-{2-[3,8-difluoro-6-(methoxy)-4-quinolinyl]ethyl}-N-([1,3]dioxolo[4,5-c]pyridin-6-ylmethyl)-4-piperidinamine dihydrochloride;
{1-[2-(9-Chloro-2,3-dihydro-[1,4]dioxino[2,3-f]quinolin-10-yl)-ethyl]-piperidin-4-yl}-(2,3-dihydro-[1,4]dioxino[2,3-c]pyridin-7-ylmethyl)-amine dihydrochloride;
N-(2,3-dihydro[1,4]dioxino[2,3-c]pyridin-7-ylmethyl)-1-{2-[3-fluoro-6-(methoxy)-1,5-naphthyridin-4-yl]ethyl}-4-piperidinamine dihydrochloride;
N-(2,3-Dihydro-1H-pyrido[3,4-b][1,4]thiazin-7-ylmethyl)-1-{2-[3-fluoro-6-(methoxy)-1,5-naphthyridin-4-yl]ethyl}-4-piperidinamine dihydrochloride;
6-{[(1-{2-[3-fluoro-6-(methoxy)-1,5-naphthyridin-4-yl]ethyl}-4-piperidinyl)amino]methyl}-2H-pyrido[3,2-b][1,4]oxazin-3(4H)-one dihydrochloride;
7-{[(1-{2-[3-fluoro-6-(methoxy)-1,5-naphthyridin-4-yl]ethyl}-4-piperidinyl)amino]methyl}-1H-pyrido[2,3-b][1,4]thiazin-2(3H)-one dihydrochloride;
3-{[(1-{2-[3-fluoro-6-(methoxy)-1,5-naphthyridin-4-yl]ethyl}-4-piperidinyl)amino]methyl}-8-hydroxy-1 (2H)-isoquinolinone dihydrochloride;
3-{[(1-{2-[3-fluoro-6-(methoxy)-1,5-naphthyridin-4-yl]ethyl}-4-piperidinyl)amino]methyl}-5H-pyridazino[3,4-b][1,4]thiazin-6(7H)-one dihydrochloride;
6-[({1-{2-[3-Fluoro-6-(methoxy)-1,5-naphthyridin-4-yl]ethyl}-4-piperidinyl)amino]methyl}-2H-pyrido[3,2-b][1,4]thiazin-3(4H)-one dihydrochloride;
N-(2,3-Dihydro[1,4]oxathiino[2,3-c]pyridin-7-ylmethyl)-1-{2-[3-fluoro-6-(methoxy)-1,5-naphthyridin-4-yl]ethyl}-4-piperidinamine dihydrochloride;
1-{2-[3-Fluoro-6-(methoxy)-1,5-naphthyridin-4-yl]ethyl}-N-([1,3]oxathiolo[5,4-c]pyridin-6-ylmethyl)-4-piperidinamine dihydrochloride;
7-Fluoro-N-(1-{2-[3-fluoro-6-(methoxy)-1,5-naphthyridin-4-yl]ethyl}-4-piperidinyl)-3-oxo-3,4-dihydro-2H-pyrido[3,2-b][1,4]thiazine-6-carboxamide dihydrochloride;
N-(1-{2-[3-fluoro-6-(methoxy)-1,5-naphthyridin-4-yl]ethyl}-4-piperidinyl)-2-oxo-2,3-dihydro-1H-pyrido[2,3-b][1,4]thiazine-7-carboxamide dihydrochloride;
N-(1-{2-[3-fluoro-6-(methoxy)-1,5-naphthyridin-4-yl]ethyl}-4-piperidinyl)-3-oxo-3,4-dihydro-2H-pyrido[3,2-b][1,4]thiazine-6-carboxamide;
N-(1-{2-[3-fluoro-6-(methoxy)-1,5-naphthyridin-4-yl]ethyl}-4-piperidinyl)-3-oxo-3,4-dihydro-2H-pyrido[3,2-b][1,4]oxazine-6-carboxamide;
(3R,4S)-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinol dihydrochloride Enantiomer 1;
6-{[((3R,4S)-1-{2-[3-fluoro-6-(methoxy)-1,5-naphthyridin-4-yl]ethyl}-3-hydroxy-4-piperidinyl)amino]methyl}-2H-pyrido[3,2-b][1,4]thiazin-3(4H)-one dihydrochloride;
6-{[((3R,4S)-1-{2-[3-fluoro-6-(methoxy)-1,5-naphthyridin-4-yl]ethyl}-3-hydroxy-4-piperidinyl)amino]methyl}-2H-pyrido[3,2-b][1,4]oxazin-3(4H)-one dihydrochloride;
(3R,4S)-4-[(2,3-dihydro[1,4]dioxino[2,3-b]pyridin-7-ylmethyl)amino]-1-{2-[3-fluoro-6-(methoxy)-1,5-naphthyridin-4-yl]ethyl}-3-piperidinol dihydrochloride;
6-{[((3S,4R)-1-{2-[3-fluoro-6-(methoxy)-1,5-naphthyridin-4-yl]ethyl}-3-hydroxy-4-piperidinyl)amino]methyl}-2H-pyrido[3,2-b][1,4]thiazin-3(4H)-one dihydrochloride Enantiomer 2;
N-((3S,4R)-1-{2-[3-fluoro-6-(methoxy)-1,5-naphthyridin-4-yl]ethyl}-3-hydroxy-4-piperidinyl)-3-oxo-3,4-dihydro-2H-pyrido[3,2-b][1,4]thiazine-6-carboxamide hydrochloride Enantiomer 2;
7-{[((3R,4S)-1-{2-[3,8-difluoro-6-(methoxy)-4-quinolinyl]ethyl}-3-hydroxy-4-piperidinyl)amino]methyl}-1H-pyrido[2,3-b][1,4]thiazin-2(3H)-one dihydrochloride Enantiomer 1;
6-{[((3R,4S)-1-{2-[3,8-difluoro-6-(methoxy)-4-quinolinyl]ethyl}-3-hydroxy-4-piperidinyl)amino]methyl}-2H-pyrido[3,2-b][1,4]thiazin-3(4H)-one dihydrochloride Enantiomer 1;
(3R,4S)-1-{2-[3,8-difluoro-6-(methoxy)-4-quinolinyl]ethyl}-4-[(2,3-dihydro[1,4]dioxino[2,3-c]pyridin-7-ylmethyl)amino]-3-piperidinol dihydrochloride dihydrochloride Enantiomer 1;
6-{[((3R,4S)-1-{2-[3,8-difluoro-6-(methoxy)-4-quinolinyl]ethyl}-3-hydroxy-4-piperidinyl)amino]methyl}-2H-pyrido[3,2-b][1,4]oxazin-3(4H)-one dihydrochloride;
N-[(4-fluoro-1H-benzimidazol-2-yl)methyl]-1-{2-[3-fluoro-6-(methoxy)-4-quinolinyl]ethyl}-4-piperidinamine;
1-{2-[3-fluoro-6-(methoxy)-4-quinolinyl]ethyl}-N-(1,5,6,7-tetrahydro-1,8-naphthyridin-2-ylmethyl)-4-piperidinamine dihydrochloride;
N-(3-cinnolinylmethyl)-1-{2-[3-fluoro-6-(methoxy)-4-quinolinyl]ethyl}-4-piperidinamine dihydrochloride;
N-(2,1,3-benzothiadiazol-5-ylmethyl)-1-{2-[3-fluoro-6-(methoxy)-4-quinolinyl]ethyl}-4-piperidinamine dihydrochloride;
1-{2-[3-fluoro-6-(methoxy)-4-quinolinyl]ethyl}-N-([1,3]thiazolo[5,4-b]pyridin-6-ylmethyl)-4-piperidinamine dihydrochloride;
N-(3,4-dihydro-2H-pyrido[3,2-b][1,4]thiazin-6-ylmethyl)-1-{2-[3-fluoro-6-(methoxy)-4-quinolinyl]ethyl}-4-piperidinamine dihydrochloride;
N-(1,3-benzothiazol-5-ylmethyl)-1-{2-[3-fluoro-6-(methoxy)-4-quinolinyl]ethyl}-4-piperidinamine dihydrochloride;
1-{2-[3-fluoro-6-(methoxy)-4-quinolinyl]ethyl}-N-([1,2,3]thiadiazolo[5,4-b]pyridin-6-ylmethyl)-4-piperidinamine dihydrochloride;
7-{[(1-{2-[3-fluoro-6-(methoxy)-4-quinolinyl]ethyl}-4-piperidinyl)amino]methyl}-1H-pyrido[2,3-b][1,4]thiazin-2(3H)-one dihydrochloride;
N-(2,3-dihydro[1,4]dioxino[2,3-b]pyridin-7-ylmethyl)-1-{2-[3-fluoro-6-(methoxy)-4-quinolinyl]ethyl}-4-piperidinamine dihydrochloride;
N-(2,3-dihydro[1,4]oxathiino[2,3-c]pyridin-7-ylmethyl)-1-{2-[3-fluoro-6-(methoxy)-4-quinolinyl]ethyl}-4-piperidinamine dihydrochloride;
4-[(2,3-dihydro[1,4]dioxino[2,3-c]pyridin-7-ylmethyl)amino]-1-{2-[3-fluoro-6-(methoxy)-4-quinolinyl]ethyl}-N-methyl-4-piperidinecarboxamide dihydrochloride;
4-[(2,3-dihydro[1,4]dioxino[2,3-c]pyridin-7-ylmethyl)amino]-1-{2-[3-fluoro-6-(methoxy)-4-quinolinyl]ethyl}-4-piperidinecarboxamide dihydrochloride;
4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-N-methyl-4-piperidinecarboxamide dihydrochloride;
4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-4-piperidinecarboxamide dihydrochloride;
1-{2-[3-chloro-6-(methoxy)-1,5-naphthyridin-4-yl]ethyl}-4-[(2,3-dihydro[1,4]dioxino[2,3-c]pyridin-7-ylmethyl)amino]-4-piperidinecarboxamide dihydrochloride;
(4-[(2,3-dihydro[1,4]dioxino[2,3-c]pyridin-7-ylmethyl)amino]-1-{2-[3-fluoro-6-(methoxy)-4-quinolinyl]ethyl}-4-piperidinyl)methanol dihydrochloride;
N-[1-{2-[3-fluoro-6-(methoxy)-1,5-naphthyridin-4-yl]ethyl}-4-(hydroxymethyl)-4-piperidinyl]-3-oxo-3,4-dihydro-2H-pyrido[3,2-b][1,4]thiazine-6-carboxamide hydrochloride;
N-[1-{2-[3-fluoro-6-(methoxy)-4-quinolinyl]ethyl}-4-piperidinyl)-3-oxo-3,4-dihydro-2H-pyrido[3,2-b][1,4]oxazine-6-carboxamide hydrochloride;
N-(1-{2-[3-fluoro-6-(methoxy)-4-quinolinyl]ethyl}-4-piperidinyl)-3-oxo-3,4-dihydro-2H-pyrido[3,2-b][1,4]thiazine-6-carboxamide hydrochloride;
7-{[((3R,4S)-1-{2-[3-fluoro-6-(methoxy)-4-quinolinyl]ethyl}-3-hydroxy-4-piperidinyl)amino]methyl}-1H-pyrido[2,3-b][1,4]thiazin-2(3H)-one dihydrochloride Enantiomer 1;
6-{[((3R,4S)-1-{2-[3-chloro-8-fluoro-6-(methoxy)-4-quinolinyl]ethyl}-3-hydroxy-4-piperidinyl)amino]methyl}-2H-pyrido[3,2-b][1,4]thiazin-3(4H)-one dihydrochloride Enantiomer 1;
(3R,4S)-1-{2-[3-chloro-8-fluoro-6-(methoxy)-4-quinolinyl]ethyl}-4-[(2,3-dihydro[1,4]dioxino[2,3-c]pyridin-7-ylmethyl)amino]-3-piperidinol dihydrochloride;
2-{4-[(2,3-Dihydro[1,4]dioxino[2,3-c]pyridin-7-ylmethyl)amino]-1-piperidinyl)-1-[3-fluoro-6-(methoxy)-1,5-naphthyridin-4-yl]ethanol Dihydrochloride Hydrate Enantiomer 1;
2-{4-[(2,3-Dihydro[1,4]dioxino[2,3-c]pyridin-7-ylmethyl)amino]-1-piperidinyl)-1-[3-fluoro-6-(methoxy)-1,5-naphthyridin-4-yl]ethanol Dihydrochloride Hydrate Enantiomer 2;
racemic,cis 4-[(2,3-dihydro[1,4]dioxino[2,3-c]pyridin-7-ylmethyl)amino]-1-(2-[3-fluoro-6-(methoxy)-1,5-naphthyridin-4-yl]ethyl}-3-piperidinyl)methanol dihydrochloride;
racemic,cis-4-[(2,3-dihydro[1,4]dioxino[2,3-c]pyridin-7-ylmethyl)amino]-1-(2-[3-fluoro-6-(methoxy)-1,5-naphthyridin-4-yl]ethyl}-3-piperidinecarboxylic acid dihydrochloride;
racemic,cis-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinecarboxamide dihydrochloride;
1-{2-[3-chloro-6-(methoxy)-1,5-naphthyridin-4-yl]ethyl}-N-[(6-oxido-2,3-dihydro[1,4]dioxino[2,3-c]pyridin-7-yl)methyl]-4-piperidinamine dihydrochloride;
6-{[(1-{2-[3-chloro-6-(methoxy)-1,5-naphthyridin-4-yl]-3-hydroxypropyl}-4-piperidinyl)amino]methyl}-2H-pyrido[3,2-b][1,4]thiazin-3(4H)-one dihydrochloride;
6-[({1-[2-(3,6-difluoro-4-quinolinyl)ethyl]-4-piperidinyl}amino)methyl]-2H-pyrido[3,2-b][1,4]thiazin-3(4H)-one dihydrochloride;
1-[2-(3,6-difluoro-4-quinolinyl)ethyl]-N-(2,3-dihydro[1,4]dioxino[2,3-c]pyridin-7-ylmethyl)-4-piperidinamine hydrochloride dihydrochloride;
6-[({1-[2-(3,6-difluoro-4-quinolinyl)ethyl]-4-piperidinyl}amino)methyl]-2H-pyrido[3,2-b][1,4]oxazin-3(4H)-one dihydrochloride;
6-{[(1-{2-[3-chloro-6-fluoro-5-(methoxy)-4-quinolinyl]-1-methylethyl)-4-piperidinyl)amino]methyl}-2H-pyrido[3,2-b][1,4]oxazin-3(4H)-one dihydrochloride;
1-{2-[3-chloro-6-fluoro-5-(methoxy)-4-quinolinyl]ethyl}-N-(2,3-dihydro[1,4]dioxino[2,3-c]pyridin-7-ylmethyl)-4-piperidinamine dihydrochloride;
1-[2-(6-chloro-3-fluoro-4-quinolinyl)ethyl]-4-[(2,3-dihydro[1,4]dioxino[2,3-c]pyridin-7-ylmethyl)amino]-N-methyl-4-piperidinecarboxamide dihydrochloride;
2-{4-[(2,3-dihydro[1,4]dioxino[2,3-c]pyridin-7-ylmethyl)amino]-1-piperidinyl)-1-[3-fluoro-6-(methoxy)-4-quinolinyl]ethanol dihydrochloride Enantiomer 2;
6-{[trans-1-{2-[3-fluoro-6-(methoxy)-1,5-naphthyridin-4-yl]ethyl}-3-hydroxy-4-piperidinyl)amino]methyl}-2H-pyrido[3,2-b][1,4]thiazin-3(4H)-one dihydrochloride Enantiomer E2;
6-{[trans-1-{2-[3-fluoro-6-(methoxy)-1,5-naphthyridin-4-yl]ethyl}-3-hydroxy-4-piperidinyl)amino]methyl}-2H-pyrido[3,2-b][1,4]oxazin-3(4H)-one dihydrochloride Enantiomer E2;
trans-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinol dihydrochloride Enantiomer E2;
6-{[trans-1-{2-[3-fluoro-6-(methoxy)-4-quinolinyl]ethyl}-3-hydroxy-4-piperidinyl)amino]methyl}-2H-pyrido[3,2-b][1,4]thiazin-3(4H)-one-dihydrochloride Enantiomer E2;
trans-4-[(2,3-dihydro[1,4]dioxino[2,3-c]pyridin-7-ylmethyl)amino]-1-{2-[3-fluoro-6-(methoxy)-4-quinolinyl]ethyl}-3-piperidinol dihydrochloride;
N-trans-1-{2-[3-fluoro-6-(methoxy)-1,5-naphthyridin-4-yl]ethyl}-3-hydroxy-4-piperidinyl)-3-oxo-3,4-dihydro-2H-pyrido[3,2-b][1,4]thiazine-6-carboxamide hydrochloride Enantiomer E2;
N-trans-1-{2-[3-fluoro-6-(methoxy)-1,5-naphthyridin-4-yl]ethyl}-3-hydroxy-4-piperidinyl)-2,3-dihydro[1,4]dioxino[2,3-c]pyridine-7-carboxamide hydrochloride Enantiomer E2;
racemic, trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]thiazin-3(4H)-one dihydrochloride;
Trans-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-methyl-3-piperidinol dihydrochloride;
6-{[trans-1-{2-[3-fluoro-6-(methoxy)-1,5-naphthyridin-4-yl]ethyl}-3-hydroxy-4-methyl-4-piperidinyl)amino]methyl}-2H-pyrido[3,2-b][1,4]thiazin-3(4H)-one dihydrochloride Enantiomer E1;
Trans-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-4-methyl-3-piperidinol dihydrochloride;
6-{[trans-1-{2-[3-fluoro-6-(methoxy)-1,5-naphthyridin-4-yl]ethyl}-3-hydroxy-4-methyl-4-piperidinyl)amino]methyl}-2H-pyrido[3,2-b][1,4]thiazin-3(4H)-one dihydrochloride Enantiomer E2;
Trans-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-4-methyl-3-piperidinol dihydrochloride;
N-(3,4-dihydro-2H-pyrano[2,3-c]pyridin-6-ylmethyl)-1-{2-[3-fluoro-6-(methoxy)-1,5-naphthyridin-4-yl]ethyl}-4-piperidinamine dihydrochloride;
{[(1-{2-[3-Fluoro-6-(methoxy-5-naphthyridin-4-yl]ethyl}-4-piperidinyl)amino]methyl}-3,4-dihydro-1,8-naphthyridin-2-(1H)-one;
7-{(1-{2-[3-fluoro-6-(methoxy)-1,5-naphthyridin-4-yl]ethyl}-4-piperidinyl)amino]methyl}-2,3-dihydro-1,5-benzothiazepin-4(5H)-one;
trans-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinol dihydrochloride Enantiomer E1;
6-{[(-1-{2-[3-fluoro-6-(methoxy)-1,5-naphthyridin-4-yl]ethyl}-3-hydroxy-4-piperidinyl)amino]methyl}-2H-pyrido[3,2-b][1,4]oxazin-3(4H)-one dihydrochloride;
trans-6-{[(1-{2-[3-fluoro-6-(methoxy)-4-quinolinyl]ethyl}-3-hydroxy-4-piperidinyl)amino]methyl}-2H-pyrido[3,2-b][1,4]thiazin-3(4H)-one Enantiomer E1;
trans-4-[(2,3-dihydro[1,4]dioxino[2,3-c]pyridin-7-ylmethyl)amino]-1-{2-[3-fluoro-6-(methoxy)-4-quinolinyl]ethyl}-3-piperidinol dihydrochloride;
trans-6-{[(1-{2-[3-fluoro-6-(methoxy)-4-quinolinyl]ethyl}-3-hydroxy-4-piperidinyl)amino]methyl}-2H-pyrido[3,2-b][1,4]oxazin-3(4H)-one dihydrochloride;
trans-N-(1-{2-[3-fluoro-6-(methoxy)-1,5-naphthyridin-4-yl]ethyl}-3-hydroxy-4-piperidinyl)-3-oxo-3,4-dihydro-2H-pyrido[3,2-b][1,4]thiazine-6-carboxamide hydrochloride Enantiomer E1;
trans-N-((3R,4R)-1-{2-[3-fluoro-6-(methoxy)-1,5-naphthyridin-4-yl]ethyl}-3-hydroxy-4-piperidinyl)-3-oxo-3,4-dihydro-2H-pyrido[3,2-b][1,4]oxazine-6-carboxamide Isomer E1 hydrochloride;
trans-N-(1-{2-[3-fluoro-6-(methoxy)-1,5-naphthyridin-4-yl]ethyl}-3-hydroxy-4-piperidinyl)-2,3-dihydro[1,4]dioxino[2,3-c]pyridine-7-carboxamide Isomer E1 hydrochloride;
6-{[trans-1-{2-[3-fluoro-6-(methoxy)-1,5-naphthyridin-4-yl]ethyl}-3-hydroxy-4-piperidinyl)amino]methyl}-2H-pyrido[3,2-b][1,4]thiazin-3(4H)-one Enantiomer E1;
6-{[(1-{2-[3-fluoro-6-(methoxy)-1,5-naphthyridin-4-yl]ethyl}-4-methyl-4-piperidinyl)amino]methyl}-2H-pyrido[3,2-b][1,4]oxazin-3(4H)-one dihydrochloride;
6-{[(1-{2-[3-fluoro-6-(methoxy)-1,5-naphthyridin-4-yl]ethyl}-4-methyl-4-piperidinyl)amino]methyl}-2H-pyrido[3,2-b][1,4]thiazin-3(4H)-one dihydrochloride;
N-(2,3-dihydro[1,4]dioxino[2,3-c]pyridin-7-ylmethyl)-1-{2-[3-fluoro-6-(methoxy)-1,5-naphthyridin-4-yl]ethyl}-4-methyl-4-piperidinamine dihydrochloride;
N-(1-{2-[3-fluoro-6-(methoxy)-1,5-naphthyridin-4-yl]ethyl}-4-methyl-4-piperidinyl)-2,3-dihydro-1,4-benzodioxin-6-sulfonamide;
cis-6-{[(1-{2-[3-chloro-8-fluoro-6-(methoxy)-4-quinolinyl]ethyl}-3-fluoro-4-piperidinyl)amino]methyl}-2H-pyrido[3,2-b][1,4]thiazin-3(4H)-one dihydrochloride Enantiomer 1;
cis-1-{2-[3,8-difluoro-6-(methoxy)-4-quinolinyl]ethyl}-N-(2,3-dihydro[1,4]dioxino[2,3-c]pyridin-7-ylmethyl)-3-fluoro-4-piperidinamine dihydrochloride Enantiomer1;
cis-1-{2-[3,8-difluoro-6-(methoxy)-4-quinolinyl]ethyl}-N-(2,3-dihydro[1,4]dioxino[2,3-c]pyridin-7-ylmethyl)-3-fluoro-4-piperidinamine dihydrochloride Enantiomer 2;
cis-6-{[(1-{2-[3,8-difluoro-6-(methoxy)-4-quinolinyl]ethyl}-3-fluoro-4-piperidinyl)amino]methyl}-2H-pyrido[3,2-b][1,4]oxazin-3(4H)-one dihydrochloride, Enantiomer 1;
cis-6-{[(1-{2-[3,8-difluoro-6-(methoxy)-4-quinolinyl]ethyl}-3-fluoro-4-piperidinyl)amino]methyl}-2H-pyrido[3,2-b][1,4]oxazin-3(4H)-one dihydrochloride, Enantiomer 2;
cis-1-{2-[3,8-difluoro-6-(methoxy)-4-quinolinyl]ethyl}-N-(2,3-dihydro-1,4-benzodioxin-6-ylmethyl)-3-fluoro-4-piperidinamine dihydrochloride, Enantiomer 1;
cis-1-{2-[3,8-difluoro-6-(methoxy)-4-quinolinyl]ethyl}-N-(2,3-dihydro-1,4-benzodioxin-6-ylmethyl)-3-fluoro-4-piperidinamine dihydrochloride, Enantiomer 2;
cis-6-([(−1-{2-[3,8-difluoro-6-(methoxy)-4-quinolinyl]ethyl}-3-fluoro-4-piperidinyl)amino]methyl}-2H-pyrido[3,2-b][1,4]thiazin-3(4H)-one dihydrochloride, Enantiomer 1;
cis-6-([(−1-{2-[3,8-difluoro-6-(methoxy)-4-quinolinyl]ethyl}-3-fluoro-4-piperidinyl)amino]methyl}-2H-pyrido[3,2-b][1,4]thiazin-3(4H)-one dihydrochloride, Enantiomer 2;
cis-N-(1-{2-[3,8-difluoro-6-(methoxy)-4-quinolinyl]ethyl}-3-fluoro-4-piperidinyl)-3-oxo-3,4-dihydro-2H-pyrido[3,2-b][1,4]thiazine-6-carboxamide hydrochloride Enantiomer 1;
6-{[((3S,4R)-1-{2-[3-chloro-8-fluoro-6-(methoxy)-4-quinolinyl]ethyl}-3-hydroxy-4-piperidinyl)amino]methyl}-2H-pyrido[3,2-b][1,4]thiazin-3(4H)-one dihydrochloride Enantiomer E2;
trans-6-({1-[2-(3-Chloro-6-methoxy-[1,5]naphthyridin-4-yl)-ethyl]-3-hydroxy-piperidin-4-ylamino}-methyl)-4H-pyrido[3,2-b][1,4] oxazin-3-one trihydrochloride Enantiomer 1;
trans-1-{2-[3-chloro-6-(methoxy)-1,5-naphthyridin-4-yl]ethyl}-4-[(2,3-dihydro[1,4]dioxino[2,3-c]pyridin-7-ylmethyl)amino]-3-piperidinol Enantiomer 1;
trans-1-{2-[3-chloro-6-(methoxy)-1,5-naphthyridin-4-yl]ethyl}-4-[(2,3-dihydro-[1,4]dioxino[2,3-c]pyridin-7-ylmethyl)amino]-3-piperidinol Enantiomer 2;
2-{4-[(2,3-dihydro[1,4]dioxino[2,3-c]pyridin-7-ylmethyl)amino]-1-piperidinyl}-1-[3-fluoro-6-(methoxy)-4-quinolinyl]ethanol dihydrochloride Enantiomer 1;
N-(2,3-dihydro-1,4-benzodioxin-6-ylmethyl)-1-{2-[3-fluoro-6-(methoxy)-1,5-naphthyridin-4-yl]ethyl}-4-piperidinamine;
(3S,4R)-4-[(2,3-dihydro[1,4]dioxino[2,3-c]pyridin-7-ylmethyl)amino]-1-{2-[3-fluoro-6-(methoxy)-1,5-naphthyridin-4-yl]ethyl}-3-piperidinol dihydrochloride Enantiomer 2;
(3R,4S)-1-{2-[3-fluoro-6-(methoxy)-1,5-naphthyridin-4-yl]ethyl}-4-[([1,3]oxathiolo[5,4-c]pyridin-6-ylmethyl)amino]-3-piperidinol dihydrochloride Enantiomer E1;
6-{[(1-{2-[3-chloro-8-fluoro-6-(methoxy)-4-quinolinyl]ethyl}-4-piperidinyl)amino]methyl}-2H-pyrido[3,2-b][1,4]thiazin-3(4H)-one;
1-{2-[3-chloro-8-fluoro-6-(methoxy)-4-quinolinyl]ethyl}-N-(2,3-dihydro[1,4]dioxino[2,3-c]pyridin-7-ylmethyl)-4-piperidinamine;
(3S,4R)-1-[2-(3,6-dichloro-4-quinolinyl)ethyl]-4-[(2,3-dihydro[1,4]dioxino[2,3-c]pyridin-7-ylmethyl)amino]-3-piperidinol dihydrochloride Enantiomer E2;
6-[({(3S,4R)-1-[2-(3,6-dichloro-4-quinolinyl)ethyl]-3-hydroxy-4-piperidinyl}amino)methyl]-2H-pyrido[3,2-b][1,4]thiazin-3(4H)-one dihydrochloride Enantiomer E2;
(3S,4R)-1-[2-(3-chloro-6-fluoro-4-quinolinyl)ethyl]-4-[(2,3-dihydro[1,4]dioxino[2,3-c]pyridin-7-ylmethyl)amino]-3-piperidinol dihydrochloride Enantiomer E2;
6-[({(3S,4R)-1-[2-(3-chloro-6-fluoro-4-quinolinyl)ethyl]-3-hydroxy-4-piperidinyl}amino)methyl]-2H-pyrido[3,2-b][1,4]thiazin-3(4H)-one dihydrochloride Enantiomer E2;
N-(1-{2-[3-fluoro-6-(methoxy)-1,5-naphthyridin-4-yl]ethyl}-4-methyl-4-piperidinyl)-2,3-dihydro[1,4]dioxino[2,3-c]pyridine-7-carboxamide dihydrochloride;
N-(1-({2-[3-fluoro-6-(methoxy)-1,5-naphthyridin-4-yl]ethyl}-4-methyl-4-piperidinyl)-3-oxo-3,4-dihydro-2H-pyrido[3,2-b][1,4]oxazine-6-carboxamide;
N-(1-{2-[3-fluoro-6-(methoxy)-1,5-naphthyridin-4-yl]ethyl}-4-methyl-4-piperidinyl)-3-oxo-3,4-dihydro-2H-pyrido[3,2-b][1,4]thiazine-6-carboxamide;
trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]oxazin-3(4H)-one dihydrochloride Enantiomer E1;
trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]thiazin-3(4H)-one dihydrochloride Enantiomer E1;
trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]oxazin-3(4H)-one dihydrochloride Enantiomer E2; trans-6-{[(1-{2-[3-fluoro-6-(methoxy)-1,5-naphthyridin-4-yl]ethyl}-3-hydroxy-3-methyl-4-piperidinyl)amino]methyl}-2H-pyrido[3,2-b][1,4]thiazin-3(4H)-one dihydrochloride Enantiomer E2;
trans-4-[(2,3-dihydro-1,4-benzodioxin-6-ylmethyl)amino]-1-{2-[3-fluoro-6-(methoxy)-4-quinolinyl]ethyl}-3-piperidinol hydrochloride Enantiomer E1;
trans 4-[(2,3-dihydro-1,4-benzodioxin-6-ylmethyl)amino]-1-{2-[3-fluoro-6-(methoxy)-1,5-naphthyridin-4-yl]ethyl}-3-piperidinol dihydrochloride Enantiomer E2;
trans-4-[(2,3-dihydro-1,4-benzodioxin-6-ylmethyl)amino]-1-{2-[3-fluoro-6-(methoxy)-1,5-naphthyridin-4-yl]ethyl}-3-piperidinol dihydrochloride Enantiomer E1;
(3S,4R)-1-{2-[3,8-difluoro-6-(methoxy)-4-quinolinyl]ethyl}-4-[(2,3-dihydro[1,4]dioxino[2,3-c]pyridin-7-ylmethyl)amino]-3-piperidinol dihydrochloride Enantiomer E2;
(3S,4R)-1-{2-[3,8-difluoro-6-(methoxy)-4-quinolinyl]ethyl}-4-[(2,3-dihydro-1,4-benzodioxin-6-ylmethyl)amino]-3-piperidinol dihydrochloride Enantiomer E2;
N-(2,3-dihydro-1-benzofuran-5-ylmethyl)-1-{2-[3-fluoro-6-(methoxy)-1,5-naphthyridin-4-yl]ethyl}-4-piperidinamine dihydrochloride;
6-{[(1-{2-[3-fluoro-6-(methoxy)-4-quinolinyl]-2-hydroxyethyl}-4-piperidinyl)amino]methyl}-2H-pyrido[3,2-b][1,4]oxazin-3(4H)-one dihydrochloride Enantiomer E1;
6-{[(1-{2-[3-fluoro-6-(methoxy)-4-quinolinyl]-2-hydroxyethyl}-4-piperidinyl)amino]methyl}-2H-pyrido[3,2-b][1,4]oxazin-3(4H)-one dihydrochloride Enantiomer E2;
6-{[(1-{2-[3-fluoro-6-(methoxy)-1,5-naphthyridin-4-yl]-2-hydroxyethyl}-4-piperidinyl)amino]methyl}-2H-pyrido[3,2-b][1,4]oxazin-3(4H)-one dihydrochloride Enantiomer E2; 6-{[(1-{2-[3-fluoro-6-(methoxy)-1,5-naphthyridin-4-yl]-2-hydroxyethyl}-4-piperidinyl)amino]methyl}-2H-pyrido[3,2-b][1,4]oxazin-3(4H)-one dihydrochloride Enantiomer E1;
6-{[(1-{2-[3-chloro-8-fluoro-6-(methoxy)-4-quinolinyl]-2-hydroxyethyl}-4-piperidinyl)amino]methyl}-2H-pyrido[3,2-b][1,4]oxazin-3(4H)-one dihydrochloride Enantiomer E1;
6-{[(1-{2-[3-chloro-8-fluoro-6-(methoxy)-4-quinolinyl]-2-hydroxyethyl}-4-piperidinyl)amino]methyl}-2H-pyrido[3,2-b][1,4]oxazin-3(4H)-one dihydrochloride Enantiomer E2;
1-[3-chloro-8-fluoro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E1;
1-[3-chloro-8-fluoro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E2;
1-[3,8-difluoro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E2;
1-[3,8-difluoro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E1;
1-[3-chloro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E2;
1-[3-chloro-6-(methoxy)-4-quinolinyl]-2-{4-[(2,3-dihydro[1,4]dioxino[2,3-c]pyridin-7-ylmethyl)amino]-1-piperidinyl}ethanol dihydrochloride Enantiomer E1;
1-[3-chloro-6-(methoxy)-1,5-naphthyridin-4-yl]-2-{4-[(2,3-dihydro[1,4]dioxino[2,3-c]pyridin-7-ylmethyl)amino]-1-piperidinyl}ethanol dihydrochloride Enantiomer E2;
2-{4-[(2,3-dihydro[1,4]dioxino[2,3-c]pyridin-7-ylmethyl)amino]-3-fluoro-1-piperidinyl}-1-[3-fluoro-6-(methoxy)-1,5-naphthyridin-4-yl]ethanol dihydrochloride Enantiomer E2;
2-{4-[(2,3-dihydro[1,4]dioxino[2,3-c]pyridin-7-ylmethyl)amino]-3-fluoro-1-piperidinyl}-1-[3-fluoro-6-(methoxy)-1,5-naphthyridin-4-yl]ethanol dihydrochloride Enantiomer E1;
7-{[(1-{2-[3,8-difluoro-6-(methoxy)-4-quinolinyl]ethyl}-3-fluoro-4-piperidinyl)amino]methyl}-1H-pyrido[2,3-b][1,4]thiazin-2(3H)-one dihydrochloride Enantiomer E2;
1-{2-[3-fluoro-6-(methoxy)-1,5-naphthyridin-4-yl]ethyl}-N-{[8-(methoxy)-2,3-dihydro-1,4-benzodioxin-6-yl]methyl}-4-piperidinamine; and
1-{2-[3-fluoro-6-(methoxy)-1,5-naphthyridin-4-yl]ethyl}-N-[(7-methyl-2,3-dihydro-1,4-benzodioxin-6-yl)methyl]-4-piperidinamine;
or a pharmaceutically acceptable salt thereof.

11. A process for preparing compounds of formula (I), and pharmaceutically acceptable derivatives thereof, which process comprises reacting a compound of formula (IV) with a compound of formula (V): 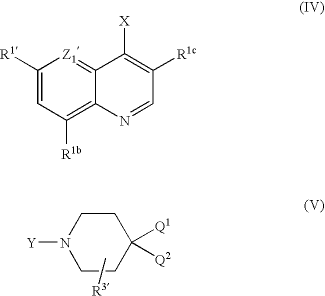
wherein Z¹′, R^{1′}, R^{1b′}, R^{1c′} and R^{3′} are Z¹, R¹, R^{1b}, R^{1c} and R³ as defined in formula (I) or groups convertible thereto; Q¹ is NHR^{4′} or a group convertible thereto wherein R^{4′} is R⁴ as defined in formula (I) or groups convertible thereto and Q² is H or R^{3′} or Q¹ and Q² together form an optionally protected oxo group;
(i) X is A′-COW, Y is H;
(ii) X is CH═CH₂, Y is H;
(iii) X is oxirane, Y is H;
(iv) one of X and Y is CO₂R^{y} and the other is CH₂CO₂R^{x};
in which W is a leaving group, e.g. halo or imidazolyl; R^{x} and R^{y} are (C₁₋₄)alkyl; A′ is A as defined in formula (I), or groups convertible thereto; and oxirane is: 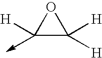
and thereafter optionally or as necessary converting Q¹ and Q² to NHR^{4′};
converting A′, Z^{1′}R^{1′}, R^{1b′}, R^{1c′}, R^{3′}, and R^{4′} to A, Z¹, R¹, R^{1b}, R^{1c}, R³, and R⁴;
converting A-B to other A-B, interconverting R¹, R^{1b}, R^{1c}, R³, and/or R⁴, and/or
forming a pharmaceutically acceptable derivative thereof.

12. A pharmaceutical composition comprising a compound according to claim 1 and a pharmaceutically acceptable carrier.

13. A method of treating bacterial infections in mammals which comprises administrating to a mammal in need thereof an effective amount of a compound according to claim 1.
